(19) United States
(12) Patent Application Publication
(10) Pub. No.: US 2023/0183783 A1
(43) Pub. Date: Jun. 15, 2023

(54) IMPROVED DETECTION ASSAYS

(71) Applicant: Sherlock Biosciences, Inc., Watertown, MA (US)

(72) Inventors: William Jeremy Blake, Winchester, MA (US); Xiang Li, Malden, MA (US); Mary Katherine Wilson, Burlington, MA (US); Christine Marie Coticchia, Brookline, MA (US); Brendan John Manning, Brighton, MA (US); Pradeep Ramesh, Brighton, MA (US)

(21) Appl. No.: 17/795,815

(22) PCT Filed: Jan. 27, 2021
(86) PCT No.: PCT/US2021/015306

§ 371 Date: Jul. 27, 2022

Related U.S. Application Data
(60) Provisional application No. 63/139,267, filed on Jan. 19, 2021.
(60) Provisional application No. 63/038,710, filed on Jun. 12, 2020.
(60) Provisional application No. 62/970,159, filed on Feb. 4, 2020.
(60) Provisional application No. 62/967,536, filed on Jan. 29, 2020.
(60) Provisional application No. 62/966,527, filed on Jan. 27, 2020.

(51) Int. Cl. C12Q 1/683; C12N 9/22; C12N 15/113; C12Q 1/6844

(52) U.S. Cl. CPC C12Q 1/683; C12N 9/22; C12N 15/113; C12Q 1/6844; C12N 2310/20; C12N 2320/10

(57) ABSTRACT

The present disclosure provides improved detection (e.g., diagnostic) assays that utilize a Cas protein collateral cleavage activity.

### CROSS REFERENCE TO RELATED APPLICATIONS

This application claims priority to each of U.S. Provisional Patent Application Nos. 62/966,527; filed Jan. 27, 2020; 62/967,536; filed Jan. 29, 2020; 62/970,159; filed Feb. 4, 2020; 63/038,710; filed Jun. 12, 2020; 63/139,267; filed Jan. 19, 2021 the entire contents of each of which are hereby incorporated by reference.

### BACKGROUND

A variety of clustered regularly interspaced short palindromic repeats—CRISPR-associated (“Cas”) proteins have been discovered to have a collateral cleavage activity useful in detection (e.g., diagnostic) systems to detect particular nucleic acids of interest. See, for example, review by Sashital Genome Med 2018:10, 32.

### SUMMARY

The present disclosure provides improved detection (e.g., diagnostic) technologies that utilize Cas-protein collateral activity.

Among other things, the present disclosure identifies the source of a problem with use of certain Cas enzymes in certain collateral activity assays. For example, the present disclosure documents that certain such assays include a step that involves incubation at an elevated temperature for a period of time, and various Cas enzymes may be insufficiently stable to maintain a sufficient level of activity (e.g., collateral activity) under such conditions. In many embodiments, such a step may be or comprise a nucleic acid extension and/or amplification step.

Alternatively or additionally, the present disclosure provides the insight that particularly desirable embodiments of various collateral activity assays are those that can be performed in a single reaction vessel (i.e., so-called “one pot”) assays. The present disclosure appreciates that Cas enzymes whose activity (e.g., collateral cleavage activity) is insufficiently stable to maintain sufficient activity through any and all elevated-temperature step(s) (which may be or include, for example, one or more nucleic acid extension and/or amplification step(s)) may not be useful in such one-pot assays. The present disclosure furthermore documents that certain Cas protein(s) (e.g., Cas13 and Cas12) are insufficiently stable at relevant temperature(s), e.g., at temperatures at which nucleic acid extension and/or amplification reactions are typically performed (e.g., above about 60-65° C.).

The present disclosure encompasses the recognition that thermostable variants of various Cas proteins (e.g., Cas9) have already been described and/or otherwise made publicly available (see, for example, Mougiakos et al. Nat Commun. 8:1647, 2017). Those skilled in the art are able to compare such thermostable variants with related non-thermostable homologs (e.g., orthologs), in order to assess sequence changes and/or elements that may be necessary and/or sufficient to achieve thermostability, and furthermore can identify such sequence changes and/or elements in other homologs (e.g., orthologs) and/or can introduce them thereinto. Still further, those skilled in the art are well aware of potential sources of naturally-occurring thermostable Cas proteins (e.g., in microbes that survive in elevated temperature conditions, such as in sea vents, or are otherwise thermophilic). Thus, those skilled in the art, reading the present disclosure, could readily identify and/or develop appropriate thermostable Cas proteins for use as described herein.

In some embodiments, a useful thermostable Cas protein is a Cas12 or Cas13 homolog (e.g., ortholog). In some embodiments, a useful thermostable Cas protein is a Cas enzyme comprising an amino acid sequence having 80%, 85%, 90%, 99% or 100% sequence identity to any one of SEQ ID Nos. 1-283.

Alternatively or additionally, in some embodiments, a useful thermostable Cas protein performs (e.g., its collateral cleavage activity functions sufficiently) at temperatures above about 50° C.; in some embodiments, above a temperature selected from the group consisting of about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., or combinations thereof. In many embodiments, useful thermostable Cas protein performs (e.g., its collateral cleavage activity functions sufficiently) at temperatures above about 60° C.

In some embodiments, a useful thermostable Cas protein performs (e.g., its collateral cleavage activity functions sufficiently) within a temperature range at which nucleic acid extension and/or amplification reaction(s) are performed; those skilled in the art are well familiar with various such reactions and the temperature ranges at which they are performed, In some embodiments, such a temperature range may be above a temperature selected from the group consisting of about 60° C., about 61° C., about 62° C., about 63° C., about 64° C., 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., or combinations thereof. In some embodiments, a temperature range may be about 60° C. to about 90° C. In some embodiments, a temperature range may be about 60° C. to about 80° C. In some embodiments, a temperature range may be about 60° C. to about 75° C. In some embodiments, a temperature range may be about 65° C. to about 90° C. In some embodiments, a temperature range may be about 60° C. to about 80° C. In some embodiments, a temperature range may be about 60° C. to about 75° C.

Thus, as is set forth herein, in some embodiments, a useful thermostable Cas protein is a Cas12 or Cas13 homolog (e.g., ortholog), e.g., a Cas enzyme comprising an amino acid sequence having 80%, 85%, 90%, 99% or 100% sequence identity to any one of SEQ ID Nos. 1-283 that is thermostable at temperatures above about 50° C., and in some embodiments above about 60° C., for example within and/or above about 60-65° C. Those skilled in the art, reading the present disclosure will particularly appreciate that, in some embodiments, a useful thermostable Cas protein is a Cas12 (e.g., SEQ ID NO 3-21, 33-47, 51-56, 68-178, and 274-283, or a variant thereof, for example having at least 90%, 95%, 99% or greater amino acid sequence identity thereto) or Cas13 (e.g., SEQ ID NO 1-2, 22-32, 48-50, 57-67, 179-273, or a variant thereof, for example having at least 90%, 95%, 99% or greater amino acid sequence identity thereto) whose activity (e.g., whose target binding and collateral cleavage activities) is sufficiently thermostable, for example at temperatures within a range of 60-65° C. to perform in assays as described herein (e.g., in some embodiments, one-pot assays). For example, in some embodiments, sufficient thermostable activity is activity that is reasonably comparable to (e.g., within about 25%) of an appropriate reference thermostable Cas protein (e.g., SEQ ID NO 15) as described herein.

In some embodiments, the disclosure describes a detection method comprising steps of: contacting a CRISPR-Cas complex comprising: a Cas protein with collateral cleavage activity that is thermostable at temperatures above at least 60-65° C.; and a guide RNA selected or engineered to be complementary to a target sequence; with a sample potentially comprising a nucleic acid of the target sequence.

In some embodiments, the step of contacting comprises contacting the CRISRP-Cas complex and sample with a reporter susceptible to cleavage by the Cas protein collateral activity. In some embodiments, the step of contacting comprises incubating for a period of time above the temperature. In some embodiments, a detection method further comprises a step of amplifying nucleic acid present in the sample. In some embodiments, the step of amplifying utilizes a thermostable nucleic acid polymerase. In some embodiments, the steps of amplifying and contacting are performed in a single vessel.

In some embodiments, the Cas protein is a Cas12 protein. In some embodiments, the Cas protein has an amino acid sequence that is at least 80% identical to that of SEQ ID NO: 15. In some embodiments, the Cas protein has an amino acid sequence having at least 80%, sequence identity to any one of SEQ ID Nos. 3-21, 33-47, 51-56, 68-178, and 274-283. In some embodiments, the Cas protein has an amino acid sequence having 80%, sequence identity to any one of SEQ ID Nos. 1-283.

In some embodiments, in a method of performing a detection assay utilizing a Cas protein with collateral cleavage activity, the improvement that comprises utilizing a Cas protein with thermostable collateral cleavage activity. In some embodiments, the Cas protein is a Cas12 protein. In some embodiments, the Cas protein has an amino acid sequence that is at least 80% identical to that of SEQ ID NO: 15. In some embodiments, the Cas protein has an amino acid sequence having at least 80%, sequence identity to any one of SEQ ID Nos. 3-21, 33-47, 51-56, 68-178, and 274-283. In some embodiments, a method of performing a detection assay is conducted in a single reaction vessel. In some embodiments, the thermostable collateral cleavage activity is thermostable above a temperature of about 60° C. In some embodiments, the thermostable collateral cleavage activity is thermostable above a temperature of about 65° C. In some embodiments, the Cas protein has an amino acid sequence having at least 80% sequence identity to any one of SEQ ID Nos. 1-283.

### BRIEF DESCRIPTION OF THE DRAWING

FIGS. 1A and 1B document the insight, provided by the present disclosure that certain Cas13 protein(s) are insufficiently stable at relevant temperature(s), e.g., at temperatures at which nucleic acid extension and/or amplification reactions are typically performed (e.g., above about 60-65° C.).

FIG. 2 documents the insight, provided by the present disclosure that certain Cas12 protein(s) are insufficiently stable at relevant temperature(s), e.g., at temperatures at which nucleic acid extension and/or amplification reactions are typically performed (e.g., above about 60-65° C.).

FIGS. 3A-3C confirms and further demonstrate the thermostability of TccCas13 collateral activity.

FIG. 4 displays an exemplary method for discovery and screening of thermostable Cas enzyme candidates (e.g., Cas12 and Cas 13 enzymes)

FIG. 5 displays an exemplary assessment of Cas12a candidate enzymes by endpoint assay.

FIG. 6 displays an exemplary assessment of Cas12a candidate enzymes by kinetic assay.

FIG. 7 displays an exemplary assessment of candidate enzymes at 58° C. by endpoint and kinetic assays.

FIG. 8 displays an exemplary assessment of candidate enzymes at 60° C. by endpoint and kinetic assays.

FIG. 9 displays an exemplary assessment of candidate enzymes at 62° C. by endpoint and kinetic assays.

FIG. 10 displays exemplary assessment of four candidate enzymes that were purified and activity was measured at varying temperatures (e.g., approximately 35° C. to approximately 65° C.).

FIG. 11 shows exemplary characterization of a subset of enzyme candidates using three different guide and target sets compared to no template control at both 58° C. and 70° C.

FIG. 12 shows exemplary characterization of a Cas12 candidate enzyme with multiple guide/target pairs at both 52° C. and 58° C.

FIG. 13 demonstrates kinetic assays for a Cas12a candidate enzyme, RS62, which shows activity at 52° C.

FIG. 14 displays characterization of Cas13 candidate enzymes by endpoint assay at both 37° C. and 52° C.

FIG. 15 displays exemplary thermostable Cas12a enzyme, RS9, requires Thermostable Inorganic Pyrophosphatase (TIPP) for amplification. Real time detection of ORF1ab amplification was completed over a range of starting concentrations of ORF1ab (4.5 copies/μL to 4,500 copies/μL in the presence or absence of TIPP

FIG. 16 demonstrates exemplary thermostable Cas12a enzyme, RS9, is specific for its target and requires TIPP for amplification. Amplification was conducted with a starting concentration of 4,500 copies/μL ORF1ab template and either primers and guides specific to ORF1ab or non-targeting primers with an ORF1ab guide. Each reaction condition was also conducted in the presence or absence of TIPP.

FIG. 17 demonstrates exemplary thermostable Cas12a enzyme, RS9, displays collateral cleavage activity.

FIG. 18 demonstrates RS9 collateral cleavage activity compared to known Cas12a, LbaCas12a.

FIG. 19 demonstrates characterization of exemplary thermostable Cas13a enzyme, TccCas13a. Optimal temperature for TccCas13a activity was determined using a Cas reaction over a range of temperatures. Temperature profiles suggest TccCas13a shows highest activity at approximately 62° C.

FIG. 20 demonstrates TccCas13a can be activated by RNA, but cannot be activated by ssDNA, even at the highest concentrations of ssDNA.

FIG. 21 displays TccCas13a activation requires a higher concentration of ssDNA than RNA, similar to that observed for LwaCas13 ssDNA activation. TccCas13a was not activated by ssDNA* at any concentration (10 nM, 100 nM, or 1,000 nM) compared to control.

FIG. 22 demonstrates that TccCas13a shows increased collateral activity at “NN” sites compared to “UU” sites, while LwaCas13a shows no preference for collateral activity of “NN” sites compared to “UU” sites.

FIG. 23 demonstrates exemplary characterization of candidate thermostable Cas enzymes, Pal1, Pal2 low MW, Pal2 high MW, and Pal3.

FIG. 24 demonstrates exemplary Pal1 and Pal2 activity at 56° C.

FIG. 25 demonstrates exemplary activity of Pal1 at 37° C., 56° C., and 70° C. with different exemplary guides.

FIG. 26 demonstrates exemplary activity of Pal1 at 56° C. and 70° C. compared to control. These data suggest activity of Pal1 is specific to target DNA.

FIG. 27 demonstrates exemplary temperature profile of Pal1.

FIG. 28 demonstrates exemplary activity of Pal2 high MW at 37° C., 56° C., and 70° C. with different exemplary guides.

FIG. 29 demonstrates exemplary activity of Pal2 high MW at 56° C. compared to control. These data suggest activity of Pal2 high MW is specific to target DNA.

FIG. 30 demonstrates exemplary temperature profile of Pal2 high MW.

### DETAILED DESCRIPTION OF CERTAIN EMBODIMENTS

### Collateral Activity Assays

Those skilled in the art are well aware of the burgeoning plethora of useful detection (e.g., diagnostic) assays that have been and are being developed using Cas protein collateral activities. See, for example, Sashital Genome Med 2018:10, 32. Furthermore, those skilled in the art are well aware that a “detailed classification of CRISPR/Cas biosensing systems” based on Cas protein collateral activity has recently been made publicly available. See review by Li et al Trends Biotechnol. 37:730, July 2019.

Formats of particular interest include Cas13-based (e.g., Cas13a- or Cas13b-based) systems, including those referenced as “SHERLOCK” and/or “HUDSON” systems (see, for example, Gootenberg et al, Science 356:438, 2017; Gootenberg et al, Science 360:339, 2018; Myhrvold et al., Science 360:444, 2018; see also U.S. Ser. No. 10/266,887) and Cas12-based (e.g., Cas12a- or Cas12b-based) systems, including those references as “HOLMES” or “DETECTR” systems (see, for example, Cheng et al. CN patent filing CN107488710A; PCT/CN18/82769 and U.S. Ser. No. 16/631,157; Li et al. Cell Disc. 4:20, 2018; Chen et al. Science 360:436, 2018; Li, L. et al. bioRxiv Published online Jul. 26, 2018. http://dx.doi.org/10.1101/362889; U.S. Ser. No. 10/253,365). Both Cas13a and Cas13b enzymes have been used in SHERLOCK and/or HUDSON systems; similarly both Cas12a and Cas12b.

As is known in the art, and described in references cited herein, typical detection assays that utilize Cas protein collateral cleavage activity involve contacting an appropriate CRISPR-Cas complex, including a Cas protein with collateral activity and a guide RNA complementary to a target sequence of interest, with a sample that may contain the target sequence. Upon recognition of the target sequence, the Cas protein's collateral activity is activated, so that it cleaves unrelated nucleic acid (DNA or RNA or both, depending on the enzyme). A reporter of the relevant cleavable nucleic acid is provided, appropriately configured (e.g., labeled) so that its cleavage as a result of the activated collateral activity is detectable (e.g., separates a fluorophore from a quencher so that fluorescence becomes detectable, etc).

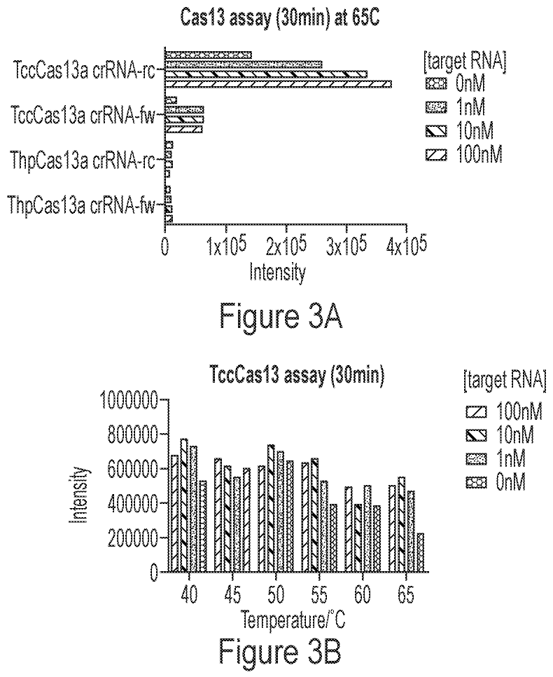

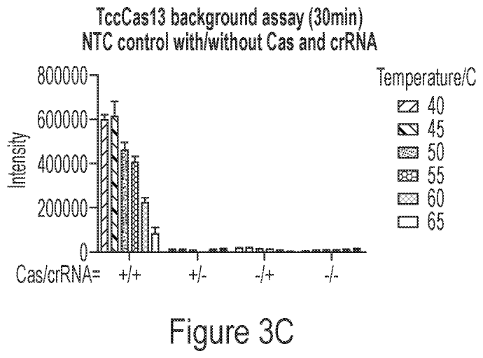

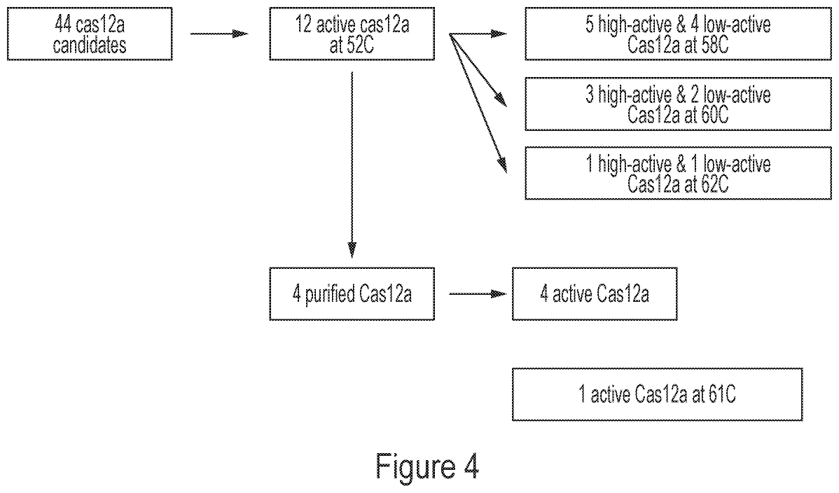

In many assays, a target sequence is generated and/or amplified (e.g., copied from RNA to DNA and/or amplified, for example by primer extension, DNA replication (e.g., by polymerase chain reaction) and/or transcription). See, for example, FIGS. 3 and 4 of the above-mentioned Li Review (Li et al Trends Biotechnol. 37:730, July 2019).

Thus, in many embodiments, a collateral activity assay includes steps of (1) target copying and/or amplification; (2) target binding; and (3) signal release and/or detection.

Typically, collateral activity assays as described herein are in vitro assays. In some embodiments, they may be cell free assays (e.g., may be substantially free of intact cells, or, in some embodiments, of cell fragments).

In some embodiments, collateral activity assays as described herein are performed on samples that are or are prepared from biological (e.g., blood, saliva, tears, urine, etc) or environmental (e.g., soil, water, etc) primary samples.

Thermostable Cas Enzymes

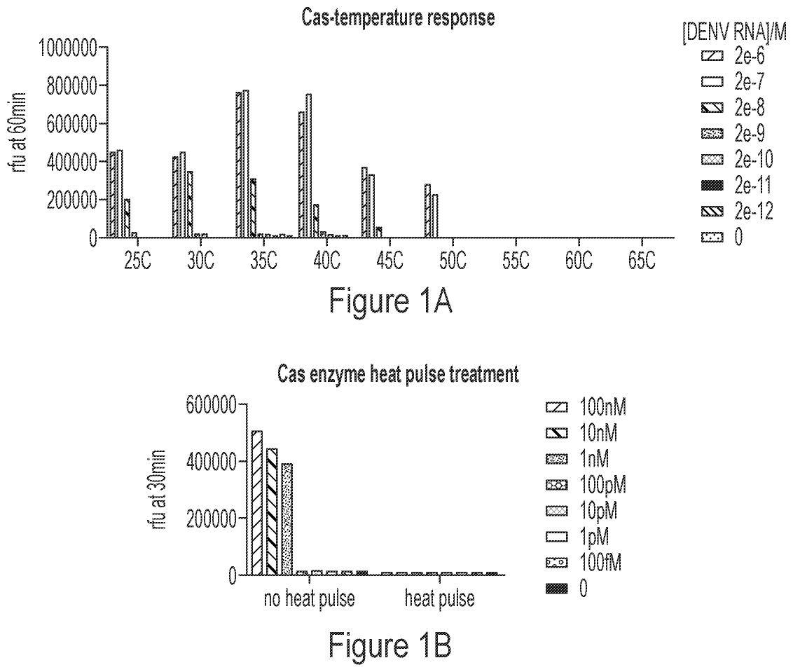

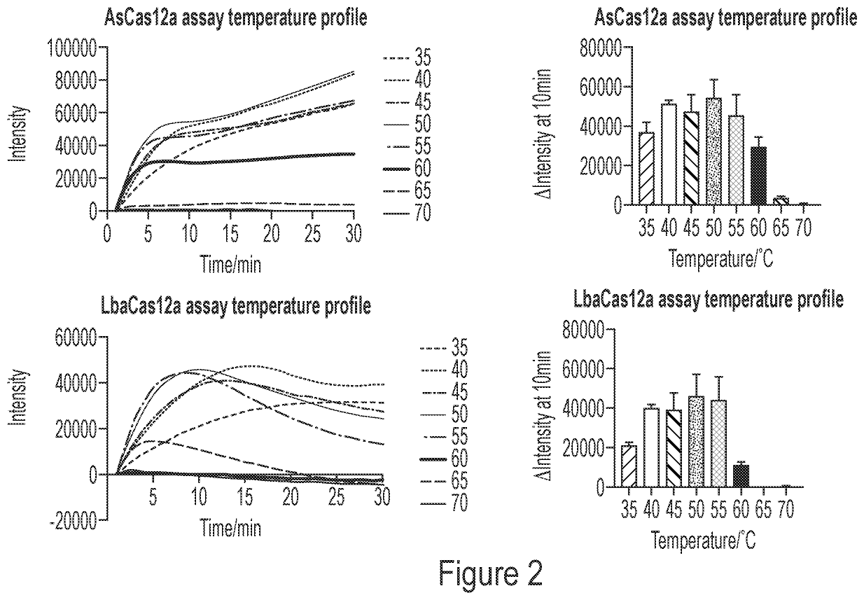

As described herein, the present disclosure identifies the source of a problem with certain detection (e.g., diagnostic assays) that utilize Cas protein collateral activity, as described above, in that certain Cas proteins with collateral activity are insufficiently stable at relevant temperatures (e.g., at temperatures at which nucleic acid extension and/or amplification are performed). Additionally, The present disclosure further surprisingly demonstrates that, for some proteins, loss of activity upon temperature elevation may be irreversible. This reality increases the significance of the insight, provided by the present disclosure, that Cas proteins with thermostable collateral activity are particularly desirable for use in assays asia described herein. FIGS. 1 and 2 document these findings.

The present disclosure therefore provides improved detection (e.g., diagnostic) assays that utilize Cas protein collateral activity, which improved assays utilize a thermostable Cas protein (e.g., whose collateral activity is thermostable) as described herein.

In some embodiments, steps of nucleic acid detection and target binding are performed in a single vessel; in some embodiments, steps of target binding an signal release are performed in a single vessel; in some embodiments, steps of steps of (1) target copying and/or amplification; (2) target binding; and (3) signal release and/or detection are performed in a single vessel; in some embodiments all steps are performed in a single vessel—i.e., provided improved assays are one-pot assays.

In some embodiments, improved collateral activity assays as described herein are in vitro assays. In some embodiments, they may be cell free assays (e.g., may be substantially free of intact cells, or, in some embodiments, of cell fragments).

In some embodiments, improved collateral activity assays as described herein are performed on samples that are or are prepared from biological (e.g., blood, saliva, tears, urine, etc) or environmental (e.g., soil, water, etc) primary sample.

In some embodiments, a Cas enzyme with thermostable collateral cleavage activity is a homolog (e.g., ortholog) of a Cas enzyme that either does not have demonstrable collateral cleavage activity, or has demonstrable collateral cleavage activity but loses such activity above a relevant temperature as described herein.

In some embodiments, a Cas enzyme with thermostable collateral cleavage activity as described herein is a Cas12 (e.g., Cas12a or Cas12b) enzyme. In some embodiments, a Cas enzyme with thermostable collateral cleavage activity as described herein is a Cas13 (e.g., Cas13a or Cas13b) enzyme.

In some embodiments, a Cas enzyme with thermostable collateral cleavage activity as described herein is a Cas enzyme comprising an amino acid sequence having 80%, 85%, 90%, 99% or 100% sequence identity to any one of SEQ ID Nos. 1-283. In some embodiments, improved collateral activity assays as described herein are performed using a Cas enzyme comprising an amino acid sequence having 80%, 85%, 90%, 99% or 100% sequence identity to any one of SEQ ID Nos. 1-283.

Target Nucleic Acid

Those skilled in the art will immediately appreciate that technologies provided herein are broadly applicable to achieve detection of a wide range of nucleic acids including, for example, nucleic acids from an infectious agent (e.g., a virus, microbe, parasite, etc), nucleic acids indicative of a particular physiological state or condition (e.g., presence or state of a disease, disorder or condition such as, for example, cancer or an inflammatory or metabolic disease, disorder or condition, etc), prenatal nucleic acids, etc.

In some embodiments, a target nucleic acid is detected by an assay comprising a Cas enzyme as described herein and a cRNA. In some embodiments, the structure of the cRNA can affect the activity of the Cas/cRNA complex. In some embodiments the structure of the Cas/cRNA complex contributes to the thermostability of the Cas collateral activity.

Typically, provided technologies will be applied to one or more samples to assess presence and/or level of one or more target nucleic acids in the sample. In some embodiments, the sample is a biological sample; in some embodiments, a sample is an environmental sample. In some embodiments, a sample is a crude sample (e.g., a primary sample or a sample that has undergone minimal processing).

In some embodiments, a sample will be processed (e.g., nucleic acids will be partially or substantially isolated or purified out of a primary sample); in some embodiments, only minimal processing will have been performed (i.e., the sample will be a crude sample).

### EXEMPLIFICATION

### Example 1: Temperature Profile for LwaCas13a

The thermostability of LwaCas13a was tested. Briefly, labeled RNA target was incubated with Rnase Inhibitor; T7 RNA Polymerase, LwaCas13a, MgCl2 and a cRNA. Individual samples were incubated at various temperatures to determine collateral activity.

FIG. 1A presents a temperature profile for LwaCas13a collateral activity. As can be seen, low activity was observed above 45° C.; activity was completely abolished about 55° C.

Further, FIG. 1B presents results of testing the reversibility of loss of LwaCas13a at higher temperatures. LwaCas13a was incubated for 5 minutes at 65° C. (“heat pulse”) while the control group (“no heat pulse”) was incubated at room temperature. The active of both enzymes was then tested at 37° C. The heat pulse group shows no activity. This reality increases the significance of the insight, provided by the present disclosure, that Cas proteins with thermostable collateral activity are particularly desirable for use in assays as described herein.

### Example 2: Temperature Profile for AsCas12a and Lbacas12a

The thermostability of AsCas12a and Lbacas12a was tested. Briefly, labeled RNA target was incubated with Rnase Inhibitor; T7 RNA Polymerase, AsCas12a or Lbacas12a, MgCl2 and a cRNA. Individual samples were incubated at various temperatures to determine collateral activity.

FIG. 2 presents temperature profiles for AsCas12a and LbaCas12a. As can be seen, low AsCas12a activity is observed at temperatures greater than 55° C. AsCas12a remains active at 60° C. for ˜5 min. AsCas12a has <10% activity at 65° C. for a few minutes. Further, LbaCas12a activity is significantly diminished at temperatures greater than 55° C.

### Example 3: Exemplary Thermostable Cas Candidates

The present Example describes certain thermostable Cas13 candidates for use in improved collateral activity assays as described herein.

In the present Example, it was determined that a Cas13 with collateral activity thermostable within a temperature range of about 62-about 68° C. would be particularly desirable, among other things, in one-pot assays with LAMP pre-amplification.

We performed a computational search for potentially thermostable Cas candidates and identified:
2 Cas13a candidates:

### TccCas13a (Thermoclostridium caenicola)

Exemplary sequences for use with TccCas13a include, but are not limited to:

(forward DR crRNAs)
(SEQ ID NO.: 284)
Agtgtctttgcaggaaagaacacagatcttgaggg
tcacaactcccatgtaggcggagactgcaacccct
atagtgagtcgtattaatt tc;
and
(reverse complement DR crRNAs)
(SEQ ID NO.: 285)
agtgtctttgcaggaaagaacacagatcttgaggg
ttgcagtctccgcctacatgggagttgtgacccct
atagtgagtcgtattaat ttc;

### ThpCas13a (Thalassospira profundimaris)

Exemplary sequences for use with ThpCas13a include, but are not limited to:

(forward DR crRNAs)
(SEQ ID NO.: 286)
Tctttgcaggaaagaacacagatcttgaggggtgt
agttcccctcaatttggggatgaacgtcgacccct
atagtgagtcgtattaat ttc;
and
(reverse complement DR crRNAs)
(SEQ ID NO.: 287)
tctttgcaggaaagaacacagatcttgagggtcga
cgttcatccccaaattgaggggaactacaccccct
atagtgagtcgtattaa tttc;

4 Cas12b candidates:
   AacCas12b (Alicyclobacillus acidoterrestris)
   AkCas12b (Alicyclobacillus kakegawensis)
   BhCas12b (Bacillus hisashii)
   LsCas12b (Laceyella sediminis)

Exemplary sequences for use with AacCas12b include, but are not limited to:

(crRNA)
(SEQ ID NO.: 288)
ttgtgagcggataaacacaggtgccacttctcaga
tttgagaagctcaacgggctttgccacctggaaag
tggccattggcacaccc gttgaaaaattctgtcc
tctagacccctatagtgagtcgtattaatttc

Exemplary sequences for use with AkCas12b include, but are not limited to:

(crRNA)
(SEQ ID NO.: 289)
Ttccggctcgtatgttgtgtggaattgtgagcgga
gtgccacttctcagaccgctcgccctatagtgagt
cgtattaatttc;
and
(tracrRNA)
(SEQ ID NO.: 290)
cgagcggtcatcttgaagccaacggggtgtttgct
cttggaaagagcacattggcacttcccgttgtcct
cgccgtcctatagacgac ccctatagtgagtcgt
attaatttc

Exemplary sequences for use with BhCas12b include, but are not limited to:

(crRNA)
(SEQ ID NO.: 291)
aattgtgagcggataaacacaggtgctaatgcctc
ccctatagtgagtcgtattaatttc;
and
(tracrRNA)
(SEQ ID NO.: 292)
gagacatcgtccagcaataggagtttctcacaccc
tgcagcacttatagctagacggttgtcctgaccaa
aagacagaacccctata gtgagtcgtattaattt
c

Exemplary sequences for use with LsCas12b include, but are not limited to:

(crRNA)
(SEQ ID NO.: 293)
Atggtcatagctgtttcctgtgtttatccgctcag
tgctaatcacatttaattcatctaccctatagtga
gtcgtattaatttc;
and
(tracrRNA)
(SEQ ID NO.: 294)
Gataaataatgtaatcctgtggttgaatggatttt
ttccatccttagcacacgcacagtattctttgccc
tttaggcaaaccctatagtg agtcgtattaattt
c.

Exemplary sequences of Cas proteins with thermostable collateral activity include those described in Table 1:

Table 1: Exemplary Sequences of Cas Proteins with Thermostable Collateral Activity

| SEQ | | |
|---|---|---|
| ID | | |
| NO. | Cas Enzyme | Putative Sequence |
| 1 | TccCas13a | MKITKRKWGEHHPPLYFYRDEDSGRLLAQNDRKQDYTD |
| | (Thermoclostridium | TLFNDIAQDTFERSLRNRLLKTPEKGDKRFYSNEIVKLVE |
| | caenicola) | KLCQGADVAEIMKSMERNEKLRPKNEKEIKNLKKQLDG |
| | | TLSEYGKRYTAPEGAMTLNDALFYLVEGNPLKQAMAK |
| | | AELGKIREALIKEKENRINRVRYSIKNNKIPLRIQEDGGIT |
| | | PNNDRAAWLLGLMKPADPAKGITDCYPLLGELEEVFDF |
| | | DKLSKTLHEKISRCQGRPRSIAMAVDEALKQYLRELWEK |
| | | SPSRQQDLKYYFQAVQEYFKDNFPIRTKRMGARLRQELL |
| | | KDKTSLSRLLEPKHMANAVRRRLINQSTQMHILYGKLY |
| | | AYCCGEDGRLLVNSETLQRIQVHEAVKKQAMTAVLWSI |
| | | SRLRYFYQFEDGDILSNKNPIKDFRDKFLRDTNKYTHED |
| | | VEACKEKLQDFFPLKELQEKIKEDAKGLQETDNKQADTT |
| | | DFKAIGHIVRDDRKLCNQLLAECVSCIGELRHHIFHYKN |
| | | VTLIQALKRIADKVKPEDLSVLRAIYLLDRRNLKKAFAK |
| | | RISSMNLPLYYREDLLSRIFKKEGTAFFLYSAKIQMTPSFQ |
| | | RVYERGKNLRREFECERMKAEASNGQNGQDGDRLKWF |
| | | RQLAAGDSADTHFNWAVEAYAESAADVENNVEFDTDV |
| | | DAQRALRNLLLLIYRHHFLPEVQKDETLVTGKIHKVLER |
| | | NRQLSEGQGPNQGKAHGYSVIEELYHEGMPLSDLMKQL |
| | | QRRISETERESRELAQEKTDYAQRFILDIFAEAFNDFLEA |
| | | HYGEEYLEIMSPRKDAEAAKKWVKESKTVDLKTSIDEK |
| | | EPEGHLLVLYPVLRLLDERELGELQQQMIRYRTSLASWQ |
| | | GESNFSEEIRIAGQIEELTELVKLTEPEPQFAEEVWGKRA |
| | | KEAFEDFIEGNMKNYEAFYLQSDNNTPVYRRNMSRLLR |
| | | SGLMGVYQKVLASHKQALKRDYLLWSEKHWNVKDEN |
| | | GADISSAEQAQCLLQRLHRKYAESPSRFTEEDCKLYEKV |
| | | LRRLEDYNQAVKNLSFSSLYEICVLNLEILSRWVGFVQD |
| | | WERDMYFLLLAWVRQGKLDGIKEEDVRDIFSEGNIIRNL |
| | | VDTLKGENMNAFESVYFPENKGSKYLGVRNDVAHLDL |
| | | MRKNGWRLEAGKTCSVMEDYINRLRFLLSYDQKRMNA |
| | | VTKTLQQIFDRHKVKIRFTVEKGGMLKIEDVTADKIVHL |
| | | KGSRLSGIEIPSHGERFIDTLKALMVYPRG* |
| 2 | ThpCas13a | MRIIKPYGRSHVEGVATEQPRRKLRLNTRPDISRDIPGFA |
| | (Thalassospira | QSHDALIIAQWISAIDKIATKPKPDQKPTQRQMNLRTTLG |
| | profundimaris) | DAAWQHLMAKNLLPAAKDPAIREKLHLIWQSKIAPWGA |
| | | SRPQEEKRGKPTPKGGWYERFCGALSPEAITQNVARQIA |
| | | KDIYDHLYVAAKRKGREPVKQGESSNKPGKFKPDRKLS |
| | | LIEERAESIAKNALRPGTHAPCPWGQDDQAIYEQAGDVA |
| | | TKIYDDARDYLEDKKRRSGNRNTSSVQYLPRDLAVKILY |
| | | AQYGRVFGPDTTIKAALDEQQSLFALHTAIKDCYHRLVN |
| | | DARKRHILRILPRNMAALFRLVRAQYDNRDINALIRLGK |
| | | VIHYHAGEQGKDEHHGIRDYWPSQQDIQNSRFWGSDGQ |
| | | ADIKRHEAFSRIWRHIIALASRTLHDWADPDSQKFTGDD |
| | | DDILMRAGAIESNVWDAGRYERKCDVLFGAQASLFCGA |
| | | EDFEKATLKQAITGTGNLRNATFHFKGKARFENELQRLA |
| | | DDVPVDVQSAIAALWQKDAEGRTRQIAETLQAVLAGHF |
| | | LSERQNRHILATLMAAMAQPGDVPLPRLRRVLARHDSIC |
| | | QRGRILPLPPCPDRAKLEESPALTCQYTVLKMLYDGPFR |
| | | AWLAQQNSTILNHYIDSTIARTNKAAQDMNGRKLAPAE |
| | | KDLITARAADIPRLSVDEKMVDFLGRLTAATATEMRVQ |
| | | RGYQSDGEKAQKQAGYIGEFECDVIARAFSDFLGQSGFD |
| | | FVLKLKADTPKPDAAQCDVAALIAPGDVPALTPQAWQQ |
| | | VLYFILHLVPVDDASRLLHQTRKWQALEKKGKDKEVKK |
| | | EKDKEVKKEDEKPDIADLQSVLMLYLDMHDAKFTGGA |
| | | ALHGIEKFAEFFVEKADFRAVFPPQSLQDQDRSIPRRGLR |
| | | EIVRFGHLPLLQHMSGTVKITHDNVVAWQTARTPDATG |
| | | TSPIARRQKQREELHALAVERPARFRNADLHNYMHALV |
| | | DVIKHRQLSAQVTLSDQVRLHRLMMGVLGRLVDYAGL |
| | | WERDLYFVLLALLYHHGVTPDDVLKGQGKRKLADGQV |
| | | VEALKPKNRKAAAPVGVFDDLDHYGIYQDDRQSIRNGL |
| | | SHFNMLRGGTAPDLSHWVNQTRRLVAHDRKLKNAVAK |
| | | SVIEMLAREGFDLDWTIEPDSGKHILRHGKIRTRQAQHFQ |
| | | KSRIRIEKKSAKPDKNDTVKIRENLHGDAMVERVARLFA |
| | | ARAQKYRDITTEKRLDHLFLKPKG* |
| 3 | AacCas12b | MAVKSIKVKLRLDDMPEIRAGLWKLHKEVNAGVRYYT |
| | (Alicyclobacillus | EWLSLLRQENLYRRSPNGDGEQECDKTAEECKAELLERL |
| | acidoterrestris) | RARQVENGHRGPAGSDDELLQLARQLYELLVPQAIGAK |
| | | GDAQQIARKFLSPLADKDAVGGLGIAKAGNKPRWVRM |
| | | REAGEPGWEEEKEKAETRKSADRTADVLRALADFGLKP |
| | | LMRVYTDSEMSSVEWKPLRKGQAVRTWDRDMFQQAIE |
| | | RMMSWESWNQRVGQEYAKLVEQKNRFEQKNFVGQEH |
| | | LVHLVNQLQQDMKEASPGLESKEQTAHYVTGRALRGSD |
| | | KVFEKWGKLAPDAPFDLYDAEIKNVQRRNTRRFGSHDL |
| | | FAKLAEPEYQALWREDASFLTRYAVYNSILRKLNHAKM |
| | | FATFTLPDATAHPIWTRFDKLGGNLHQYTFLFNEFGERR |
| | | HAIRFHKLLKVENGVAREVDDVTVPISMSEQLDNLLPRD |
| | | PNEPIALYFRDYGAEQHFTGEFGGAKIQCRRDQLAHMHR |
| | | RRGARDVYLNVSVRVQSQSEARGERRPPYAAVFRLVGD |
| | | NHRAFVHFDKLSDYLAEHPDDGKLGSEGLLSGLRVMSV |
| | | DLGLRTSASISVFRVARKDELKPNSKGRVPFFFPIKGNDN |
| | | LVAVHERSQLLKLPGETESKDLRAIREERQRTLRQLRTQ |
| | | LAYLRLLVRCGSEDVGRRERSWAKLIEQPVDAANHMTP |
| | | DWREAFENELQKLKSLHGICSDKEWMDAVYESVRRVW |
| | | RHMGKQVRDWRKDVRSGERPKIRGYAKDVVGGNSIEQI |
| | | EYLERQYKFLKSWSFFGKVSGQVLRAEKGSRFAITLREHI |
| | | DHAKEDRLKKLADRIIMEALGYVYALDERGKGKWVAK |
| | | YPPCQLILLEELSEYQFNNDRPPSENNQLMQWSHRGVFQ |
| | | ELINQAQVHDLLVGTMYAAFSSRFDARTGAPGIRCRRVP |
| | | ARCTQEHNPEPFPWWLNKFVVEHTLDACPLRADDLIPTG |
| | | EGEIFVSPFSAEEGDFHQIHADLNAAQNLQQRLWSDFDIS |
| | | QIRLRCDWGEVDGELVLIPRLTGKRTADSYSNKVFYTNT |
| | | GVTYYERERGKKRRKVFAQEKLSEEEAELLVEADEARE |
| | | KSVVLMRDPSGIINRGNWTRQKEFWSMVNQRIEGYLVK |
| | | QIRSRVPLQDSACENTGDI |
| 4 | AkCas12b | MAVKSIKVKLRLSECPDILAGMWQLHRATNAGVRYYTE |
| | (Alicyclobacillus | WVSLMRQEILYSRGPDGGQQCYMTAEDCQRELLRRLRN |
| | kakegawensis) | RQLHNGRQDQPGTDADLLAISRRLYELLVLQSIGKRGDA |
| | | QQIASSFLSPLVDPNSKGGRGEAKSGRKPAWQKMRDQG |
| | | DPRWVAAREKYEQRKAVDPSKEILNSLDALGLRPLFAVF |
| | | TETYRSGVDWKPLGKSQGVRTWDRDMFQQALERLMSW |
| | | ESWNRRVGEEYARLFQQKMKFEQEHFAEQSHLVKLARA |
| | | LEADMRAASQGFEAKRGTAHQITRRALRGADRVFEIWK |
| | | SIPEEALFSQYDEVIRQVQAEKRRDFGSHDLFAKLAEPKY |
| | | QPLWRADETFLTRYALYNGVLRDLEKARQFATFTLPDA |
| | | CVNPIWTRFESSQGSNLHKYEFLFDHLGPGRHAVRFQRL |
| | | LVVESEGAKERDSVVVPVAPSGQLDKLVLREEEKSSVAL |
| | | HLHDTARPDGFMAEWAGAKLQYERSTLARKARRDKQG |
| | | MRSWRRQPSMLMSAAQMLEDAKQAGDVYLNISVRVKS |
| | | PSEVRGQRRPPYAALFRIDDKQRRVTVNYNKLSAYLEEH |
| | | PDKQIPGAPGLLSGLRVMSVDLGLRTSASISVFRVAKKEE |
| | | VEALGDGRPPHYYPIHGTDDLVAVHERSHLIQMPGETET |
| | | KQLRKLREERQAVLRPLFAQLALLRLLVRCGAADERIRT |
| | | RSWQRLTKQGREFTKRLTPSWREALELELTRLEAYCGR |
| | | VPDDEWSRIVDRTVIALWRRMGKQVRDWRKQVKSGAK |
| | | VKVKGYQLDVVGGNSLAQIDYLEQQYKFLRRWSFFARA |
| | | SGLVVRADRESHFAVALRQHIENAKRDRLKKLADRILM |
| | | EALGYVYEASGPREGQWTAQHPPCQLIILEELSAYRFSD |
| | | DRPPSENSKLMAWGHRGILEELVNQAQVHDVLVGTVYA |
| | | AFSSRFDARTGAPGVRCRRVPARFVGATVDDSLPLWLTE |
| | | FLDKHRLDKNLLRPDDVIPTGEGEFLVSPCGEEAARVRQ |
| | | VHADINAAQNLQRRLWQNFDITELRLRCDVKMGGEGTV |
| | | LVPRVNNARAKQLFGKKVLVSQDGVTFFERSQTGGKPH |
| | | SEKQTDLTDKELELIAEADEARAKSVVLFRDPSGHIGKG |
| | | HWIRQREFWSLVKQRIESHTAERIRVRGVGSSLD |
| 5 | BhCas12b | MATRSFILKIEPNEEVKKGLWKTHEVLNHGIAYYMNILK |
| | (Bacillus | LIRQEAIYEHHEQDPKNPKKVSKAEIQAELWDFVLKMQK |
| | hisashii) | CNSFTHEVDKDEVFNILRELYEELVPSSVEKKGEANQLS |
| | | NKFLYPLVDPNSQSGKGTASSGRKPRWYNLKIAGDPSW |
| | | EEEKKKWEEDKKKDPLAKILGKLAEYGLIPLFIPYTDSNE |
| | | PIVKEIKWMEKSRNQSVRRLDKDMFIQALERFLSWESW |
| | | NLKVKEEYEKVEKEYKTLEERIKEDIQALKALEQYEKER |
| | | QEQLLRDTLNTNEYRLSKRGLRGWREIIQKWLKMDENE |
| | | PSEKYLEVFKDYQRKHPREAGDYSVYEFLSKKENHFIWR |
| | | NHPEYPYLYATFCEIDKKKKDAKQQATFTLADPINHPLW |
| | | VRFEERSGSNLNKYRILTEQLHTEKLKKKLTVQLDRLIYP |
| | | TESGGWEEKGKVDIVLLPSRQFYNQLFLDIEEKGKHAFTY |
| | | KDESnCFPLKGTLGGARVQFDRDHLRRYPHKVESGNVG |
| | | RIYFNMTVNIEPTESPVSKSLKIHRDDFPKVVNFKPKELT |
| | | EWIKDSKGKKLKSGIESLEIGLRVMSIDLGQRQAAAASIF |
| | | EVVDQKPDIEGKLFFPIKGTELYAVHRASFNIKLPGETLV |
| | | KSREVLRKAREDNLKLMNQKLNFLRNVLHFQQFEDITER |
| | | EKRVTKWISRQENSDVPLVYQDELIQIRELMYKPYKDW |
| | | VAFLKQLHKRLEVEIGKEVKHWRKSLSDGRKGLYGISLK |
| | | NIDEIDRTRKFLLRWSLRPTEPGEVRRLEPGQRFAIDQLN |
| | | HLNALKEDRLKKMANTIIMHALGYCYDVRKKKWQAKN |
| | | PACQIILFEDLSNYNPYEERSRFENSKLMKWSRREIPRQV |
| | | ALQGEIYGLQVGEVGAQFSSRFHAKTGSPGIRCSWTKE |
| | | KLQDNRFFKNLQREGRLTLDKIAVLKEGDLYPDKGGEK |
| | | FISLSKDRKCVTTHADINAAQNLQKRFWTRTHGFYKVY |
| | | CKAYQVDGQTVYIPESKDQKQKIIEEFGEGYFILKDGVY |
| | | EWVNAGKLKIKKGSSKQSSSELVDSDILKDSFDLASELK |
| | | GEKLMLYRDPSGNVFPSDKWMAAGVFFGKLERILISKLT |
| | | NQYSISTIEDDSSKQSM |
| 6 | LsCas12b | MSIRSFKLKIKTKSGVNAEELRRGLWRTHQLINDGIAYY |
| | (Laceyella | MNWLVLLRQEDLFIRNEETNEIEKRSKEEIQGELLERVHK |
| | sediminis) | QQQRNQWSGEVDDQTLLQTLRHLYEEIVPSVIGKSGNAS |
| | | LKARFFLGPLVDPNNKTTKDVSKSGPTPKWKKMKDAGD |
| | | PNWVQEYEKYMAERQTLVRLEEMGLIPLFPMYTDEVGD |
| | | IHWLPQASGYTRTWDRDMFQQAIERLLSWESWNRRVRE |
| | | RRAQFEKKTHDFASRFSESDVQWMNKLREYEAQQEKSL |
| | | EENAFAPNEPYALTKKALRGWERVYHSWMRLDSAASEE |
| | | AYWQEVATCQTAMRGEFGDPAIYQFLAQKENHDIWRG |
| | | YPERVIDFAELNHLQRELRRAKEDATFTLPDSVDHPLWV |
| | | RYEAPGGTNIHGYDLVQDTKRNLTLILDKFILPDENGSW |
| | | HEVKKVPFSLAKSKQFHRQVWLQEEQKQKKREVVFYD |
| | | YSTNLPHLGTLAGAKLQWDRNFLNKRTQQQIEETGEIGK |
| | | VFFNISVDVRPAVEVKNGRLQNGLGKALTVLTHPDGTKI |
| | | VTGWKAEQLEKWVGESGRVSSLGLDSLSEGLRVMSIDL |
| | | GQRTSATVSVFEITKEAPDNPYKFFYQLEGTELFAVHQRS |
| | | FLLALPGENPPQKIKQMREIRWKERNRIKQQVDQLSAILR |
| | | LHKKVNEDERIQAIDKLLQKVASWQLNEEIATAWNQAL |
| | | SQLYSKAKENDLQWNQAIKNAHHQLEPVVGKQISLWRK |
| | | DLSTGRQGIAGLSLWSIEELEATKKLLTRWSKRSREPGV |
| | | VKRIERFETFAKQIQHHINQVKENRLKQLANLIVMTALG |
| | | YKYDQEQKKWIEVYPACQVVLFENLRSYRFSYERSRRE |
| | | NKKLMEWSHRSIPKLVQMQGELFGLQVADVYAAYSSR |
| | | YHGRTGAPGIRCHALTEADLRNETNIIHELIEAGFIKEEHR |
| | | PYLQQGDLVPWSGGELFATLQKPYDNPRILTLHADINAA |
| | | QNIQKRFWHPSMWFRVNCESVMEGEIVTYVPKNKTVHK |
| | | KQGKTFRFVKVEGSDVYEWAKWSKNRNKNTFSSITERK |
| | | PPSSMILFRDPSGTFFKEQEWVEQKTFWGKVQSMIQAYM |
| | | KKTIVQRMEE |
| 7 | Cas12 | MFKKKLFDDEEFISLAQNQEESNALNAFKGFTTHFKDFQENRKNMYS |
| | | EDKESTAIAYRIIHENLPVFITNNIRFEKIINELDRSNIHSIEKELKEELANN |
| | | KLKDIFNIEYFQNTLTQNDITRYNTIIGGKVKADGKKVQGLNEYINLFN |
| | | QHNKDKKLPLLKPLYKQILSEENSASFIVPAFEKDNEVLQSIFDFWNKCI |
| | | IDAKGPISGKKYNLLSKIQSLLQNLDKLKNNQLEEMYFENENLSTISNDV |
| | | YGQWNLIRDALGNFYNSIDAKKNKKDYYSWKEIQDALVYYKQTNDEY |
| | | KDIDQKAFLIYFKEMKVNDGEENTNNNIINLINERYKRIEPLLKEDRDN |
| | | RKDLHQDKGKVAIIKEFLDSLKLLQNTIKLLYVDDSLDNMNYDFYNQLT |
| | | DYYETLRPLNTLYNRVRNYMTRKPFSEEKFVLTFNSPTLLDGWDLNKE |
| | | EANLGVILRKDNKYYLGIMNKGDNKIFKKYDEEPGDDYYEKMVYKLLP |
| | | GPNRMLRKVFFSNKNIEYYKPNQDIQNLYNKGEFKKGESLNKESLHKLI |
| | | DFYKNSISKNGDWSVFNFKFKKTTAYDDISQFYKDVENQGYKLFFKTIK |
| | | TSYIDQLVNEGKLYLFQIYNKDFSENKKRKDESNPNLHTIYFKNLFSEDN |
| | | LKNVVYKLNGKAEVFYRKKSIEYPEEIRRKGHHYNELKDKFDYPIIKDKR |
| | | YSEDKFLFHVPITLNFLAKSDEKVNEMVKNYIAATNEKIHIIGIDRGERN |
| | | LLYLSLIDSNGNIVKQQSLNIIELPKYQKQIDYHAKLNEKEKQRLAARQN |
| | | WDVIENIKELKEGYLSQVIHQIARLMVDYKAILVMEDLNFGFKRGRFK |
| | | VEKQVYQKFEKMLIDKLSYLVFKEKNLCEPGGSLRAYQLSAPFKSFKAL |
| | | GKQSGMIFYVPAQYTSKIDPTTGFYNFLNIDVSNLARSKETFSKFDKIVY |
| | | NKKEDYFEFYCKMINFESANQLTKKSQNKANAELKEFQWILCSTHHDR |
| | | FKVERKNNQINYCKINVNEELKKLLNSKGINYEKSNDLKSEILNIDESKFF |
| | | KELGYLLKILVSLRYNNGKKGSEEQDFILSPVKNASGKFFCTLDNNNTLP |
| | | LDADANGAYNIALKGLMIVQRVKAGGKLDLSISKDDWINFLIMNKKLP |
| | | K |
| 8 | Cas12 | MSNQSVFKDFTNLYELSKTLRFELKPVGKTLRMLEDAKVFKTDELIQKK |
| | | YEQTKPFINKLHQEFVKESLEGRSLEGLESYQDILKEWQKDKKDKIAQK |
| | | NLGIKEKELYKQVTQLFNAKAKEWSEPYAHLGLKKKDIGILFEEGVFKIL |
| | | KEKYNNDKDAKITNKVTGEIFFEDFWKGFVGYFQKFFETRKNFYKDDG |
| | | TSTAIATRIVAQNLKRFCDNIGLFEKIKDQIDSSEVEQSFGISMEKVFSLD |
| | | FYNQCLLQGGIDKYNEILGGKTLENGEKFKGINELINKYRQDNKGDKSS |
| | | FLKILDKQILSEKESFIDEIKNDKELEETLKNLHETAKVKTKIFGTLFEDFIG |
| | | NNTKYDLAKIYISKEAFNTISHKWTGGTDLFAENLFNALKDEQILKSSAK |
| | | KKDGSYVFPDFIEFLHIKTALENVPKDINFWKERYYVNKEGENKEFFLG |
| | | NGEIWQQFLQIFNFEFNELFQKEIIDNQTGKKMHIGYKVYKEEISKLLE |
| | | DFKVDKDSTVIIKHFADSVLWIYQMAKYFALEKKRTWRDEYDLDTFYT |
| | | DPKNGYLAFYENAYEEIVQIYNKLRNYLTKKPYSTEKWKLNFQNSTLAS |
| | | GWDKNKEADNFTVILRKDGKYFLGLMRKGANKLFDKRYGSEFSQGLE |
| | | KGKYEKMNYKYFPSPSKMIPKTSTQVHEVKKHFKNSSEPFFLEESSSLG |
| | | KFIKQLKITKEVFDLNNFEYKKSYLSTLNGESPDESQRVKADSKKTGQV |
| | | KLFQKEFLNLSQNELLYKKSLFAWVDFCKEYLDCFPSTGDGFLQFKKYI |
| | | QDTEKYESIDQFYKDIERGGYKISFQNISEEYISCKNQNSELYLFKIHNKD |
| | | WNLKDGKPKTGMKNLHTMYFESLFSSENIAQNFPMKLNGQAEIFYRP |
| | | KTDINKLEMKKDSKGKNVVDHKRYEEDKIFFHLPMTLNRGKSLFNFNV |
| | | QLNNFLADNPEINIIGVDRGEKHLAYYSVINQNQEILDGGTLNVVKGG |
| | | NGKDIDYHKKLEDKAEKREQARKDWQDVEGIKDLKKGYISQVVRKLA |
| | | DLAIEHNAIIVFEDLNMRFKQIRGGIEKSVYQQLEKALIEKLSFLVRKNE |
| | | KNPEEAGYLLKAYQLSAPFETFQRIGKQTGIIFYTQASYTSKIDPLTGWR |
| | | PNLYLKYSNAKKAKADISKFSEIEFINNRFEFTYDLQEFRSQKDKKKEYP |
| | | KKTLWTLCSSVERYRWNRKLNDNKGGYEHYSDLTSDFKKLFKKYNINI |
| | | NEDILGQIENMDTDDRKNNARFFSGFMFFWNLICQIRNTNSDVISGE |
| | | SDNDFILSPVEPFFDSRKASQFGSDLPENGDDNGAFNIARKGIMILKKI |
| | | SQYVEENENCDKLKWGDLYISHTDWDNFI |
| 9 | Cas12 | MTNYTDFIGLYPVQKTLRFELRPQGKTAEKMRESGLLEQDREKAKNYI |
| | | VMKALIDDYHRRFINELLEKASFDWQPLFEALNNVKVNKDDKSKKELE |
| | | KEQLHMRKELIGLFEKDERFKYLFSEKLFSELLNKEISERNDPDEMEAM |
| | | RSFDRFSGYFIGFHENRRNIYSNEDKHNSLAYRVVAENFPKFADNCRK |
| | | YSLIKENMQEAVVEFKKEIASVVDIDVDQMFDISYFNKVLTQKGIDDY |
| | | NTMLGGVSEEGSVKIRGLNEFLNLYYQKVTDNKRIKMAPLYKQILCESK |
| | | TKSFIPYMFENDEEVISSINQYYDSVKYDILQRSVYLLSNYKEYDASKIFI |
| | | DQKSISSISIVLFGSWETLGGLMQIYKADQIGDPGLEKTRKKVDKWLSS |
| | | SYFTLKEVFEAIGEQDPFRVYVEKLSLVLKNIEEFDKSCLLEGTHFSGDEL |
| | | LTQDIKGFLDLLMEVQHLMKPFNAKEDLDKDAAFYSEYNEIYEALSEIIP |
| | | LYNKVRNYATKKKYSTYKIKMNFGNPTLAAGWDLNKERDNTAVILLR |
| | | GNNYYLGIMNPKKKTKFEELPSGEDNDCYRKMVYKLLPGPNKMLPKV |
| | | FFSKKGIGTFNPSKEILEGYETGKHKLGDSFDIDYCHSLIDFFKENIPKYG |
| | | DWGTYEFKFSPTEEYSDISQFYKEVSEQGYKITFQNISRKAIDDLVNNG |
| | | ALFLYQIYNKDFSEHSKGKNNLHTMYWKAAFSEENLRNVVIKINGEAE |
| | | LFYRDKSDISKTEHSAGTILVNRTDRKDNPIPNSIYYELFKYKTGQIKSVS |
| | | DEAKQYLDDLVTHEAKYPITKDRRYTEDRMFFHIPITLNFGSSGNTNIN |
| | | KAVIDHVLNSKDVHIIGIDRGERNLLYVSVIDRKGNIIKQRSLNVIDGIDY |
| | | HEKLDQREKENISARKSWSNVEKIKDLKEGYLSYVIH |
| 10 | Cas12 | MKDFYQFTNLYALSKTLRFSLIPTPATKQMLEDAKVFEKDETIQKKYEA |
| | | TKPYFDRLHREFALEALQDQKLDFKNYLELYRKYKADKKASGKLLINIEK |
| | | DLRKEVVKLFDKQGEKWAKQYPGLKNKNIGVLFKEAVFTVILKERYGN |
| | | EKETQILDESSGQLVSIFDSWKGFIGYFKKFHETRKNFYKDDGTSTALA |
| | | TRIIDQNLKRFCDNILIFESTKEKVDFSEVEISFGKPLSEVFTLEFYNTCFL |
| | | QNGIDFYTKILGGETLQNGEKVKGLNECINLHKQKTGEKLPFFKSLDKQ |
| | | ILSEKDKFFIDEISNETQLLEVLKSFVASAESKTDTIKTLVDDFVKDQDKY |
| | | DLNYIYFSNDGLNTITRKWTTETQVFEEALYTALKAAKVVSSSAKKNEG |
| | | GYSFPDFIPFAHLKTALESIKIDGTIWRDNFNAIENFEEKSIWAQFLAIY |
| | | NFELSNLFETEIKNPEIGNCPTIGYNVYKQDFEELLKSFVYDPNAKVTIK |
| | | NFADNVLSIYQMAKYFAVEKKRGWNTDYELDVFYTDPQNGYLQYYE |
| | | NAYEEIVQVYNKLRNYLTKKPYSEEKWKLNFDSGTPIKYTTRAIIFNNTT |
| | | NERYYLGLLKKGVAKPREFEPINNNIISSGEFRRMIIQQLKFQTLAGKGY |
| | | VRDFGVKYSEDKDGVKHLQQLIKKQYLSKYPCLKKIADGVYNDKKAFD |
| | | ADIKDVLLETYNLDFQPISEEFILNKNRLGEIYLFEIHNKDWNLKDGKNK |
| | | SGSKNLHTMYFESLFVDKTTFKLNNEGAEVFYRPATNEGKLGTKKDRN |
| | | GKIIINHKRYATDKILFHCPIGLNKDAGKSYTFNAKINNMLANNPDINII |
| | | GVDRGEKHLAYYSVITQKGKILDRGSLNKVEGGDKQEIDYAKKLEETAK |
| | | NREQARKDWQAVEGIKDLKRGYISQVVRKLADLAIEHNAIIVFEDLNM |
| | | RFKQIRGGIEKSVYQQLEKALIDKLSFLVMKGEADPEKAGHLLKAYQLV |
| | | APFESFQSMGKQTGIIFYTQANYTSKIDPITGWRPNLYLKYTSAEKAKA |
| | | DILKFSKIEFVNNRFELTYDIKNFVLDKKVVLSNKTKWTVCSSVERFRW |
| | | NRRLESNQGNYEHYENLTENLSSLFKDFGFEIEQNIIRQVEQLATKGNE |
| | | QFFRSFIFYVNLIFQIRNTDAKAKDQNKEDFILSPVEPFFDSRTPEKFGE |
| | | NLPENGDDNGAFNIARKGIIMLNKISAYKQEVGNVDKIIWKDLFISAAE |
| | | WDNFTQE |
| 11 | Cas12 | MDSYEQFTKLYPIQKTIRFELKPQGRTKEHFDNSNFLEKDRERDDNYKI |
| | | LKEVIDDYHREFIDECLSNIQLNWDDLKKFSEEYRRSKEKKNNRDSESE |
| | | QKRMSTTSETRAINKKNLEAEQKRMRGEIVSAFKKDDRFKHLFSEKLFS |
| | | ILLKNQIYEKGTLEEIEAFDCFNKFSGYFKSFHENRKNMYSDEDKETAIS |
| | | YRIINENFPKLLDNFEKYQYVCREYPEQIREAESTLAEAGCYIKMDEIFSI |
| | | DNFNNVMMQGGKESGISRYNLAIGGIVQGTGEKPKGLNEFLNLAYQ |
| | | NEPNGRKKIRMEPLYKQILSKEESFSYRLEAFTDDSQLLSAIRSFFDIVEK |
| | | DKNGNIFDRAVNLMSSFSNYDTSKIYIRKAYLNQVSKEIFGYRGKSDSK |
| | | PAKTADESLNKSGGWEKLGQMLRDYKADSIGDRNLEKTCKKVDKWL |
| | | DSDEFTLSDILGAISLAGSNETFEAYVSEICVARRNIDKEKEKEKNINVEK |
| | | ISGDTESIQIIKALLDSVQEFFHLLSPFQLHPNTPHDWTFYAEFNDIYDK |
| | | LSAITPLYNQARNHLTKKNLDTSKIKLNFNNPTLANGWDVNKEYENTA |
| | | VILIRDGKYYLGIMNPKNKRKIKFDEGSGAGPFYQKMVYKLLPGPYRM |
| | | LPKVFFAKKNIDYYNPSQEIREGYKAGKHKKGKEFDKGFCHKLIDFFKES |
| | | IQKNENWKVFDFKFSPTESYDDISEFYQEVEKQGYRMYFVNIPSDTIDR |
| | | YVEGGDMFLFQIYNKDFAKGAKGNKDMHTLYWNAVFSEENLQKGV |
| | | MKLSGEAELFYRKKSDIKDPPHREGEILVNRTYIDRTHVSGVMGEQNT |
| | | VKESRIPVPDEIHKNLFDYYNHGRELTKEEKEYCDKVGSFKAYYGIVKD |
| | | RRYLENKMYFHVPLTLNFKAIGEKRINKMAIEKFLTDENACIIGIDRGER |
| | | NLLYYSIIDRNGKIIDQKSLNVIDGFDYHEKLSQRQTEREVARQSWNSI |
| | | GKIKDLKEGYLAKAVHEISKMAIKYNAIVVLEDLHFGFKKGRLKVEKQIY |
| | | QKFEEMLINKLNYLVFKDVSDSSDAGGVLNAYQLTAPLESFSKLGKQS |
| | | GILFYVPAAFTSVIDPTTGFVDLFNSSSITSTQKKKEFLQRFESIVYSARD |
| | | GGIFAFTFDYRNFSKIATDHRNMWTVYTHGERIRYVRDEKCYKTTDPT |
| | | KRIKEALSGIEYDDGSDIRDKITQSGDNNLINTVYHSFMDTIKMRNKD |
| | | GRIDYIISPVKNRNGEFFRSDYKHRDFPVDADANGAYHIALKGELLMR |
| | | MIGKTYDSNSDKMPKLEHKDWFEFMQTRGDQ |
| 12 | Cas12 | MKKEKEFKSFGDFTNLYEISKTLRFELKPVENTQTMLDEADVFGKDKVI |
| | | KDKYTKTKPFIDKLHREFVDESLKDVSLSGLKKYSEVLENWKKNKKDKD |
| | | IVKELKKEEERLRKEVVEFFDNTAKKWANEKYKELGLKKKDIGILFEESV |
| | | FDLLKEKYGEEQDSFLKEEKGDFLKNEKGEKVSIFDEWKGFVGYFTKFQ |
| | | ETRKNFYKNDGTETALATRIIDQNLKRFCDNIDDFKKIKNKIDFSEVEKN |
| | | FNKTADVFSLDFYNQCLLQKGIDSYNEFIGGKTLENGKKLKGVNELVN |
| | | EYRQKNKNEKVSFLKLLDKQILSEKEKLSFGIENDEQLLVVLNSFYETAE |
| | | EKTKILRTLFGDFVEHNENYDLDKTYISKVAFNTISHKWTNETHKFEELL |
| | | YGAMKEDKPIGLNYDKKEDSYKFPDFIALGYLKKCLNNLDCDTKFWKE |
| | | KYYENNADKKDKDKGFLTGGQNAWDQFLQIFIFEFNQLFNSEAFDNK |
| | | GKEIKIGYDNFRKDFEEIINQKDFKNDENLKIAIKNFADSVLWIYQMAK |
| | | YFAIEKKRGWDDDFELSEFYTNPSNGYSLFYDRAYEEIVQKYNDLRNYL |
| | | TKKPYKEDKWKLNFENPTLANGFDKNKESDNSTVILRKKRKYYLGLMK |
| | | KGNNKIFEDRNKAEFIRNIESGAYEKMAYKYLPDVAKMIPKCSTQLNE |
| | | AKNHFRNSADDLEIKKSFSNPLKITKRIFDLNNIQYDKTNVSKKISGDNK |
| | | GIKIFQKEYYKISGDFDVYKSALNDWIDFCKDFLSKYDSTKDFDFSILRKT |
| | | KDYKSLDEFYVDVAKITYKISFTPVSESYIDQKNKNGELYLFEIYNQDFAK |
| | | GKMGAKNLHTLYFENVFSPENISKNFPIKLNGNAELFFRPKSIESKKEKR |
| | | NFVREIVNKKRYSEDKIFFHCPITLNRETGSIYRFNNYVNNFLSENNINII |
| | | GVDRGEKHLAYYSVIDKNGVKIGGGSFNEINKVDYAKKLEERAGEREQ |
| | | SRKDWQVVEGIKDLKKGYISQVVRELADLAIKHNAIIVLEDLNMRFKQI |
| | | RGGIEKSIYQQLEKALIDKLSFLVEKGEKDPNQAGHILKAYQLAAPFTSF |
| | | KDMGKQTGIVFYTQASYTSKTCPNCGFRKNNNKFYFENNIGKAQDAL |
| | | KKLKTFEYDSENKCFGLSYCLSDFANKEEVEKNKNKKRNNAPYSDIEKK |
| | | DCFELSTKDAVRYRWHDKNTERGKTFFEGESVYEEKEEKEIGQ.TKRGL |
| | | VKEYDISKCLIGLFEKTGLDYKQNLLDKINSGKFDGTFYKNLFNYLNLLFE |
| | | IRNSISGTEIDYISCPECQFHTDKSKTIKNGDDNGSYNIARKGMIILDKIK |
| | | QFKKENGSLDKMGWGELFIDLEEWDKFAQKKNNNIIDK |
| 13 | Cas12 | MKSFDSFTNLYSLSKTLKFEMRPVGNTQKMLDNAGVFEKDKLIQKKY |
| | | GKTKPYFDRLHREFIEEALTGVELIGLDENFRTLVDWQKDKKNNVAM |
| | | KAYENSLQRLRTEIGKIFNLKAEDWVKNKYPILGLKNKNTDILFEEAVFG |
| | | ILKARYGEEKDTFIEVEEIDKTGKSKINQISIFDSWKGFTGYFKKFFETRK |
| | | NFYKNDGTSTAIATRIIDQNLKRFIDNLSIVESVRQKVDLAETEKSFSISL |
| | | SQFFSIDFYNKCLLQDGIDYYNKIIGGETLKNGEKLIGLNELINQYRQNN |
| | | KDQKIPFFKLLDKQILSEKILFLDEIKNDTELIEALSQFAKTAEEKTKIVKKL |
| | | FADFVENNSKYDLAQIYISQEAFNTISNKWTSETETFAKYLFEAMKSGK |
| | | LAKYEKKDNSYKFPDFIALSQMKSALLSISLEGHFWKEKYYKISKFQEKT |
| | | NWEQFLAIFLYEFNSLFSDKINTKDGETKQVGYYLFAKDLHNLILSEQID |
| | | IPKDSKVTIKDFADSVLTIYQMAKYFAVEKKRAWLAEYELDSFYTQPDT |
| | | GYLQFYDNAYEDIVQVYNKLRNYLTKKPYSEEKWKLNFENSTLANGW |
| | | DKNKESDNSAVILQKGGKYYLGLITKGHNKIFDDRFQEKFIVGIEGGKY |
| | | EKIVYKFFPDQAKMFPKVCFSAKGLEFFRPSEEILRIYNNAEFKKGETYSI |
| | | DSMQKLIDFYKDCLTKYEGWACYTFRHLKPTEEYQNNIGEFFRDVAED |
| | | GYRIDFQGISDQYIHEKNEKGELHLFEIHNKDWNLDKARDGKSKTTQK |
| | | NLHTLYFESLFSNDNVVQNFPIKLNGQAEIFYRPKTEKDKLESKKDKKG |
| | | NKVIDHKRYSENKIFFHVPLTLNRTKNDSYRFNAQINNFLANNKDINII |
| | | GVDRGEKHLVYYSVITQASDILESGSLNELNGVNYAEKLGKKAENREQ |
| | | ARRDWQDVQGIKDLKKGYISQVVRKLADLAIKHNAIIILEDLNMRFKQ |
| | | VRGGIEKSIYQQLEKALIDKLSFLVDKGEKNPEQAGHLLKAYQLSAPFET |
| | | FQKMGKQTGIIFYTQASYTSKSDPVTGWRPHLYLKYFSAKKAKDDIAK |
| | | FTKIEFVNDRFELTYDIKDFQQAKEYPNKTVWKVCSNVERFRWDKNL |
| | | NQNKGGYTHYTNITENIQELFTKYGIDITKDLLTOISTIDEKQNTSFFRD |
| | | FIFYFNLICQIRNTDDSEIAKKNGKDDFILSPVEPFFDSRKDNGNKLPEN |
| | | GDDNGAYNIARKGIVILNKISQYSEKNENCEKMKWGDLYVSNIDWDN |
| | | FVTOANARH |
| 14 | Cas12 | MNTQKKEFNPKSFKDFTNLYSLNKTLRFSLTPNKKTAEILEFNKQKEVK |
| | | CFSNDRKIAGAYQEIKKYLNKLHQEFIQEAMKFFAFSEEELKGFEKEYLN |
| | | LLNFTDKDNFKKKNKIRNEYEQERKILTIKIATYFSKFKSEKYQSFNLANI |
| | | TGKKVFSILEQKYKEDKKTLKIIHIFKYKPTKDEKKEGEAVNFSTYLTGFN |
| | | ENRKNFYKSEDKAGQFATRTIDNLAQFIKNKKLFEDKYQKNYSKIGILD |
| | | EQIKIFNLDYFNNLFLQEGLDEYNGILGNNKGEENKSNEGINQKINIFK |
| | | QKEKARLKKEKENFNKSDFPLFKELYKQIGSIRKENDVYVEIKTDKELVE |
| | | ELNNFPKNVENYLKDIQSFYKTFFEKLQNEEYELDKIYLPKSVGTYFSYIA |
| | | FSDWNKLAFIYNKRYKNEKIKIVEGGDVNVQYRSLEVLKNRIDELKDED |
| | | NLNFNKFFIDKLKFNEAKKENNWQNFWFCIEYYINSQFIGGEKNILNKE |
| | | KNEYEILPFGSLKELKEKYFEAVKKYKEKMVDTESGLTDDEEKEIKETLK |
| | | NYLDRIKEIERIAKYFDLKKSFEEIKQEDLDSNFYGEYQKVVDKTNELKIY |
| | | QYYSEFRNYLTQNNSVEEKIKLNFNSGLLLDGWDLNKEKVKFSIIFQEN |
| | | GKYYLGIINKEKDKTILDKDKHPEIFTKNSDFRKMEYKLFPSPSKMLPKIS |
| | | FSETAKKGDEDVGWSEEIQKIKDEFAEFQEYKKKSKDNWKDEFNRGK |
| | | LNKLIDYYKQVLEKHSEGYMNTYNFELKDSSKYKNLGEFNDDIARQNY |
| | | KVKFVGIDKNYIDEKVANGELFLFQIYNKDFSEDKKEGSTNNLETIYFKE |
| | | LFSKENLENPVFKLSGGAEMFFRNKIEKKKEKKKLDKDGKPMISKKGEK |
| | | VVDKRRFSENKILFHLPIEINYGKGKMPNFNKKINEYISKNPENIKIIGID |
| | | RGEKHLLYYSIIDQNGNNIESMSLNAVDEFGNFVNPEKLEEYEIDNNG |
| | | KKERRWKYIVNDKEIKVTNYQRKLDELEKERQKSRQSWQNINKIKNLK |
| | | KGYISFVVKKIVDLAIENNAIIILEDLNFGFKSFRQKIEKNVYQQFEKALID |
| | | KLGFVVDKQKQNQRFAPQLSAPFESFQKIGKQTGIVYYVLANNTSKVC |
| | | PSCQWIKNFYLKYEKKNTIFNLQKNQKLKVFFEQEKNRFRFEYQMSKE |
| | | YISVYSDVDRQRYDKTKNQNKGGYLEYKNSNQKEIIDKDGVIQKQSITL |
| | | QLKELFKENHIDLEKEILKQLDNKKEKNSGYTGVYNKFIYLFNLILQIRNA |
| | | ISFREKDYIQCPSCHFDTRKENYLKINDGDGNGAYNIALRGLYLLKGKN |
| | | GIINNLEKIKLIFSNNDYFQWAKKLKNKK |
| 15 | Cas12 | MEEKMLKSYDYFTKLYSLQKTLRFELKPIGKTLEHIKNSGIIESDETLEEQ |
| | | YAIVKNIIDKLHRKHIDEALSLVDFTKHLDTLKTFQELYLKRGKTDKEKEE |
| | | LEKLSADLRKLIVSYLKGNVKEKTQHNLNPIKERFEILFGKELFTNEEFFLL |
| | | AENEKEKKAIQAFKGFTTYFKGFQENRKNMYSEEGNSTSIAYRIINENL |
| | | PLFIENIARFQKVMSTIEKTTIKKLEQNLKTELKKHNLPGIFTIEYFNNVLT |
| | | QEGISRYNTIIGGKTTHEGVKIQGLNEIINLYNQQSKDVKLPILKPLHKQI |
| | | LSEEYSTSFKIKAFENDNEVLKAIDTFWNEHIEKSIHPVTGNKFNILSKIE |
| | | NLCDQLQKYKDKDLEKLFIERKNLSTVSHQVYGQWNIIRDALRMHLE |
| | | MNNKNIKEKDIDKYLDNDAFSWKEIKDSIKIYKEHVEDAKELNENGIIK |
| | | YFSAMSINEEDDEKEYSISLIKNINEKYNNVKSILQEDRTGKSDLHQDKE |
| | | KVGIIKEFLDSLKQLQWFLRLLYVTVPLDEKDYEFYNELEVYYEALLPLNS |
| | | LYNKVRNYMTRKPYSVEKFKLNFNSPTLLDGWDKNKETANLSIILRKN |
| | | GKYYLGIMNKENNTIFEYYPGTKSNDYYEKMIYKLLPGPNKMLPKVFFS |
| | | KKGLEYYNPPKEILNIYEKGEFKKDKSGNFKKESLHTLIDFYKEAIAKNED |
| | | WEVFNFKFKNTKEYEDISQFYRDVEEQGYLITFEKVDANYVDKLVKEG |
| | | KLYLFQIYNKDFSENKKSKGNPNLHTIYWKGLYDSENLKNVVYKLNGE |
| | | AEVFYRKKSIDYPEEIYNHGHHKEELLGKFNYPIIKDRRYTQDKFLFHVPI |
| | | TMNFISKEEKRVNQLACEYLSATKEDVHIIGIDRGERHLLYLSLIDKEGNI |
| | | KKQLSLNTIKNENYDKEIDYRVKLDEKEKKRDEARKNWDVIENIKELKE |
| | | GYMSQVIHIIAKMMVEEKAILIMEDLNIGFKRGRFKVEKQVYQKFEK |
| | | MLIDKLNYLVFKNKNPLEPGGSLNAYQLTSKFDSFKKLGKQSGFIFYVP |
| | | SAYTSKIDPTTGFYNFIQVDVPNLEKGKEFFSKFEKIIYNTKEDYFEFHCK |
| | | YGKFVSEPKNKDNDRKTKESLTYYNAIKDTVWVVCSTNHERYKIVRNK |
| | | AGYYESHPVDVTKNLKDIFSQANINYNEGKDIKPIIIESNNAKLLKSIAEQ |
| | | LKLILAMRYNNGKHGDDEKDYILSPVKNKQGKFFCTLDGNQTLPINAD |
| | | ANGAYNIALKGLLLIEKIKKQQGKIKDLYISNLEWFMFMMSR |
| 16 | Cas12 | MNNYDEFTKLYPIQKTIRFELKPQGRTMEHLETFNFFEEDRDRAEKYKI |
| | | LKEAIDEYHKKFIDEHLTNMSLDWNSLKQISEKYYKSREEKDKKVFLSE |
| | | QKRMRQEIVSEFKKDDRFKDLFSKKLFSELLKEEIYKKGNHQEIDALKSF |
| | | DKFSGYFIGLHENRKNMYSDGDEITAISNRIVNENFPKFLDNLQKYQE |
| | | ARKKYPEWIIKAESALVAHNIKMDEVFSLEYFNKVLNQEGIQRYNLALG |
| | | GYVTKSGEKMMGLNDALNLAHQSEKSSKGRIHMTPLFKQILSEKESFS |
| | | YIPDVFTEDSQLLPSIGGFFAQIENDKDGNIFDRALELISSYAEYDTERIYI |
| | | RQADINRVSNVIFGEWGTLGGLMREYKADSINDINLERTCKKVDKWL |
| | | DSKEFALSDVLEAIKRTGNNDAFNEYISKMRTAREKIDAARKEMKFISE |
| | | KISGDEESIHIIKTLLDSVQQFLHFFNLFKARQDIPLDGAFYAEFDEVHSK |
| | | LFAIVPLYNKVRNYLTKNNLNTKKIKLNFKNPTLANGWDQNKVYDYAS |
| | | LIFLRDGNYYLGIINPKRKKNIKFEQGSGNGPFYRKMVYKQIPGPNKNL |
| | | PRVFLTSTKGKKEYKPSKEIIEGYEADKHIRGDKFDLDFCHKLIDFFKESIE |
| | | KHKDWSKFNFYFSPTESYGDISEFYLDVEKQGYRMHFENISAETIDEYV |
| | | EKGDLFLFQIYNKDFVKAATGKKDMHTIYWNAAFSPENLQDVVVKLN |
| | | GEAELFYRDKSDIKEIVHREGEILVNRTYNGRTPVPDKIHKKLTDYHNG |
| | | RTKDLGEAKEYLDKVRYFKAHYDITKDRRYLNDKIYFHVPLTLNFKANG |
| | | KKNLNKMVIEKFLSDEKAHIIGIDRGERNLLYYSIIDRSGKIIDQQSLNVI |
| | | DGFDYREKLNQREIEMKDARQSWNAIGKIKDLKEGYLSKAVHEITKM |
| | | AIQYNAIVVMEELNYGFKRGRFKVEKQIYQKFENMLIDKMNYLVFKD |
| | | APDESPGGVLNAYQLTNPLESFAKLGKQTGILFYVPAAYTSKIDPTTGF |
| | | VNLFNTSSKTNAQERKEFLQKFESISYSAKDGGIFAFAFDYRKFGTSKTD |
| | | HKNVWTAYTNGERMRYIKEKKRNELFDPSKEIKEALTSSGIKYDGGQN |
| | | ILPDILRSNNNGLIYTMYSSFIAAIQMRVYDGKEDYIISPIKNSKGEFFRT |
| | | DPKRRELPIDADANGAYNIALRGELTMRAIAEKFDPDSEKMAKLELKH |
| | | KDWFEFMQTRGD |
| 17 | Cas12 | MASSHFISLDNSFSKFTNLYSLSKTLRFELVPTENTTVMLENNNVFKKD |
| | | QIIQVKYEKTKPFIDRLHREFIKEALSNYAVSGLQEYFEILRAGGKKANLD |
| | | SAKKQLRKHVVDQFNATASLWVSRHKDVGFKGEGIELLFKEAVFKLLK |
| | | EKYGTDMNALIEDNHGKQISIFDSWKGFTGYFDKFQQTRRNLYKDDG |
| | | KEGRVATRIIDQNLTRFCDNIFVYEKIKDKVSFIDVEKSFGKTCSEVFIPD |
| | | YYNTCLLQDGIDSYNEFIGGKPLENGEKVQGLNELINLYRQTTGDKVPY |
| | | FKKLEKQILGEKDEVFIDEITDEDFVPRVLAFYRTVDAKYKLFLKLLDDFV |
| | | TNQDVYELSQIYISKKGLQEKLYRWLTPSAREVYDEELFEVLKKAKKVN |
| | | NKDKQKVSGYVPDFVEVLYIKQALENIDAKLIWSDRYYSDGENEGIIDK |
| | | GFSSWKQFLVILNHEYRQLLSFEDHVIIDKELDFDKEVKQLTDTVEIVSQ |
| | | DKNARTVTYRGGYDVYKAKLAELGQSFEKDTCTKKVIKNFADSVLSMY |
| | | HFAMMFAVWDDTYPLDVFYTNNEFGYLLYYEDAYKNIVQEYNKLRN |
| | | YLTKKPYSTEKWKLNFENPTLAAGFDKNKESDNSTVILRQGDKYFLGV |
| | | MKKGFNKlFDNSQISQTGNSPEAYFEKMVYKYTKDVVTGIPKSSTQVK |
| | | EVQEHFRNSDEDFFLEECSSVGNFIVPLKITKEIFDLNNKVYAKEDISQA |
| | | MYRWALNTDEEKNYVKSFQKSYLSLGGSPELYCKSVTLWIGFCLNFLK |
| | | SYPSAAYFDYSQLRQASDYESVDECYQELNNAGYTILFQNVSEKYVRV |
| | | KNKNGELYLFQIKNKDWNEGSTGKKNLHTLYFESLFSKENAKQGFPFK |
| | | LSGNAELFFRPGSIEQTYERRNFPREIPLKRRYSKDGIFFHIPVQVNRTK |
| | | VGSPNQFNKEVNDFLAGNPNINIIGVDRGEKHLVYYSVISQNGEKIDG |
| | | GSFNEINGQDYHDKLEKRAKEREQQRRDWETVEGIKDLKKGYISQVV |
| | | KKLADLAIEHNAIIVMEDLNMRFKQIRGGIEKSVYQQLEKALIDKLSFLV |
| | | NKGEVDPQKAGHLLKAYQLTAPIDAFKDMGKQTGIMFYTQAAYTSKI |
| | | DPVTGWRPHLYLKYSSVEKAKDDISRFTKIAYKNDRFEFTYNITDFRTQ |
| | | KEWPLKTEWTVCSCVERFRWNKKLANGKGDYEHYPNVTDDFKKLFD |
| | | SVGINYLQENIKSQVVNLDENTNVEFFREFIKLFALVCQIRNTNSEEAG |
| | | NLNDFILSPVEPFFDSRSAEDFGKGLPSNGDENGAYNIARKGMIILNTL |
| | | STFKNDHGSCEGLSWGDLYISDTQWDDFAQSFHG |
| 18 | Cas12 | MDAKEFTGQYPLSKTLRFELRPIGRTWDNLEASGYLAEDRHRAECYPR |
| | | AKELLDDNHRAFLNRVLPQIDMDWHPIAEAFCKVHKNPGNKELAQD |
| | | YNLQLSKRRKEISAYLQDADGYKGLFAKPALDEAMKIAKENGNESDIE |
| | | VLEAFNGFSVYFTGYHESRENIYSDEDMVSVAYRITEDNFPRFVSNALI |
| | | FDKLNESHPDIISEVSGNLGVDDIGKYFDVSNYNNFLSQAGIDDYNHII |
| | | GGHTTEDGLIQAFNVVLNLRHQKDPGFEKIQFKQLYKQILSVRTSKSYI |
| | | PKQFDNSKEMVDCICDYVSKIEKSETVERALKLVRNISSFDLRGIFVNKK |
| | | NLRILSNKLIGDWDAIETALMHSSSSENDKKSVYDSAEAFTLDDIFSSVK |
| | | KFSDASAEDIGNRAEDICRVISETAPFINDLRAVDLDSLNDDGYEAAVS |
| | | KIRESLEPYMDLFHELEIFSVGDEFPKCAAFYSELEEVSEQLIEIIPLFNKA |
| | | RSFCTRKRYSTDKIKVNLKFPTLADGWDLNKERDNKAAILRKDGKYYLA |
| | | ILDMKKDLSSIRTSDEDESSFEKMEYKLLPSPVKMLPKIFVKSKAAKEKY |
| | | GLTDRMLECYDKGMHKSGSAFDLGFCHELIDYYKRCIAEYPGWDVFD |
| | | FKFRETSDYGSMKEFNEDVAGAGYYMSLRKIPCSEVYRLLDEKSIYLFQI |
| | | YNKDYSENAHGNKNMHTMYWEGLFSPQNLESPVFKLSGGAELFFRK |
| | | SSIPNDAKTVHPKGSVLVPRNDVNGRRIPDSIYRELTRYFNRGDCRISD |
| | | EAKSYLDKVKTKKADHDIVKDRRFTVDKMMFHVPIAMNFKAISKPNL |
| | | NKKVIDGIIDDQDLKIIGIDRGERNLIYVTMVDRKGNILYQDSLNILNGY |
| | | DYRKALDVREYDNKEARRNWTKVEGIRKMKEGYLSLAVSKLADMIIE |
| | | NNAIlVMEDLNHGFKAGRSKIEKQVYQKFESMLINKLGYMVLKDKSID |
| | | QSGGALHGYQLANHVTTLASVGKQCGVIFYIPAAFTSKIDPTTGFADLF |
| | | ALSNVKNVASMREFFSKMKSVIYDKAEGKFAFTFDYLDYNVKSECGRT |
| | | LWTVYTVGERFTYSRVNREYVRKVPTDIIYDALQKAGISVEGDLRDRIA |
| | | ESDGDTLKSIFYAFKYALDMRVENREEDYIQSPVKNASGEFFCSKNAG |
| | | KSLPQDSDANGAYNIALKGILQLRMLSEQYDPNAESIRLPLITNKAWLT |
| | | FMQSGMKTWKN |
| 19 | Cas12 | MVNKQNERGDFDDLTNLYEISKTLRFELVPVGETDRMLKEENVFKVD |
| | | ENIKRKYQQTKLFFDRIHREFAKEALSVEGILSELEEYLAIFIEWRKDKKI |
| | | HEKTLNQKEKELRKQVVSAFNAMANKWIERYGDVNLKKKNVEFLFEE |
| | | GIFRVLKERYGEEDGSTITASDTGEVFSIFDSWKGFTGYFAKFFETRKNF |
| | | YKDDGTATAIATRIVDENLRRFCDNLIVAQRLTENIDFSEVENNFQIKIK |
| | | EVLFMEFYNKCLLQDDIDFYNKVIGGETLKTGEKLKGINELVNLHRHKT |
| | | GEKLPFLKTLDKQILGRKEQFLDEIESEEELLEKLKDFQNVATKKIKVIKSL |
| | | FGDFVENNENYDLEKIYISKKAFNTISRKWTGETEQFEKLLFESMKSDK |
| | | PAGLKYDKKENNYKFPDFIAVSYIKDALENFSGEQKFWKDRYYIELELD |
| | | NQVVWKQFLDIFYWEFSSLFKRSFVNKETGEISEVGCDIFEKKFINLIDD |
| | | FEYNQKSKILIKDFADSVLSVYQMANYFSLEKKRKWSTEFETDSKFYDD |
| | | SEIGFRNCFYEDVFEGIVQ.VYNKLRNYLTKKPFSEEKWKLNFENPTLAA |
| | | GWDKNKEKDNSTVILRKDEKYFLAIMKKGNNVIFDDRNKALFSQNLE |
| | | HGKYEKWYKFAKDVTLGIPKSTTQTKSVIAHFKNSDEDYQITNGSAVG |
| | | DFLEPLVVTKRIFELNNKIYSKNNLGKVLYRSEVSKDKQKEYIKLFQKKYL |
| | | VLGGNKNLYRDAVKEWIDFCKSFIKVYPSYKYFDFSLLKEAVEYNSVDE |
| | | FYKELNSYGYAISFQDISCDYIEEKNKNGELYLFQIKNKDWNKGSTGMK |
| | | NLHTLYFESLFSEENIKNNFVTKLNGGAEIFYRPKTSKEKLGRKKIVRNG |
| | | QEVFVVNHKRYSEDKIFFHCSIALNRGKGKLLKFNARINDLLANNPDIN |
| | | VIGVDRGEKHLAYYSIIDQKCKILDSGTLNEVGAKVDYHEKLSNRAKKR |
| | | EDGRRDWGWGQIEDIKNLKKGYVSQVVHKLAELIIKYNAILVFEDLN |
| | | MRFKQIRGGIEKSIYQQLEKALIDKLNFLVKKGEKDSKSAGHLLKAYQL |
| | | AAPFETFDKMGKQTGVIFYTQASYTSKIDPITGWRPNLYLKHSNANDS |
| | | QKKIAKFSRIEFINDRFEFEYDLKKFIEMKEVPENTKWTLCSCVQRYRW |
| | | NRKLNANKGGYDSYNDLTKNFKALFESVGIDIKKNIKEQIVKMEIKGNE |
| | | KFFKSFIFYWQLLCQIRNTDELKKGDDNDFILSPVEPFFDSRKKNGDDL |
| | | PKNGDDNGAYNIARKGVIVLNKISEFSKQNGNCEKCGWKELYVSAKD |
| | | WDDFVQAK |
| 20 | Cas12 | MQNKQSFADFTNLYSLSKTLRFELKPIGQTQAMLDENKIFEVDENRKK |
| | | AYDKTKPYFDRLHREFINESLSNAQLKGISEYFETFKQFRSNQNNKDLK |
| | | ELINKQQKFLRHQIVTLFDENGKHWATTKYAHLKIKKKNLDILFDEQVF |
| | | YILKERYGSEKETQLVDKETGAVTSIFDNWKGFTGYFTKFFETRKNFYK |
| | | SDGTSTALATRIIDQNLNRFFDNLETFHKIKDKIDVKEVEIFFKLKADNV |
| | | FSIDFYNQCLLQNGIDKYNDFLGGQTLENGEKQKGINEIINKYRQDNK |
| | | DQKLPFLKKLDKQLSEKDRFINEIESKEEFFQVLTEFYQSATVKVTIIKTL |
| | | LNDFVHNTDKYKLEKIYLTKEAFNTIANKWTDETQIFEDNLDLVLKNKK |
| | | ITAKQDFIPLAYIKEALEVIEKDRKFFKDRYYNDPQIGFFPDQSYWEQFL |
| | | AILNFEFMTHFQRVAKDKITGKKIELGYFVFEKRIKELLDSDPSLNSQSKI |
| | | IIKEFADEVLHIFQMAKYFALEKKREWKGDYYQLDDQFYNHIDYGFKD |
| | | QFYENAYEKIVQPYNKIRNYLTKKPYSDVKWKLNFGNPTLANGWDKN |
| | | KEADNTAVILKKDGNYYLGVMKKGKNKIFSDQNKEKYKAYNSAYYEKL |
| | | VYKLFPDPSKMFPKVCFSKKGLNFFQPSEEILRIYKNNEFKKGNTFSISS |
| | | MQKLIAFYIDCLGLYEGWKHYEFKNIKDVRQYKENIGEFYADVAESGY |
| | | KLWFEKISEEYITQKNQLGELFLFQIYNKDFAKKTTGRKNLHTIYFEELFS |
| | | QTNIDNNFPFKLNGQAELFYRPKSLEKIEEKRNFKRSIVNKKRYTQNKIF |
| | | FHVPITLNRTSENIGRFNVRVNNFLANNSNVNIVGVDRGEKNLAYYSII |
| | | KQNGEVLKSGSLNIINGVDYHALLTDRAQRREQERRNWQDVESIKDL |
| | | KRGYISQVVHELVSLAIKYNAIIVMEDLNMRFKQIRGGIEKSTYQQLEK |
| | | ALIEKLNFLVNKEETDSNQAGNLLNAYQLTAPFKTFKDMGKQTGIIFYT |
| | | QASYTSKIDPLTGWRPNIYLRYSNAKQAKADILMFTNIYFSEKKDRFEF |
| | | TYDLEKIDDKRKDLPIKTEWTVCSNVERFSWEKSLNNNKGGYVHYPIQ |
| | | DSNGEESITSKLKKLFMDFGIDLTDIKTQIESLDTNKKDNANFFRKFIFYF |
| | | QLICQIRNTQVNKSDDGNDFIFSPVEPFFDSRFADKFRKNLPKNGDEN |
| | | GAYNIARKGLIILHKISDYFVKEGSTDKISWKDLSISQ.TEWDNFTTDK |
| 21 | Cas12 | MDKQKNKLQNFTNLYELSKTLRFELKPVGETQHLLEENKVFGIDGNIK |
| | | KKYEATKPFFDRLHRKFVKEALVNIALGGLDNYLEVYKKFTNDRKDKEN |
| | | QKELEKQEKLLRKQIKIFFDSQANQWKEKYNKINFKKSGLNILFEESIFQ |
| | | LLKEIYGKEDDAFLKNDDNEFIFDKDGNKISIFDSWKGFTGYFKKFFETR |
| | | KNFYKDDGTSTAIATRIIDQNLRRFCDNIFIYNKIKNKLDFSSLEKEQDV |
| | | VLEEIFTTAYYMDCILQDDIDLYNGVLGGETLDDGTKIKGLNEIINKYRQ |
| | | DNKGDKIPFFKKLDKQILSEKDRKFLDEIESEEELAELLKIFINNTEAKVKV |
| | | FDELVNQLCVNDSDFELDKIYISKEAFNTISHKWTNQTHEFERVLFEEM |
| | | KPDKITGLDYKKAEDKYKFPDFIALKYIIKSLNTLDKDSEFWKSHYYKTEE |
| | | NQNAILSLEEKVGEQFLQIYKYELQRLHSRNVNVENKDGKMKEKEIGL |
| | | DYSLTTVKELLKNFKLTDKSKIIIKDFADNVLQYYQLAKYFSVEKNREWN |
| | | YTKLELADFYINPDFGYEIFYGNAYEEIIQJYNKLRNYLTKKPFSEEKWKL |
| | | NFENPTLAGGWDKNKERGNATVILRKNEKYYLGIMAKGYNDIFTDKN |
| | | KDKFDGEGYEKMVYKLFPGPNKMMPKVCFSKKGLDFFEPSEKIIDIYK |
| | | DGKFKQGDTFSIDSMQQLIDFYKRALREYNGWKMYDFSKLKDTNDYT |
| | | TNIGEFYNDVACAGYKVWFDNISEEYIQEKNENGELYLFEIHNKDWNL |
| | | KDEKKKTGTKNLHTLYFESLFSDENALRDFVMKLSGEAELFFRPKTNAD |
| | | KLGYRKDKKGNKVVKNKRYSEDKMFLHLSINLNRGKGQAFWFNRNIN |
| | | NFLANNSDINVIGIDRGEKHLAYYSVISQQGEILDNGSLNEIAGVDYYA |
| | | KLSKRAKEREGQRKDWQAVSDIKNLKKGYISQVVRKLADLAIEHNAIIV |
| | | LEDLNMRFKQIRGGIEKSIYQQLEKALIEKLNFLVNKKEIDSDKAGNLLR |
| | | AYQLTAPFETFQKMGKQTGIIFYTQASYTSKIDPLTGWRPNLYLKKGN |
| | | AKINKEQIEKFSKIEFTNNRFEITYDLKNFGDKKKKYPQKTKWTLCSSVE |
| | | RWRWDRKLNNNKGGYIHYEDLTTEFKSLFEKFEIDIEGDILEQIKTIDEN |
| | | DRNNARLFSGFIYLWGLLSQIRNTDGELDEKIKKLEREDKNEEISEKEKF |
| | | DVDFILSPVEPFFDSRTPEKFGENLPKNGDDNGAYNIARKGHTLERIKK |
| | | FYELSDKEREKLKYPDLFITNAEWDDFATKRDS |
| 22 | Cas13 | MDNNTTLEKTELGLGITYNHDKVEDKHYFGGFFNLAQNNIDLVAQEF |
| | | KKRLLVQGKDSINIFSNYFSDQCSITNLERGIKVLSEYFPVIFYFDLDENN |
| | | KSKSIRQH11LLLDTINNLRNYYTHYYHKKVIIDDALYPLLDTILLKVVLEIK |
| | | KKKLKEDKTKQLLKKGLEKEMAILFNLMKKEQKEKKIKGWNIDKNIKG |
| | | AVLNRAFSHLLYNDGISDYRKSKSNTEDENLKDTLSESGILFLLSFFLNKK |
| | | EQEQLKANIKGYKGKIASIPDEEITLKNNSLRNMATHWTYSHLTYKGLK |
| | | HRIKTDHEKETLLVNMVDYLSKVPNEIYQNLSEQNKSLFLEDINEYMR |
| | | DNEENNDSSEASRVIHPVIRKRYENKFAYFAIRFLDEFAEFPTLRFMVN |
| | | VGNYIHDNRKKDIGGTSLITNRTIKQQINVFGNLTEIHKKKNDYFEKEE |
| | | NKEKILEWELFPNPSYHFQKENIPIFIDLEKSKETNELAKEYAKEKKKIFG |
| | | SSRKKQQNTAKKNREAIINLVFDKYKTSDRKTVTFEQPTALLSFNELNA |
| | | FLYAFLVENKTGKELEKIIIEKIANQYQILKNCSSTVDKTNDSIPKSIKKIA |
| | | HPTTDSFYSEGKKIDIEKLERDIKIEIEKTNEKLETIKENETSAKNYKRNER |
| | | DIQKRKLYRKYVFFTNEIGIEATWITNDILRFLDNKENWKGYQHSELQK |
| | | FISQYDNYKKEALGLLESEWNLESEAFFGQKLKRIFQSNFTFETFYKKYL |
| | | DNRKDTLETYLSAIENLKTMTDVPPKILKKSWAELFRFFDKKIYLLSTIET |
| | | KINELITKPINLSRGVFDEKPTFINGKSPNKENDQHLFANWFIHAKEQTI |
| | | FQDFYNLALETPKEINNLKKQNYKLERSINNLKIEDIYIKQMVDFLYQKL |
| | | FEQSFKGSLQDLYTSKEKREVEKSKAKNEQ.TPDESFIWKKQVEINALN |
| | | GRIIAKTKIKDIGKFKNLLTDNKITHLISYDNRIWNFSLDNDGDTTKKLYS |
| | | LNTELESYERIRREKLLKQIQEFEQFLLKQETEYSAERKHPEKFEKDGNP |
| | | NFKKYIIEGMLNKITPVNEIEELEILKSKEDVFKIDFNEIVKLNNESIKKGY |
| | | LLIMIRNKFAHNQLIDKNLFTFSLQLYSKNENENFSEYLDKVCQKIIQEFI |
| | | EKLK |
| 23 | Cas13 | MNETDYLAKRLEYNYASIEDKHYFGGYFNLAQNNINDLSKAFKEKFG |
| | | MKPKSCILDFFTQDKAIAEYQLGVEFLQKNLPVIRYLYLPTSHKRFENVP |
| | | KNQLISEQRNYFKNSLKVLKNLIRDYRNFYTHHFHKPIPVFPETYKLLDD |
| | | LFLAVANDVKKHRMKTDASKQLLKKGLIEELAQLEKLKLEDLKKLKREG |
| | | KKVNLNDKEAITNAILNDSFSHLLPKENTISKYYSAVPTEDIDTENGVTIS |
| | | ESGIIFLLGLFLTKKQSEDLRSRVKGFKAKLIVNPENPINKKNNSLKYMA |
| | | THWVFGYLGFKGLKNRFTTTFTKDTLLAQIVDELSKVPDELYQVLPEEL |
| | | KNEFLEDMNEYLKEENSESLDKATVIHPVIRKRYENKFAYFALRFLDEFV |
| | | DFPTLRFQLHLGNYVHDKREKPIEGTKYVTERIVKEKIKAFAKLSEAAQL |
| | | KQKYFEEKENHQSIGLQLYPNPSYNFVGNNIPIHLNLNEHFFPKEVKIV |
| | | AGRLKKRNSSYKSDHPEEYKVRTDNKIKPDAILQDLGKPEKLAPVAML |
| | | SLNELPALLHLVLTKKTPEEIEIIIAQKIAERYNVLTNYKAGDDISKGQITK |
| | | NLLKAKQKKEVNLDKLQLAIEKEIAVTNDKLQTIALHIKERNDPKQKRK |
| | | YVFTNKEIGLQVTWLANDLKRFMPKGSRQNWRGQHHSQLQKSLAFY |
| | | DIQPKEPLSLLEEVWDFKNEAYLWNNGIRRSFDKRDFISFYTSYLNNRK |
| | | ETFQRFKDQLNGIRSNKKILDKFIKQQHLWNLFHKRLYVIDTIEEQVEKL |
| | | LVKPMQFPKGVFDHKPTYIKGKSIQENPECFADWYVAWNQHTDYQK |
| | | FYSWDRDYKSAYLSGEQEKTEKRFIRVQGSKINKVKQQDVLLAKMASII |
| | | FNELYLPEDAEHLDLNLSDIYKTQTERKAEIEAALIQSHKTTGDNSANIIK |
| | | STSAWTLTVPYCSKNIYEPQVKLKELGKFKKFIASQKVQTLFEYKPQKI |
| | | WNKTELEEVLELKANSYEVIRRDYLLKSIQEFEKYMIKKLPTLIDTNEHP |
| | | NFNKYLTTFLKSLELVSEEDAKWLISKKDFDTTPIDELKKQSKIMEKAFLL |
| | | VMIRNKFSHNQLPRKIYYDEIYKNVPNAVSINFNELFLEYTNQTILEFK |
| 24 | Cas13 | MESIIGLGLSFNPYKTADKHYFGSFLNLVENNLNAVFAEFKERISYKAK |
| | | DENISSLIEKHFIDNMSIVDYEKKISILNGYLPIIDFLDDELENNLNTRVKN |
| | | FKKNFIILAEAIEKLRDYYTHFYHDPITFEDNKEPLLELLDEVLLKTILDVK |
| | | KKYLKTDKTKEILKDSLREEMDLLVIRKTDELREKKKTNPKIQHTDSSQIK |
| | | NSIFNDAFQGLLYEDKGNNKKTQVSHRAKTRLNPKDIHKQEERDFEIP |
| | | LSTSGLVFLMSLFLSKKEIEDFKSNIKGFKGKVVKDENHNSLKYMATHR |
| | | VYSILAFKGLKYRIKTDTFSKETLMMQMIDELSKVPDCVYQNLSETKQK |
| | | DFIEDWNEYFKDNEENTENLENSRVVHPVIRKRYEDKFNYFAIRFLDEF |
| | | ANFKTLKFQVFMGYYIHDQRTKTIGTTNITTERTVKEKINVFGKLSKMD |
| | | NLKKHFFSQLSDDENTDWEFFPNPSYNFLTQADNSPANNIPIYLELKN |
| | | QQIIKEKDAIKAEVNQTQNRNPNKPSKRDLLNKILKTYEDFHQGDPTAI |
| | | LSLNEIPALLHLFLVKPNNKTGQQIENIIRIKIEKQFKAINHPSKNNKGIP |
| | | KSLFADTNVRVNAIKLKKDLEAELDMLNKKHIAFKENQKASSNYDKLLK |
| | | EHQFTPKNKRPELRKYVFYKSEKGEEATWLANDIKRFMPKDFKTKWK |
| | | GCQHSELQRKLAFYDRHTKQDIKELLSGCEFDHSLLDINAYFQKDNFE |
| | | DFFSKYLENRIETLEGVLKKLHDFKNEPTPLKGVFKNCFKFLKRQNYVTE |
| | | SPEIIKKRILAKPTFLPRGVFDERPTMKKGKNPLKDKNEFAEWFVEYLE |
| | | NKDYQKFYNAEEYRMRDADFKKNAVIKKQKLKDFYTLQMVNYLLKEV |
| | | FGKDEMNLQLSELFQTRQERLKLQGIAKKQMNKETGDSSENTRNQTY |
| | | IWNKDVPVSFFNGKVTIDKVKLKNIGKYKRYERDERVKTFIGYEVDEK |
| | | WMMYLPHNWKDRYSVKPINVIDLQIQEYEEIRSHELLKEIQNLEQYIY |
| | | DHTTDKNILLQDGNPNFKMYVLNGLLIGIKQVNIPDFIVLKQNTNFDKI |
| | | DFTGIASCSELEKKTIILIAIRNKFAHNQLPNKMIYDLANEFLKIEKNETY |
| | | ANYYLKVLKKMISDLA |
| 25 | Cas13 | MEDKTTGAGISYDHTLMEDKHFFGGFLNLAQNNIDALLKAFKERFNV |
| | | RYQSKQFAEVCFSDKLPDQDYLDRTLFLETHLPFIKYIGGKEANNRGTF |
| | | RKNITLFFESIEQLRNFYTHYYHKPILFPEELYENLDRIFVEVSKEVKTHKV |
| | | KNDQTRHLLTKNLANELDIRYKKNVEKLKELKAQGKKVNIHDKEAIKNS |
| | | VLNNAFNHLIYKKEEDVFATEAYKSKYNLEDPSKNGISLSQSGLLFLLSM |
| | | FLNKKDIEALKSRVKGFKAKIIRDGEENISGLKFMATHWVFSSLSFKNV |
| | | KHKLSTDFHKETLLIQIVDELSKVPDEVYKTFDKQTQEEFIEDINEYMKV |
| | | GNKDLSLEESTVIHPVIRKRYDNKFNYFALRFLDEFAGFPTLRFQVHIGN |
| | | YIHDRRIKNIDGTAFQTERSVKERIKVFGKLSQMSNLKAEYVSGLMDEP |
| | | VDTGWEIFPNPSYNIIENNIPIYIEMGDHFNDEVLQSKMARKKQKPEE |
| | | LKDRNSAKASKESMIQTLQNDKGLMDVITVSPTAQLSLNELPAILYELL |
| | | VKKTPAKTIEKKLVGKLNQRLKEIKNYNPEKPLPASQISKRLRLNREEGSI |
| | | NTKKIIALLQKELNYTQEKLDLLEKNRKEYGKKVDGKILRKYVFGLKEIG |
| | | NLATDMAMDIKRFMPANVRKEWKGYQHSQLQQSLAFYDKRPEEAF |
| | | NILQEVWDINREKSLWDTWILNAFQTSGNFERFFELYHEGRKKYIQQ |
| | | QLENIDRYTDNKKFLQKFINQQFPTNFLEKRLYTLESLEIEKLKILSKPFIL |
| | | PRGTFDEKPTFIMGEKVTENPELFADWYTYGYQQHEFQKFYSWPRDY |
| | | KDLLQNEQKRDPDFAENKKGLSDLKQLELLQLKQD111KKIKTQDLYLKLI |
| | | MDALFIEVFGQEADISLNDLYLTQEERLEKEKLALKQHQRVEGDDSPN |
| | | VIKDNFIWSKTMPYKHDKIYEPQVRLKDFGKFKHFLLDDKVAKILSYDL |
| | | QETWNKNELEIQINTGQDSYEVIRREELLKEIQLLEKQILETFSHTLDEH |
| | | PKEFEDEKGNPNFKMYMANGVIRKGSSTTAKDEADWLEHEKDFDNL |
| | | SLEIFNSKSEITQLTFLIVLIRNKFGHNQLPIKQFYEIIQNEYSITGETISRLY |
| | | LNFIIYAKARLKDLM |
| 26 | Cas13 | MEEKLGKGVEYNPFKKEDKYYFGGYFNLAENNINEVFKEVKKRLGETN |
| | | SSSNIELLNNVFRKEMSLVDYEKWVNAFADYFPIVNYLDRETIKKGEKV |
| | | VEVPREKRIECFRDMFKGLINTISQLRHYYTHYHHEPIEIDDKILSFLDEV |
| | | LFNTIITTKNKYLKTDKTKELIKDSLQEELDILCKLKVKYLESKRKRFDRKD |
| | | KGAIENAVYNDVFRRFIYKDEKGNESLKDIIRTKQIKVHQNSSYLELPISS |
| | | SGIIFLLSLFLNKKEVESLKSNIRGYKGKSKSEETTPEKNGLLFMTTHRIYS |
| | | VLAYKGLKKRIKTSVKGDKETLLMQMIDEVSKVPHCIYQNLDQTLQAT |
| | | FIEDWNEYFKDNEENEENLENSRVLHPVIRKRYEDKFNYFAIRFLDEYA |
| | | EFPSLRFQVNLGNYVHHKATKKFGNSEVTTERVIKDKITVFGRLSEVNK |
| | | AKADFFKNETELDPAWELFPNPSYEFPKEKGNNDKDAGKIGIQVKLLN |
| | | KDIEAVLNESKNTLNNKTRKSDKISKKEIINKIVQINDDTKYNNKNIIYQ |
| | | GNAIAYLSLNDIHSLLYELLVIGTKGDKLERKVVEKIQQQVTEIRNKDTS |
| | | AKILSKYKDSEESNTIDKKKLVIDLKYEYDKLQDLLKEHKNREEDYIQTKK |
| | | KKKDSPKRKYILYHNEKGQVAVWLSNDIKRFMPQNFKEKWKGYQHS |
| | | EFQKSLAYYETNKEMLKIILQDLDLEQFPFDIKSCFYKNTLEDFYNRYLSL |
| | | RISYLENVIDRVECFSNEPKAFKSVLKECFVFLKKQNYTNHSLDEQVKKI |
| | | LANPIFIERGFLDTKPTMIQGVKFSENKGCFADWFVHYKEYEHYOKFY |
| | | DTNLYPVESIEDKERQKLEATIKKQQKNDVFTLLMIKKIFNDLFNQDFE |
| | | ANLYEMYQSKEEREKNQLVAKETQNRNLNFIWNKPIAIDLFDGKVKID |
| | | EVKLKDVGSFRKYENDKRVQTFITYIPEIQWIPYLPNTWEGINLPVNVIE |
| | | ROIDRYEKVRSEELLKEVQAIEKYIYEQVNDKTELLONGNQNFKNYLVN |
| | | GLLKQIQGIDVSNFKFINQQKFETINVKDLDNEASALEQKVYVLINIRN |
| | | QFSHNQFPKSAFYQFCQKILSIEEDELFADYYLRLFKLLRNELLD |
| 27 | Cas13 | MNTRVTGMGVSYDHTKKEDKHFFGGFLNLAQDNITAVIKAFCIKFDK |
| | | NPMSSVQFAESCFTDKDSDTDFQNKVRYVRTHLPVIGYLNYGGDRNT |
| | | FRQKLSTLLKAVDSLRNFYTHYYHSPLALSTELFELLDTVFASVAVEVKQ |
| | | HKMKDDKTRQLLSKSLAEELDIRYKQQLERLKELKEQGKNIDLRDEAGI |
| | | RNGVLNAAFNHLIYKEGEIAKPTLSYSSFYYGADSAENGITISQSGLLFLL |
| | | SMFLGKKEIEDLKSRIRGFKAKIVRDGEENISGLKFMATHWIFSYLSFKG |
| | | MKQRLSTDFHEETLLIQIIDELSKVPDEVYHDFDTATREKFVEDINEYIR |
| | | EGNEDFSLGDSTIIHPVIRKRYENKFNYFAVRFLDEFIKFPSLRFQVHLG |
| | | NFVHDRRIKDIHGTGFQTERVVKDRIKVFGKLSEISSLKTEYIEKELDLDS |
| | | DTGWEIFPNPSYVFIDNNIPIYISTNKTFKNGSSEFIKLRRKEKPEEMKM |
| | | RGEDKKEKRDIASMIGNAGSLNSKTPLAMLSLNEMPALLYEILVKKTTP |
| | | EEIELHKEKLDSHFENIKNYDPEKPLPASQISKRLRNNTTDKGKKVINPE |
| | | KLIHLINKEIDATEAKFALLAKNRKELKEKFRGKPLRQTIFSNMELGREA |
| | | TWLADDIKRFMPDILRKNWKGYQHNQLQQSLAFFNSRPKEAFTILQD |
| | | GWDFADGSSFWNGWIINSFVKNRSFEYFYEAYFEGRKEYFSSLAENIK |
| | | QHTSNHRNLRRFIDQQMPKGLFENRHYLLENLETEKNKILSKPLVFPR |
| | | GLFDTKPTFIKGIKVDEQPELFAEWYQYGYSTEHVFQNFYGWERDYN |
| | | DLLESELEKDNDFSKNSIHYSRTSQLELIKLKQDLKIKKIKIQDLFLKLIAG |
| | | HIFENIFKYPASFSLDELYLTQEERLNKEQEALIQSQRKEGDHSDNIIKD |
| | | NFIGSKTVTYESKQISEPNVKLKDIGKFNRFLLDDKVKTLLSYNEDKVW |
| | | NKNDLDLELSIGENSYEVIRREKLFKKIQNFELQTLTDWPWNGTDHPE |
| | | EFGTTDNKGVNHPNFKMYVVNGILRKHTDWFKEGEDNWLENLNET |
| | | HFKNLSFQELETKSKSIQTAFLIIMIRNQFAHNQLPAVQFFEFIQKKYPEI |
| | | QGSTTSELYLNFINLAVVELLELLEK |
| 28 | Cas13 | METQILGNGISYDHTKTEDKHFFGGFLNTAQNNIDLLIKAYISKFESSPR |
| | | KLNSVQFPDVCFKKNDSDADFQHKLQFIRKHLPVIQYLKYGGNREVLK |
| | | EKFRLLLQAVDSLRNFYTHFYHKPIQLPNELLTLLDTIFGEIGNEVRQNK |
| | | MKDDKTRHLLKKNLSEELDFRYQEQLERLRKLKSEGKKVDLRDTEAIRN |
| | | GVLNAAFNHLIFKDAEDFKPTVSYSSYYYDSDTAENGISISQSGLLFLLS |
| | | MFLGRREMEDLKSRVRGFKARIIKHEEQHVSGLKFMATHWVFSEFCF |
| | | KGIKTRLNADYHEETLLIQLIDELSKVPDELYRSFDVATRERFIEDINEYIR |
| | | DGKEDKSLIESKIVHPVIRKRYESKFNYFAIRFLDEFVNFPTLRFQVHAG |
| | | NYVHDRRIKSIEGTGFKTERLVKDRIKVFGKLSTISSLKAEYLAKAVNITD |
| | | DTGWELLPHPSYVFIDNNIPIHLTVDPSFKNGVKEYQEKRKLQKPEEM |
| | | KNRQGGDKMHKPAISSKIGKSKDINPESPVALLSMNEIPALLYEILVKK |
| | | ASPEEVEAKIRQKLTAVFERIRDYDPKVPLPASQVSKRLRNNTDTLSYN |
| | | KEKLVELANKEVEQTERKLALITKNRRECREKVKGKFKRQKVFKNAELG |
| | | TEATWLANDIKRFMPEEQKKNWKGYOHSQLQQSLAFFESRPGEARS |
| | | LLOAGWDFSDGSSFWNGWVMNSFARDNTFDGFYESYLNGRMKYFL |
| | | RLADNIAQQSSTNKLISNFIKQQMPKGLFDRRLYMLEDLATEKNKILSK |
| | | PLIFPRGIFDDKPTFKKGVQVSEEPEAFADWYSYGYDVKHKFQEFYAW |
| | | DRDYEELLREELEKDTAFTKNSIHYSRESQIELLAKKQDLKVKKVRIQDL |
| | | YLKLMAEFLFENVFGHELALPLDQFYLTQEERLKQEQEAIVQSQRPKG |
| | | DDSPNIVKENFIWSKTIPFKSGRVFEPNVKLKDIGKFRNLLTDEKVDILL |
| | | SYNNTEIGKQVIENELIIGAGSYEFIRREQLFKEIQQMKRLSLRSVRGMG |
| | | VPIRLNLK |
| 29 | Cas13 | MENQTQKGKGIYYYYTKNEDKHYFGSFLNLANNNIEQIIEEFRIRLSLK |
| | | DEKNIKEIINNYFTDKKSYTDWERGINILKEYLPVIDYLDLAITDKEFEKID |
| | | LKQKETAKRKYFRTNFSLLIDTIIDLRNFYTHYFHKPISINPDVAKFLDKN |
| | | LLNVCLDIKKQKMKTDKTKQALKDGLDKELKKLIELKKAELKEKKIKTW |
| | | NITENVEGAVYNDAFNHMVYKNNAGVTILKDYHKSILPDDKIDSELKL |
| | | NFSISGLVFLLSMFLSKKEIEQFKSNLEGFKGKVIGENGEYEISKFNNSLK |
| | | YMATHWIFSYLTFKGLKQRVKNTFDKETLLMQMIDELNKVPHEVYQT |
| | | LSKEQQNEFLEDINEYVQDNEENKKSMENSIVVHPVIRKRYDDKFNYF |
| | | AIRFLDEFANFPTLKFFVTAGNFVHDKREKQIQGSMLTSDRMIKEKINV |
| | | FGKLTEIAKYKSDYFSNENTLETSEWELFPNPSYLLIQNNIPVHIDLIHNT |
| | | EEAKQCQIAIDRIKCTTNPAKKRNTRKSKEEIIKIIYQKNKNIKYGDPTAL |
| | | LSSNELPALIYELLVNKKSGKELENIIVEKIVNQYKTIAGFEKGQNLSNSLI |
| | | TKKLKKSEPNEDKINAEKIILAINRELEITENKLNIIKNNRAEFRTGAKRK |
| | | HIFYSKELGQEATWIAYDLKRFMPEASRKEWKGFHHSELQKFLAFYDR |
| | | NKNDAKALLNMFWNFDNDQLIGNDLNSAFREFHFDKFYEKYLIKRDEI |
| | | LEGFKSFISNFKDEPKLLKKGIKDIYRVFDKRYYIIKSTNAQKEQLLSKPIC |
| | | LPRGIFDNKPTYIEGVKVESNSALFADWYQYTYSDKHEFQSFYDMPRD |
| | | YKEQFEKFELNNIKSIQNKKNLNKSDKFIYFRYKQDLKIKQIKSQDLFIKL |
| | | MVDELFNVVFKNNIELNLKKLYQTSDERFKNQLIADVQKNREKGDTSD |
| | | NKMNENFIWNMTIPLSLCNGQIEEPKVKLKDIGKFRKLETDDKVIQLLE |
| | | YDKSKVWKKLEIEDELENMPNSYERIRREKLLKGIQEFEHFLLEKEKFDG |
| | | INHPKHFEQDLNPNFKTYVINGVLRKNSKLNYTEIDKLLDLEHISIKDIET |
| | | SAKEIHLAYFLIHVRNKFGHNQLPKLEAFELMKKYYKKNNEETYAEYFH |
| | | KVSSQIVNEFKNSLEKHS |
| 30 | Cas13 | MEKTQTGLGIYYDHTKLQDKYFFGGFFNLAQNNIDNVIKTFILKFFPER |
| | | KDKDVNAAQFLDICFKDNDADSDFLKKTKFLRMHFPVIGFLASNNDK |
| | | AGFKRKFSLLLKAISELRNFYTHYYHQPIEFPSELFELLDDIFVETTSEIKKL |
| | | KKKDDKTQQLLNKNLSEEYDIRYQQQIERLKELNAQGKKIPLNDETAIR |
| | | NGVFNAAFNHLIYKDGGDLKPSRVYQSSYSEPDPAENGTSLSQSSILFL |
| | | LSMFLERKETEDLKSRVKGFKAKFIKNGEEKISNLKLTATHWVFSYLCFK |
| | | GIKQKLSTEFHEETLLIQIIDELSKVPDEVYSAFGAKTKQKFVEDINEYMK |
| | | EGNADLSLEDSKVIHPVIRKRYENKFNYFAIRFLDEYLSSTSLKFQ.VHVG |
| | | NYVHDRRIKNINGTDFQTERVVKDSIKVFGRLSKISNLKADYIKEQLSLP |
| | | NDSNGWEIFPNPSYVFIDNNVPIHIQTDEATKNGIKLFKDTRRKEQPEE |
| | | LQKRKGKLSKHNIVEIIFKETKGKDKPRVDEPLALLSLNEIPALLYQILEKG |
| | | ATPEDIELIIKNKLAERFEKIKNYDPETPAPASQISKRLRNNTTAKGQETL |
| | | NAEKLSILIEREIEDTETKLDAIEEKRRKAKKEYRRNSPQKSIFSNSELGRI |
| | | AAWLADDIKRFMPAELRKNWKGYQHSQLQQSLAYFEKRPQEAFLLLK |
| | | EGWDTSDGSSYWNIWVINSFSETEDFEKFYENYLRKRAKYFSELAGNI |
| | | KQHTHNAKFLRKFIKQQMPADLFPKRHYILKDLETEKNKVLSKPLVFSR |
| | | GLFDSNPTFIKGVKVTENPELFAEXXNGIATGTKRNIPSSISMAGKETI |
| | | MSF |
| 31 | Cas13 | MEQNKLGKGIDYNPFKTVDKHYFGGFFNLADNNIQEVFDEINIRYKN |
| | | GNLKPKVAIERYTTENTSLVEYEKFVAILTEYFPIVKEIDQKNKKDSNDK |
| | | VIEKTRIERITDFRDAFILFIETIEKLRSYYTHYQHDDITIDNQLFIHLDKILL |
| | | NTVLETKKKYLKTDKTKELLKNSLQAELKELYHLKINQLEQKKNEVDALI |
| | | KEQKSKGKKTDKPFKYSKDRDQIINSIYNDAIRPFLYENANKVELSDKKK |
| | | TAFNEKDASASERDFNLPISSSGIIFLLSCFLNRKEIEDLKANIKGYKGKVI |
| | | KGETFDLEKNSIRFMATHRIYSVMCYKGLKNKIRTSESATKETLLMQMI |
| | | DELSKIPDIVYKNISTDLQNTFTEDWNEYYKDNIENNENLENSKVIHPVI |
| | | RKRYEDKFNYFAIRFLDEFVDFPSLRFQVHLGNYIKHSMPKNIGSVTTT |
| | | REIKNKIFVFGKLNEINQSKNDFFNKNKEEEQETNWEIFPNPNYHFPM |
| | | ENSDELKNANKIGIYIDLKDKRKKDTLNEAIKKREKETSIYKKDLVHQIID |
| | | KNLDMHIGQPVAYLSMNDIHAIIFSILSQNVFTKDNKLNGGDIEKKIKD |
| | | QINNQITEITEKDASIKILKNHSDNNSNYPNTHKLYDDISNEIEXXXXXX |
| | | XXXXXXXXXXXXXXXXXXXXXXXXXXXXXXXXXXXXXXXXXXXXXXXXX |
| | | XXXXXXXXXXXXXXXXXXXXXXXXXXXXXNEIEVLDKLMQKHEKRVKEY |
| | | INTQEDKKYKPARKHILYNSEKGEIATWLANDIKRFFPKEFKENWKGH |
| | | YHSEFQRNLAYYETNKKEVKTILNDLDYRKEIPFIDFSKNTLADFYFEYLK |
| | | KRKIYHKNLWVEVNKLIKGENINKEKLFDNCFRIYKRKNYVSNVIDEKV |
| | | NTILSNPIFIERGFIDEKPTIIPKMPLEGNEEHFAAWFVAFKSFKNNEFQ |
| | | NFYDTNKYPLETKDKTNSELKKIQTKTYNQKKNDWATWLIVQYIFKDIF |
| | | STDLQNVKLSELFQTREQRIQNQVKALDGERNQNFIWNRTIDLQLNE |
| | | KIKIPNVKLKDIGNFRKYVNDSRVEAFLRYNDITQWMAYLPSNWQKE |
| | | DESKPKPVNVIQLQLDDYEKIRREELLKEVQKLEKTIYNNTNVKTVLLQ |
| | | DGNPNFKNYVLNGLLEEIKGINISAFTVLHEKTNFDKIDFNVLENCSEIE |
| | | QSATLIILIRNKFAHNQLPSSDCYQFCSKILTRDTEQTYANYYLKLFMILK |
| | | DKL |
| 32 | Cas13 | MEETTTMGKGVAYDHTLFKDKHYFAGYLNLAVNNIENVFKTVYKNRF |
| | | DIKQHNLYKILDSLDGQISEPDYIERVSFLKQYFPVLHYLDLHPDNKRFT |
| | | KEEDKVKARRRYLINNLRLLIETLSKLRDFYTHYYHKPLSIEQNTFSLIDNI |
| | | FLNVVIDVKRQKKKNDHTRQLLKDSLKEEMDILYQKTKASLKEKQKEN |
| | | TRIKLDSETINNTIFNNSFSHLIYRRKKADNDILSASCKSEYKGEPTENGI |
| | | NVSVDGLLFFLGIFLSRKESNDLRGRIKGFKGTVIKDLPDFPNEKNNSLK |
| | | FMATHWVFTYLNIKPIKQKLNTNFSRETLLLQIVDELTKIPNEIYRNLCF |
| | | KKQQEFVEDINEYIKEGDDIDTLNSSTVIHPVIRKRYENKFNYFVLRYLD |
| | | EFVSFNSLRFQIYLGNYVHHIQRKKLSGTEYETERVIKEKINVFGKLSEVS |
| | | NIKGDYFIQNNPDNEALGWEIYPNPSYNFTGNNIPIYFDINDQDKEKIN |
| | | EYKSIRNFSEKRILRKKNKKNKQEIFDLINNTLTTRVFTAEPTAILSLNELP |
| | | ALLYTILCENKTASEIENLLRRTYLKRLNTIKNYQPGTLPQSKITKNLNKS |
| | | TNQESLDVSKLIKAMKHEISISNEKLTLIKKNQNEVKDTSHRRKYVFNSK |
| | | ELGIEATWLANDLKRFMPKKVRENWKGYMHSQLQNSIAYYSQKPKE |
| | | ALSILSSVWNFNDDNYIWNEGIKKAFNEKEFEKFYCKYLASRNKTLEKL |
| | | KENLDNLEYKTDKRKLDKFIKQQNLDCLFHIRTYTIDSTQEQINKLLAKP |
| | | LVFPRGIFDSKPTFVKNESVTEKPELFADWYTYTYKEHPLQEFYSFTKDY |
| | | ECNFKKEKLTVKEFVKNQEQLNPEEQLNLFKLKEDLSIKCIKNQDLFLKL |
| | | VVDNIYNKIFEYNIDISLKNLYISRKERIAIGLKAKELNQINDSYIWGKTIL |
| | | YQDKQIRETKVQLKDINKIKRFLEEDKVKQILSYDINKQWEIEELKYELYI |
| | | KPNSYEVIRREKLFKAIQEFESYILTINNFDGSNHPSILEYNSNPRFKHYV |
| | | VNGLLLKKGLATNEEIEWLLAKGQKEFNTFDKSIVEKPEIIQKAFLLVLIR |
| | | NKFAHSQLPIKEYYEMIRSYTKNIENLNTTEIIFQFTTNTINELKR |
| 33 | cas12b | MATRSFILKIEPNEEVKKGLWKTHEVLNHGIAYYMNILKLIRQEAIYEH |
| | | HEQDPKNPKKVSKAEIQAELWDFVLKMQKCNSFTHEVDKDVVFNILR |
| | | ELYEELVPSSVEKKGEANQLSNKFLYPLVDPNSQSGKGTASSGRKPRW |
| | | YNLKIAGDPSWEEEKKKWEEDKKKDPLAKILGKLAEYGLIPLFIPFTDSN |
| | | EPIVKEIKWMEKSRNQSVRRLDKDMFIQALERFLSWESWNLKVKEEY |
| | | EKVEKEHKTLEERIKEDIQAFKSLEQYEKERQEQLLRDTLNTNEYRLSKR |
| | | GLRGWREIIQKWLKMDENEPSEKYLEVFKDYQRKHPREAGDYSVYEF |
| | | LSKKENHFIWRNHPEYPYLYATFCEIDKKKKDAKQQATFTLADPINHPL |
| | | WVRFEERSGSNLNKYRILTEQLHTEKLKKKLTVQLDRLIYPTESGGWEE |
| | | KGKVDIVLLPSRQFYNQIFLDIEEKGKHAFTYKDESIKFPLKGTLGGARV |
| | | QFDRDHLRRYPHKVESGNVGRIYFNMTVNIEPTESPVSKSLKIHRDDF |
| | | PKFVNFKPKELTEWIKDSKGKKLKSGIESLEIGLRVMSIDLGQRQAAAA |
| | | SIFEVVDQKPDIEGKLFFPIKGTELYAVHRASFNIKLPGETLVKSREVLRK |
| | | AREDNLKLMNQKLNFLRNVLHFQQFEDITEREKRVTKWISRQENSDV |
| | | PLVYQDELIQIRELMYKPYKDWVAFLKQLHKRLEVEIGKEVKHWRKSL |
| | | SDGRKGLYGISLKNIDEIDRTRKFLLRWSLRPTEPGEVRRLEPGQRFAID |
| | | QLNHLNALKEDRLKKMANTIIMHALGYCYDVRKKKWQAKNPACQIIL |
| | | FEDLSNYNPYEERSRFENSKLMKWSRREIPRQVALQGEIYGLQVGEVG |
| | | AQFSSRFHAKTGSPGIRCSVVTKEKLQDNRFFKNLQREGRLTLDKIAVL |
| | | KEGDLYPDKGGEKFISLSKDRKLVTTHADINAAQNLOKRFWTRTHGFY |
| | | KVYCKAYQVDGQTVYIPESKDQKQKIIEEFGEGYFILKDGVYEWGNAG |
| | | KLKIKKGSSKQSSSELVDSDILKDSFDLASELKGEKLMLYRDPSGNVFPS |
| | | DKWMAAGVFFGKLERILISKLTNQYSISTIEDDSSKQSM |
| 34 | cas12b | MATAVDTSTTRAYTLRLSGGNNWRELLWQTHVAVNRGAWVWGD |
| | | WLLTLRGGLPASLADGDAERRVVLALSWLSVESPASLAPQAHIVAYGS |
| | | DARDERNRKVTERFRDILRRMGIKQQQEQEWLDACLPALMASIREDA |
| | | VWVDRSACFAEAQQCYRGLSSEWARKTLFDFLGGEDDYFKPSAKEG |
| | | ASSKAKDFVQKAGRWLSRHWGAGKKSDPRDISTRLGKLAGVDPKAID |
| | | GHTGRAALEDLLRTLGSRPAQNADAEKLYRQLKRAVGWKGRPSKGA |
| | | VALKKIRDAERVPNDLWKEIASTLREEAAVQSSQTSDHAAVPDWRSH |
| | | WPAEITGLPMPYRVDRDYIWEHGVMLDHALRRVSSAHTWIKRAEAE |
| | | RRRFQQDAAKMGSIPEEARNWLDAFRERRSSSSGATGDYLIRERAIN |
| | | GWDKVVQAWETLGPNSTRDQRIAAARDVQANLDEDEKFGDIQLFA |
| | | GFGDEHVDDPERCLADDRATCVWRNSSGRADGRILKDYVAATVAEH |
| | | NQRRFKVPAYRHPDPLRHPVFVDYGKSRWSINYSALTAAQQRRKTTO |
| | | KLAQAKTDNTRAKLQQQLASTADLRSVTLGVWDGNRIVKISQRWRS |
| | | KRFWRDLDLDHFGSHPSAAVSRADRLGRVAARQDPGAAVYVAKVFE |
| | | QQDWNGRLQVPRRELNRLADVVYGKGADPDFGKLERLDPRARRLW |
| | | ERLSWFLTTSATVQPQGPWLDYVAAGLPSGIQYTKSRAGYYLNYDAN |
| | | HGRKGRARLCLARLPGLRVLSLDLGHRYAAACAVWQTLTIEQMTNEC |
| | | RQAAHPAPSNDDLFIHLRHPTHKPQKSGRKKGRPVTKTTIYRRIGPDK |
| | | LPDGTDHPAPWARLERQFLIKLQGEDRPARYASQKEIDEVNQFRNFV |
| | | GLEPIVDRPRVDDLHSDAVRVARLGLRRLADAARIAFAMTAAKKPISG |
| | | GHEVELTTAQRIEFLQDALLLWQSLAASRRYRDDWAEKLWQSWVVE |
| | | KLGGPQPAEIADDLPRSQRAASLKTARQSLRKVAEKLSDGQSPSAAEL |
| | | HRLWAERWQQRQTEWRRHLRWLRRLILPRRKDHQQEDRPLORVG |
| | | GLSVKRIQTIRQLYQVLKAFRMRPEPSDLRKNIPAPGDRSLASFGRRIL |
| | | NHLERLREQRIKQLASRVVEAALGAGRISKPPGRDRRRPQQPVDRPC |
| | | HAVVIENLQHYKPEDSRLRRENRQLMDWQARNLRKYIVEGCELHGLL |
| | | FVEVSPAYTSRQDSRTGAPGLRCEDVSRTALQEAARRMHASHSRPSN |
| | | SSPGGSQTQFEREVCRWINEFKRVEGSSSSLSARQAVLKAFLHHQASI |
| | | PTSLSTILLPRRGGELFVSADPDSPLACGLQADLNAAANIGLKALTDPD |
| | | WMGAWWFVLVDRASGQPVEEQVQGCPIWLSCGPLSNSNPATIDPS |
| | | DSPTAARRSNGTGAKGRARANEYWWSSLSATTLPDHKAWQPTQDY |
| | | WRDIEQRVVKRLLRLLDGSEWSED |
| 35 | cas12b | MNRIYQGRVSKIEIKDSEGNFRNVPVGSPDTCPLWRHHRIFQDAVNY |
| | | YLVALGALAGTGSENAFVGLGSKDRVIHDLYSRLFDSWERFPRDMHG |
| | | ASSLRDSLRRTLPGLSERASLQDAFDAILSGNEANARERVLSLLSLIQDL |
| | | GGDIQKGSKRYFPFFCEPATKATFPRARVGLLKVEGKDFVPRLLWSSD |
| | | LEIAPDQVVEQLKFEYFANPNESVQPIEGNEARVRLIEALDNPQLGIEL |
| | | PIEILSDLRKRVHLIETDIRIPRYFFGGAGAELRKFRLDLFLIAAYVTPDPSI |
| | | LRALRNSFKEPSASKSSKKKDETEEVENLLRSLGDDPLILARGERGFVFP |
| | | SFTSLPTWVGANAQKPIWRDFDIAAFAEALKSLNQFTAKTEEREEKLK |
| | | KAEETLHYMLGISDAIPRSSDSETEEQAPSRPGKDPRWPLVAQLEKEL |
| | | GENLSEGTWQLSRSAMRGLRDIIGLWRKHPGASVVTLQKDVKTYQA |
| | | DEKHKREIGSVQLFLLLCEERYHALWQTETDDERGDESEENDDPARILS |
| | | DAIEVHQIRREVERFREPIRLTPAEPVFSRRLFMFSDLTDKLAKVKFGET |
| | | TEENSEVKSQFVEAAIALKEGENLKEARVRITFSAPRLHRDELLGGAESR |
| | | WLQPITAALGFSNPAPSVKFDSAVALMPDHMDDGRIRHLLNFPVNF |
| | | DSAWLHQSIGKADLWKSQFNGTKDKNLHLHWAGTARDTTRKNTW |
| | | WENRTIIENGFTVLSNDLGQRSAGAWALLKVTCSRPDTKHPVRSIGH |
| | | DGTREWFATVLATGIHRLPGEDQRILKNGKWATEQSGKKGRNATFSE |
| | | YEAACVLAKNLGCESVENWLGMSGEKSYPALNDQLVKIANRRITRLGT |
| | | YHRWSCFSPEKFEDPARRANVIGGQLAELSAYQDENVTVSADILKSGD |
| | | FEGFRHRAGAAFEALRTELEVHLVNLANLTAPLRQKVWSWQKRPDSS |
| | | GYGDLLMVDLDDCHPKIRGQRGLSMARLEQLEGLRRLFLRYNRSLDR |
| | | SPGIPAKFGREDVGRTSGEPCQALLVKIDRMKEQRVNQTAHLILAQAL |
| | | GVRLCPHRIEENERKSRDLHGEYEKIPGREPVDFIVIEDLSRYLSSQGRA |
| | | PSENSRLMKWAHRAVRDKLKMLAEEPFGIPVVETVPAYSSRFHALNG |
| | | QAGSRLHELHELEAYQQQSLINLAAKTDFQNRDRSKAAGELFEQFQA |
| | | LAKLNERRRAEGKKVPRTLYYPKSGGPLFLASRDGDTIHADVNAAINL |
| | | GLRAIAAPACIDIHRRLRATKEKEVYRPRVGNAREKSAFSKDDIIQPSGA |
| | | PSKKFASSSSPNFFYEPEDLKQANGEPLFDRAMFGEYSLVSGVSLWSM |
| | | VNNAIYIRCVELNRTRLHGKDPDDQIPM |
| 36 | cas12b | MSITRSIKVKLIVPRDASLEARQLREGLWATHLFVNDGCHYYERLLLEF |
| | | RQRDVCVGKDDAGKDVIVPAAEWADRLRARLGRNGMVPSHIEAALP |
| | | IFRELYENMVPSALKAKSGTGQAGRSWHSKLVSPTSRGGEASAARIDV |
| | | LRPLLPVSGDDPAFEPAARALIEEAGDELLTSTGRCPAWVTAYRKGPE |
| | | GSAWVEKLRIQLREAVEAGDFDPPSDPQILAAGAVPAAPPLGAGIDAL |
| | | RPLLPLLGGDPAFEPAARALVEDIGDELFTSTGRPPTWVTAHPTWVRA |
| | | HRKDAECLEAADDFKWVERLRQRLRDDAKAGKFEQPLHERLGALGAL |
| | | PVAKPIGAGRVVSRADLTVFERGAMELAIEHLIGWESAGHRARAQYV |
| | | ERKKRHDDLLQWIEAEAPDALLAVRAYEAARTIHLATLGELGAAPQYT |
| | | LRLREIRPWRKLREWLLQNPDATIDERRRRLATMQTNDPRGYGGEAL |
| | | AWLAAPERRALVEHPAGDVVTRIAVLNIRKSILDRSRLFPTCTLADPVE |
| | | HPRFAKFGKPGDKNSAGYALAVDGVRREAIIKILVPRQDGLLVPTDLR |
| | | VPFAPSGQMRDLRASGLDISYERQDGRGRQAAKLQGGNLMFDRTHF |
| | | ARCGAPGPEALGSVWIKVALDLSSPAASLAMKTATPVRTYLSTAVRGR |
| | | PESTKYEKAAPPEGFRVLSVHMGLRTAATASMLRFGAPEEGGHEVPV |
| | | SGLAGETLVAFHERTVTMKLPGEDPDTRTEANRGVAKRELRGLGRGI |
| | | GCLKAIRRASASATPEDRAEALVIIETHVGQGDRHGWAPAEAVGRLD |
| | | PHGDPDDWKTACAALYAAVEADLGVAISSWRKAARAGGATGMLGG |
| | | KSLWAVDHLERSFRFLRSWDLRARPHDGDPRRPRPGYASKLLHHIDG |
| | | VKDDRVKTTADRIVQAACGRAWIGGPTVKRGTQDVRLPGRWEQRG |
| | | PRADLILLPDLTHFRFRSDRPRAENSRLMRWAHRQLAIYVRMQAEVE |
| | | GILVADTGAAFTTRFDAWTGAPGVRCEPVTADHLRGIAKREDYWLAR |
| | | LLREGALKHLRIDPASLRVDDLVPMDHGKILVALDGVDLPGLRILDTDV |
| | | NASQGLGRRYIEGHGLAYRLPGARVPRGEGEREAAVVHIKGKRLASA |
| | | MGGTVVVLRASEGPGDITWTAEVYDRPQGARKALGLSLAAFNSIATA |
| | | AVDDEGPAPENDDEALEEEAEEALGIATGERIVFFRDPSGAVAGGGW |
| | | LEASAFWGIANRMVTDRLRELGRLG |
| 37 | cas12b | MSLNRIYQGRVAAVETGTALAKGNVEWMPAAGGDEVLWQHHELF |
| | | QAAINYYLVALLALADKNNPVLGPLISQMDNPQSPYHVWGSFRRQG |
| | | RQRTGLSQAVAPYITPGNNAPTLDEVFRSILAGNPTDRATLDAALMQL |
| | | LKACDGAGAIQQEGRSYWPKFCDPDSTANFAGDPAMLRREQHRLLL |
| | | PQVLHDPAITHDSPALGSFDTYSIATPDTRTPQLTGPKARARLEQAITL |
| | | WRVRLPESAADFDRLASSLKKIPDDDSRLNLQGYVGSSAKGEVQARLF |
| | | ALLLFRHLERSSFTLGLLRSATPPPKNAETPPPAGVPLPAASAADPVRIA |
| | | RGKRSFVFRAFTSLPCWHGGDNIHPTWKSFDIAAFKYALTVINQIEEKT |
| | | KERQKECAELETDFDYMHGRLAKIPVKYTTGEAEPPPILANDLRIPLLRE |
| | | LLQNIKVDTALTDGEAVSYGLQRRTIRGFRELRRIWRGHAPAGTVFSSE |
| | | LKEKLAGELRQFQTDNSTTIGSVQLFNELIQNPKYWPIWQAPDVETAR |
| | | QWADAGFADDPLAALVQEAELQEDIDALKAPVKLTPADPEYSRRQYD |
| | | FNAVSKFGAGSRSANRHEPGQTERGHNTFTTEIAARNAADGNRWRA |
| | | THVRIHYSAPRLLRDGLRRPDTDGNEALEAVPWLQPMMEALAPLPTL |
| | | PQDLTGMPVFLMPDVTLSGERRILLNLPVTLEPAALVEQLGNAGRWQ |
| | | NQFFGSREDPFALRWPADGAVKTAKGKTHIPWHQDRDHFTVLGVDL |
| | | GTRDAGALALLNVTAQKPAKPVHRIIGEADGRTWYASLADARMIRLP |
| | | GEDARLFVRGKLVQEPYGERGRNASLLEWEDARNIILRLGQNPDELLG |
| | | ADPRRHSYPEINDKLLVALRRAQARLARLQNRSWRLRDLAESDKALDE |
| | | IHAERAGEKPSPLPPLARDDAIKSTDEALLSQRDIIRRSFVQIANLILPLR |
| | | GRRWEWRPHVEVPDCHILAQSDPGTDDTKRLVAGQRGISHERIEQIE |
| | | ELRRRCQSLNRALRHKPGERPVLGRPAKGEEIADPCPALLEKINRLRDQ |
| | | RVDQTAHAILAAALGVRLRAPSKDRAERRHRDIHGEYERFRAPADFVV |
| | | IENLSRYLSSQDRARSENTRLMQWCHRQIVQKLRQLCETYGIPVLAVP |
| | | AAYSSRFSSRDGSAGFRAVHLTPDHRHRMPWSRILARLKAHEEDGKR |
| | | LEKTVLDEARAVRGLFDRLDRFNAGHVPGKPWRTLLAPLPGGPVFVP |
| | | LGDATPMQADLNAAINIALRGIAAPDRHDIHHRLRAENKKRILSLRLGT |
| | | QREKARWPGGAPAVTLSTPNNGASPEDSDALPERVSNLFVDIAGVAN |
| | | FERVTIEGVSQKFATGRGLWASVKQRAWNRVARLNETVTDNNRNEE |
| | | EDDIPM |
| 38 | cas12b | MPTRTINLKLQISPKTDEGRKIRSALWTTHSEINKAVAEIEKLLLLCRGE |
| | | KYYTTNSKDEEVEVPEPQVKTDALEMARAVQAKNGKAGTGSDEEVLS |
| | | ALRMLYEATVPSSVLDDKGKPLSGDAQSIGGSYAGPICDPETCRIKDV |
| | | DRLFESGPFAETASKKFTQLPAWFNEVTKKNFNKDEPEKFVKVGKDK |
| | | DEKFYEIDLRQADAWYESPEVKDIVSKNKAFNKDKWWKNKRDGVDT |
| | | WAAEFVKKQFDLRKDVRVSIREELWDRLGLLPLGSLYFKKPVGNKWN |
| | | RMAFRLAIAHLLSWESWNHQTLAEYTKYTKYKDGLIELAGASRSLEVR |
| | | FEPLRQYQKERHEELSRTSFVDDDRPFTIGARMIRAWGRVREAWRNK |
| | | GDGIDERRQILADLQ.TELKGKFGDPHLFLWLAEAGRESLWRDEDVLTT |
| | | FVEINIAQRDLERHRPYSLMTFADARLHPRWAMYEALGGTNLRNYEL |
| | | TPEGKVKIPLLICEKDKLSEKTFTIPLAPSGQLKSLEIKSLPKKKVKISYASA |
| | | HQFYAGIPGGSEILFDRLFMENRASSALANGSCGPAWLKLTVDVESKA |
| | | PPEWLDKKGRVQTPPTVHHFKTGLANKSKHTDKLEPSLRVLSVDLGLR |
| | | TFASCSVFELVDEKPAKGLFFETDHPHLWAKHERSFKLTLPGEEAGDD |
| | | PKVAQARREAMDEVYSLRRDMYRLKDILRLKIISAPNERREKLESKIAE |
| | | MREKQDARAVVTSNFFERLSEKCDLNPPMWEHSCNEIHRDAEKAFS |
| | | ARIGEWRKRTRKRPGSWEEWRETRSYHGGKSYWMIEYLEAVRKLLIG |
| | | WSTHGRDYGEINRQNKKRYGTVASKLLKHINKLKEDRTKAGTDLIIQA |
| | | ARGYIPLPGKGWMEKYRPCRVILFEDLARYRFKVDRPRRENSQLMKW |
| | | GHREIINEATLQGEIYGMVVETAGAGFSSRFHAKTGAPGVRCRYLKED |
| | | DFENGAPKEFLVRQMKNLMKGDRLEPGLLVPWDGGELFATVDNGK |
| | | PIVIHADINAAQNLQRRFWTRFADAYRVNAVEENDNWVVTDTGVRV |
| | | LGALEMAVHGEADRKPRTGFTLHGTLQSGAELKAEGKKTDIKDVEED |
| | | KDDSISSEIIELQDEKERKGRETFFRDPSGGILDPGKWYGSKRFWGRAK |
| | | GAVTEALLDNQGANNALEEKPGNDELPF |
| 39 | cas12b | MATRSFILKIEPNEEVKKGLWKTHEVLNHGIAYYMNILKLIRQEAIYEH |
| | | HEQDPKNPKKVSKAEIQAELWDFVLKMQKCNSFTHEVDKDEVFNILR |
| | | ELYEELVPSSVEKKGEANQLSNKFLYPLVDPNSQSGKGTASSGRKPRW |
| | | YNLKIAGDPSWEEEKKKWEEDKKKDPLAKILGKLAEYGLIPLFIPYTDSN |
| | | EPIVKEIKWMEKSRNQSVRRLDKDMFIQALERFLSWESWNLKVKEEY |
| | | EKVEKEYKTLEERIKEDIQALKALEQYEKERQEQLLRDTLNTNEYRLSKR |
| | | GLRGWREIIQKWLKMDENEPSEKYLEVFKDYQRKHPREAGDYSVYEF |
| | | LSKKENHFIWRNHPEYPYLYATFCEIDKKKKDAKQQATFTLADPINHPL |
| | | WVRFEERSGSNLNKYRILTEQLHTEKLKKKLTVQLDRLIYPTESGGWEE |
| | | KGKVDIVLLPSRQFYNQIFLDIEEKGKHAFTYKDESIKFPLKGTLGGARV |
| | | QFDRDHLRRYPHKVESGNVGRIYFNMTVNIEPTESPVSKSLKIHRDDF |
| | | PKVVNFKPKELTEWIKDSKGKKLKSGIESLEIGLRVMSIDLGQRQAAAA |
| | | SIFEVVDQKPDIEGKLFFPIKGTELYAVHRASFNIKLPGETLVKSREVLRK |
| | | AREDNLKLMNQKLNFLRNVLHFQQFEDITEREKRVTKWISRQENSDV |
| | | PLVYQDELIQIRELMYKPYKDWVAFLKQLHKRLEVEIGKEVKHWRKSL |
| | | SDGRKGLYGISLKNIDEIDRTRKFLLRWSLRPTEPGEVRRLEPGQRFAID |
| | | QLNHLNALKEDRLKKMANTIIMHALGYCYDVRKKKWQAKNPACQIIL |
| | | FEDLSNYNPYEERSRFENSKLMKWSRREIPRQVALQGEIYGLQVGEVG |
| | | AQFSSRFHAKTGSPGIRCSVVTKEKLQDNRFFKNLQREGRLTLDKIAVL |
| | | KEGDLYPDKGGEKFISLSKDRKCVTTHADINAAQNLQKRFWTRTHGFY |
| | | KVYCKAYQVDGQTVYIPESKDQKQKIIEEFGEGYFILKDGVYEWVNAG |
| | | KLKIKKGSSKQSSSELVDSDILKDSFDLASELKGEKLMLYRDPSGNVFPS |
| | | DKWMAAGVFFGKLERILISKLTNQYSISTIEDDSSKQSM |
| 40 | cas12b | MYRGFCTVTATSGGWQSTTFLAGAQMADTTTRAYTLKLQGDRLAL |
| | | WRNHVIFNNGVKAWGEWLLCLRGGLPASLADHRDSLDVSKGEISRTF |
| | | KERTAAITPATIRQELKFKAATEKKVREEVASRRKKVTETAVAKELLAAR |
| | | RSELRRILALSWLCPETPVQLVPQAAIVAAADDSDREQKVLDGFRQILK |
| | | RKGVSDVAGWVQDCDATLRATIRSDAVWVDRTACFCSMPRAVRPS |
| | | EVDAAKHLFRLFGSMSDYFATASASSGPAEPKDFANTCRDWVSSFW |
| | | GGGEKSNKASILAALSAIAQIKPTRVVGKRGPAALAVIAGVLEQKPVDD |
| | | SVEALARAIGWLSGRPSAARLAINAIAASPRVSQKLWDRLVLACEKDC |
| | | GRQKSKLAFEGSASTIASALEPRLAGLTGMPYASTGRELIGEYATMLAF |
| | | AMRRVSQIHTKAKQAEAERRSFAPEQARLALVPSAARKWLEDYVEAR |
| | | TAASGAVDGYQLRKRALGGWADVVAAWSRCETSEDRIAAVRELQAD |
| | | WEKAGDVQLFEALAADDAICVWQSANGKTAASILTDYVRAAVADQN |
| | | ATRFKVPAYRHPDPLRSPTFVGFGNSQWSIAYSAQGEARERRKLLDRA |
| | | SGSAKDAERAREGLAREAVLQNVSLDLWAGDKMVPTQFRWQSRRLL |
| | | SDLALHSVPAMKGAKVTRATRFGRARIAAGPVLLDGIADDTPWNGRL |
| | | QAPRRQLEDLARILDAKGLPFDDESKWPPKVRSRLKHLGWFLTHSAKL |
| | | TPSGPWLDYVAGGLANGWKWAEGREGACLFREDNKDRKGRAKLILS |
| | | RLPGLRLLSVDLGLRTSAAAAVWQVVSKRQLTAAKDGAKSVSDTDLF |
| | | CLVRTGDRTQVYRRIGLSAWARLERQFLIRLDGEKAAARPATTNEWES |
| | | LQSFRAWLGCGIERRPEKLPPVDSLQQSAERLCRLGLRRLSDLARVAYL |
| | | LTAKERPIMGGRTAPLDEEGTVQAAQDALSILHALGSSEDFSDARLQG |
| | | IWRTAIGDTPPLAARLTKKQRQELREALRPAAEKLRGKAALGKELADL |
| | | WKERSAAWAKHLRWLRDWVIPRFDKRKNGERVRSARGVGGLSLDRI |
| | | ATIRGVYQIMRAYASRAEPTNLRAGVERLEKAAAKKLRPEFGRRMLAK |
| | | MERLRENRVKQIASRIVEAALGVGSEDRLHWERGRRRPTAAISDPRFA |
| | | PCHAVVIENLENYRPDEKRTRRENRGLMSWAARAVGKYLAEGCQLH |
| | | GLYLRQVSPAYTSRQDSRTGCPGLRCNDVRAQELLNPEGWIGRLVAR |
| | | AAEAVKEGKATPRQRLLVTLAESARAGIAESAAVRIIAPGGQLFIAADP |
| | | QSPASNGIHADMNAAANIGLVALLDPDWPAAWWRLPCKAATGYVD |
| | | ESKVGGSEAVPLGRAILEVGAEAGKVYVNAWSDPQDSAVSRREWTD |
| | | TKRYWRDVEERVVEILLASNRGGRRGKPGAVPF |
| 41 | cas12b | MATKSFEAKIVCKPDEKYTAEQKKQFLWFTHQVFNDGVRKVIPYVFK |
| | | MKRGELGPEFQAIYYAITSSQDAIGKLEAVINPDWTSGKIGKSDPNKW |
| | | KELLKYQELEKGFRQRLKEEGIKSTKKFRKELEDEKKKLAKEIGQKDIWA |
| | | DAAAILRNKNLLLFNRDELLPNLPSEFRRKIYEMTIQLIHGHQELVANW |
| | | EDEHAEWLIEKDKWEEEHPEYMNVRPIFEKFEKEQGKVKGSRIRWLA |
| | | YLDFLSSKPELANWRGKAKETIPLTKEERAGFRKPGQHFAAFFNKNPEL |
| | | QELDRLHKEYQEKFARTQSKRTPHPDGFKHRPTFTLPDAMRHPVWYS |
| | | FKGATDPTKGSTYRNLDLENCTLDLKVLTAMEGEGRNPGGMIQYAFE |
| | | PDERIKGFRYVGTTEKGKRAKGYIYYDPILEKERPAKIQGIKLVFRPPRP |
| | | DGTAYLIFSCQIEDEKPKIKIWKDKEEESPGEITKRKKTEVYPPELITLAID |
| | | FGQRHLGAITICKNNNGRPEPIRFIPAYPKRRKDRESKPVSAWLAKIPG |
| | | LTFNAVGMHEKEISAGMSRRFQDPKSIRQAGEKEGRKSKGQHIPETET |
| | | PWAHLREHIANMKEDHYKKAANLIIRTALQNGAQVILIENLRNYRPML |
| | | ERTNLENRRRMQWAVRQTAKFLEDTARPLGLIVRQVSSAYTSRFCSSC |
| | | GHPGARVSLPGQKNWEKFYAEKYGKERKMIAVAGGQFFCCPACKKII |
| | | NADINASLNMHKVFYQNFIWPGKIDKKDTKNFIWQGKNYNWDQIA |
| | | DDVQSFLDQKAGIKKEDDIPY |
| 42 | cas12b | MAYGSEAPDERNRKVTERFRIILSRMGINQQQEQEWLDACRPALTAS |
| | | IREDAVWIDRSACFAEAQQHYPGLSSEWARETLFDFLGGENDYFALP |
| | | DPEAAPSSEAKDFVQKAGGWLSRHWGAGKKSDSTAISTNLNRLAGV |
| | | ESKAIVGRCGCDALAVLLTTLGGWPAKNADSGTLYHQLKQAVGWKG |
| | | RPSRAAKALEKVRDAPEVTDALWRQTADTLRQEAVAQSSRAAGGSG |
| | | VPAWMPAWREDMEARLGMPYRGARDYIWEHSVMLDHALRRVSSA |
| | | HTWIKRAEAKRRRFQQDADKIGSIPAKAREWLDAFRERRFSASGALR |
| | | GYLIRERAIDGWDRVVQAWASLGPNCTREQRIAAARDVQANLDEDE |
| | | KFGDIQLFAGVGDEDEGDPQPCLADDDAICVWRDLNGRADSNILKDY |
| | | VAATVAKHDQQRFKVPAYRHPDPLRHPVYVDYGNSRWSIEYSALKAA |
| | | HQRRKTTEKLVQAKTDRARAKFQQKPADTPDLRGVTLGVWTGSSIEK |
| | | VSLHWHGKRFWKDLDLDHFGRDPSATVSRADRLGRVAASQHPEAA |
| | | VHVAKVFEQQDWNGRLQVPRHELQRLADLVYGKGGDPDFAKLGSL |
| | | DERRTRRQWEHLSWFLTTSTTIOPRGPWLDYVAQGLPQGIQYKKGR |
| | | NGYYLEYAANQGRKRRARLCLARLPGLRVLSLDLGDRYAAACAVWET |
| | | LTREQITQECHQAGHPGPSQDDLFIHLRHRTGKPQKSGRNKGKPVTK |
| | | TTIYRRIGPDLLPDGTPHPAPWARLQRQFLIRLQGEDRPARFASQHEID |
| | | GSNRFREFLGLPPLADRPRVDDLHRDMVRLARLGLRRLADAARIAFA |
| | | MTATKKPISGGREETLATEQRIEFLQDALVRWQALAASSRYRDDWAR |
| | | QAWQEWIVEKLGGPQPAEIADELPRSQQATRVETARRSLREVAAKLS |
| | | NPQSSSATELHGLWAARWQERQTKWRQYLRWLRRLILPRRKDYQQ |
| | | ANRQVHRVGGLSVKRLQTIRQLYQVLKAFRMRPEPSDLRKNIPAPGD |
| | | PSLASFGRRILHHRERLRQQRIKQLASRLVEAALGAGRISKRLGRDRRR |
| | | PRQSVDAPCHAVVIENLERYKPEDSRLRRENRQLMNWQARNLRKYIV |
| | | EGCELHGLLFVEVWPAYTSRQDTRTGAPGVRCEDVPRSVLEEATRRIR |
| | | ALGSAPSGSSRGRSETRFEREVCRWIHEFNRVVGSSSGLSPRQSVLKAF |
| | | LDHQAAIPTWRSTVRLPRRGGELFVSADANSPLANGLQADLNAAANI |
| | | GLKALTDPDWMGAWWFVLVKRDSGQPVPQQVQGSPIWESCTRLSS |
| | | PATVDSSDSPAGARRSKGRGARGRARATEYRWSPLSAMTMPDNKT |
| | | WWPTRDYWPEIERQIADRLLREQIDPENRF |
| 43 | cas12c | MKKTSPLKRSALRTARRQIARGCLPIGNRDISTTRTRVLPLADSVADAV |
| | | WNQARTAALTLRGFGSGSLFDLLLDLHASGLRLFSSNGEREGFLLKQK |
| | | FDAGKFDRAAAKDVGEDMPKFTAANLRAALVAIPRGGGPDTDAKAL |
| | | ATRLARAVGVKATKLDKPPKLLKDMAKELAMAFPTWKELSTANGEV |
| | | GAVIDDVARMYGLRWPSLRRGWAFRLPEVTRELGSPTLAFDPDAPVI |
| | | DETSATARFAAIVARYLPECGGLTDSAAAKGVQARITTTNANGLSWLF |
| | | GVGLRGMRDLPVDTVADTLAIDVTRGRDALRALVNDIKALPRLGEFG |
| | | DRVYVESRATLQGAVDSLIANYVGRLADLVASADALEHDQPRPPVLD |
| | | DADWKPAIFDGMGFTPWEVEDMLDARPVEVARLRLALGVLAGTTPA |
| | | VAGDFARALADVEAFGAWAARTEAVAALINARVKVLKAPESLRLRGV |
| | | LGGGRWKAVVSIHPDEGEPAQVIPQLDTQLQALLDDGQRAFDVLVA |
| | | DYTPTFAAALEHARSDMRASLADKGREAPSAESIDLLARRKLLDMVAR |
| | | VTRRGSPSLGHAFLAACAVQGLTRPGTATERSLRGHILSGEQALFVHP |
| | | YARARSIVRLEHAGLLRLDLDALLTAMERDAEQRADVREQIVLRFTRQ |
| | | SLLLGGLPGRIRLAKVPWTQEAAAASGVRGAPWLKLHPDDAGTVARS |
| | | EVIKAFTARFHLSANGLLYRLNRMRFLERYDIRCFIGDTLLFAPKAGAW |
| | | TPPEQYRHGKYAHWLSHPDLPRTEGGAVDVVPAARWLTEASRRADE |
| | | DGRASAVALLAQFPHEWVAACEFEGAPVYEGVFPCEGKIGGWMKRR |
| | | GYRLAPPRHFAGELLAAFKDASVSPHGLTFEREMLREGTTVRELSRRV |
| | | VAAYPIAVPTHPDAERPWSPLHLMGLDLGEAGLGVCLRHIGTGAETTL |
| | | LLPVRKTRLLAHREEHYRRKVQPRQAFRKGYGDAMELAVKAAIGEVC |
| | | GIIDNLIVRYRAVPVFESAVAQARGSNKMIQRVFAGVVQHYTFVANN |
| | | GAAQTVRQSHWFGAGRWSYTYGADLLPAARQMTEKQLLKAKAEAV |
| | | FRPAMGFPGVMASGYRTSLVCACCGEDVLDAVDAAAEGGQVALTT |
| | | DAEGSGVLDLGGRSLRIKLEAPSPNPIVQKAARRKRRRTPWEALADRT |
| | | WTLTHKTDRADLVATLRRGLRRPPASVQGHATSGWEFHCAACGHIA |
| | | QADVNAATNLVRRYDDRVRKMEQARAHWDDPSVRAKLASELAERA |
| | | AARS |
| 44 | cas12c | MLTTKFKLELPAGCPLREDAATFDECRKLYDVVEGCGNGTLTGFLFSVI |
| | | LSGFRIFPDGKTAEIFANRSVYDEDEFRSALVEAVGAPLPRFTVKALIKR |
| | | LQMEVRARGNKDNRFVAEVMMKEYRQTLCGKTLPKGVDESYVDRLF |
| | | EEMARELTSRYRSWNELKGDLLGACKAVDAALRGFGDFPSLATMVTR |
| | | AAARRLPKDSTIVFDPQSPCIDVQTIGVDAMPYAAVSTILSYPESVGEK |
| | | RRDFVQNHLTTPSAAGLSWLFNRGLELFSEESVEELCRLFHVPEDQRT |
| | | RIVQIQNAARATPRQSFFLKKGGAPLGYHDFRSAFAGRINSWTANYL |
| | | NRLEELQGLLHDLTDELRLPDLVRNGEDFLATTDCRREEVEILCRSFSRE |
| | | RDRAQTAVEHLIGADPLQVVSDVAAIEEYSRIVNRLCAIKEQIVNSLRQ |
| | | AEDDKASRWTALWSEVKDEFQPWEKLIRLPKLNGMSGGVPPAQDEL |
| | | ETILARYSDVGRGASEHFDAVMEWAAKTGAEGDVLKKFAETEQQRA |
| | | DQRAPGKYDGRELALRLVLQRVARVVRDRSDACAENVRQWFLKENV |
| | | FAERKDFNKFFFNRLGNLYVSPFSNRRHAGYKLSDGLVERSGAVWREL |
| | | LALVKEMRGAYASFSEAGETFLRLESLLMGMRIGALTKNIPAEVAALRL |
| | | DDETALESVSEGLKLQLQQAEVPPSVLAKAFNVYVSLLSGCLIALRRERF |
| | | FLRTKFSFVGNTALVYVPKEKSWPMPSRYEASPSWTPIFENDVLVRLS |
| | | TGEVEVAETFRRAVALWGRTTDPVLKKALRELFHQLPHDWCCQVSVR |
| | | SSGDMTPAKRKEDDRDVLIVEKKGKYDSTIISKKIAATALVRLVGPSTH |
| | | KERLNRLLLDVGEVACDMTLLADQEILQKVEDDRVHLSPGKLQFSLSV |
| | | PISTPAEQCEDEVKSERKSTHFRRIVAIDQGERGFAFAVFRLEDAGKKG |
| | | AQPIAQGFVNIPSIRRLIARVHSYRKGKQSVQKFSQRFDSTMFTLRENV |
| | | AGDVCGAIAGLMSRYRAFPVLERQVSNLASGGKQLELVYKMVNARFL |
| | | DDRIPMHSLERTSWWCGTSDWVIPDLWVEVPESYAVKAKKDEILEKD |
| | | GKFYRTLRITPGAGVNAKWTSRICSQCGGNAMELIEKAREEKVKTVTL |
| | | DANGEVTLFGRTLRLYKRPSEERSREARRRNERAPWTEPRADVRLSLD |
| | | DFRRAVAENMRRQPKSLQSRDTSQSRYFCVFTDCRCHNKEQHADIN |
| | | AAVNIGRRFLESLLRE |
| 45 | cas12c | MRRQHHGGQNARDWRRKVAAAALRQKESVFTYKFGLSVNDGDFDF |
| | | DAAARTYDITEGIERGSLIGLVCAVHLSGFRLFSKVAETRQFLNRSRYPE |
| | | NEFAQALAAHTEIENPSVTVQSIESVFVTPPRKQDGVARLWSADELAK |
| | | RLFQTWNNRSPREGERNHPELLLAQGIARAVTKAFSGWKELADNAV |
| | | HALTCADNYLATLGNRFPKLSDLPPLTAGSTQTGTLAFDPESPFLNMT |
| | | GNEDIWLHQVVAVCAGRLKRYMPEIDPSSRKFASRLTDSIVSSQNNGL |
| | | SWLFGNGLRFLRQSSIAQIAETLSVSQNEHRRVEQLKEFADAIPVNPFF |
| | | ATDGYAEFRGSVGGKISSWVSNYWKRICELTVLHSQPPDITIPEGLLAS |
| | | ENATLFSGQHTAAAGLVALSARLPSQVRDAGKALFVLSGDGVPRADD |
| | | IATVEDVAGELAELTGQLAMLDNRIQQEIERAQDANDEGRVGSLASL |
| | | RPNPTKELKEPPKLNRISGGTADAAGELARLETSLNDLIRARREHFYRLA |
| | | EWTGNTASLDPLPALAERERKALTDRGMDPTLAEADEYALRRLLHRIA |
| | | GMARRLSPNEAKRVRETMTPLFLKKREANLYFHNRAGALYRHPFSNS |
| | | RHQPYSIDLNRARATDWLAWLEERAREMLGLLGSGAPANHEYLRDLL |
| | | SIETFVFTTRLSGLPAQVPGYLAKPKSDLTNIPPLLAAQLDVDEVSRDVA |
| | | LRAFNLFNSAINGLSFRAFRDSFIVRTKFLRLGHDELFYVPKARAWKPP |
| | | ADYRSAKGKISKGLALPAVKRNEAGSILPRETTQGLSRAKFPEGSHALLS |
| | | QAPHDWFVELDLRHDKMPQLAGLPVKMNADGLKGWRARRRPTFR |
| | | LAGPPSFKTWLDRALTSTAVKLGDYTLILDQSFKQSLRVEDGEVRLSAE |
| | | PAGIKAEIAVPVIDARPFPETEAEALFDNIIGIDLGERRIGYAVFSLPALLK |
| | | SGNPTRVKPTVVGSVAIPAFRRLMAAVRRHRGSRQPNQKVSQTYSTA |
| | | LQQFRENVVGDVCNRIDTLCERYRAFPVLESSVANFETGANQLKLIYG |
| | | TVLRRYTFSNVDAHKSARSAYWYSANRWQHPYLFVREWNKAQRTFT |
| | | GSAKPLAIYPGVTIHPAGTSQICHRCGRNALRALRNMPDRTIRVGKDG |
| | | LIVLADSTIRLLERADYSDRELKTFKRRKQRPPLNMPVPEGARPRDQLE |
| | | RVLRRNMRQQPQSEMSPDTTQARFTCVYTDCGFEGHADENAAVNI |
| | | GRRFLERIDIEASSRT |
| 46 | cas12c | MTHAKKIPFPVLKRSTLRKARQRIAAGSITAGERPFNSTVTRVVPVKDP |
| | | VSDQVWAVAREAAMTLRGFGQGSLFDMLIHLHADGFRLFPSGRERE |
| | | AFFLKDLFDPTEFDDGARRAFGDVMPGFTANSLREILGAPARKCGKVT |
| | | SVEILLPRLSKGLGVKKSAAPPEVLSSLAAALCEAFPTWSLLTAVDGGV |
| | | GKVIDDVLRTHGSRLPSLEKAWSTNLPEVPKGLGVPTLAFDDQAPAQS |
| | | EQTPTGRFAGVVARYLAETFASNPEATAGDASKAVQAKVTTPNGNAL |
| | | SWLFAVGRRAMCSTTLDELAIGLNITSPRGRHALSSLKERMMALPALS |
| | | VLGERAYPDSRATLQGTVDSLIANYVNRLFELSSSATSIAQTKLILPAAIQ |
| | | GDTAVFDGMPFSAEDVGALFEQLPSEIAKLEHAVKVLVGKERTSTLGY |
| | | QKAVDDVDEFGVWASSVDAVIGQINARLKTLERAQEPLGKLMGDGK |
| | | LKRLVNIHEPEGPAVEIIPVLDQELQDVLTSCRTAFADLEARYPMTVAK |
| | | AQRHAEAEVRNALELASRKEGGLSLASADVPALAKRKILEPIISIARRSS |
| | | PAMATAVLTECLRQKLIVKGTGSERSLRGYVLSGEQVIYAHPLSRRRSI |
| | | VRLDREGLQNFDALEFLDALQKDATQRTNVRESLIVEMARQSLLLSAL |
| | | PDRIEIGAISWQTPSQNQHAPWANLRPVNGTVGRSETIKSFTAVFHS |
| | | RISGLLYRLNRQKFMEKYDLRCFIGSTLLFSPKNADWAPPPQYRHGRF |
| | | SALLARSDFPWEGAEGTHANAVRLAKFLIDETRNATDLQQAIAAKALL |
| | | AQLPHDWVVCCDFDGAPSYEGAFVSAGEVSAWAKRSGYLLTPPRHF |
| | | AGAFLEGFKSTKISPHGLTFERMLERDGDSVIETGRRVTAAFPITQEVA |
| | | PAAQPWKPRHLAGLDLGEAGLGVCLKNLDNGHEQTLLLKTRKTRLLA |
| | | HSAEHYRRKDQPRQVFRKQYNQSSENAIKAAIGEVCGLIDNLIARYDA |
| | | VPVFESQAAAARGSNRMVARVYAGVLQRYTYVVGNGAADATRTSH |
| | | WLGANRWSYSFGADVIPKVRDLSPEVLRSIKKPENVFRDALGFPGVLA |
| | | NAWRTSMICSVCGTDPIGALEEAIAANQISFVTDNEGEGSLDLGDGRK |
| | | VTLRVEVPTSSALTKREASRRKRRAPWEAKVGTVWTLTRKSHRDDLLT |
| | | TIRRSLRRPSSTFQGSTTKQWEFHCPCCGQIQQADVNAASNLVRRYF |
| | | VRASDNARARQHWADDSKRLAFIASMGPDRSAREEKVS |
| 47 | cas12d | MRKKLFKGYILHNKRLVYTGKAAIRSIKYPLVAPNKTALNNLSEKIIYDYE |
| | | HLFGPLNVASYARNSNRYSLVDFWIDSLRAGVIWQSKSTSLIDLISKLE |
| | | GSKSPSEKIFEQIDFELKNKLDKEQFKDIILLNTGIRSSSNVRSLRGRFLKC |
| | | FKEEFRDTEEVIACVDKWSKDLIVEGKSILVSKQFLYWEEEFGIKIFPHFK |
| | | DNHDLPKLTFFVEPSLEFSPHLPLANCLERLKKFDISRESLLGLDNNFSA |
| | | FSNYFNELFNLLSRGEIKKIVTAVLAVSKSWENEPELEKRLHFLSEKAKLL |
| | | GYPKLTSSWADYRMIIGGKIKSWHSNYTEQLIKVREDLKKHQIALDKL |
| | | QEDLKKWDSSLREQIEAQREALLPLLDTMLKEKDFSDDLELYRFILSDF |
| | | KSLLNGSYQRYIQTEEERKEDRDVTKKYKDLYSNLRNIPRFFGESKKEQF |
| | | NKFINKSLPTIDVGLKILEDIRNALETVSVRKPPSITEEYVTKQLEKLSRKY |
| | | KINAFNSNRFKQITEQVLRKYNNGELPKISEVFYRYPRESHVAIRILPVKI |
| | | SNPRKDISYLLDKYQISPDWKNSNPGEVVDLIEIYKLTLGWLLSCNKDFS |
| | | MDFSSYDLKLFPEAASLIKNFGSCLSGYYLSKMIFNCITSEIKGMITLYTR |
| | | DKFVVRYVTQMIGSNQKFPLLCLVGEKQTKNFSRNWGVLIEEKGDLG |
| | | EEKNQEKCLIFKDKTDFAKAKEVEIFKNNIWRIRTSKYQIQFLNRLFKKT |
| | | KEWDLMNLVLSEPSLVLEEEWGVSWDKDKLLPLLKKEKSCEERLYYSL |
| | | PLNLVPATDYKEQSAEIEQRNTYLGLDVGEFGVAYAVVRIVRDRIELLS |
| | | WGFLKDPALRKIRERVQDMKKKQVMAVFSSSSTAVARVREMAIHSL |
| | | RNQIHSIALAYKAKIIYEISISNFETGGNRMAKIYRSIKVSDVYRESGADT |
| | | LVSEMIWGKKNKQMGNHISSYATSYTCCNCARTPFELVIDNDKEYEK |
| | | GGDEFIFNVGDEKKVRGFLQKSLLGKTIKGKEVLKSIKEYARPPIREVLLE |
| | | GEDVEQLLKRRGNSYIYRCPFCGYKTDADIQAALNIACRGYISDNAKD |
| | | AVKEGERKLDYILEVRKLWEKNGAVLRSAKFL |
| 48 | cas13a | MPIVKKFGRSQ.TSLSDRKIVLKMETAARNIPDFLLSDPEAVIGQWASA |
| | | MDKIAKKPKGKDKPSSYQRKFRERLGKAIWADLTGPEGPLRDVPAAEL |
| | | EDLRKRWDRRVHPYPDGTKDGPKPATPKGRLYTRFAGEVGYGKADA |
| | | VAIARDIRIHLLETEFKTGGGTRDAGRAVRRASSIEKNVLKKARVPQRP |
| | | KPPQEAAWSKEDEDRYFIPHDVARKIVLAAKAQEKEDHRVAWRTAA |
| | | AVLFEHFGRIFQQDGRALSFAEAEKQMPGLLALHRAVEGYYRQALKR |
| | | HRKDRREHEARPGREKGTGRRKVSAILPKDKTALLALIGHQHQNREIA |
| | | ALIRLGRILHYEAGRRGNSDMVANINRNWPADVSESHYWTSAGQIEI |
| | | KRNEAFVRIWRSALSHANRTLGDWLSPDEVANDITMSWESKHEKSG |
| | | KRKTGKLEENREEAEAHAPVIFGGSAERLGTGDDFQKTLEAICEVFSQL |
| | | RHSSFHFRGLDGFKDALTKTVKTCDPGAVARLQDLHAEDQANREARL |
| | | KEDLRGAHAELFLDEGRLAEIWALLHPKSTEKTLPPLPRYSRVVTRAEN |
| | | TCNGLKLPKSVNRESMKVPAIHCRYILTRLLYQSGFRTWIAEAPAAQLN |
| | | RWIETATERAQKATVGITKNEADRARMVGQIKVPEGQGIRRFLDDLA |
| | | GLTATEFRVQAGYESDREAARDQAAFLENLNCDVMALAFDKYLSDHK |
| | | LGWLAGIDAESRPSETPLSNVDELPSSGSLGTPERWEAALYAVCHLIPV |
| | | SEVGRLLHQLRRWSNGQKATPDGGRLERLFELYLDMHDAKFDGSTPL |
| | | RDHDDLAVIFETTGIRDRVLPSSLQHGEHERLPLRGLREMLRFGNLRVL |
| | | APIFATAKVDQAMIGELEGLEARIGDAPSQVDRAQALRTEMHAALCK |
| | | KRKLAHDDKKSVKDYLTSLQTVIRHRRLANHVRLTNHVRTNEILMSV |
| | | MGRLADFSGIWERDLYFVTNALLYQAGLTPCDVFSKEPPKENRRSPLQ |
| | | EFENGQIVFALRKMQAQCDTHAGLVDQIKGETARLFHIAEGAPGNDP |
| | | RIQNRNWFAHFNALKPKTGDRLDLTADMNRARDLMAYDRKLKNAV |
| | | VSAIVTLLERENIVIAWTMKDHQLTDAVLAAKSIEHLKQNKIRENLRDE |
| | | RSLGYVAALFGGRVAEEAPDIMHDRTVFHLVGALTEEMEPAE |
| 49 | cas13a | MRIVRPYGESRTDLGGERGQTRVLVDNTAARARHEIPDFAQSHDALV |
| | | IAQWISVLDRIATKPQGTQGATRAQHAFRDRLGRAAWAQMCAADRI |
| | | SAAAQADPYVAALWRFKTHPYGDAKYRPRKGKDGKPLGEPKPQGR |
| | | WYGRFAANAEPEQADVAAIAALMDHHLHVAELRIDPKRPEKRKGLIE |
| | | ARAKSIEGNVLVAEPRKRPVGSWSREAITRYFMRQDVAAEIFAAARD |
| | | REQGLNDVPRGPVRLALAAKILHGHWTRLFHAPGTRTAYSIREAEEKE |
| | | PELFALHMAVKDAYAKLLKRRTQPKTLKKGVKPPQQAPVTTVLPKNA |
| | | GELLRLVQHRSRNRDLSALIRRGKLIHYTAFDIAAAAAEAESKTPDVPD |
| | | ADRLAYVLTHWPDDLSASRYLTSDGQSAIKRSEAFVRVWRHTIAMAS |
| | | LTLRDWASMNNDLGDVLGSANKVDQAIGRANFDPAWHDKKVRLLF |
| | | GARAALFPSDDDGRKALLASVIRAGLALRNSSFHFTGRGGFLAALKKL |
| | | GSEEVMVPSILAAAHALWREDATARAGRLRAALTGAHAAHYFEEDQ |
| | | NASILTLLDEAPPKESLPIPRFRRVLGRAENTWKGKEALVLPPTANRRQ |
| | | LEDPARRCRYTILKALYERPFRSWLIARAPEEVNAWIDRAIERTTRAAK |
| | | DMNAKRGEDDKRSVIAAKAESLPRLSGERGIGDFFFDLSSATASEMRV |
| | | QRGYGHDGEAAKEQAGYIDDLLCDVVALAFDAWLRNPQANGRPLTF |
| | | ICDLKPETPLPAAPKCTLQEIGSAAEPVRPEDWQAALYLLLHLVPVGEA |
| | | GRLLHQLAKWTVTSRLADDLLNANVTDDPSKAERTADEEDLKRLVHT |
| | | LIQHLDMHDAKFEGGDALTGCEPFAALFASRPGFARIFPAEADERLDR |
| | | RVPKRGLREIMRFGHHGLVASFAEDTRITDKEVGDYLRLEIEERPDNVA |
| | | ALQARKEEAHERWVKAKEKRKTVDPKHLEDYVTALCGIARHRRLASR |
| | | VTLTDQVQVHRLLMTVLGRLVDFSGMFERDLYFAMLGLLDEKGARP |
| | | DEVFSGPIDEPKSRLALLANGRVLAALREQIPHSKDLAEELRKDLERLFG |
| | | MDCSGIRLLEADERGDTCLRDIRNDLSHFNLLHDDSFALDLTTLVNRTR |
| | | GLMSYDRKLKNAVSKSIKELLAREGLTLSWDMTDRHDLENARIGAKP |
| | | AVHLGGRKLAFRGGDRRPEPVRENLHSPTHLEAVARLFGGKVVEEDD |
| | | VTNLDLSSIDWAAEPHNSKETHRHRPAGPRKSPPKRRAYHAPR |
| 50 | cas13a | MRIIKPYGRTLVEHDGAGERKRVLTLRPDHDSKLDIEAFARDHDELVV |
| | | AQWVSTIDKIAAKPGPRKGATEEQRAFRDRIGKAAWALLVRNALLPG |
| | | LADADRADRLAKIWRRKIAPYGDLRPNERPASAKGRWYGAFAGEAD |
| | | VADVDAGEIAAKIHEHLYDAEYRISGDGRKPDGCIAARARSIAVNVLRP |
| | | ADSSACGQPEWSDRDLQAYRVADVAKQIWDAALSRENGRDGAGTK |
| | | RVTNSVAGGVLFEHWARIFPGPDGKALSIREAIEKEPGLFALHMAVKD |
| | | CYARILKHHKKKAPGRRERENGDVSPIRKVLPRDMDELFARIISGRGNR |
| | | DLNALVRLGKVIHYTASDPNADHPESITENWPGDLAGSHYWTSAGQ |
| | | AEIKRNEAFVRVWRHVVVLAARTLTDWGDPHGEIGSDILGKANDAT |
| | | GAKFDEAAFNRKCALLFGKRASHFTAAPDLAFKKAVLKTAIKGMAALR |
| | | HKSFHFAGRGGFVKALEGIGGLNEIDRFPDVTRALRTLLVEDIEDQSRQ |
| | | VRATMVGAHFGVYLSKGQVEAIYRAVTGAEPGSLPLPRFSRVLRRAKG |
| | | AWEAEDVLPPPVNRLDLEQRGRLCQYTGLKLLYERPFRRWLEGRSAA |
| | | KLNGFIYRAVTRASDAARTLNTKESDDWRDIIVARAEKLGKVPDGGDI |
| | | HGFFFELSAETASEMRVQQAYESDGERARQQAEYIEDLKCDVVGLAY |
| | | RSFLETEGFDFLRTLDPEAAIAEAHRFDPAELPDPAVDTDAEDWEAVL |
| | | YFLVHLVPVDEIGRLLHQMRKWDLLAHDRTAPVADGGQARLVDKVQ |
| | | RVFTLYLDLHDAKFEGGEALTGIEPFRKLFEESDGFDTIFPPQQGYEEDR |
| | | RVPLRGLREIMRFGDLPPLLSIYGRRPATKSNIERYRRAEVADAGGRSEI |
| | | ARLQARREELHAKWVEAKKEGLGPEDRRAYVEALAEIVRHRHLAAHV |
| | | TLTNHVRLHRLMMAVLGRLADFSGLWERDLYFATLALLHRAGKTPRE |
| | | VFENEGIDLLRNGQIVYALRKLNGSSNASALRSGLFPHFGSAFKRGDPI |
| | | GGIRNAFAHFNMLRAAQPPNLTECINRARQLMKHDRKLKNAVSKSVI |
| | | DLLAREGLNIAWAVHTRAGAHDLAEAVLSSRQAQHLGKLRLFPVSGD |
| | | GRDGKGFFIMEDLHGADFVEMAAELFGGRVSDRWRGKGCVSELRLD |
| | | SIDWSRQREQKKHGGGKKPTGRARKANRGHKNRHRRA |
| 51 | cas12f | MSARNIKVKIDTKGNPELRLGLWKTHQVTNEGVKYYTEWLIKLRQQD |
| | | IYRQSREDASPRVIISASDLKADLLCHARQLQKERLPRITGSDAEILGTLR |
| | | QVYELIVPSSVGKSGDSKTLARKFLSPLTDPGSAGGRDQSASGRKPTW |
| | | MKMKSEGNPRWEETFRKWKDRKDNDPTPLVLNQIADYGLLPLIPLFT |
| | | DVGENIFDPKSKSQFVRTWDRSMFQQAIERLMSWESWNQRVRRE |
| | | WEALNQKHSAFYREQFTADPDAALYRVAQSLEEEMRKEHQGFASDA |
| | | PEAFRIRRVALKGFDRLLERWQKTLGKNGQSATLLDDIRRVQSDLGDK |
| | | FGSAPLYQKLLDERWQRLWAVDPTFLQRYAAFNDLTQRLQRAKRVA |
| | | NLTLPDAVAHPIWSRYEGANASSGNRYHIHLPTKGQPGSVTFDRILW |
| | | PDGNGGWYERKRVTVFLRPSHQVDRIHEAPTDSVVDNFPLVVEDQS |
| | | ARTILRASWGGAKLEYDRNRLPRQLKKGVPDSIYLSLTLNLDTNKPSGL |
| | | FHTQQNGRVWIRKDVLMQYYNETPGDNVQFKPLYVMSVDLGIRSAA |
| | | AVSIFSVQLKAGIEEHRLTYPVADCPGLVAVHERSVLLTMPGERREQW |
| | | DRRYEQQRQGLRELRTDMRGMNDLLRGAYMDGDRREEFLARLSKLE |
| | | ETSPELWGPVYRSLNDSKVASATEWERLVVYCHRQVEQSLSSRIQNLR |
| | | SGRSAYRMSGGLSLDHVQDLERIRGIIASWTNHPRIPGSVVRWQQGR |
| | | SHTVALGRHILELKRDRVKKVANYLIMTTLGYAYDSKRARGEKWVRRY |
| | | PACHLMVFEDLTRYRFRTDRPRSENRQLMRWTHQELIAVTGIQAEPH |
| | | GISVGTMYAGFSSRFDAVTKAPGVRGATVRQILRTRGMVRLKEIAAD |
| | | VGIDINTLRPHDVLPTGDGEYLLSVVRHGESYRLKQVHADINAAHNLQ |
| | | RRLWTQDEVFRVSCRLALNSGRWAMPPPSYNKRYGKGFFEKGDNG |
| | | VYIWKTGGKIKISDTLEEDMDIPEDTAELLRGNSVTLFRDPSGTIAGGN |
| | | WLEAKEFWGRVNSLVNKGVRDKILGGIPVDNSSAHAE |
| 52 | cas12f | MPMIKITECVTWGTTCDGLWDARPHLEVRRSWSPPVQGGRTNRLD |
| | | APPASVTLNIHGRVEHPRDADALSVAPLRVRHMFERTTTKAAFLSPLD |
| | | LRPTQATDLERFAGTTRWAFNWANALLEAHHQAYEGRRQQAARHL |
| | | FGLGPEQLDELRVLANGTRDENGKKAKGDPVKRREYESIQKATKKAVS |
| | | EENKALGAEMKLWDEHRSLVVHKGRPLLTPGDEPALDAPPLAHRLYA |
| | | RRVELAGIQKTDPDYYAEQRKKEREAITPNVVAMKRDLMAKGAYFPS |
| | | EYDLQYIWRTVRDLPKEEGGSPWWPECPTILFYDGINRARTAWKNW |
| | | MDSASGARKGPPVGMPRFKSKYKAKDTFTITNPNRSVIKFETYRRIAIT |
| | | GIGSMRLHRGAKLLARRIAAGQAEITSATISRSGTAWYVSVLCTVHTTA |
| | | RTAPSKAQRSRGAVGVDWGVRALATTSKPIALTPGKPASRTVPAEKY |
| | | GAAMSQKIARAQRQLARMPKGSSRRRKAARHVADLQHLVAQRRAS |
| | | SVHQLSKALAQSFEIVAIEGLNVRGMTKSAKGTVENPGKNIRQKAGLN |
| | | RAILDATPGELKRQLEYKTKKYGSRLVELDTWYPSSKTCSRCGWVHPK |
| | | LKLSMRTFRCQQCGLVEDRDFNAAVNIERQGITHIVKENEGTDDREE |
| | | G |
| 53 | cas12f | MSTPMGWTAVNGGDATSPTTRVSSPPGEPRTGACPRAAAADATRA |
| | | ESSPRRTSSPARPGERHARARTSRYPIPNTYVVDRPSAEGDRHGQSSL |
| | | DCGPCPVRRSGALHQSSQAAHRRSMTGAKQKTPIRVVRFSIDHSALT |
| | | PAQVVAFARHAGAARQTWNWALGRWMDWRNNTKFYVDYKVFKA |
| | | AGMGPGLSTDDLIQVIERAVSIRQDDKWMDAAWDEARQIHGEWD |
| | | QFQKASTLQSLYLAGAQEPFDPSRDDGINPYHWWVTEGDKSGLPKA |
| | | ERHNVNSGATYTAPLRAFEEAVGRFYKLPGKKGTPKFKSKHDDEQGF |
| | | CIQRLTETGLSPWRAIEGGHRIKVPSIGSIRVVQSTKRLRQLIKRGGKTT |
| | | SARFTRRGGKWFVSVSVAFDLSAPRVQRPARLSRRQRAGGSTGVDLG |
| | | VNRLATLSSGDQFPNRRLLRKSMAEIKRLQRKFDRQHRAGSPECFNE |
| | | DGTHKKRCRWGREDGPAMSRSAQTTKRQLRRIHDLTARRRAGVLHE |
| | | ITKDLATRFELIGVEDLNVAGMTAKSKPKPDPDRPGHFLPNRRAAKAG |
| | | LNRAILDVGFYEFKRQLGYKTEWYGSTMQMVHRYAATSKTCSGCGW |
| | | VKPKLTLAERTFNCTQCGLAMDRDHNAAVNIRALALEGAAPMEREQ |
| | | PAPVGAAEKRHRDPVSHRRRPKSLAPCESTRPVRDLSPPATQEETA |
| 54 | cas12f | MAQAEAPRRLRAYKFALDPTEAQLREFEQHAGSARWAYNHANAILS |
| | | RYSDTLRNRWNAWIAQHHGLSREQLYALPDRERTAIQAAARAAVKA |
| | | ENAQLAAELRIIDDHRKRVTHKGKPSVEPGEQPAEDAPERAYQLWRE |
| | | RVELARLHAEDPQAYRAERKRILDEIRPLVNATKRKLIEQGAYRPTAM |
| | | DISTLWREIRDLPPDEGGSPWWPEVSIYAFTSGFAHAETAWKNYLESL |
| | | AGRRAGRPVGKPRFKKKRRSRRSFTLYGSVKLVTYRRIQVPSIGSVRLH |
| | | GSAKRLHRALERRGGIIKSITISQGGHRWYASVLVDELDITPGRETQRG |
| | | PSRRQRDRGAVGVDLGVHHLVALSDPNEKTLDNPRHLRKARKRLLKA |
| | | QRAMSRRRGPDKRTGQEPSRRWVKARNRVARLHHELAVRRAGHLH |
| | | EITKRLATSYELVAIEDLNVAGMTRSARGTIDQPGRGVRAKAGLNRSIL |
| | | DTSPAEFRRQLQYKASWYGATVAVIDRWAPTSRTCSSCGAVKAKLSL |
| | | AERTFFCEHCGMELDRDINAARNILAFAQSAYPGEGKALNACGGSVS |
| | | PGSQSVVQAGADEAGRPARKPRRSSRGSDPPATPTTRA |
| 55 | cas12a | MTSSSPTQRAYTLRLKSAAQGDKSWAEKLWDTHEIVNKGARAFGDW |
| | | LLTLRGGISHKLENLNDKETGEEGKKRRRILLALSWLSVESKDFAPEKYI |
| | | VEKDGEDKHRTKEALEAILKSRNLEDEEVESWVNDCKDSLTSSIRDDA |
| | | VWVNRSRAFDDAVRKIGDSLTREEIWDVLGRFFGKKEAYLAPRTIDEK |
| | | NGKTKKEEPKDLARKAGGWLSKRFGKGKGTDFSKLSKVYSEIVKWAEE |
| | | PRKSEPRTLANLASALKEDSLQGILNLIKNSGSKSGTRNFLEEIGEGEVS |
| | | KENLAILKAKAEGNRNYCKKEIGGKGRREWSDRILKSIEETLDGKFTYL |
| | | QEKGPARHWEFAVMLDHAARRISAGHTWIKLAEARRRNFEEDSQKI |
| | | NEVPENARQWLETYREDRSKSSGAIEGYLISKRAVTEWETVVKAWKN |
| | | CKTEEDRIAAAGALQDNLGIDQFGDINLFRALASEDVRCVWQVDGKP |
| | | DANILLNYVAATKAEFDKRRFKVPAYRHPDPLLHPVFCDYGNSRWEIR |
| | | FDVHEVNRTGKKAKQNKKTIETADVHGLKMDLWTGSKIENVSLRWQ |
| | | SKLLEKDLAVKQLDGKEDGKKEVSRASRLGRAAVGAGWETPVSASSV |
| | | FAQKHWNGRLQASRKELSRIARRVKTRGWDEKANSMKKNLKWFITF |
| | | SPKLKLQGPWISYVDNSEDKRPFTFTSKGEPILDEVFSIENKNRKGRAR |
| | | LILSRLPGLRVLSMDLGHRHAAACAVWETLSSRQLEDACAEGGYDKP |
| | | APDAMYHHIKSNRGKRVIYRRIGADELSDDSIHPTPWARLERQFLIKLQ |
| | | GEERKARMATADEIWEVHELERALGRKTPLVDRLTKSGWGSDSGTPR |
| | | QRQLLGELNQWGWEPDEAQENSEDDEITSRESLLVDKLMSRTVDTV |
| | | RKGLRRHGNRARIANFLVAREKTVPGGQMDTLNNEGRKEIIADALAF |
| | | WYELANGGEWKDTEALDWWKIHIEPELSVEELPDIAGTGIAPKERKR |
| | | KKKELKEKLKPVAERLLTSGAKKLSDQWCERWKQDDKEWQKTLRWL |
| | | RDWILPRGVRGKSELIRNVGGLSLDRLTTIQSLYQAQKAYFTRITPKGIQ |
| | | MDKDKPLTAVMNFGGHILNDLENMREQRVKQLASRIVEAALGVGRV |
| | | KIPKKSKDPKRHYERVDAPCHAVVIENLTNYRPEETRTRRENRQLMT |
| | | WCSGKVKKYLSESCSLHGLFLWEVPPSYTSRQDSRTGSPGIRCEEVSVE |
| | | KFFKTPFRQREVARAEEKDSKNKASAYEQYLIDLKERWKSRGEETALLR |
| | | IPRKGGEIFVSANSNSPASKGLQADLNAAANIGLKAITDPDWSGSWW |
| | | YVPCSSKDFVPIKDKIGGSRAFENITTPMPNPDDAKEATGKKRSGKKEII |
| | | NLWRNPACSPLERDEWERTAKYWNMVEYHVIKRLKRQMG |
| 56 | cas12a | MKNFQDFTNLYELSKTLRFELKPIWGTKKLIEEKNILKLDKKKRENYEKV |
| | | KPYFNKIHQEFINFALRNPNFDFSQFEEKYLNWLKDKKNKDLLKEKESI |
| | | DKIFLEKIWKLFENSVKDFLKENGFESIVKSEDQNLKFFRRKEIFEVLQEK |
| | | YGSELETQMVNKDWEIKSIFNGWEKWLWYFDKFFNTRDNFYKTDW |
| | | TSTAIATRIIKDNLKIFLENTIIFEKVKNKKIDFSEVEKNFSVSIDTFFEINNF |
| | | NNCFLQDWIDFYNKVIWGETLENWEKLKWLNEIINKYRQDTGEKIPY |
| | | FKKLQKQILSEKDWVFIDKIEDDGGFYEVLKNFYKNAAEKEWFLKNIFE |
| | | NFYTISDKNLEKIYFNKIAFNTISHKFWSALEFERILYEEMKKEKADWIKF |
| | | EKKENKYKFPDFIQIIFIKRSLENYDSENLFWKERYYKSEENVDWFLEKN |
| | | NNNIWEQFCKILNFEFLNILKRRIIDEAWEEYEVWFEISKNILWEKLENF |
| | | ELNQENKWIIKDFADYSLALYSFWKYFAVEKWRNWDLNIDISDDFYG |
| | | WEDWYIEKFYNTGYDEIVKPYNLMRNYISKKPWEDSKKWKINFETSSL |
| | | LSWWDKNLESNWSYIFQKWNKYYIWIINWSKPAKEVLEKLYSWNGE |
| | | KIKRFIYDFQKPDNKNTPRMFIRSKKDSFSPAVGKYNLPVEDILEIYDN |
| | | WLFKTENKDNSNYKESLSKLIDYFKLGFSKHESFKHFNFVWKDSKEYEN |
| | | IADFYRDVEKSCYQITSEFLDFEELKKLTFKKHLYLFQIYNKDFELDESLQ |
| | | KNWYNFRDEWQKNIHTKYFEALFLEENILRKSWAVFKLSWGWEVFF |
| | | RKESIKAEKEKRKNIEVTKNRRYTEEKYFLHFPIQVNFKNEISWNFNQEI |
| | | NKFLANNPDINVIWIDRWEKHLAYFSVINQKWEILESWSFNKIENYNK |
| | | NWEKLLFPEREIKEIHKDWSLIDLELVETWRKVDYVDYKLLLEYKERKRL |
| | | LQRQSWKEVEQIKDLKKWYISALVRKIADLIIKHNAIVIFEDLNFRFKQI |
| | | RGWIEKSIYQQLEKALIDKLNFLVNKNEINLEKAGSILKAYQLTVPVDSL |
| | | KEIWKQTWVIFYTEAAYTSKIDPIKWWRPNLYLKKQNAEINKENILKFD |
| | | NIIFNSKENRFEFTYDLKKFFWKDSKFPAKTVNTVCSCVERFKWNRNL |
| | | NNNKWGYIHYENLTDWKLANKEQKEDEFSNFKELFEKYFIDINWNILE |
| | | QIKNLDTKNNEKFFSSFIDLFTLVCQIRNTNQNAKWDENDFILSPVEPF |
| | | FDSRKSQNFWKSLPKNWDENWAFNIARKGLIILNRISENPEKPDLLIFN |
| | | ADWDNFARNI |
| 57 | cas13b | MTEQNEKPYNGTYYTLEDKHFWAAFFNLARHNAYITLAHIDRQLAYS |
| | | KADITNDEDILFFKGQWKNLDNDLERKARLRSLILKHFSFLEGAAYGKK |
| | | LFESQSSGNKSSKKKELTKKEKEELQANALSLDNLKSILFDFLQKLKDFR |
| | | NYYSHYRHPESSELPLFDGNMLQRLYNVFDVSVQRVKRDHEHNDKV |
| | | DPHRHFNHLVRKGKKDKYGNNDNPFFKHHFVDREEKVTEAGLLFFVS |
| | | LFLEKRDAIWMQKKIRGFKGGTEAYQQMTNEVFCRSRISLPKLKLESL |
| | | RTDDWMLLDMLNELVRCPKSLYDRLREEDRARFRVPVDILSDEDDTD |
| | | GTEEDPFKNTLVRHQDRFPYFALRYFDLKKVFTSLRFHIDLGTYHFAIYK |
| | | KNIGEQPEDRHLTRNLYGFGRIQDFAEEHRPEEWKRLVRDLDYFETGD |
| | | KPYITQTTPHYHIEKGKIGLRFVPEGQLLWPSPEVGATRTGRSKYAQDK |
| | | RFTAEAFLSVHELMPMMFYYFLLREKYSEEASAEKVQGRIKRVIEDVYA |
| | | VYDAFARDEINTRDELDACLADKGIRRGHLPRQMIAILSQEHKDMEEK |
| | | VRKKLQEMIADTDHRLDMLDRQTDRKIRIGRKNAGLPKSGVIADWLV |
| | | RDMMRFQPVAKDTSGKPLNNSKANSTEYRMLQRALALFGGEKERLT |
| | | PYFRQMNLTGGNNPHPFLHETRWESHTNILSFYRSYLKARKAFLQSIG |
| | | RSDREENHRFLLLKEPKTDRQTLVAGWKSEFHLPRGIFTEAVRDCLIEM |
| | | GYDEVGSYKEVGFMAKAVPLYFERACKDRVQPFYDYPFNVGNSLKPK |
| | | KGRFLSKEKRAEEWESGKERFRDLEAWSHSAARRIEDAFVGIEYASWE |
| | | NKKKIEQLLQDLSLWETFESKLKVKADKINIAKLKKEILEAKEHPYHDFKS |
| | | WQKFERELRLVKNQDIITWMMCRDLMEENKVEGLDTGTLYLKDIRT |
| | | DVQEQGSLNVLNHVKPMRLPVVVYRADSRGHVHKEEAPLATVYIEER |
| | | DTKLLKQGNFKSFVKDRRLNGLFSFVDTGALAMEQYPISKLRVEYELAK |
| | | YQTARVCAFEQTLELEESLLTRYPHLPDESFREMLESWSDPLLDKWPD |
| | | LQREVRLLIAVRNAFSHNQYPMYDETIFSSIRKYDPSSLDAIEERMGLNI |
| | | AHRLSEEVKLAKEMVERIIQA |
| 58 | cas13b | MESIKNSQKSTGKTLQKDPPYFGLYLNMALLNVRKVENHIRKWLGDV |
| | | ALLPEKSGFHSLLTTDNLSSAKWTRFYYKSRKFLPFLEMFDSDKKSYEN |
| | | RRETTECLDTIDRQKISSLLKEVYGKLQDIRNAFSHYHIDDQSVKHTALII |
| | | SSEMHRFIENAYSFALQKTRARFTGVFVETDFLQAEEKGDNKKFFAIG |
| | | GNEGIKLKDNALIFLICLFLDREEAFKFLSRATGFKSTKEKGFLAVRETFC |
| | | ALCCRQPHERLLSVNPREALLMDMLNELNRCPDILFEMLDEKDQKSFL |
| | | PLLGEEEQAHILENSLNDELCEAIDDPFEMIASLSKRVRYKNRFPYLML |
| | | RYIEEKNLLPFIRFRIDLGCLELASYPKKMGEENNYERSVTDHAMAFGR |
| | | LTDFHNEDAVLQQITKGITDEVRFSLYAPRYAIYNNKIGFVRTGGSDKIS |
| | | FPTLKKKGGEGHCVAYTLQNTKSFGFISIYDLRKILLLSFLDKDKAKNIVS |
| | | GLLEQCEKHWKDLSENLFDAIRTELQKEFPVPLIRYTLPRSKGGKLVSSK |
| | | LADKQEKYESEFERRKEKLTEILSEKDFDLSQIPRRMIDEWLNVLPTSRE |
| | | KKLKGYVETLKLDCRERLRVFEKREKGEHPVPPRIGEMATDLAKDIIRM |
| | | VIDQGVKQRITSAYYSEIQRCLAQYAGDDNRRHLDSIIRELRLKDTKNG |
| | | HPFLGKVLRPGLGHTEKLYQRYFEEKKEWLEATFYPAASPKRVPRFVN |
| | | PPTGKQKELPLIIRNLMKERPEWRDWKQRKNSHPIDLPSQLFENEICR |
| | | LLKDKIGKEPSGKLKWNEMFKLYWDKEFPNGMQRFYRCKRRVEVFD |
| | | KVVEYEYSEEGGNYKKYYEALIDEVVRQKISSSKEKSKLQVEDLTLSVRR |
| | | VFKRAINEKEYQLRLLCEDDRLLFMAVRDLYDWKEAQLDLDKIDNML |
| | | GEPVSVSQVIQLEGGQPDAVIKAECKLKDVSKLMRYCYDGRVKGLMP |
| | | YFANHEATQEQVEMELRHYEDHRRRVFNWVFALEKSVLKNEKLRRFY |
| | | EESQGGCEHRRCIDALRKASLVSEEEYEFLVHIRNKSAHNQFPDLEIGKL |
| | | PPNVTSGFCECIWSKYKAIICRIIPFIDPERRFFGKLLEOK |
| 59 | cas13b | MESIKNSQKSTGKTLQKDPPYFGLYLNMALLNVRKVENHIRKWLGDV |
| | | ALLPEKSGFHSLLTTDNLSSAKWTRFYYKSRKFLPFLEMFDSDKKSYEN |
| | | RRETAECLDTIDRQKISSLLKEVYGKLQDIRNAFSHYHIDDQSVKHTALII |
| | | SSEMHRFIENAYSFALQKTRARFTGVFVETDFLQAEEKGDNKKFFAIG |
| | | GNEGIKLKDNALIFLICLFLDREEAFKFLSRATGFKSTKEKGFLAVRETFC |
| | | ALCCRQPHERLLSVNPREALLMDMLNELNRCPDILFEMLDEKDQKSFL |
| | | PLLGEEEQAHILENSLNDELCEAIDDPFEMIASLSKRVRYKNRFPYLML |
| | | RYIEEKNLLPFIRFRIDLGCLELASYPKKMGEENNYERSVTDHAMAFGR |
| | | LTDFHNEDAVLQQITKGITDEVRFSLYAPRYAIYNNKIGFVRTSGSDKIS |
| | | FPTLKKKGGEGHCVAYTLQNTKSFGFISIYDLRKILLLSFLDKDKAKNIVS |
| | | GLLEQCEKHWKDLSENLFDAIRTELQKEFPVPLIRYTLPRSKGGKLVSSK |
| | | LADKQEKYESEFERRKEKLTEILSEKDFDLSQIPRRMIDEWLNVLPTSRE |
| | | KKLKGYVETLKLDCRERLRVFEKREKGEHPLPPRIGEMATDLAKDIIRM |
| | | VIDQGVKQRITSAYYSEIQRCLAQYAGDDNRRHLDSIIRELRLKDTKNG |
| | | HPFLGKVLRPGLGHTEKLYQRYFEEKKEWLEATFYPAASPKRVPRFVN |
| | | PPTGKQKELPLIIRNLMKERPEWRDWKQRKNSHPIDLPSQLFENEICR |
| | | LLKDKIGKEPSGKLKWNEMFKLYWDKEFPNGMQRFYRCKRRVEVFD |
| | | KVVEYEYSEEGGNYKKYYEALIDEVVRQKISSSKEKSKLQVEDLTLSVRR |
| | | VFKRAINEKEYQLRLLCEDDRLLFMAVRDLYDWKEAQLDLDKIDNML |
| | | GEPVSVSQVIQLEGGQPDAVIKAECKLKDVSKLMRYCYDGRVKGLMP |
| | | YFANHEATQEQVEMELRHYEDHRRRVFNWVFALEKSVLKNEKLRRFY |
| | | EESQGGCEHRRCIDALRKASLVSEEEYEFLVHIRNKSAHNQFPDLEIGKL |
| | | PPNVTSGFCECIWSKYKAIICRIIPFIDPERRFFGKLLEQK |
| 60 | cas13b | MDTPNFSERIPVSLQSHPYYFAHYLNMARHNAYVILEYVNRELIKPGK |
| | | NLDEDNLIQSTVLKDGYFDRKPDELSHRNRLLVQHFPFLREAENEGAR |
| | | TCNPVSYKLKTALAALNQWRNNASHYPLNQNHEKDFDLQPFFSFAIE |
| | | ACKKRMREVFQPDDFYLLETNEKQFYTLHNENGFTEKGLYCFICFFLEK |
| | | KYAFQFLAGIKGFKNTTDNKFRATLETFTEHCCRLPKPKLDSSDIKLDM |
| | | LGELSRCPAPLFDLLDIEERKKFIREPEEVKPDESGDREEVQQVLMKRY |
| | | DDRFPYFALRYFEEKNLLKGISFHIHIGRWIKSEHTKKIMGAERDRRLLK |
| | | DIRTFGELKEFSPEHAPDYWLRDGITPDDVDQFSPQYRIVGNRIGIKLN |
| | | YNGHNRWSVPDKEINVKPDAIISTYEFLNLFLYEHLYQKKLTGLSPAEFI |
| | | QDYLDRFNNFLSEFKAGHIRPVGDFSLEKRRGQGDEPDLTARRKSLQK |
| | | ELDRFVLKGKDLPDKIREYLLGYKQKSEKKQAKWILGGMIKETVYWRN |
| | | KAEQSPEKMRSGDMAQQLARDIIFLTPPHTVKEHKQKLNSLEYDVLQ |
| | | YALAYFSSNREKLYSFFKEHQLTVKGDRAHPFLYKIRLDECQGILDFFIVY |
| | | MQQKEKWLGWLDRNLKSPRLNEEEFFNTYSYFIKTDTKRAIEMDYES |
| | | CPNYLPRGIFNEPIAKALQKAGVKIKDEDNASYALSVYSNGKTQPFYNK |
| | | ERYYNKGIFRMEELPEKLQPKELLGKIQWTIKSSGKDTEEFRSLQNLKN |
| | | RILNTEKEIRYVQSTDRALWIMVADLFPETFELRPDDLECIGHDLSDDLL |
| | | SRPYQMKEKVYNYTITDYLPIKRYGEFRRFLKDRRLENLLTYFEEGVPLH |
| | | REALVAELEAYDLQRKNLLEIIYRFEKLVFDRHRHELTFSGEGENQYVN |
| | | HWDYLDFVARKYGLSAEVKELNSERFTELRNKMLHNQIPYQLWIKEAl |
| | | AAREENTVCGRIMGMIGEIYERMTTEIEKQMQV |
| 61 | cas13b | MFDNEQKNLEKEPYWGVFLNQARLNAYIALRDISERLEENAADEDSLS |
| | | EWPVLKYLDNDTDAVKSRRIFDLVEKHFSMLKIIYGGEKEGDLVKRSKE |
| | | YKIILKCLFRALNFYRNKFCHMYSGNRARKYNEKELIKYLEDCFDASVRK |
| | | IKELRRLDEKDVLHLRRKIAEGKDANKRVIDNPQFRYPFKNEKGELNEK |
| | | GLYFLASIFLDKKEAHEFLKKQEYFKNDSEPKYRATLESFYHYRIKLPRPV |
| | | lESDVDKNGLALDMLNELKKCPKELFDLLSKEQQEKFRVVDSEDADEE |
| | | GNEILMRRYSDRFPYLALRYCDENQVFERIRFQIDLGRYYFKFYPKETID |
| | | GKTQQRSLDKRLKIFGRIKDVKSKVEQEWSGIIKSPDTIEENPNEPYKLK |
| | | TTPRYNIVDNQIGFVITGDKNLPDVKRPDGRIELEKPDGWLSIYELPGM |
| | | LFHGLKYGFDKTERMIKIYIEKQRKICKEICEKGTITPDDGESMPEALKG |
| | | GAKAAKRNYSEKKLERMLQDTEQRIRAIQTTQKRMDEPGNKPGKKKF |
| | | FDIRAGKLADFLARDIMALQRFDPAKHGKDKLTAINFQVLQATLAFYG |
| | | AKKDVIEDMFKGIGLLEGDNPHPFLNQIDPAQYNSIAGFYQAYLQKKR |
| | | SYLEDYRKEEEYDEQFLRPKRQRYAQEKREIKTVARQLLDNPVNVPKN |
| | | FFKKEIEEFVFSQDPSLKKSKMNTAYMIQALFEKHYGRQQPFYSYNRT |
| | | YPVVSKAIEYGKKGKNKKIAKVLMAIEPKLNYMEIKKIVNEMPDGQYE |
| | | PENLKRNLYEGYKDYEKDERIIRRCKVQDVVSFMMVEETLKDQLDFN |
| | | GNVLTLEKITPWEASPFKKPVLCHTIISIPFNTKGGHTDKDYVDFIKNNF |
| | | EGSYDCEPNKIILKYKVTSKDTKLKDIGKYRMYSHDRRLPGLLIWKYRP |
| | | NDQNGNEIKFTEIEQEIKAFERRRIEIAQCLYTLEKKVIDSWFTQDELGE |
| | | EHIPFNKVIDVIKAKMPNFEDKCNVLLKIRNAINHNQFPVYEQAIQTAP |
| | | GKEIAGKMLRITESYIEQIMAKIDPDFGRTEDAESSR |
| 62 | cas13b | MDTPNFSERIPVSLQSHPYYFAHYLNMARHNAYVILEYVNRELIKPGK |
| | | NLDEDNLIQSTVLKDGYFDRKPDELSHRNRLLVQHFPFLREAENEGAR |
| | | TCNPVSYKLKTALAALNQWRNNASHYPLNQNHEKDFDLQPFFSFAIE |
| | | ACKKRMREVFQPDDFYLLETNEKQFYTLHNENGFTEKGLYCFICFFLEK |
| | | KYAFQFLAGIKGFKNTTDNKFRATLETFTEHCCRLPKPKLDSSDIKLDM |
| | | LGELSRCPAPLFDLLDIEERKKFIREPEEVKPDESGDREEVQQVLMKRY |
| | | DDRFPYFALRYFEEKNLLKGISFHIHIGRWIKSEHTKKIMGAERDRRLLK |
| | | DIRTFGELKEFSPEHAPDYWLRDGITPDDVDQFSXPQYRIVGNRIGIKL |
| | | NYNGHNRWSVPDKEINVKPDAIISTYEFLNLFLYEHLYQKKLTGLSPAE |
| | | FIQDYLDRFNNFLSEFKAGHIRPVGDFSLEKRRGQGDEPDLTARRKSL |
| | | QKELDRFVLKGKDLPDKIREYLLGYKQKSEKKQAKWILGGMIKETVYW |
| | | RNKAEQSPEKMRSGDMAQQLARDIIFLTPPHTVKEHKQKLNSLEYDV |
| | | LQYALAYFSSNREKLYSFFKEHQLTVKGDRAHPFLYKIRLDECQGILDFFI |
| | | VYMQQKEKWLGWLDRNLKSPRLNEEEFFNTYSYFIKTDTKRAIEMDY |
| | | ESCPNYLPRGIFNEPIAKAVQKAGVKIKDEDNASYALSVYSNGKTQPFY |
| | | NKERYYNKGIFRMEELPEKLQPKELLGKIQWTIKSSGKDTEEFRSLQNL |
| | | KNRILNTEKEIRYVQSTDRALWIMVADLFPETFELRPDDLECIGHDLSD |
| | | DLLSRPYQMKEKVYNYTITDYLPIKRYGEFRRFLKDRRLENLLTYFEEGV |
| | | PLHREALVAELEAYDLQRKNLLEIIYRFEKLVFDRHRHELTFSGEGENQY |
| | | VNHWDYLDFVARKYGLSAEVKELNSERFTELRNKMLHNQIPYQLWIK |
| | | EAIAAREENTVCGRIMGMIGEIYERMTTEIEKQMQV |
| 63 | cas13b | MKTLGALSSHNYNNKKYYFSGLLNTAQYNFNLALQEVNDRLGKKGKN |
| | | PGKTMIKNIFDQKDSFSTQERAMYYLEEFFPWIFLVMKQSGINIPTEE |
| | | QETKLHKEEIQLIQEHLISLYELLDDLRNEQTHYMHDPVIIPEEVSKMLD |
| | | ALLLQILKNTRKKCKDDEYRTFIVKKYQEEFQKEIKVQVKDRFGKEKEKI |
| | | VTGEVKENYVINRCFRKWIQKEGEEETLRYSTVQEEQGKYVWSSSGFV |
| | | FFLSLFLRRKELEDVMNHVPYFKDSRKLLFYLTRKTFSSYCFRDLRKSLRS |
| | | DYSNDSLLMQMIEELYKCPGELYEVLLKEQKQEFIEDINEYYKDNPEFE |
| | | GSANEAQVIHPVIRKRYQDKFPYFALRFIDEYFNFPTLRFQLVLGEYVT |
| | | DRRTKELQGTALFTDRVISQRISYVGKLSEAEMNKKREGYTETGWKEY |
| | | PNPYYKIENNRIPLYIEFSKNEELIFKEKKFKYNTLAKWENREIDKRTGEF |
| | | NQVNKQRRITQLEEFKIDNPKKMKTPNVFLSIYELPALLHALLIEKKTEA |
| | | EIEDIIKAKIKKQLTEIAEGRRNLSGLPKGIKKMRNCNSDFEKKKLISDID |
| | | NEIKKGEKILEEVQQWLNPVINKKGTGKQENNKPFFSNTYRGKYATW |
| | | LAYDIKRFTGKDHIQNWKGYQFSELQTLLSLYTLRKEELKNFLEKDLQLT |
| | | SHPFLKEALKAVNLEDFMGAYLRGRQFFLEKAKKQIGIKGVKKSIFQYF |
| | | EERKYKIYSSNLDYWEELWKHPVNLDRGLFDERGTVYNKNKELNDLQ |
| | | NRAAWFSFAETNPKQQFYHFPRIYSDEDITKPVTDRYGKTKEKLILFKLS |
| | | PQKGFMEQIPSDLKKKYQEDKGKVEHPEVQKEKKYEEKKHPGINAFIK |
| | | NAYKNEQKIRRISRNDIFLYEMVKYMLNKISPATEFSSLDKVWLTRIERE |
| | | KQATEAREQSFKEKGDTSENKIRQDYLLSFPITLTLFNDIIKEKVKIKDIG |
| | | RFRKLEKDERVQTMISYYTSGLWKNDQPSLTIKELEAELESYHKIRLQEI |
| | | FKEVHKLEKEIYEFTPEEDKSKLLARESFPKFKYYISFYFIPKEDQEVFNEI |
| | | QFDKYKNLEQIPGRKPEYDPYYLLIFIRNKFAHNQLPAEPIYKTALTFLP |
| | | NNFNTLAEYYHKLFILLNNKNYNN |
| 64 | cas13b | MNILPAAPEKEKIAYSTATAPWFFGAFLNQARHNLFLTVNDLAIRLGE |
| | | KVIDYDDQLLNSNVVRMLVNEKASPLQLEILMKYLDRHLPFLIPMQVA |
| | | LKGHQGDASDNPVIGSPADYGAILSKLIVCLNAARNHFSHYHSTSGWS |
| | | GYNEVIEWMEHVFTRNIETVVKRFTLTEEEVQHLKKPVDKSPKGTIPPY |
| | | YFSFCKGDIWTDTGLAFFICLFLTREEAYLFLKKLRGFKRGEERFHKATLE |
| | | AFCVGSLKVPRERLESNNSPQSAFLDMCNELVRCPKSLFDLLEPEKQEL |
| | | FRRDPEPEDAEDNGIEEEEDQPQALLVRKENRFSYFALRYLDIAKAFPR |
| | | LRFGVDLGTYFFSVYPKTFAGIEETRQLSKRLIGYGKLEEFAREKRPEHIA |
| | | ALFRSKEEANAAPTEPFIRETAPHYHLDGNNVYLYMSGDGEAQWPAV |
| | | ELEEVTGKSYPRKLVKKSTLLPFAVLTVNELPALLFYHLLHKEKGAGDAA |
| | | ERVIINHMERVKRFFKALQDDKVDQVAGQPIRKPDVDADESLHMEY |
| | | DRRWKLLKKKLSEYQLRASYIPEKIINYLLNIEAVDLGDKAMAQLKNLQ |
| | | RQAQDDIAAIERRMEHLMKKGADGRKTLKVGNLAQQLAEDMLQM |
| | | QPVQIGTDGEPVPASKANNLAFRLLQSHLAYFAENRHNLPAVFEACGL |
| | | IGASNKHPFLDNINIESCKGVVDFFILNFRNKLDFLDRCLQEGEWHRYH |
| | | FISAAKLKSGAKVTIKKYLNEAFESKGRNHIPFTLPPSLFLDASLDWLAKF |
| | | GDGKAKKVLAENEYVNSVFLIRRLFADGGLQPFYAWKREYRLFEKKAG |
| | | KAVFLDEAGRMRKADKIGIEVERHREFLARPVKKGKQYDIKKAAAEQF |
| | | LRSYRFYLQEEKYIRLLAAQDMLLFRCICDLLTYHVGDIGLEELAEAKAG |
| | | TFSLANITPEKTETAKSLLNYRPAGGVVLDRHFYATDEKGAFVKQEGKL |
| | | VPGGQVRIFDNTLKIKNAGNFRKLLKDRRMNNLFFYFKQHADEPVVL |
| | | HRMVLENELRAYDRMRLKVLPVIAEFEKKLYQHCTDVEKERLVVNGS |
| | | MHHRCYLDVYREKYQPDWGWEAAGNLLRIRNAFVHNQFPLMEGD |
| | | GFKLEVAHWKKINADFVPSEQGSSLGYGIIDRLGQLAVEGYEGLIKNIH |
| | | V |
| 65 | cas13b | MGEEEQAHILENSLNDELCEAIDDPFEMIASLSKRARYKDRFPYLMLRY |
| | | IEEKNLLPFIRFRIDLGCLELASYPKKMGEENNYERSVTDHAMAFGRLT |
| | | DFHNEDEVLQQITKGITDEVRFSLYAPRYAIYNNKIGFVWTSRSKKKSF |
| | | PTLKKKEGEGHRVAYTLQNEESFGFISIYDLRKILLLSFLDEGKNIVSGLF |
| | | KQSKANWENLSENLFDAIRTELQKEFPVPLIRYTLPRSKGGKFVDPKLA |
| | | DKQEKYESEFERRKEKLSEILSEKGFDLSQIPRRMIDEWLNVLPTSKEKK |
| | | LKGYVETLKLDCRERLRVFEKREKGEHPVPPRIGEMATDLAKDIIRMVI |
| | | DQGMKQRITSAYYSEIQRCLAQYAGDDNRRHLDSIIRELGLKDRKKGH |
| | | PFLGKVLRPDLDHTEKLYQRYFKEKKEWLEATFYPAANPKRVPRFVNP |
| | | PAEKQKELPLIIHNLMKERPEWRDWKQRKNSHPIDLPSQLFENEICRLL |
| | | KDKIGKESSGKLKWNEMFKLYWDKEFPNGMQRFYRCKRRVEVFDKV |
| | | VEYEYSEEGGNYKKYYEALINEVVRQKISSSKENSKLQVEDLTLSVRRAF |
| | | KRAINEKEYQLRLVCEDDRLLFMAVRDLYDWKEVQLDLNKIDNMLGE |
| | | PVSVSQVIQLENGQPDAVIKAECKLKDVSKLMRYCYDGRVKGLMPYF |
| | | ANHEATQEQVEVELRHYEDHRRRVFDWVFALEKSVLKNEKLRRLYEK |
| | | SQEGCEHRRCIDALRKATLVSEEEYKFLVHIRNKSAHNQFPDLEFGKLT |
| | | PNVTSGFCECIWSKYKAIICRIIPFIDPERRFFGKLLEOK |
| 66 | cas13b | MRIPKLIEEHKSVFGAYSTMALSNVETVLNHIAERAGLDGYERDRGPG |
| | | VEDYWEHPVMQCLCRKDKPRSIPSDVLLDVRNRLFRSFPFLKIMAEN |
| | | QRDYRNAKGKVECVEINESDIFVVLNNSFRVLKAYRDTCTHYLIENRIW |
| | | EDNSPMLMYNECPLAAMVNQYYTAALRVTKERYGYETRDLTFIQKRR |
| | | FKQEPEKEASGNVKKKAVPDLAFFLSLVALNGDGRKWLHLSGWGVVL |
| | | LICLFLEKKYVNVFLSKLPNPGNYPPSSKERRIIRRSMGVCSVVLPKERIH |
| | | SETGDLSVALDMLNELKRCPRELFDTLSPGDQERFRTISSDHNEVLQM |
| | | RSKDRFAQLVLQYIDHNRLFENLRFHVNMGKLRYLFNPKKYCIDGQTR |
| | | VRVLEHPLNGFGRLQEMEEKRLQENGPFARSGIKVRCFDEVRRDDAN |
| | | ESNYPYIVDTYTHYVLENDMVEMFFCPEGSGMKMPEVTSREGKWYV |
| | | DKKVPHCRMRMSVLELPAMLFHLLLCGAKNTEVHIGKVCDNYCHLFS |
| | | DMAQGNLTEENILSYGIKKEDIPQKVWDCVRGVHMGKDSRAYREKEI |
| | | RERYEDVTRRLERLEADRKAVLGGENKIGKRGFVQIVPGRLAAYLATDI |
| | | CRLQPSLRKGDGYGTDRLTGLNFRLLQSSIATYNCGESDILYGRFRDVF |
| | | CSAGLIGGDNPHPFLDKVLPEAYSVCCPRNTIEFYERYLEEYQRYLKPLV |
| | | IKLEKGKVPSLSFVNEGQRRWARRDDAYYHELGNLYLSQAIELPRQMF |
| | | DDEIKDKLREMPEMRDVDFDHANVTFLIGEYLKRVRHDESQEFYSWP |
| | | RHYKYVDMLKCILNPKNGSLQAVYIQMGEREGLWQERSELEEKYAKIR |
| | | LRDLGRKGLDKDEANERIKTGLGNRKKEYQKAEKVIRRYKVQDALLFM |
| | | LAKNTLFNSVEVDDERFKLKDIMPDGEKGILSEVVPMDFCFRSGNSAT |
| | | RKLMGTIHSDNTKIKNYGDFFALANDKRMVTLLPLVGEQCLVKEEVKE |
| | | EFDKYDDCRPEMISMVFDFEQWAYSAYPELKELVSNEAIKGRLFSNLL |
| | | QELLGRGELTYEEKYALVGIRNAFLHNSYPKDGGVVKVRTLPDIAKSLK |
| | | DVFKEYIRLE |
| 67 | cas13b | MKMFYKSVLTAFFTAVDSLRNKYTHYSHKDLNIREIKIECTLGGKDYCI |
| | | GLLNALDCIYDSAVNLLKLRFMAGEDEVAHLRRCKAVNKKVVVRTEKD |
| | | GFYYRLSDNGGVTEKGVIFIASMFLNRKYGFLFLKQLEGFKRSDEKRYR |
| | | LTLEAFLAFSNIKPVDRLKSDKLDRASLGLDMLNELTKIPKELSETLSVDC |
| | | LYKYLASDGEDDLRSRIRYQDRFVPLALEFISQSDEFKDFRFYTYVGNYV |
| | | YKGYIKRLIDGTDKERYLSDRLCGFYKSVNDASSDAIAQKYGVEIKDSNE |
| | | PDYMLPDSFRPHVLRATPHFVINTNNIGIKICGNDCLPIVNGKGVESPE |
| | | PDYWLSIYELPAMLFYAYLREKNGKRFKDYKSIRELIEGVEKKADEKND |
| | | RDKGALMARHIDKEIIWTQTKLDEVKRLEEKKVAAYGKKGRVVLKAGR |
| | | MADLLAHDMVRLQPATKGSDKITGANFQALQVSLAYFKRDILADVFS |
| | | RAMLTTGNHRHPFLYRIDVSHCSSLRDFYVAYLGERRKYFEDVAKKIAK |
| | | NKLNTPCHILRRLQREGSGEEAGKDVKPKFLPRGIFTDSIKNCLEQSKL |
| | | NIYIRNARNDVKPAINAAYLILMYYKEIEKGEFQGFYGEKRRYDILEEGK |
| | | PLDLAERKKALASIKPAKIDVSEANMPMSKEEHLMRKRYHAVCNNES |
| | | AIRMYQVQDILLLLMAKDIFKKALSEGVMSKKIGLENLNGIFDAPVNFV |
| | | KNFDNIKLTATGIKIKDYGKVCRLGTDFKFNSLIKAFHKVYSKSVEMDYS |
| | | DYLKEEEEFEKYRLNMVKLCREVERGITEDLHLSLDGKSHLSFNDDVIKP |
| | | YNDKYNVFNGGDLTFFINARNMFMHGDYKYECVKYVVSEHFKGSLN |
| | | DVSFAKETYGHFCNLLESMRKKTGLRIDI |
| 68 | cas12g | MLPTRYKPARTLVRPLGRLPHEPRKEFVEKCRRVRMHFEQFNIDVADL |
| | | CQWLMSLRPNTRIGDAQSTVFWDFFLNPSILTVEADEKERDRWRLAA |
| | | FDELLQIRFGHDPNAPPWSEEFRSAIRHVAQRPKSATAQRLFDRLRSL |
| | | TAPHRLVLLKSAAEWIIARYQRGMENWQRQFAEWQREKEEWEAAH |
| | | PNLTPEVRDAFTRVFKNLFENPDGDGKIGVRRKNPRICSWERLKLNKD |
| | | NCVYAGQKGHGPLCWEFSKFVKAQKNAGTIKTFFVDVANKYLHVRR |
| | | NLSKPGVKLKKSPRQEAFKRLYNQKGMEKARNWFTDAWSGYLTALN |
| | | LNEKTILDHGCLKHCGAIGAEFEKSLCQFNPHTHLCVQ.YRNALESLEPA |
| | | IRELEGDYREWRRLFLAPPRKPSFRYPSSRRLPMPKIFGEHFHQIDFDQ |
| | | SILRLRLEDMAEGEWIEFGFKPWPKDYRPGKDEVRVTSVHVNFHGNR |
| | | MRAGFHFEAPAKPSRFACTQDELDDLRSKQFPRQSQDRQLLEVARRR |
| | | LLESFDGMLESDLRILAVDLGEKGAAAAVYQGHGHEADVAIPIVKIDRL |
| | | YDHVPDVLDVESARVPPPKFDDSRDPRGVRKEHVGRHLGQLQRGAQ |
| | | TLAQHRQQDESAPAALRRHDFRSLTRHIRWMIRDWTRHNAAQITAA |
| | | AETHRCHLIVFESLRGFKPRGYDQMDFAQKARLAFFAYGRVRRKVVE |
| | | KAVERGLRVVTVPYGFTSQICSECGHRQRNKGRLRKNKYQRRFVCEC |
| | | GEPKKSANKTAAPDRSATVSPCTCRLQLGSDVNAARVLARVFWDEIV |
| | | LPTREEMREPAVDSAPPSK |
| 69 | cas12g | MCLCTLSGRTRQEEEIIGSTQYTEARSLVRRIRRPRGESRRQFKSNVLLL |
| | | RRHFEQFNVDASEICQWLMGIRPGGRHADESTGPFWEFFLDPGRFLR |
| | | ETGRGPEDADERIDAYRRIAFDVVAGIEDESRMSDPSIPRQIVESLHAV |
| | | SMATRTESARRLFERLAGLEPSHRQILLKAAAEWIVSRYWRSVQGWP |
| | | DRYKHWSDEKEEWEKAHPRLTESLREEFTGIFRDLGIRRKKPRVCPWE |
| | | RLEKGMDNCMYAGERIKVGYSRQSHSQLCAKYERFSYKQRQRTKSGK |
| | | NFKSYFVKNAELYLKLRRKNRSLIKKDVMKLFRKKVPQALWFEKAWDE |
| | | YLKALGVDEATLTKDGKLPHCTQFADDKECLFNRHTELCLQYRERLLRL |
| | | PHLQELEQLYREWRDKYLSGPRRPSLRYPSKRTLPMPKVFGRGYFCAD |
| | | FTNSLLDLRLEGMGEGDFVRFGFAPWPADYDAQPSDATVTSVHIHFV |
| | | GTRARAGFRFQAPHKTSRFASSQDEIDDLRSRKFPRAAQDGEFLDAA |
| | | RKLLLESFTGDAEREMKLLAVDLGDRGAGAAVFEGRCFKEAMPLKIIK |
| | | TDTLIDKPPPVTKTPRKGKPGKRESKRARGLDKYHVARHLDTWRKGA |
| | | RKIAERRAKGEADPVKLGAHDMRSLSLHVRWMIRDWVRLNASQIIKT |
| | | AESHKTDLIVLESLRGFSAPGYHKLDDEKKRTLAFFAYGRIRRKLTEKAV |
| | | ERGMRVVVAPYLRSSQVCAECGREQIDRNKLMKDKRKRRFICEYSDC |
| | | TWQCDSDQNAACVLGRVFWGEIELPSERKKD |
| 70 | cas12g | MHPSRYKTARTLVRRLCRLPGEDRSAFRSKVGLLRGHFEQFNVDVSEL |
| | | CQWLMSLRKRNKVPENPATFGALGDFLLOPGLPGEETDEKEADRLRL |
| | | AVFDAVAGFRMLEDRLAASIPASLSDAIRDEAVFLAGVRAAGKPSGLA |
| | | RVLARLEACAPAQRLVLLKSAAEWIVARFLRGTENWMRQRAEWEKE |
| | | KAAWEAAHPHLTPEVRAQFNKIFESLHDPENSGKPGVSRKNPRICPW |
| | | DRLKQNLDNCCYGEKGHSALCWRYQDFLKQRMGENRRDKKNFSAT |
| | | AMDLAQICREWKIQHSRNALNNPRVLDRLFAEHERRKQDKTKKESRS |
| | | PKPRQGGYKANPKADYLRSFKAHWKAYLEHMKLNDTTVLERGCLPH |
| | | CLSIKKNGKESTCKWNKHTELCLEYKRSLAPLPDSVLELEPEYREWRRL |
| | | YLHGPGRPHFRYPSAGELPLPKVFGEGFHQVDLDRSIVRLRLEGAAEG |
| | | EWLEFGFIPWPRGYQPSRREVLITSVQVHFVGTRPRAGFRFDVSHRTS |
| | | RFGCSQDELDELRSRRYPRQAQDKEFLAAARAQLIQTFEGGEGAARQ |
| | | QMRVMSVDLGEGGACASIYEGRTHQKDESLKVIKIDRRYDQHPEVLE |
| | | KDVGAAKPQKFEKSDPRGVRKEHVARHLNRIAAGASAIAEHRRKERS |
| | | DAECSVGELQEHDFRSLKRHIAWMIRDWVRLNAAQIIDVAKQHCCDL |
| | | IVFESQRGFRLPGYDELDRGKKQRFAILAFGRIRRKVVEKAVEHGMRV |
| | | VTVPYFASSQVCSACKRVQENRGSWRENKKKRVFACEFCKLKLNSDA |
| | | NASRVLARVFWGEIELPEPTRAHLPSKA |
| 71 | cas12g | MPVSRYSESRTLVRPLARLPHEERQDVTPKVARLRRHFERFNVDVAEL |
| | | CQWLMGLRNQFGPKESPASFGPLGDFLIEPALDNIDADETERDRWRL |
| | | AVFDAVAGFRPIRGLGDHPVPDTLRLAMQQAASLSPTPTTARLLERLR |
| | | PLSPAHRLVLLKSAAEWIVARYQRGMENWVIQHAAWHKEKEAWER |
| | | EHPALTPAVRERFTALYKQLSDSKPTDRPVSRRKNPRICEWERLRQNID |
| | | NCCYAGEKGHGPLCRKYANFVKARKAVDGKFNDLLFWDTATSFIALC |
| | | RKFNVTRARNALQSQLDALFAEDQRRKAERDQAKGRQPRPLHPQAA |
| | | ARAKSDFLRIFKDGWNAYLSAMGLNDSTAIEKGRLPHCQKIGGTFENS |
| | | KCEWNPHTDLCHQYRRLAGQLDDATLALEKDYREWRRLYLAGPRKPS |
| | | FQYPSSRDLPMPKIFGAGFFELDMDRSILRLRLDDMVEGEWLEFGFKP |
| | | WPREYTPSRAQVARPGRITSVHVNFIGSRCRVGFRFEAPHAGSRFGCS |
| | | QDEIDQLRRDHPRERDDQPFLEAARKRLVETFAGDARRDLRLLAVDV |
| | | GEKGCCAAVYQGTRYVADALLPIIKINQLYTEPPTELKPDSHNRPAPDR |
| | | RPFNDEKDPRDPRGVRKEHVARHLKRMADKAPEVAAYRLAQREKAA |
| | | PSPSASPPPVTLGVHDFRRLKRHVTWMIRDWARHNAARIVAEAQRH |
| | | GCDLIVFESHRGRRPPGYHEVGDDAERRKLDNATFAFGRIRRKVTEKA |
| | | VERGLRVVTVPYHCSSKVCSRCGRLQENDGLLRRNKKERKFICEQCKF |
| | | ETNSDGNAARVLARVFWGEIMLPSPEERRKKREGSGGRSPTPANPG |
| | | GLVDAPPSRRNLR |
| 72 | Cas12 | MEDYSGFVNIYSIQKTLRFELKPVGKTLEHIEKKGFLK |
| | | KDKIRAEDYKAVKKIIDKYHRAYIEEVFDSVLHQKKK |
| | | KDKTRFSTQFIKEIKEFSELYYKTEKNIPDKERLEALSE |
| | | KLRKMLVGAFKGEFSEEVAEKYKNLFSKELIRNEIEKF |
| | | CETDEERKQVSNFKSFTTYFTGFHSNRQNIYSDEKKST |
| | | AIGYRIIHQNLPKFLDNLKIIESIQRRFKDFPWSDLKKN |
| | | LKKIDKNIKLTEYFSIDGFVNVLNQKGIDAYNTILGGK |
| | | SEESGEKIQGLNEYINLYRQKNNIDRKNLPNVKILFKQI |
| | | LGDRETKSFIPEAFPDDQSVLNSITEFAKYLKLDKKKK |
| | | SIIAELKKFLSSFNRYELDGIYLANDNSLASISTFLFDD |
| | | WSFIKKSVSFKYDESVGDPKKKIKSPLKYEKEKEKWL |
| | | KQKYYTISFLNDAIESYSKSQDEKRVKIRLEAYFAEFK |
| | | SKDDAKKQFDLLERIEEAYAIVEPLLGAEYPRDRNLK |
| | | ADKKEVGKIKDFLDSIKSLQFFLKPLLSAEIFDEKDLGF |
| | | YNQLEGYYEEIDSIGHLYNKVRNYLTGKIYSKEKFKL |
| | | NFENSTLLKGWDENREVANLCVIFREDQKYYLGVMD |
| | | KENNTILSDIPKVKPNELFYEKMVYKLIPTPHMQLPRII |
| | | FSSDNLSIYNPSKSILKIREAKSFKEGKNFKLKDCHKFI |
| | | DFYKESISKNEDWSRFDFKFSKTSSYENISEFYREVERQ |
| | | GYNLDFKKVSKFYIDSLVEDGKLYLFQIYNKDFSIFSK |
| | | GKPNLHTIYFRSLFSKENLKDVCLKLNGEAEMFFRKK |
| | | SINYDEKKKREGHHPELFEKLKYPILKDKRYSEDKFQF |
| | | HLPISLNFKSKERLNFNLKVNEFLKRNKDINIIGIDRGE |
| | | RNLLYLVMINQKGEILKQTLLDSMQSGKGRPEINYKE |
| | | KLQEKEIERDKARKSWGTVENIKELKEGYLSIVIHQIS |
| | | KLMVENNAIVVLEDLNIGFKRGRQKVERQVYQKFEK |
| | | MLIDKLNFLVFKENKPTEPGGVLKAYQLTDEFQSFEK |
| | | LSKQTGFLFYVPSWNTSKIDPRTGFIDFLHPAYENIEKA |
| | | KQWINKFDSIRFNSKMDWFEFTADTRKFSENLMLGKN |
| | | RVWVICTTNVERYFTSKTANSSIQYNSIQITEKLKELFV |
| | | DIPFSNGQDLKPEILRKNDAVFFKSLLFYIKTTLSLRQN |
| | | NGKKGEEEKDFILSPVVDSKGRFFNSLEASDDEPKDA |
| | | DANGAYHIALKGLMNLLVLNETKEENLSRPKWKIKN |
| | | KDWLEFVWERNR |
| 73 | Cas12 | MKKFTNLYSLSKTLRFELIPQGKTLENIQKSGILEQDNSRAEKYEKIKKI |
| | | IDDYHKFFIEKSFTGKKIDDYFLNQYFELFKIKDKDEEQKKDFKSIQEN |
| | | LRKNIISFFDKNKLKRLFEKEIIKEDLPNFVKEEEDKKLISEFDKFTTYFV |
| | | GFHENRKSMYSEEEKSTSIAYRTINENLPKFINNIFVFEKISKTPISENF |
| | | RELYKDLEEYLNVNDIQDIFKLNYFSNVITQKQIDVYNLVIGGKTLEN |
| | | GTKIKGLNEYINLYNQNQTDKKNKLPLLTVLFKQILCDRDTISFLPEQF |
| | | ENDIDVLDNIKNTYSNMEKSIKDIKDLLSNLKDFDLSKIYITNDIALTDI |
| | | SQQVFNNYSIIINAIKENIKKENPKKKTENEEKYGERIDKIFKSNNSFSI |
| | | KYINDCIKEKNIEIYFMDFGKKENNKKVKNLFDELQNNYSMVKDLLE |
| | | YKKIQSLIQDEKSIELIKNFLDSIKNIQHFLKPLYVKDNDIVKDISFYRDF |
| | | EELYLNIDKITPLYNKVRNYVTQKPYSVKKIKLNFENSTLLAGWDLNK |
| | | ERDNTCAILRKDDLYYLAIMDVNNRNVFNEKGIDGIGYEKMEYKLLP |
| | | GANKMLPKVFFSKSRIKDFNPSEQIIRNYEKETHKKGSNFSLKDCHKL |
| | | IDFFKSSINKHEDWKNFNFKFSNTDKYEDLSGFYREVEQQGYKITFR |
| | | NISKEYVDKLVEEGKIYLFQIYNKDFSKYSKGTPNMHTLYWKALFDE |
| | | DNLKNVVYKLNGQAEIFYRKGSIEKENIVIHKANNAIENKNMDNKKK |
| | | QSKFEYDIIKDRRYTVDKFQFHVPITLNFKAIGNERINEQVNQYIKDN |
| | | NIKHIIGIDRGERHLLFLSLIDLKGNIIKQFSLNEIVNEYNGNSYKTNYH |
| | | MLLEKREEERDKARKSWKTIENIKELKEGYISQVIHKITQLMIEYNAIV |
| | | VLEDLNFGFMRGRQKVEKQVYQKFEKMLIDKLNYLVDKKKDKNEA |
| | | GGLLKAHQLTNKFESFQKMGKQNGFLFYIPAWNTSKLDPITGFVNL |
| | | FDTHYTNVDNAKKFFENFEDIRFNEKKNYFEFIVNDYTKFNTKAEGT |
| | | KLNWTICSNEDRIKTFRSSSKNNQWVSETVNLTDSLIELFKKYDIDYK |
| | | LELKEQIISKSEKNFFETLLYLFKLTLQMRNSITGTETDYLISPVADKTG |
| | | NFFDSRKGIENLPNNADANGAYNIARKGLWVIEQIKKAKDLKKVKLA |
| | | ISNKEWLQFVQGK |
| 74 | Cas12 | MAKNTIFSQFTGLYPVSKTLRFELKPMGKTLEKIKETGVIENDKKRHN |
| | | DYFDAKKIIDKYHKYFIDAALSKFPRIDWSPLKEAIERSLDRSDASKKKL |
| | | EKTQTEFRKKIAKALTTHDHYKELTASTPKDLFLKVFPDHFGKQPAID |
| | | TFDGFSSYFTGFQENRQNIYSDEAISTAIPYRLVHDNFPKFLSNIEVYK |
| | | TLKDNAPSVLSDAENELRDFLNGKSLANIFELNAYNEVLTQSGIDFFN |
| | | QVIGGISDEGGEKKTRGINEFSNLYRQQHPEFAQKRLATKMIPLYKQ |
| | | ILSDRETKSFILESYSNDSQVQNSVKEFFESQILNWDIAGRRVNVLNE |
| | | LTSLVKRISEFDLGNIYVNQEELSNISLKLFDNWNSINGLLFKHAENRI |
| | | GSAEKSANKKKIDAWMKNKEFSIATLNLAIAESNSEEISRVKIESYWN |
| | | NFEAKVQSILCGDNRRNLDEFISATFNENNALREDSKIIEKLKAFLDAL |
| | | lEIMHSIKPLISDAENRDLSFYNELIPLYDQLSLVVPLYNKIRNYATQKL |
| | | TESEKFKLNFDNPTLADGWDQNKEEANTAILLLKNGLYYLGIMNAK |
| | | NKPKIKDFKTSESEDCYDKMVYKLLPGPNKMLPKVFFSEKGLATFKP |
| | | PKDILDGYNAGKHKKGDLFDIGFCHQLIDFFKESIAKHPDWKKFDFN |
| | | FSDTSSYEDISGFYKEVTDQGYKITFSKIPTSQIDEWVKEGKLFLFQIY |
| | | NKDFAPGAKGSPNLHTLYWKSVFSPENLKDVVVKLNGEAELFYRPS |
| | | SVKKPYSHKVGEKLVNRIGKDGLPLPESVFGELFRYFNGKLEGELSDE |
| | | AKRYLDVAVVKDVKHEIVKDRRYTQDKFEFHVPLTLNFKADSKNEY |
| | | MNERVRHFLKDNPDVNIIGIDRGERHLLYMTLINQKGEILKQKSFNV |
| | | VESVNYQAKLVQREKERDAARRSWSSVGKIKDLKEGFLSQVIHEITT |
| | | TMIENNAIVVLEDLNFGFKRGRFCVERQVYQKFEKMLIDKLNYLVFK |
| | | NKPEGDVGGVLKGYQLAEKFDSFQKLGKQSGFLFYIPAAYTSKIDPT |
| | | TGFANLFNMTELTSAEKKKEFLSHFEDITYDGKNDRFLFSFDYKNFKC |
| | | FQTDYIKKWTVYSQGKRIVYDKESKSAKEISPVEIIKAALAKQNIALTD |
| | | QLDVLSAINSVEASPKSASFFGDICYAFEKTLQMRNSIPNTDEDYLAS |
| | | PVMNKRGEFYDSRSCDDALPQNADANGAYHIALKGLYLIKNVFDA |
| | | GGKELKISHEDWFKFAQSRNC |
| 75 | Cas12 | MSKGKIWENFINQYSVSKTLRFELKPVGKTLENINAKGLIEEDEQRAE |
| | | DYKKAKKIIDEYHKYFIEGALGSCSLDLNILNEFLQLYNKAQKTDADKK |
| | | EYEKIQTTLRKNIAESFGKNADKKTKEQYENLFKKELLRNDLPDWVE |
| | | DEEDAKIIERFKTFTTYFTGFHENRKNIYDNEEKSTAIGYRIVHENLPK |
| | | FIDNMNAFEKISKALDLSEIDRDFQSELGEIKAEEFFTIEFFNQCLNQF |
| | | GIDRYNTLLGGISEGENIKKKQGLNERINLYNQQLKGERKKERLPKLK |
| | | VLYKQILSDSSSHSFSIDEFENDNELLESLEIFYKNELIGFNHSGVDSNI |
| | | FDLVKDLLLKIDESEQSSIYLKNDKGLTEISQRIFGDWNIIKSALEEYYD |
| | | EHYPPKKDTFNKKELDERSRWLKENHSIGVIEKALANYENEIVREHLK |
| | | QNSAPIVSYFKSLEVDGENLIDKIYSAYGNISDLLNSSYPDEKKLVSDR |
| | | TSKDKIKVFLDSLMSLLHFLKPLDVKDLGNKDSAFYGDYDFIVEQLSK |
| | | LVRLYNKTRNYLTRKPYSIEKIKLNFENSTLLAGWDVNKERDNNCVIF |
| | | KRQDGDRELFYLGIMDKSHNKIFTKIEEAKSDDVYQKMNYKLLPGP |
| | | NKMLPKVFFSKKSIDFYAPGEELLKNYKNGTHKKGENFNLQHCHELI |
| | | DFFKRSINKHEDWSQFNFKFSDTSEYEDTSFFFKEVSQQGYSITFKNI |
| | | DRETIEKFVDEGKLYLFQIYNKDFSPKSKGRPNLHTLYWKMLFDERN |
| | | LANTVYQLNGEAEVFYRKKSISEKDRVVHRADEPIGLKNSENSAQKS |
| | | LFPYDIVKDRRFTVDKFQFHVPITLNFKSEGNERLNISVNKFLKDNPD |
| | | VNIIGLDRGERHLIYLTLINQKGEILHQESLNEVMGVNYQQKLHRVE |
| | | KDRTEERRNWDRIENIKELKSGYLSQVVHKISQLMVEYNAIVVMEDL |
| | | NFGFKRGRIKVEKQVYQKFEKTLIDKLNYLVFKDREPEEPAGVLNAL |
| | | QLTNKFESFKKLGKQCGFLFYVTSDYTSKIDPATGFVNLLYPKYESVEK |
| | | SQNFFRKFDNICFNSGAGYFEFDFDYSNFTDRADGTRTRWKVCTVG |
| | | NERFGYNPKTKASETVNVTESLKELLLQHEIAFENGESLVESISKNTTK |
| | | YFHKSLLNFLRLTLTLRHSKTGTDIDYILSPVANEEGVFFDSRNASDK |
| | | MPKDADANGAYNVALKGLMVLERINAAEDLSQFKFKDMSIKNKD |
| | | WLKFVQDRQG |
| 76 | Cas12 | MKNLANFTNLYSLQKTLRFELKPIGKTLDWIIKKDLLKQDEILAEDYKI |
| | | VKKIIDRYHKDFIDLAFESAYLQKKSSDSFTAIMEASIQSYSELYFIKEKS |
| | | DRDKKAMEEISGIMRKEIVECFTGKYSEVVKKKFGNLFKKELIKEDLL |
| | | NFCEPDELPIIQKFADFTTYFTGFHENRENMYSNEEKATAIANRLIRE |
| | | NLPRYLDNLRIIRSIQGRYKDFGWKDLESNLKRIDKNLQYSDFLTENG |
| | | FVYTFSQKGIDRYNLILGGQSVESGEKIQGLNELINLYRQKNQLDRR |
| | | QLPNLKELYKQILSDRTRHSFVPEKFSSDKALLRSLLDFHKEVIQNKNL |
| | | FEEKQVSLLQAIRETLTDLKSFDLDRIYLTNDTSLTQISNFVFGDWSKV |
| | | KTILAIYFDENIANPKDRQRQSNSYLKAKENWLKKNYYSIHELNEAIS |
| | | VYGKHSDEELPNTKIEDYFSGLQTKDETKKPIDVLDAIVSKYADLESLL |
| | | TKEYPEDKNLKSDKGSIEKIKNYLDSIKLLQNFLKPLKPKKVQDEKDLG |
| | | FYNDLELYLESLESANSLYNKVRNYLTGKEYSDEKIKLNFKNSTLLDG |
| | | WDENKETSNLSVIFRDTNNYYLGILDKQNNRIFESIPEIQSGEETIOK |
| | | MVYKLLPGANNMLPKVFFSEKGLLKFNPSDEITSLYSEGRFKKGDKF |
| | | SINSLHTLIDFYKKSLAVHEDWSVFNFKFDETSHYEDISQFYRQVESQ |
| | | GYKITFKPISKKYIDTLVEDGKLYLFQIYNKDFSQNKKGGGKPNLHTIY |
| | | FKSLFEKENLKDVIVKLNGQAEVFFRKKSIHYDENITRYGHHSELLKG |
| | | RFSYPILKDKRFTEDKFQFHFPITLNFKSGEIKQFNARVNSYLKHNKD |
| | | VKIIGIDRGERHLLYLSLIDQDGKILRQESLNLIKNDQNFKAINYQEKL |
| | | HKKEIERDQARKSWGSIENIKELKEGYLSQVVHTISKLMVEHNAIVVL |
| | | EDLNFGFKRGRQKVERQVYQKFEKMLIEKLNFLVFKDKEMDEPGGI |
| | | LKAYQLTDNFVSFEKMGKQTGFVFYVPAWNTSKIDPKTGFVNFLHL |
| | | NYENVNQAKELIGKFDQIRYNQDRDWFEFQVTTDQFFTKENAPDT |
| | | RTWIICSTPTKRFYSKRTVNGSVSTIEIDVNQKLKELFNDCNYQDGED |
| | | LVDRILEKDSKDFFSKLIAYLRILTSLRQNNGEQGFEERDFILSPVVGS |
| | | DGKFFNSLDASSQEPKDADANGAYHIALKGLMNLHVINETDDESLG |
| | | KPSWKISNKDWLNFVWQRPSLKA |
| 77 | Cas12 | MNLIENETKSEEIKSKLDSIMEIMHWTKMFIIEEEIEKDVNFYNEIEEI |
| | | YDELQPLVTIYNRIRNYVTQKPYSEEKIKLNFGIPTLANGWSKTKEYD |
| | | NNAIIMIRDGKYYLGIFNAKNKPDKKIMEGHQSEENGDYKKMIYRLL |
| | | PGPNKMLPKVFMSKTGIAEYKPSQYILECYEQNKHIKSDKNFDIKFCR |
| | | DLIDFFKTSINRHPEWSKFNFKFSETSEYEDISTFYREVEKQGYKIEWT |
| | | YISEKEIKELDENGQLYLFQIYNKDFSEKSKGKENLHTMYLKNLFSEEN |
| | | LKNIVLKLNGEAEVFFRKSSIKKPIIHKKGSVLVNKTYNENGERKSIPEE |
| | | QYTEIYKYLNSIGTNELSEKSKKLMEEGKVEYYKANYDIVKDYRYSVD |
| | | KFFIHLPMTINFKAAGFSPINNIALKSIALKEDMHIIGIDRGERNLIYVS |
| | | VIDTKGNIVEQRNFNIVNGIDYKEKLKQKELDRDNARKNWKEIGKIK |
| | | DLKEGYLSLVVHEIAKLVVKYNAIITMEDLNQGFKRGRFKVERQVYQ |
| | | KFETMLINKLNYLVDKDLAVDQEGGLLRGYQLTYIPESLKVLGRQCG |
| | | YIFYVPVAYTSKIDPTTGFVAIFNYKGMTDKDFVTSFDSIKYDDERGLF |
| | | AFEFDYENFVTHKVEMARNKWTVYTYGERIKRKFKNGLWDTAEKV |
| | | DLTYQMRSILEKYEIEYNKGQDILEQIEELDEKAQNGICKEIKYLVKDI |
| | | VQMRNSLPDNAVEDYDAIISPVINNNGEFFDSTRGDEDKPLDADAN |
| | | GAYCIALKGLYEVMQIKKNWNEETEFPRKELKIRHQDWFDFIQNKR |
| | | YL |
| 78 | Cas12 | MENRYQVLQGLTAAQKKAAAAAKKRSSFSIVELNAATRSRVPDEKY |
| | | VPVQNYFSAMGKVCSQGEPKRENFVTRICAAYQELEEYIPSIRKSLLO |
| | | EKRATELIKNYLDAVNDLLRFIKPLLGRGNETDKDANFYGEFSFLTDC |
| | | LFAIVPLYNEVRNYLTQKPYSTEKFKLNFRGSTLLNGWDKNKERDNL |
| | | GVILRKEGKYFLAIMNKKHNTLFTEGKLQQHTGGECYQKMEYKLIPG |
| | | SKMLPKVFFSKKGISTFQPSEELLLNYRIGTYKKGEKFNLEHLHKLIDFY |
| | | KHSIAVHEDWSKFDFHFSDTSSYRDISGFYKEVEQQGYKLTFRNVSV |
| | | SYINRLVEEGKLYLFQIYNKDFSEYSKGTPNLHTLYWKMLFDPENLKD |
| | | VVYKLSGEAEVFFRKKSLDVSHPTHPKNEPIEKKNINNKGEKSLFSYD |
| | | LIKDRRFTVDKFQFHVPITMNFKGEQGDRVNQMVQSYVRNNKGL |
| | | NVIGIDRGERNLLYLVVINEHGEILEQFSLNEIRNAYNGKEHKIDYHTL |
| | | LEERSKKRQDARQSWQTIEGIKDLKTGYLSQVIHVITQLMVKYNAIV |
| | | VLEDLNFGFKSSRQKFEQSVYQQFERKLIDKLNFLVNKKAAPNEVGG |
| | | LLNAYQLTAPLGNSRKMGKQNGFLFYVPAWHTSKIDPRTGFVNLLD |
| | | TRYENVAKAKEFFAKFASITYNPEKKWFEFAFDYKAFGNRADGSRTK |
| | | WTICSYGERIETFRNPENNNQWDTKSVPLTERLTELFSKYGIDYTTNL |
| | | KEQILNQTDKAFFVELLGALRLTLQLRNSRKSTGEDFLFSPVADENGC |
| | | FFDSREANDNEPKDADANGAYHIALKGLWVLDTIRNTEEGKNPKLA |
| | | ITNKEWLSFAQAKPFAHE |
| 79 | Cas12 | MSDSYDELTKAQKEKQEKRKHVALTEVVAALEKYTIALDNGHEHKN |
| | | AVNTFKNYFQNYFFHFDTDKKKTAKTLDCQIKDEYNGLKGILNTPW |
| | | DKNKKLQQDKKLVQQIKSFLDSIQELLWFIKPLVLTDNTLEKDERFYG |
| | | EFMPLYDEISNIIKLYNKIRNYLTKKPYSIEKYKLNFENGSLLSGWDVN |
| | | KEKDNTSVLLCKDNQYYLAIMHIDHNKVFELDELIKHAGKGYQKINY |
| | | KLLPGANKMLPKVFFSGKNISYYDPSKEILKIRNYGTHTKNGDPQPG |
| | | FSKRDFSVDDCRKMIDFFKNSIAKHEDWKNFDFKFQPTKNYNSIDEF |
| | | YREVEEQGYKITYSNVSEDYIDSLVEYGKIYLFHIYNKDFSDKRDESKK |
| | | HTDNMHTLYWKALFDAKNLKDVVYKLNGEAEIFYRKKSIDIKKPTHE |
| | | KGKPIDNKNPNARKKTSVFKYDLIKDKRFTVDKFFFHVPITLNFKSKS |
| | | GYLSNDDVNAAIKKNNDIKIIGLDRGERNLIYLSLINSKGEIAYQESLN |
| | | VVSTDKGFDVNYHKLLDDKEGNRDEARKNWDKIENIKELKAGYLSQ |
| | | VIHKIAKLMIDNNAIVVMEDLNFGFKRGRFKVEKQIYQKFEKMLIDK |
| | | LNYLVFKNVHPEQAGGLYKAYQLTAQFESFKKLGKQSGFLFYIPAW |
| | | NTSKIDPTAGFVDFLKPRYESVTQAKSFLQRFDKINYNKTKDYFEFAF |
| | | DYKNFTDKANDTKTDWVVCTYGTERYYYDVRTKTTQKIDITAELKKL |
| | | LEKSEINYLNGKDIKELIIAVDSKEFHSALLKYLAIVLALRYSDSQSGRD |
| | | FILSPVANEQGHFFNSDKTDDTLPKDADANGAYHIALKGLWAINQI |
| | | RKTKNGDKLKLTISNKDWLNFVQKKEYRKGV |
| 80 | Cas12 | MQTLFENFTNQYPVSKTLRFELIPQGKTKDFIEOKGLLKKDEDRAEK |
| | | YKKVKNIIDEYHKDFIEKSLNGLKLDGLEKYKTLYLKQEKDDKDKKAFD |
| | | KEKENLRKQIANAFRNNEKFKTLFAKELIKNDLMSFACEEDKKNVKE |
| | | FEAFTTYFTGFHQNRANMYVADEKRTAIASRLIHENLPKFIDNIKIFEK |
| | | MKKEAPELLSPFNQTLKDMKDVIKGTTLEEIFSLDYFNKTLTQSGIDIY |
| | | NSVIGGRTPEEGKTKIKGLNEYINTDFNQKQTDKKKRQPKFKQLYKQ |
| | | ILSDRQSLSFIAEAFKNDAEILEAIEKFYVNELLHFSNEGKSTNVLDAIK |
| | | NAVSNLESFNLTKMYFRSGTSLTDVSRKVFGEWSIINRALDNYYATT |
| | | YPIKPREKSEKYEERKEKWLKQDFNVRLIQTAIDEYDNETVKGKNSG |
| | | KVIADYFAKFCDDKETDLIQKVNEGYIAVKDLLNTPYPENEKIGSNKD |
| | | QVKQIKAFMDSIMDIMHFVRPLSLKDTDKEKDETFYSLFTPLYDHLT |
| | | QTIALYNKVRNYLTQKPYSTEKIKLNFENSTLLGGWDLNKETDNTAII |
| | | LRKDNLYYLGIMDKRHNRIFRNVPKADKKDFCYEKMVYKLLPGANK |
| | | MLPKVFFSQSRIQEFTPSAKLLENYANETHKKGDNFNLNHCHKLIDF |
| | | FKDSINKHEDWKNFDFRFSATSTYADLSGFYHEVEHQGYKISFQSIA |
| | | DSFIDDLVNEGKLYLFQIYNKDFSPFSKGKPNLHTLYWKMLFDENNL |
| | | KDVVYKLNGEAEVFYRKKSIAEKNTTIHKANESIINKNPDNPKATSTF |
| | | NYDIVKDKRYTIDKFQFHIPITMNFKAEGIFNMNQRVNQFLKANPDI |
| | | NIIGIDRGERHLLYYALINQKGKILKQDTLNVIANEKQKVDYHNLLDK |
| | | KEGDRATARQEWGVIETIKELKEGYLSQVIHKLTDLMIENNAIIVME |
| | | DLNFGFKRGRQKVEKQVYQKFEKMLIDKLNYLVDKNKKANELGGLL |
| | | NAFQLANKFESFQKMGKQNGFIFYVPAWNTSKTDPATGFIDFLKPR |
| | | YENLNQAKDFFEKFDSIRLNSKADYFEFAFNFKNFTEKADGGRTKWT |
| | | VCTTNEDRYAWNRALNNNRGSQEKYDITAELKSLFDGKVDYKSGK |
| | | DLKQQIASQESADFFKALMKNLSITLSLRHNNGEKGDNEQDYILSPV |
| | | ADSKGRFFDSRKADDDMPKNADANGAYHIALKGLWCLEQISKTDD |
| | | LKKVKLAISNKEWLEFVQTLKG |
| 81 | Cas12 | MQLTDNLSDKYKEAAPLLNENYSNEKGLKNDDKSISLIKNFLDAIKEI |
| | | EKFIKPLSETNITGEKNDLFYSQFTPLLDNISRIDILYDKVRNYVTQKPF |
| | | STDKIKLNFGNSQLLNGWDRNKEKDCGAVWLCKDEKYYLAIIDKSN |
| | | NSILENIDFQDCDESDCYEKIIYKLLPGPNKMLPKVFFSEKCKKLLSPS |
| | | DEILKIRKNGTFKKGDKFSLDDCHKLIDFYKESFKKYPNWLIYNFKFKK |
| | | TNEYNDIREFYNDVASQGYNISKMKIPTSFIDKLVDEGKIYLFQLYNK |
| | | DFSPHSKGTPNLHTLYFKMLFDERNLEDVVYKLNGEAEMFYRPASIK |
| | | YDKPTHPKNTPIKNKNTLNDKKTSAFPYDLIKDKRYTKWQFSLHFPIT |
| | | MNFKAPDRAMINDDVRNLLKSCNNNFIIGIDRGERNLLYVSVIDSN |
| | | GTIIYQHSLNIIGNKFKGKTYKTNYREKLATREKDRTEQRRNWKAIESI |
| | | KELKEGYISQAVHVICQLVVKYDAIIVMEKLTEGFKRGRTKFEKQVYQ |
| | | KFEKMLIDKLNYYVDKKLDPDEEGGLLHAYQLTNKLESFDKLGTQSG |
| | | FIFYVRPDFTSKIDPVTGFVNLLYPRYENIDKAKDMISRFDEIRYNAGE |
| | | DFFEFDIDYDKFPKTASDYRKKWTICTNGERIEAFRNPANNNEWSY |
| | | RTIILAEKFKELFDNNSINYRDSDDLKAEILSQTKGKFFEDFFKLLRLTL |
| | | QMRNSNPETGEDRILSPVKDKNGNFYDSSKYDEKSKLPCDADANG |
| | | AYNIARKGLWIVEQFKKADNVSTVEPVIHNDKWLKFVQENDMTNN |
| 82 | Cas12 | MLPNEKERNEFKNSNAKQYIREISNIITDTETAHLEYDEHISLIESEEKA |
| | | DEMKKRLDMYMNMYHWAKAFIVDEVLDRDEMFYSDIDDIYNILE |
| | | NIVPLYNRVRNYVTQKPYNSKKIKLNFQSPTLANGWSQSKEFDNNA |
| | | IILIRDNKYYLAIFNAKNKPDKKIIQGNSDKKNDNDYKKMVYNLLPGA |
| | | NKMLPKVFLSKKGIETFKPSDYIISGYNAHKHIKTSENFDISFCRDLIDY |
| | | FKNSIEKHAEWRKYEFKFSATDSYNDISEFYREVEMQGYRIDWTYISE |
| | | ADINKLDEEGKIYLFQIYNKDFAENSTGKENLHTMYFKNIFSEENLKDI |
| | | IIKLNGQAELFYRRASVKNPVKHKKDSVLVNKTYKNQLDNGDVVRIP |
| | | IPDDIYNEIYKMYNGYIKENDLSEAAKEYLDKVEVRTAQKDIVKDYRY |
| | | TVDKYFIHTPITINYKVTARNNVNDMAVKYIAQNDDIHVIGIDRGER |
| | | NLIYISVIDSHGNIVKQKSYNILNNYDYKKKLVEKEKTREYARKNWKSI |
| | | GNIKELKEGYISGVVHEIAMLMVEYNAIIAMEDLNYGFKRGRFKVER |
| | | QVYQKFESMLINKLNYFASKGKSVDEPGGLLKGYQLTYVPDNIKNLG |
| | | KQCGVIFYVPAAFTSKIDPSTGFISAFNFKSISTNASRKQFFMQFDEIR |
| | | YCAEKDMFSFGFDYNNFDTYNITMSKTQWTVYTNGERLQSEFNNA |
| | | RRTGKTKSINLTETIKLLLEDNEINYADGHDVRIDMEKMDEDKNSEF |
| | | FAQLLSLYKLTVQMRNSYTEAEEQEKGISYDKIISPVINDEGEFFDSD |
| | | NYKESDDKECKMPKDADANGAYCIALKGLYEVLKIKSEWTEDGFDR |
| | | NCLKLPHAEWLDFIQNKRYE |
| 83 | Cas12 | MSNLYSNLHNLYPVQKTLRFELKPQGKTKENMEKAGILKADEHRAE |
| | | VYGKVKKYCDEYHKTFIDRCLSNIELNEIDKYYELYSINNRDDKQKEEL |
| | | DQLETGLRKQISDAFKKSAEYKGLFQKDMITSYLVTMYKENQEKMQ |
| | | DIGEFNRFTTYFTGYNKNRENMYSEEDKSTAISYRLINENLPTFIDNIK |
| | | IYKKIVSLMPENIEKIYKDLEEYIQVNSVDEIFNISYYNDVLTQRGIECY |
| | | NILISGRTKNDGDKIKGLNEYINEFNQTHNEKIPKLQELYKQILSDAES |
| | | ASFKVDIIENDKELLNLIEVYYANILPTLNKIEDLFTRISNYNLELILVNN |
| | | DGSLSTLSNMVFNEWSYIKGIISQKYDAEYSGKEKYGTEKYAQKKQE |
| | | YLKKQKIYSLKFLNDCIGNNAICEYLKNYIIQNKNIETIKEDYNEVQNIK |
| | | AEDDTKELIKDEKSIEKIKKFLDDVKSLQEFVKLVIPKDRTVEKDAKFYS |
| | | ELTPYYEKIKEIIPLYNKVRNYVTQKPYSTEKIKLNFECPTLLNGWDAN |
| | | KEEANLGVILLKEGKYYLGIMNPYCKKIFEVYEKDSNEQNNYKKMEY |
| | | KLLPGPNKMLPKVFFSNSRIEEFNPSKELQEKYNKGYHKKGKDFDIN |
| | | FCHELIDFYKQSLNKHEDWKKFNFKFKDTSEYNDISEFYREVEEQGY |
| | | KIEYTEYSEKYINELVDRGELYLFQIYNKDFSEYSKGKENLHTLYWKAV |
| | | FDPDNIMNPVYKLNGNAEIFYRKKSLEMKVTHPANQPIANKNISTIE |
| | | AGRSTSTFKYDLIKDKRYTMDKFQFHVPITVNFKSERLFNINQIVNKY |
| | | LKYNDDIHVIGIDRGERNLLYVCVIDKNEKIVYQKSLNEIVSEYNNNR |
| | | YTTDYHGLLDRKEKEREIAREDWKNIENIKELKEGYMSQIIHILVELM |
| | | KKYNAIIVIEDLNKGFKNSRIKVEKQVYQKFEKMFIDKLNYLVFKDED |
| | | KMDEGGVLNAYQLTNKFESFTKLGKQSGILYYIPAWCTSKIDPTTGFI |
| | | NRFYLKYENFDKSKEFVNRIDDIRYNEKENLFEFDIDYSKFTDRLNDTK |
| | | NKWTLCSYGERILTQKNANGEWFDRRIQLSIEFKNLFEKYVINLNNIK |
| | | DSILKLDKDNIEFYKGNGENLGFIQLFKLMVQMRNSLTGKEEDNLIS |
| | | PVKNQHGKFFNTSERVEGLPIDADANGAYNIARKGFMLVEQMKNV |
| | | EDEKLNKIKYNITEKEWLNYVQNRGMWWKRQYLYHI |
| 84 | Cas12 | MAKNTIFSQFTGLYPVSKTLRFELKPMGKTLEKIKETGVIENDKKRHN |
| | | DYFDAKKIIDKYHKYFIDAALSKFPRIDWSPLKEAIERSLDRSDASKKKL |
| | | EKTQTEFRKKIAKALTTHDHYKELTASTPKDLFLKVFPDHFGKQPAID |
| | | TFDGFSSYFTGFQENRQNIYSDEAISTAIPYRLVHDNFPKFLSNIEVYK |
| | | TLKDNAPSVLSDAENELRDFLNGKSLANIFELNAYNEVLTQSGIDFFN |
| | | QVIGGISDEGGEKKTRGINEFSNLYRQQHPEFAQKRLATKMIPLYKQ |
| | | ILSDRETKSFILESYSNDSQVQNSVKEFFESQILNWDIAGRRVNVLNE |
| | | LTSLVKRISEFDLGNIYVNQEELSNISLKLFDNWNSINGLLFKHAENRI |
| | | GSAEKSANKKKIDAWMKNKEFSIATLNLAIAESNSEEISRVKIESYWN |
| | | NFEAKVQSILCGDNRRNLDEFISATFNENNALREDSKIIEKLKAFLDAL |
| | | lEIMHSIKPLISDAENRDLSFYNELIPLYDQLSLVVPLYNKIRNYATQKL |
| | | TESEKFKLNFDNPTLADGWDQNKEEANTAILLLKNGLYYLGIMNAK |
| | | NKPKIKDFKTSESEDCYDKMVYKLLPGPNKMLPKVFFSEKGLATFKP |
| | | PKDILDGYNAGKHKKGDLFDIGFCHQLIDFFKESIAKHPDWKKFDFN |
| | | FSDTSSYEDISGFYKEVTDQGYKITFSKIPTSQIDEWVKEGKLFLFQIY |
| | | NKDFAPGAKGSPNLHTLYWKSVFSPENLKDVVVKLNGEAELFYRPS |
| | | SVKKPYSHKVGEKLVNRIGKDGLPLPESVFGELFRYFNGKLEGELSDE |
| | | AKRYLDVAVVKDVKHEIVKDRRYTQDKFEFHVPLTLNFKADSKNEY |
| | | MNERVRHFLKDNPDVNIIGIDRGERHLLYMTLINQKGEILKQKSFNV |
| | | VESVNYQAKLVQREKERDAARRSWSSVGKIKDLKEGFLSQVIHEITT |
| | | TMIENNAIVVLEDLNFGFKRGRFCVERQVYQKFEKMLIDKLNYLVFK |
| | | NKPEGDVGGVLKGYQLAEKFDSFQKLGKQSGFLFYIPAAYTSKIDPT |
| | | TGFANLFNMTELTSAEKKKEFLSHFEDITYDGKNDRFLFSFDYKNFKC |
| | | FQTDYIKKWTVYSQGKRIVYDKESKSAKEISPVEIIKAALAKQNIALTD |
| | | QLDVLSAINSVEASPKSASFFGDICYAFEKTLQMRNSIPNTDEDYLAS |
| | | PVMNKRGEFYDSRSCDDALPQNADANGAYHIALKGLYLIKNVFDA |
| | | GGKELKISHEDWFKFAQSRNC |
| 85 | Cas12 | MYKDKTDKTKIIDSDLIKFINIAESTQLDSMSQDEAKELVKEFWGFTT |
| | | YFVGFYDNRKNMYTAEEKSTGIAYRLVNENLPKFIDNMEAFKKAIAR |
| | | PEIQANMEELYSDFSEYLNVESVQEMFQLDYYNMLLTQKQIDVYNA |
| | | IIGGKTDDEHDVKIKGINEYINLYNQQHKDDKLPKLKALFKQILSDRN |
| | | AISWLPEEFNGDQEVLNAIKDCYERLSENVLGDKVLKSLLGSLSDYSL |
| | | DGIFIRNDLQLTDISQKMFGNWCVIQNAIMQNIKHVAPARKHKESE |
| | | EDYEKRIAGIFKKVDSFSISFINDCLNEADPNNAYFVENYFATFGAVN |
| | | TPTMQRENLFALVQNAYTEVAALLHSDYPTAKHLAQDKVNVAKIKA |
| | | LLDAIKSLQHFVKPLLGKGDESDKDERFYGELASLWAELDTVTPLYN |
| | | MIRNYMTRKPYSQKKIKLNFENPQLLGGWDANKEKDYATIILRRDG |
| | | LYYLAIMNKESKKLLGKAMPSDGECYEKMVYKLLPGANKMLPKVFF |
| | | AKSRMEDFKPSKELVEKYNNGTHKKGKNFNIQDCHNLIDYFKQSISK |
| | | HEDWGKFGFNFSDTSTYEDLSGFYREVEQQGYKLSFARVSVSYISQL |
| | | VEEGKMYLFQIYNKDFSEYSKGTPNMHTLYWKALFDERNLADVVYK |
| | | LNGQAEMFYRKKSIENTHPTHPANHPILNKNKDNKKKESLFDYDLIK |
| | | DRRYTVDKFMFHVPITMNFKSSGSENINQDVKAYLRHADDMHIIGI |
| | | DRGERHLLYLVVIDLQGNIKEQYSLNEIVNEYNGNTYHTNYHDLLDV |
| | | CEEERLKARQSWQTIENIKELKEGYLSQVIHKITQLMVKYHAIVVLED |
| | | LNMGFMRGRQKVEKQVYQKFEKMLIDKLNYLVDKKADASVSGGLL |
| | | NAYQLTSKFDSFQKLGKQSGFLFYIPAWNTSKIDPVTGFVNLLDTRY |
| | | QNVEKAKVFFSKFDAIRYNKDKDWFEFNLDYDKFGKKAEGTRTKW |
| | | ALCTRGMRIDTFRNKEKNSQWDNQEVDLTAEMKSLLEHYYIDIHG |
| | | NLKDAISAQTDKAFFTGLLHILKLTLQMRNSITGTETDYLVSPVADEN |
| | | GIFYDSRSCGDELPENADANGAYNIARKGLMMIEQIKDAKDLDNLK |
| | | FDISNKSWLNFAQQKPYKNE |
| 86 | Cas12 | MNEADPNNAYFVENYFATFGAVNTPTMQRENLFALVLNAYTEVAS |
| | | LLHSYYPAEKNLAQDKANVAKIKALLDAIKSLQHFVKPLLGKGDESDK |
| | | DERFYGELASLWAELETVTPLYNMIRNYMTRKPYSQKKIKLNFENPQ |
| | | LLGGWDANKEKDYATIILRRNGLYYLAIMDKDSRKLLGKAMPSDGE |
| | | CYEKMVYKLLPGANKMLPKVFFAKSRMEDFKPSKELVEKYNNGTHK |
| | | KGKNFNIQDCHNLIDYFKQSISKHEDWGKFGFNFSDTSTYEDLSGFY |
| | | REVEQQGYKLSFARVSVSYISQLVEEGKMYLFQIYNKDFSEYSKGTP |
| | | NMHTLYWKALFDERNLADVVYKLNGQAEMFYRKKSIENTHPTHPA |
| | | THPILNKNKDNKKKESLFEYDLIKDRRYTVDKFMFHVPITMNFKSVG |
| | | SENINQGVKEYLHHADDMHIIGIDRGERHLLYLVVIDLQGNIKEQYSL |
| | | NEIVNEYNGNTYHTNYHDLLDAREDERLKARQSWQTIENIKELKEGY |
| | | LSQVIHKITQLMVKYHAIVVLEDLNMGFMRGRQKVEKQVYOKFEK |
| | | MLIDKLNYLVDKKADASVSGGLLNAYQLTSKFDSFQKLGKQSGFLFYI |
| | | PAWNTSKIDPVTGFVNLLDARYQNVEKAKAFFSKFDAIRYNKDKD |
| | | WFEFNLDYDKFGKKAEGTRTKWTLCTRGMRIDTFRNKEKNSQWD |
| | | NQEVDLTAELKSLLEHYYIDIHGNLKEAISTQTDKAFFTGLLHILKLTLO |
| | | MRNSITGTETDYLVSPVADENGIFYDSRTCGDELPENADANGAYNI |
| | | ARKGLMMIEQIKNAEDLGNLKFDISNKAWLNFAQQKPYKNG |
| 87 | Cas12 | MDNNNLHAVDGYFATLGAVNTPTMQRENLFALIQNAYTDISDLLD |
| | | TPYPENKNLAQDKTNVAKVKALLDAIKSLQHFVKPLLGMGDESDKD |
| | | ERFYGELASLWTELDTVTPLYNMIRNYMTRKPYSEKKIKLNFENPQL |
| | | LGGWDANKEKDYATIILRRNGMYYLAIMDKDSKKLLGKTMPSDGE |
| | | CYEKMVYKLLPGANKMLPKVFFAKSRINDFKPSKKIVENYNNGTHKK |
| | | GKNFNINDCHDLIDYFKQSIDKHEDWSKFGFNFSDTSTYEDLSGFYR |
| | | EVEQQGYKLSFTNISVSFIDKLVDEGKMYLFQIYNKDFSDYSKGTPN |
| | | MHTLYWKALFDERNLADVVYKLNGEAEMFYRKKSINNTHPTHHAN |
| | | HPIQNKNKDNKKKESVFEYDLVKDRRYTEDKFLFHVPITMNFNSVG |
| | | AENINQQVRKYLQQADDTHIIGIDRGERHLLYLVVIDMQGNIKEQFS |
| | | LNEIVNEYNGNTYRTNYHDLLDVRADKRLKASQSWQTIENIKELKEG |
| | | YLSQAIHKITQLMVKYHAVVVLEDLNKGFMRGRQKVEKQVYQKFEK |
| | | MLIDKLNYLVDKHKDANETGGLLHALQLTSEFKNFKKSEYQNGFLFY |
| | | IPAWNTSKIDPVTGFVNRFDTRYTNAVEAQKFFRKFDEIRYNEEKD |
| | | WFEFEFDYDKFTQKAHGTRTRWTLCTHGKRLRSFRNPAKQYNWDS |
| | | EVVALTDEFKRILGEAGIDIHENLKDAISNLEGKRRKYLEPLMQFMKL |
| | | LLQLRNSRKNPEEDYILSPVADENGVFYDSRSCGDKLPENADANGA |
| | | YNIARKGLMLIRQIKEAKELGKVKYDISNKAWLNFAQQKPYKNE |
| 88 | Cas12 | MQKKAFEENQQNLRSIIAKKLTEDKAYANLFGKNLLESYKDKTDKTKI |
| | | IDSDLIKFINTAESTQLDSMSQDEAKEIVKEFWGFTTYFVGFFDNRK |
| | | NMYTAEEKSTGIAYRLINENLPKFIDNMEAFKKAIARPEIQADMEELY |
| | | SNFSEYLNVESIQEMFLLDYYNMLLTQKQIDVYNAIIGGKTDDEHDV |
| | | KIKGINEYINLYNQQHKDDKLPKLKALFKQILSDRNAISWLPEEFNSD |
| | | QEVLNAIKDCYERLSENVLGDKVLKSMLGSLADYSLDGIFIRNDLQLT |
| | | DISQKMFGNWSVIQNAIMQNIKHVAPARKHKESEEEYENRIAGIFK |
| | | KADSFSISYIDACLNETDPNNAYFVENYFATLGAVDTPTMQRENLFA |
| | | LVQNAYTEITALLHSDYPTEKNLAQDKANVAKIKALLDAIKSLQHFVK |
| | | PLLGKGDESDKDERFYGELASLWAELDTMTPLYNMIRNYMTRKPYS |
| | | QKKIKLNFENPQLLGGWDANKEKDYATIILRRNGLYYLAIMNKDSKK |
| | | LLGKAMPSDGECYEKMVYKLLPGANKMLPKVFFAKSRMEDFKPSK |
| | | ELVEKYNNGTHKKGKNFNIQDCHNLIDYFKQSIDKHEDWSKFGFKF |
| | | SDTSTYEDLSGFYREVEQQGYKLSFARVSVSYINQLVEEGKMYLFQIY |
| | | NKDFSEYSKGTPNMHTLYWKALFDERNLADVVYKLNGQAEMFYRK |
| | | KSIENTHPTHPANHPILNKNKDNKKKESLFEYDLIKDRRYTVDKFMF |
| | | HVPITMNFKSVGSENINQDVKAYLRHADDMHIIGIDRGERHLLYLVV |
| | | IDLQGNIKEQFSLNEIVNDYNGNTYHTNYHDLLDVREDERLKARQS |
| | | WQTIENIKELKEGYLSQVIHKITQLMVKYHAIVVLEDLNMGFMRGR |
| | | QKVEKQVYQKFEKMLIEKLNYLVDKKADASVSGGLLNAYQLTSKFDS |
| | | FQKLRKQSGFLFYIPAWNTSKIDPITGFVNLLDTRYQNVEKAKAFFSK |
| | | FDAIRYNKDKEWFEFDLDYDKFGRKAEGTRTKWTLCTRGMRIDTFR |
| | | NKEKNSQWDNQEIDLTAEMKSLLEHYYIDIQGNLKEAICTQTDKAFF |
| | | TGLLHILKLTLQMRNSITGTQTDYLVSPVADENGIFYDSRSCGDSLPE |
| | | NADANGAYNIARKGLMLIEKIKNAEDLDTIKFDISNKAWLNFAQQK |
| | | PYKNG |
| 89 | Cas12 | MLENEQEAEKIKNKLDDIMNIYHWIKIFLVDEEIEKDMDFYSEIEDIY |
| | | EELSPLVSLYNRVRNYVTQKPYSQEKMKLNFGSPTLADGWSKSKEFS |
| | | NNAIIMLKDGKYYIGIFNIRNKPNKEVIEGRNNRINDSDYKKMVYRLL |
| | | PGANKMLPKVMFSKKGIEYYNPSQYILSGYNSKKHIKSNENFDINFC |
| | | HDLIDFFKESINKNEEWKNFDFKFSDTESYNDISEFYREVEQQGYKIE |
| | | WVYISEQDIEQLEKNGQLYIFQIYNKDFAKKSIGNKNLHTMYLENLFS |
| | | EENLKDVVLKLNGEAEIFFRKSSIKKPIVHKAGSILVNKCIEDETGNKV |
| | | SFPDDIYNEIYQYMNGMTDVLSERAQNYYEKVKHSVSKQDIVKDYR |
| | | YTVDKYFIHLPITINFKASSFMPINDIALKYIAKRDDIHIIGIDRGERNLI |
| | | YVSVIDLQGNIVYQKNYNVVNGYDYKAKLRETEIQRDNARKNWKEI |
| | | GKIKQLKEGYLSLVVHEIAQLIVKYNAIVVMEDLNMGFKRGRFKVER |
| | | QVYQKFENMLINKLNYLVDKNKKVDEDGGLLRGYQLTYVPGQKEH |
| | | VGKQCGFIFYVPAAYTSKIDPTTGFVSIFNNKVNAKEFVTKFDSIKYN |
| | | KNMKMFELKFDYNNFETYNITLAKSKWTIYTNGIRLKREYNNGRWN |
| | | KITEVDLTKEMANTLKKYDIEFENNEEILKSISQLDEKNQRNICNEIKEI |
| | | IKLIVQLRNSMPDNGSKDNEYDKIISPVLNENNYFYDSSEVCDNSAP |
| | | ENADANGAYCIAMKGLYQVIQIKENWSQDSNPKNILGIKHYEWFD |
| | | FMQNKRYL |
| 90 | Cas12 | MAKNTIFTQFTGLYPVSKTLRFELKPMGKTLEKIKETGVIENDKKRHN |
| | | DYFDAKKIIDKYHKYFIDAALSKFSRIDWNPLKEAIEGSLDRSDASKKK |
| | | LEKTQTEFRKKIAKALTTHDHYKELTASTPKDLFLKVFPDHFGKQPAI |
| | | DTFDGFSSYFTGFQENRQNIYSDEAISTAIPYRLVHDNFPKFLSNIEVY |
| | | KTLKDNAPSVLSDAENELKDFLNGKPLANIFELNAYNDVLTQSGIDFF |
| | | NQVIGGISGEGGEKKTRGINEFSNLYRQQHPEFAQKRLATKMIPLYK |
| | | QILSDRETKSFILESYSTDSQVQESVKEFFESQILNCDIAGRKVNVLNE |
| | | LTSLIKRIAEFDLGSIYINQEELSNISLELFKSWNTINAVLFKNAENRIGS |
| | | AEKAANKKKIDAWMKSNEFSIATLNLAIAESDSEEISRVKIESYWNDF |
| | | EAKVQSILCGDNRRNLDEFLSATFNENNALREDSEIIGKLKAFLDALIE |
| | | IMHSIKPLISDAENRDLSFYNELMPLYDQLSLVVPLYNKIRNYATQKL |
| | | TESEKFKLNFDNPTLADGWDQNKEDANTAILLLKNGLYYLGIMNAK |
| | | NKPKIKDFKTSESEDCYDKMVYKLLPGPNKMLPKVFFSEKGLATFKP |
| | | PKDILDGYNAGKHKKGDLFDIGFCHQLIDFFKESIAKHPDWKKFDFK |
| | | FSDTSSYEDISGFYKEVTDQGYKITFSKIPTSQIDEWVNEGKLFLFQIY |
| | | NKDFAPGAKGSPNLHTLYWKSVFSPENLKDVVVKLNGEAELFYRPS |
| | | SVKKPYSHKVGEKLVNRIGKDGLPLPESVFGELFRYFNGKLDGELSDE |
| | | AKRYLDVAVVKDVKHEIVKDRRYTQDKFEFHVPLTLNFKADSKNEY |
| | | MNERVRHFLKDNPDVNIIGIDRGERHLLYMTLINQKGEILKQKSFNI |
| | | VESVNYQAKLVQREKERDAARKSWSSVGKIKDLKEGFLSQVIHEITT |
| | | TMIENNAIVVLEDLNFGFKRGRFCVERQVYQKFEKMLIDKLNYLVFK |
| | | NKPEGDVGGVLKGYQLAEKFDSFQKLGKQSGFLFYIPAAYTSKIDPT |
| | | TGFANLFNMTELTSAEKKKDFLSHFDDITYDGKNDRFLFGFDYKNFK |
| | | CFQTDFIKKWTVYTQGKRIVYDKESKSAKEIFPVEIIKAALAKQNIALT |
| | | DQLDVLSAINSVEASPKSASFFGNICYAFEKTLQMRNSIPNTDEDYLV |
| | | SPVMNKRGEFYDSRSCDDALPQNADANGAYHIALKGLYLIKNVFDA |
| | | GGKDLKISHEDWFKFAQSRNC |
| 91 | Cas12 | MRPVLQLTDTEDKLSQNKPAVGKIKALLDAFKDLQHFIKPLLGSGEE |
| | | NEKDELFYGAFQLIWDELDTVTPLYNKVRNWLTRKPYSTEKIKLNFD |
| | | NAQLLGGWDVNKEPDCTGVLLRKDGFYYLGIMNKKSNRIFDADVT |
| | | PADGICYEKIDYKLLPGANKMLPKVFFSKSRIDEFAPSEAILSSYKRGT |
| | | HKKGADFSLSDCHRLIDFFKASINKHEDWSKFGFQFSDTKTYEDISGF |
| | | YREVEQQGYMLSSHQVSEAYINQMVEEGKLFLFRIWNKDFSEYSKG |
| | | TPNMHTLYWRMLFDERNLADVVYKLNGQAEVFYRKASIKAENQIM |
| | | HPAHHPIENKNTLNEKRSSTFDYDLVKDRRYTVDKFQFHVPITINFKA |
| | | IGQ.TNVNPIVHETIRRGGFTHVIGIDRGERHLLYLSLIDLKGHIVKQM |
| | | TLNEIINEYNGLAHKTNYYDLLVKREGERTTARRSWDTIENIKELKEG |
| | | YLSQVIHIISKMMVEYNAIVVLEDLNMGFMRGRQKIERQVYEKFEK |
| | | MLIDKLNCYIDKQADSQSEGGLLHPIQLANKFESFRKLGKQSGCLFYI |
| | | PAWNTSKIDPVTGFVNLFDTRYETREKAKLFFSHFQRICFNAEKDWF |
| | | EFSFDYNDFTTKAEGTRTQWTLCSYGTRIRNFRNPLQNHQWDDEEI |
| | | VLTEAFKALFDKYDIDIHANLKEAINAQTDAQFFKDLMGLMKLLLQ |
| | | MRNSKTNSEVDYLLSPVADEHGRFFDSRAGAGSLPDNADANGAYN |
| | | IARKGLWVIRKIQETPEGEKLSLAITNKEWLEFAQTKPYLND |
| 92 | Cas12 | MNLNTYFSQFTGLYPVSKTLRFELKPMGKTLEKIKETGIIENDKKRHN |
| | | DYFDAKKIIDKYHKYFIDAALSKFPCIDWNPLKEAIERSLDRSDASKKK |
| | | LEKTQTEFRKKIAKALTTHGHYKELTASTPKDLFLKVFPDHFGKQPAI |
| | | DTFDGFSSYFTGFQENRQNIYSDEAISTAIPYRLVHDNFPKFLSNIEVY |
| | | NILKDNAPSVLSDAENELKDFLNGKPLANIFELNAYNDVLTQSGIDFF |
| | | NQVIGGFSGEGGEKKTRGINEFSNLYRQQHPEFAQKRLATKMIPLYK |
| | | QILSDRETKSFILESYSTDSQVQESVKEFFESQILNCDIAGRKVNVLKEL |
| | | SSLIKRITEFDLGSIYVNQEELSSISLELFKSWNTINAILFKNAENRIGSA |
| | | EKAANKKKIDAWMKSNEFSIATLNLAIAESDSEEISRVKIESYWNNFE |
| | | AKVQSILCGDNRRNLDEFISATFNENNALREDSKVIEKLKAFLDALIEI |
| | | MHSIKPLISDAENRDLSFYNELMPLYDQLSLVVPLYNKIRNYATQKLT |
| | | ESEKFKLNFDNPTLADGWDQNKEEANTAILLLKNGLYYLGIMNAKN |
| | | KPKIKDFKTSESEDCYDKMVYKLLPGPNKMLPKVFFSEKGLATFKPPK |
| | | DILDGYNAGKHKKGDLFDIGFCHQLIDFFKESIAKHPDWKKFDFKFS |
| | | DTSSYEDISGFYKEVTDQGYKITFSKIPTPQIDEWVNEGKLFLFQIYNK |
| | | DFAPGAKGSPNLHTLYWKSVFSPENLKDVVVKLNGEAELFYRPSSVK |
| | | KPYSHKVGEKLVNRIGKDGLPLPESVFGELFRYFNGKLDGELSDEAKR |
| | | YLDVAVVKDVKHEIVKDRRYTQDKFEFHVPLTLNFKADSKNEYMNE |
| | | RVRHFLKDNPDVNIIGIDRGERHLLYMTLINQKGEILKQKSFNIVESV |
| | | NYQAKLVQREKERDTARRSWSSVGKIKDLKEGFLSQVIHEITTTMIE |
| | | NNAIVVLEDLNFGFKRGRFCVERQVYQKFEKMLIDKLNYLVFKNKPE |
| | | GDVGGVLKGYQLAEKFDSFQKLGKQSGFLFYIPAAYTSKIDPTTGFA |
| | | NLFNMTELTSAEKKKEFLSHFEDITYDGKNDRFLFSFDYKKFKCFQTD |
| | | YIKKWTVYSQGKRIVYDKESKSAKAISPVEIIKAALAKQNIALTDQLDV |
| | | LSAINSVEASRETASFFGDICYAFEKTLQMRNSIPNTDEDYLVSPVM |
| | | NKKGEFYDSRSCGDSLPKNADANGAYHIALKGLYLIKNVFDAGGKDL |
| | | KISHEDWFKFAQSRNR |
| 93 | Cas12 | MQTLFENFTNQYPVSKTLRFELIPQGKTKDFIEQKGLLKKDEDRAEK |
| | | YKKVKNIIDEYHKDFIEKSLNGLKLDGLEEYKTLYLKQEKDDKDKKAFD |
| | | KEKENLRKQIANAFRNNEKFKTLFAKELIKNDLMSFACEEDKKNVKE |
| | | FEAFTTYFTGFHQNRANMYVADEKRTAIASRLIHENLPKFIDNIKIFEK |
| | | MKKEAPELLSPFNQTLKDMKDVIKGTTLEEIFSLDYFNKTLTQSGIDIY |
| | | NSVIGGRTPEEGKTKIKGLNEYINTDFNQKQTDKKKRQPKFKQLYKQ |
| | | ILSDRQSLSFIAEAFKNDTEILEAIEKFYVNELLHFSNEGKSTNVLDAIK |
| | | NAVSNLESFNLTKIYFRSGTSLTDVSRKVFGEWSIINRALDNYYATTY |
| | | PIKPREKSEKYEERKEKWLKQDFNVSLIQTAIDEYDNETVKGKNSGK |
| | | VIVDYFAKFCDDKETDLIQKVNEGYIAVKDLLNTPYPENEKLGSNKD |
| | | QVKQIKAFMDSIMDIMHFVRPLSLKDTDKEKDETFYSLFTPLYDHLT |
| | | QTIALYNKVRNYLTQKPYSTEKIKLNFENSTLLGGWDLNKETDNTAII |
| | | LRKENLYYLGIMDKRHNRIFRNVPKADKKDSCYEKMVYKLLPGANK |
| | | MLPKVFFSQSRIQEFTPSAKLLENYENETHKKGDNFNLNHCHQLIDF |
| | | FKDSINKHEDWKNFDFRFSATSTYADLSGFYHEVEHQGYKISFQSIA |
| | | DSFIDDLVNEGKLYLFQIYNKDFSPFSKGKPNLHTLYWKMLFDENNL |
| | | KDVVYKLNGEAEVFYRKKSIAEKNTTIHKANESIINKNPDNPKATSTF |
| | | NYDIVKDKRYTIDKFQFHVPITMNFKAEGIFNMNQRVNQFLKANPD |
| | | INIIGIDRGERHLLYYTLINQKGKILKQDTLNVIANEKQKVDYHNLLDK |
| | | KEGDRATARQEWGVIETIKELKEGYLSQVIHKLTDLMIENNAIIVME |
| | | DLNFGFKRGRQKVEKQVYQKFEKMLIDKLNYLVDKNKKANELGGLL |
| | | NAFQLANKFESFQKMGKQNGFIFYVPAWNTSKTDPATGFIDFLKPR |
| | | YENLKQAKDFFEKFDSIRLNSKADYFEFAFDFKNFTGKADGGRTKWT |
| | | VCTTNEDRYAWNRALNNNRGSQEKYDITAELKSLFDGKVDYKSGK |
| | | DLKQQIASQELADFFRTLMKYLSVTLSLRHNNGEKGETEQDYILSPV |
| | | ADSMGKFFDSRKAGDDMPKNADANGAYHIALKGLWCLEQISKTD |
| | | DLKKVKLAISNKEWLEFMQTLKG |
| 94 | Cas12 | MIDNTKEKKEGSVFDGFTRKYQLSKTLRFELRPILNTPKMLDDEQVIK |
| | | NDETRRKKYEAVKPWFDQLHREFIEDALKSFKFKNLAIYQDTFQTW |
| | | QKDRKSKQKKDTLVKIEVGLREEIVRRFEEVANIWVRSEQYKLLGIKK |
| | | EGLGMLFEAGVFRLLKERFKNEKDTTVDGNNIFDEWTRWTGYFKKF |
| | | FETRKNFYKSDDTSTAIAYRVINQNLRRFCENIQIFEKISEKIEFSEVEK |
| | | SFDISCAGIFSLAYYNACLLQGGIDTYNKIIGGEVDEKDKKIPGINELIN |
| | | KYRQDNSGEKIPFLKQLDKQIHSAKEAFIESIETNKELVGKLKTFYENA |
| | | EVKIQSFRNLIADIVTDYSGYDIDKIYLTKEAVSHNASRWFASFESFER |
| | | DLFAVVAEKQNKLVYELLRTHKNDSKISDKDGKFSFPDFIKCSHIKRA |
| | | LEKQEGRIWKGEYYEDIVDFEKIKDVFTQFLCVFKFELEQQFFRKTTS |
| | | AQTGEQTKIGYEIFVTKINELITRENPVIDLEEKIAIKNFADATLLIYOIA |
| | | KYFAVEKRRGWLDNYDLDDRFYKSSDIGYLNFYRDAFEQIVRPYNLF |
| | | RNYLTKKPYNTNKWVLSFENPTLADGWDKNKEKTNAAVILRKDGR |
| | | YYLGIIKEDCKSLFADRYSKEMSEGIESGSFQKMAYKFFPEASKMIPK |
| | | CSTQTKNVKEHFRKSSSDYNLFHEKDYKISVAITKNIYELNNVFYRKD |
| | | NIEESFVPKNDFEKKLGVKKFQRQYLEISRDNNGYKQALAQWIEFCI |
| | | RFLKAYKSTTIFDYSRLREAKEYESLDAFYQDINALTYNISFVPISEQYIK |
| | | EKNDNGELFLFEIYNKDWSLGPMDKNRKRTKNLHTLYFEQLFSKEN |
| | | EQENFLFQLNGEAELFFRPKTEEKRLGYKVWDAGEKKWVKAKEKED |
| | | GAVIDRKRYAKDIILFHCPITLNRVSESKTKREMDVEIREVLSSTPGVH |
| | | IIGVDRGEKHLAYYSVIDQNGKIIETDTLNSIGKDGRGKPVEYASKLEK |
| | | RAQEREASRRDWEEVEAIKDLKKGYISQVIRNLADLIIKHNAIIVFEDL |
| | | NMRFKQIRGGIEKSAYQQLERALIDKLSFLVKKGEEDPKQTGHILRAY |
| | | QLAAPVIAFKDMGKQTGLIFYTQAGYTSKTCPECGYRRNIKCLFENIE |
| | | QAKTLIENLESINYNKKEDVFQISYSLEKLSSKDQKKEKKVSNELYAKT |
| | | LKKDIFILTTKNALRYKWYDRYSEKAKVAKRGIDEYKGEVNESETKKG |
| | | VVKEFNLTEYLKGLLKTYEIDYEHGGIREQILSVARGREFYKDFLYALFL |
| | | LTETRHSISGRNTDYIQCPECEFDSRKGFKDIKEFNGDANGAYNIARK |
| | | GIMILEKIKQFKKDNDGNLEKMGWGDLSISIEEWDKFTQKE |
| 95 | Cas12 | MKNFQDFTNLYELSKTLRFELKPIGGTKKLIEEKNILKLDKKKRENYEK |
| | | VKPYFNKIHQEFINFALRNPNFDFSQFEEKYLNWLKDKKNKDLLKEK |
| | | ESIDKIFLEKIGKLFENSVKDFLKENGFESIVKEEDQNLKFFRRKEIFEVL |
| | | QEKYGSELETQMVNKDGEIKSIFNGWEKWLGYFDKFFNTRDNFYKT |
| | | DGTSTAIATRIIKDNLKIFLENIVAFGKIKNKKIDFSEVEKNFSVSIDTFF |
| | | EINNFNNCFLQDGIDFYNKVIGGETLENGEKLKGLNEIINKYRQDTGE |
| | | KIPYFKKLQKQILSEKDGVFIDKIEDDGGFYEVLKNFYKNAAEKEGFLK |
| | | NIFENFYTISDKNLEKIYFNKIAFNTISHKFGSALEFERILYEEMKKEKA |
| | | DGIKFEKKENKYKFPDFIQIIFIKRSLENYDSENLFWKERYYKSEENVD |
| | | GFLEKNNNNLWGQFCKILNFEFLNILKRRIIDEAGEEYEVGFEISKNIL |
| | | GEKLENFELNQENKGIIKDFADYSLALYSFGKYFAVEKGRNWDLNIDI |
| | | SDDFYGGEDGYIEKFYNTGYDEIVKPYNLMRNYISKKPWEDNKKWK |
| | | INFETSSLLSGWDKNLESNGSYIFQKGNKYYLGIINGSKPAKEILEKLYS |
| | | GDGEKIKRFIYDFQKPDNKNTPRMFIRSKKDSFSPAVEKYNLPINDIL |
| | | EIYDNGLFKTENKGNPNYKESLRKLIDYFKLGFSRHESFKHFNFVWK |
| | | DSKSYENIADFYRDVEKSCYKIDFEFLNFEELKKLTFEKHLYLFQIYNKD |
| | | FELDESLQEKGYNFKGEGQKNIHTKYFEALFLEENISRKSGAVFKLSG |
| | | GGEVFFRKKSIKAKKEKRNSVEVIKNKRYTECKYFLHFPIQVNFKEEIS |
| | | GNFNQEINKFLANNPDINVIGIDRGEKHLAYFSVINQKGEILESGSFN |
| | | KIENYNKNGEKLLFPEREIKEIHKDGSLIDLELVETGRKVDYVDYKLLLE |
| | | YKERKRLLQRQSWKEVEQIKDLKKGYISALVRKIADLIIKHNAIVIFEDL |
| | | NFRFKQIRGGIEKSIYQQLEKALIDKLNFLVNKNEINLEKAGSILKAYQ |
| | | LTVPVDSLKEIGKQTGVIFYTEAAYTSKIDPITGWRPNLYLKKNNSKIN |
| | | KENILKFDNIVFNSKENRFEFTYDLKKFFGKDSKFPAKTVNTVCSCVE |
| | | RFKWNRNLNNNKGGYIHYENLTDGKLANKEQKEDEFSNFKELFEKY |
| | | FIDINGNILEQIKNLDTKNNEKFFSSFIDLFTLVCQIRNTNQNAKGDE |
| | | NDFILSPVEPFFDSRKSQNFGKSLPKNGDENGAFNIARKGLIILNRISE |
| | | NPEKPDLLIFNADWDNFARNI |
| 96 | Cas12 | MSNNFQEFTQKYALSKTLRFELKPVGKTKEILEKEMPMYQIINADKN |
| | | IKAKYIQTKPFFDQLHRDFIKEAFENVELSGLSDFFENWKIYKQDKKA |
| | | NEKIYKKSAENLRKEVVSFLNAKGKDWAEKYHSSGLKKADIEILFEEGI |
| | | FKVLEIRYGTDTNSFITNATTGEITSIFQGWKGFTGYFLKFWNTRENY |
| | | YKTDGTSTAIATRIVDQNLPGYLENLEIFEKMKGKIDFESVRGDFSDF |
| | | EKIGTVEYYSTCLLQEGIDGYNRIIGGYTYENGEKIKGINEIINLYRQTH |
| | | KDEKVPFLKTLDKQIGSEKIAFMETIDTPEEFRKIFEEFVLKSSEKVVLL |
| | | KQCLNHLFENELTDGVFLSKESLNTISHKWIDIGNMKLFHESLFTILKK |
| | | EGAKYDSKEDEYKFPDFIRISDIKTALVKITTESFFWKNRYLYEKDENP |
| | | TGFLTSDNSLWEGFIQIFSHEFSSLFERTEKDEEGKDIQWGYDISLLNI |
| | | QKLLENNEYNPNDEKNKIIIKSFADDILRIYQMGKYFALEKKRQWNP |
| | | DNLEIGEFYSHPEIGYDKFYFDSYKIIVQGYNDIRNYLTKNPWSEEKW |
| | | KLNFENPTLANGWDKNKETDNSCIFLKRDNKFFLALMSRGNNQVF |
| | | DERNIQKFAQNIEQGKYEKMVYKYMKDVALGIPKATTQLNAVQEH |
| | | FFQSDKDYIITKGGSSIGEFIKPLRVTKRIFELNNRIYPKDNLGISFLRN |
| | | QVNKKEQKNYIKIFQKEFITLGGDEVVYKKAVHDWIDFCKEYTKSYPS |
| | | CAYFDYSGLKDTKEYSSIDEFYNDLDSFGYQISWQDISSSYIDELVESG |
| | | KLYLFEIYNQDFSNGKTGAKNLHTLYFEHIFSKENQEVNFPLKLNGQ |
| | | AELFFRPKSIEAKGENRKFNREIIAKKRYTEDKIFFHVPLTLNRTEGDIY |
| | | GFNTEINNFLAHNPDINIIGIDRGEKHLAYYSVIDQKGNIIESDSLNTV |
| | | NEINYGEKLTDTAEKRKQARQDWQAVEGIKNLKKGYISAVVHKLTD |
| | | LIIKYNAIVIFEDLNMRFKQIRGGIEKSVYQQLEKALIEKLNYLVEKGEI |
| | | NPEKAGHLLNAYQLTAPFETFKDMGKQTGIVFYTQAAYTSKIDPVT |
| | | GWRPHLYLKYSSAEQVKKEIAKFSNIIWNNTEKRFDFMYDIRNFSTQ |
| | | KEYPKNNIWTVCSSVERYRWDKTLNQNKGDYVHYKSITPEFEKLFSD |
| | | FQIDGTKNILEQINRMETKGNEKFFKSFIFFFGLICQIRNTNKADSDE |
| | | NKQDFILSPVVPFFDSRDSENTKNGLPRNGDENGAYNIARKGLIILQ |
| | | KINEFSDENGNCDKLGWKELSISQVDWDNYIKT |
| 97 | Cas12 | MEYDEHISLIESEEKADEMKKRLDMYMNMYHWAKAFIVDEVLDRD |
| | | EMFYSDIDDIYNILENIVPLYNRVRNYVTQKPYTSKKIKLNFQSPTLAN |
| | | GWSQSKEFDNNAIILIRDNKYYLAIFNAKNKPDKKIIQGNSDKKNDN |
| | | DYKKMVYNLLPGANKMLPKVFLSKKGIETFKPSDYIISGYNAHKHIKT |
| | | GENFDISFCRDLIDYFKNSIEKHAEWRKYEFKFSATDSYNDISEFYREV |
| | | EMQGYRIDWTYISEADINKLDEEGKIYLFQIYNKDFAENSTGKENLH |
| | | TMYFKNIFSEENLKNIVIKLNGQAELFYRKASVKNPVKHKKDSVLVN |
| | | KTYKNQLDNGDVVRIPIPDDIYNEIYKMYNGYIKESDLSGAAKEYLDK |
| | | VEVRTAQKEIVKDYRYTVDKYFIHTPITINYKVTARNNVNDMAVKYI |
| | | AQNDDIHVIGIDRGERNLIYISVIDSHGNIVKQKSYNILNNYDYKKKLV |
| | | EKEKTREYARKNWKSIGNIKELKEGYISGVVHEIAMLMVEYNAIIAM |
| | | EDLNYGFKRGRFKVERQVYQKFESMLINKLNYFASKGKSVDEPGGLL |
| | | KGYQLTYVPDNIKNLGKQCGVIFYVPAAFTSKIDPSTGFISAFNFKSIS |
| | | TNASRKQFFMQFDEIRYCAEKDMFSFGFDYNNFDTYNITMGKTQ |
| | | WTVYTNGERLQSEFNNARRTGKTKSINLTETIKLLLKDNEINYADGH |
| | | DVRIDMEKMDEDKNSEFFAQLLSLYKLTVQMRNSYTEAEEQEKGIS |
| | | YDKIISPVINDEGEFFDSDNYKESDDKECKMPKDADANGAYCIALKG |
| | | LYEVLKIKSEWTEDGFDRNCLKLPHAEWLDFIQNKRYE |
| 98 | Cas12 | MFGNWGVIQNAVMQNIKRVAPARKHKESEEDYEKRIAGIFKKADS |
| | | FSISYINDCLNEADPNNAYFVENYFATFGAVNTPTMQRENLFALVQ |
| | | NAYTEITALLHSDYPTEKNLAQDKANVAKIKALLDAIKSLQHFVKPLL |
| | | GKGDESDKDERFYGELASLWAELDTMTPLYNMIRNYMTRKPYSQK |
| | | KIKLNFENPQLLGGWDANKEKDYATIILRRNGLYYLAIMNKDSKKLL |
| | | GKAMPSDGECYEKMVYKLLPGANKMLPKVFFAKSRMEDFKPSKEL |
| | | VEKYNNGTHKKGKNFNIQDCHNLIDYFKQSIDKHEDWSKFGFKFSD |
| | | TSTYEDLSGFYREVEQQGYKLSFARVSVSYINQLVEEGKMYLFQIYNK |
| | | DFSEYSKGTPNMHTLYWKALFDERNLADVVYKLNGQAEMFYRKKSI |
| | | ENTHPTHPANHPILNKNKDNNKKESLFEYDLIKDRRYTVDKFMFHV |
| | | PITMNFKSSGSENINQDVKAYLCHADDMHIIGIDRGERHLLYLVVIDL |
| | | QGNIKEQFSLNEIVNDYNGNTYHTNYHDLLDVREDERLKAROSWQ |
| | | TIENIKELKEGYLSQVIHKITQLMVKYHAIVVLEDLNMGFMRGRQKV |
| | | EKQVYQKFEKMLIEKLNYLVDKKADASVSGGLLNAYQLTSKFDSFQK |
| | | LGKQSGFLFYIPAWNTSKIDPVTGFVNLLDTRYQNVEKAKSFFSKFD |
| | | AIRYNKDKEWFEFNLDYDKFGKKAEGTRTKWTLCTRGMRIDTFRNK |
| | | EKNSQWDNQEVDLTAEMKSLLEHYYIDIHSNLKDAISAQTDKAFFT |
| | | GLLHILKLTLQMRNSITGTETDYLVSPVVDENGIFYDSRSCGDELPEN |
| | | ADANGAYNIARKGLMMIEQIKDAKDLDNLKFDISNKAWLNFAQOK |
| | | PYKNG |
| 99 | Cas12 | MSNLYSNLHNLYQVQKTLRFELKPQGKTKENMEKVGILKADEHRAE |
| | | IYSKVKKYCDEYHKLFIDKSLSNIELNGIDRYYELYSINNRDDKOKEELD |
| | | QLEASLRKQISDAFKKSAEYKGLFQKDIITSYLVTMYKENQEKMQDI |
| | | GEFNRFTTYFTGYNKNRENMYSEEDKSTAISYRLINENLPTFIDNIKIY |
| | | KKIVSLMPEDIEKIYKDLEEYIQVDSIDEIFNISYYNDVLTQRGIECYNIL |
| | | ISGRTKNDGDKIKGLNEYINEFNQTHNEKIPKLQELYKQILSDAESASF |
| | | KIDVIKNDKELMNLIEVYYANILPILNKIEDLFTRISNYNLELILVNNDG |
| | | TLSTLSNMVFNEWSYIKGAISEKYDEEYSGKEKYGTEKYAQKKQEYLK |
| | | KQKIYSLKFLNDCIGNNAICEYLKNYIIQNKNIETIKEDYNEVQNIKVE |
| | | DDTKELIKDEKSIEKIKKFLDDVKSLQEFVKLVIPKDRTVEKDAKFYSEL |
| | | TPYYEKIKEIIPLYNKVRNYVTQKPYSTEKIKLNFECPTLLKGWDANKE |
| | | EANLGVILLKEGKYYLGIINPYCKKIFEVDEKDSNEQNNYKKMEYKLL |
| | | PGPNKMLPKVFFSNSRIEEFNPSKELQEKYNKGYHKKGKDFDINFCH |
| | | ELIDFYKQSVNKHEDWKKFNFKFKDTSEYNDISEFYREVEKQGYKIEY |
| | | TEYSEKYINELVDRGELYLFQIYNKDFSEYSKGKENLHTLYWKAVFDP |
| | | DNIMNPVYKLNGNAEVFYRKKSLEMKVTHPANQPIANKNISTIEAG |
| | | RSTSTFKYDLIKDKRYTMDKFQFHVPITMNFKSERLFNINQIVNKYLK |
| | | YNDDIHVIGIDRGERNLLYVCVIDKNEKIVYQKSLNEIVSEYNNNRYT |
| | | TDYHGLLDRKEKEREIAREDWKNIENIKELKEGYMSQVIHILVELMKK |
| | | YNAIIVIEDLNKGFKNSRIKVEKQVYQKFEKMFIDKLNYLVFKDEDKM |
| | | NEGGVLNAYQLTNKFESFTKLGKQSGILYYIPAWCTSKIDPTTGFINR |
| | | FYLKYENFDKSKEFVNRIDDIRYNEKENLFEFDIDYSKFTDRLNDTKNK |
| | | WTLCSYGERILTQKNANGEWLDRRIQLSIEFKKLFEKYGINLNNIKDSI |
| | | LKLDKDNLEFYKGNGESLGFIQLFKLMVQMRNSLTGKEEDNLISPVK |
| | | NQHGKFFNTSEKIEGLPIDADANGAYNIARKGFMLVEQMKNVEDE |
| | | KLNKIKYNITEKEWLNYVQNRGM |
| 100 | Cas12 | MARIFEDFKRLYPLSKTLRFEAKPIGATLDNIVKSGLLDEDEHRAESYV |
| | | KVKKLIDEYHKVFIDRVLDNGCLPLKNEGHNNSLTEYYDSYVSKSQN |
| | | EDAKKAFEENQQNLRSIIAKKLTEDKAYANLFGKNLIESYKDKTDKTKI |
| | | IDSDLIQFINTAESTQLNSMSQDEAKELVKEFWGFTTYFVGFFDNCK |
| | | NMYTAEEKSTGIAYRLINENLPKFIDNMEAFKKAIARPEIQANMEEL |
| | | YSNFSEYLNVESIQEMFLLDYYNMLLTQKQIDVYNAIIGGKTDDEHD |
| | | VKIKGINEYINLYNQQHKDDKLPKLKALFKQILSDRNAISWLPEEFNS |
| | | DQEVLNAIKDCYERLSENVLGDKVLKSLLGSLADYSLDGIFIRNDLQLT |
| | | DISQKMFGNWGVIQNAIMQNIKRVAPARKHKESEEDYEKRIAGIFK |
| | | KADSFSISYINDCLNEADPNNAYFVENYFATFGAVNTPTMQRENLF |
| | | ALVQNAYTEVASLLHSYYPAEKKLAQDKANVAKIKALLDAIKSLQHFV |
| | | KPLLGKGDESDKDERFYGELASLWAELDTVTPLYNMIRNYITRKPYS |
| | | QKKIKLNFENPQLLGGWDANKEKDYATIILRRNGLYYLAIMDKDSRK |
| | | LLGKAMPSDGECYEKMVYKLLPGANKMLPKVFFAKSRMDDFKPSK |
| | | ELIEKYNNGTHKKGKNFDIQDCHNLIDYFKQSIDKHEDWSKFGFNFS |
| | | DTSTYEDLSGFYREVEQQGYKLSFARVSVSYINQLVEEGKMYLFQIY |
| | | NKDFSEYSKGTPNMHTLYWKALFDERNLADVVYKLNGQAEMFYRK |
| | | KSIENTHPTHPANHPILNKNKDNKKKESLFEYDLIKDRRYTVDKFMF |
| | | HVPITMNFKSVGSENINQGVKEYLHHADDMHIIGIDRGERHLLYLVV |
| | | IDLQGNIKEQYSLNEIVNEYNGNTYHTNYHDLLDAREDERLKARQS |
| | | WQTIENIKELKEGYLSQVIHKITQLMVKYHAIVVLEDLNMGFMRGR |
| | | QKVEKQVYQKFEKMLIDKLNYLVDKKADASVSGGLLNAYQLTSKFD |
| | | SFQKLGKQSGFLFYIPAWNTSKIDPVTGFVNLLDARYQNVEKAKAFF |
| | | SKFDAIRYKRIRTGLSLISTMTSLVKKQKVQGLSGFYAPEECVLILSEIK |
| | | KKTHNGIIRKLT |
| 101 | Cas12 | MISLNYFQNQYAVAKTLCLELRPIEKTMEYIISSGILKEDEHRNESYKL |
| | | VKKIIDDYHKAYIELSLSRFELKITSCSKNDALEDFYCQYLANSQEEKDK |
| | | NIFKKTQDNLRKQIAKHLTQGEAYKRIDKKELIQEDLLEFVAADPDAA |
| | | NKKILINEFRDFTTYFTGFYENRKNMYSEEAQSTAIAYRIIHENLPKFID |
| | | NMGTFKQLMQSSITDILPQIFDNFKKDLEVSSIQEIFDLNYFNKVLTQ |
| | | KQIDIYNAIIGGKSLNENSRIQGLNEYINLYNQQHKENKLPLLKLLFKQ |
| | | ILSDRNSLSWLPEAFETDKQVLHAVRKCYANLKESVLHEAGLVQLLSS |
| | | LPSYDSTRIYIRNDQALTTISQKLFGDWGIIPHAIKERLKKDISAKRKET |
| | | EEAYLERIEKAFKQADSYTIAYINDSLKEIGVDKKNIEDYFIHLGAICTE |
| | | GQEQENILQRIASAYSQAQPLLEEKVPVHKNLMQDKDSVELIKSLLD |
| | | ELKNLQHFIKPLLGKGSESDKDERFYGEFVGLWNELDQITTLYNKVR |
| | | NYVTRKPYSIEKFKINFQNATLLKGWDRNKERDNTSIILRKNGLYYLAI |
| | | MRKEYNKVFEKYPAGTEENCYEKMEYKLLPGANKMLPKVFFSKSRI |
| | | NEFNPSPQLLQNYQMGTHKKGDQFKKEDCHALIDFFKTSIEKHED |
| | | WKNFNFQFSPTSVYEDMSGFYREVEQQGYKLVFRSIDAEYIDKLVEE |
| | | GKIFLFQIYNKDFSPFSKGTPNLHTLYWKMLFDERNLNNVVYKLNGE |
| | | AEIFFRKKSITYTHPTHPAEIPIKNKNVQNKKKESVFQYDLIKNHRFTI |
| | | DSFQFHVPITMNFKNAGLSNLNEQ.VYTYLRENKDAHIIGIDRGERHL |
| | | LYLVVIDRYGRIVKQFSLNEIVNEYHGNTYTTNYHDLLDKREEARQQ |
| | | ARQSWQSIENIKELKEGYLSQVVHKIANLMVEYHAIVVLEDLNIGFM |
| | | RGRQKVEKQVYQKFEKMLIDKLNYLVDKKKAPEADGGLLKAFQLTN |
| | | QFESFQKLGKQSGFLFYVPAWNTSKIDPCTGFTNLLDTRYENIAKAQ |
| | | KFFRTFDAIRYNAVKDYFELELDYDKFHKRAEGTQ.TKWTLCTYGTRIK |
| | | TFRNPENNNQWDNVEINLTEEFKKLFKQFGINLSGDLQQAICAQTE |
| | | KSFFESLLRLLKLTLQMRNSITGTDVDYLLSPVQNAEGYFYDSRKGDK |
| | | SLPANADANGAYNIARKGLWVIQQIKQTPQGQKAKLSISNKEWLKF |
| | | AQEKPYLKD |
| 102 | Cas12 | MKTFENFTNLYSLPRTLRFELKPLYKTKELIDSKQELFPKDKRIDEIYQN |
| | | IIKPCLNELHSDFIEKSMENKDFQNIPDNILKIYSNEKNIDDFKNIEKDL |
| | | IKQINWFLKSNKTFFAENYSDLLGKNSIDIIIKVFWEKIYKKDDSWKIF |
| | | LYNDLLWKSYEELINIYFKWFSTYLSNFNKNRENLYDKKNEAKVWSV |
| | | SGRTIWENFPRFLQNCINFRDKLEKLNLSIEQKDIFITNNFWKCISQK |
| | | QIDYYNKIIWQINSKTNEFNQKNGLKWNKKLPKLLLLHKQILWKSEN |
| | | ENILNFINNIIQ.TDFELEQ.EIKIINKDIFQ.RIDFIKKSIVSNIEDFELEKIFIK |
| | | KNRLKDISSLLMDNYSVLEKLLPEFNEEWKIIKENELVNLSKIKKSFENI |
| | | DLKDLKNIFKKEYFDESKDWFKLFLNWIYNHFSDLENNIKHTHKLVQ |
| | | DKLISWNFSENIQKSEKNINLRDEIFVSSKWLLKAYLDSILALDRFVH |
| | | MFDYWEQKDFDSNFYNNIEEYSINFSPFKTYNAVRNYLTKKNYSTD |
| | | KIKLNFDYPDFLGSNSLWKYAFIYKDSKWFYYLWVLDHSNSQSKYKP |
| | | QILKNNTEFYQLEYKQIKFNTLAWKWYIRDFWVKYSEDENCIINLKTL |
| | | IKKQYLERYPVLKEIVDFQTDDKKIFDAKVKTILEQAYSINFVNIDKNYI |
| | | LEENNNWNLHFFQIYNKDFSENKKINSMENLHTMYFKALFEKENFN |
| | | WWACFKLNSQWAEIFFREKSINEKKVKDLKTRNENAIEKKRYTENK |
| | | VFLHLPITLNFINKWYSKYSFWYINDSVKKYIKENKISIIWIDRWEKNLI |
| | | YFSMINENLEIIELKSLNSLILKVSDLEEKEVNYFEKLSKKEWNRNKER |
| | | KDWDEIETIKELKEWYISQIVDNLVKLIVKHNAIVVMEDLNSGFKRW |
| | | RQKIEKQIYQKFELALAKKLNFTVDKNKKHDELWWIYKAYQLTPQIE |
| | | NFQDIYSQTWIIFYTQAAYTSVTCPNCSFRKNIYQKYENESKFKEFFK |
| | | KYILEIKFEDNCFIIKYKIDEKIDKKKNKLKKLEFQVNTKNQIRLKFEKSL |
| | | KWKWWETKEFNITEKFKEIFEKHKLDLWNLKEELLAWNWEIQLYKD |
| | | FMFYFNLLLQLRNSKENDNWWYISCPSCWFHSGNWFQWFSYNW |
| | | DANWAYNIARKWRIILDKIKKDEKNLWITNVEWDNYYOK |
| 103 | Cas12 | MAVRSVKLKLLVPRDGSAESVRKRKALWATHQFVNDAAAAYAELLL |
| | | EMRQEDVCRGTDDHGKDVIEPAAHWQAKLRARLAAKQLPPVAVA |
| | | EALPLLKAFYGSRLIKSFVANDKGVAGTGNATDLNTWLSGLVDPASV |
| | | AGEKTELRKQLLAELPLCEAADADFEGAARKMLAKSDAREALLEGP |
| | | GTGVGWPAAYNANPTDSVWLDMLHKAAAKARLELADTTVSELKK |
| | | LGVFPLLQAASSNRVFGSGVLNPFERMAAAQAAAALLPWETKRHE |
| | | MRKRRDKFADQLNQWDTEFGASHATALAAIRAFEAEESERARRESL |
| | | GNEGTGYRIGGRELRDAWTLLRDWLKGHSTATAAAREDKVRELQA |
| | | KQGRSFGSHRLLSWLAKPAQQWLADHSAGDVVTRIAVRNARQRK |
| | | LDTARTLPIWTGADAVKHPRFANFDPPNNTNQPGFDLRAGTOKGR |
| | | LTLRLSLLTERADGLLLAQDHDFQLVPSRQMAEIVLHKDGKERALSW |
| | | QSQDGIGRQVGDVGGSALLFSRDHAECLLERKQITRLERGAWPAAL |
| | | PVWFKLSLDIGAEHKALLKQRFKWGVWLNSALVTRNAKDAKGVPP |
| | | PVGTRVLAVDLGLRSAATVSVWQVVDAATPVVAGKWRVPLSDTLS |
| | | AVHERSAMLALPGEHVDAGVLAARRAANEKLAGLLAATSHLSTVFK |
| | | LGRAEQGDRRRELLERLGEGDDRRARAAVATTAAERDGLRAVLGA |
| | | TQDAWAGAVAAVWRRLETDLAGAIAAYRKQQREDVQLRREARHG |
| | | PGASQLPKQAAAERLLGGKSAWQIEYKERVRKLLTRWIMRQRPGD |
| | | TAVRRLARKDLGKYCGGLLDHLTALKEDRAKTTADLIVQAARGRVR |
| | | AHKDAHGRQQDRELWLAKYAPCDLIVMEDLGRYRFATDRPPSENR |
| | | QLMQWTHREVFRLVQMQAEVEGIQVLETGAEFSSKFDARTWAPG |
| | | VRCEPITKLWVERYRNGEMPWLADKADEWRREGIELAQLVPGQLL |
| | | PTGSGEQFVAVSATGGLRVRHADLNAAQCIALRALTGHGTAFRLTA |
| | | RRLGDVFVSAKGLGKRPQGALWREFGSALPPAVVVLRPAGEVRYAL |
| | | RPFASARDAAAALGLQLGALRNVDATDAESDAEDGDLAELLAGAD |
| | | PDRATFFRDPSGDVHGGAWVQAKVFWAEVRRHVRLGLQAQGLLP |
| | | AAARSSEPRQMQLPLAGALPGDDIPL |
| 104 | Cas12 | MLDKFASLYPVTKTLRFRLLPQGRTEENMovAKVLENDLERSEAAA |
| | | WKGLIKKYHLQFISDTLSGSTLSWQALTETLDKFKADHTATAELDSA |
| | | LAAYRCKLAELFTKSPKYKVMATPVSIIKEILKTETDPENIAALNKLNG |
| | | YTYIIFDYVSTRMLTYSADAKATSLAYRLVDENYLRFYQD1SAAAEISA |
| | | VLEEAGFDNAEVEAFIRTDYNTCLTSEGIASFNAAAGSINQFVNVLL |
| | | QQNPVLQSEPALRRHLQPLYKMLLDEAESKIIKFEDYGQLRDAVENF |
| | | RRNFQDLPQSLIDIFAGRYDYSKIYVGYKYLNEASSQIAGGYNWKLLE |
| | | NALEDFYSKPYLVNGKLPVKYKTVVNKKMNQLAYSFTELQEALDAG |
| | | DSGSSITDLFGKYAELHAAYAAADGNVFYKEYDRKSIASLKNYLDAV |
| | | NAIARFIKIFAAPEVYVKDEGFYGIVDGAADKLRDFDLLYNMVRNYIT |
| | | KKPYKKSKVALTFNSSSFGRGWDENKIYDELTTIFTYNGKYYLGVINK |
| | | NDKPDLAAAVSKDEGGYKRMVYKTFDIVKQLPRLSFTKAVKAHFAE |
| | | SDEDFIFDGPKFAKPLRVPKEIYLQSFTDNGDKLADSAKKYTKAYLD |
| | | MSGDYKGYYEAIIKRIDYTKEFLSAYKSTSIYDLAFLKPAGKAAGSLCW |
| | | TRHI |
| 105 | Cas12 | MKTFENFTNLYSLPRTLRFELKPLYKTKELIDSKQELFPKDKRIDEIYQN |
| | | IIKPCLNELHSDFIEKSMENKDFQNIPDNILKIYSNEKNIDDFKNIEKDL |
| | | IKQINWFLKSNKTFFAENYSDLLGKNSIDIIIKVFWEKIYKKDDSWKIF |
| | | LYNDLLWKSYEELINIYFKWFSTYLSNFNKNRENLYDKKNEAKVWSV |
| | | SGRTIWENFPRFLQNCINFRDKLEKLNLSIEQKDIFITNNFWKCISQK |
| | | QIDYYNKIIWQINSKTNEFNQKNGLKWNKKLPKLLLLHKQILWKSEN |
| | | ENILNFINNIIQ.TDFELEQ.EIKIINKDIFQ.RIDFIKKSIVSNIEDFELEKIFIK |
| | | KNRLKDISSLLMDNYSVLEKLLPEFNEEWKIIKENELVNLSKIKKSFENI |
| | | DLKDLKNIFKKEYFDESKDWFKLFLNWIYNHFSDLENNIKHTHKLVQ |
| | | DKLISWNFSENIQKSEKNINLRDEIFVSSKWLLKAYLDSILALDRFVH |
| | | MFDYWEQKDFDSNFYNNIEEYSINFSPFKTYNAVRNYLTKKNYSTD |
| | | KIKLNFDYPDFLGSNSLWKYAFIYKDSKWFYYLWVLDHSNSQSKYKP |
| | | QILKNNTEFYQLEYKQIKFNTLAWKWYIRDFWVKYSEDENCIINLKTL |
| | | IKKQYLERYPVLKEIVDFQTDDKKIFDAKVKTILEQAYSINFVNIDKNYI |
| | | LEENNNWNLHFFQIYNKDFSENKKINSMENLHTMYFKALFEKENFN |
| | | WWACFKLNSQWAEIFFREKSINEKKVKDLKTRNENAIEKKRYTENK |
| | | VFLHLPITLNFINKWYSKYSFWYINDSVKKYIKENKISIIWIDRWEKNLI |
| | | YFSMINENLEIIELKSLNSLILKVSDLEEKEVNYFEKLSKKEWNRNKER |
| | | KDWDEIETIKELKEWYISQIVDNLVKLIVKHNAIVVMEDLNSGFKRW |
| | | RQKIEKQIYQKFELALAKKLNFTVDKNKKHDELWWIYKAYQLTPQIE |
| | | NFQDIYSQTWIIFYTQAAYTSVTCPNCSFRKNIYOKYENESKFKEFFK |
| | | KYILEIKFEDNCFIIKYKIDEKIDKKKNKLKKLEFQVNTKNQIRLKFEKSL |
| | | KWKWWETKEFNITEKFKEIFEKHKLDLWNLKEELLAWNWEIQLYKD |
| | | FMFYFNLLLQLRNSKENDNWWYISCPSCWFHSGNWFQWFSYNW |
| | | DANWAYNIARKWRIILDKIKKDEKNLWITNVEWDNYYOK |
| 106 | Cas12 | MSFLVPHLPTVVSHRLGGYSSAMDQTPTRLTPAVKNTAAATPKPEV |
| | | PLTQRAYTLRLRGANDGDQSWREAVWATHEAVNMGAKVFGDWL |
| | | LTLRGGLDRELADAKVKAGNNNPDRNPTPEERRGRRVLLALSWLSV |
| | | ESAPKKGDAYEKFVIASGKKDSQSIRDEKVVRALREILAKRGVANQD |
| | | LEGWIVDCEPSLSAAIRDDAVWVNRSAAFDAAQCRVGTSLTREEIW |
| | | DLLKPFFGSCESYLASLTTDEDSDTEAAATDDKAKDLVQKAGQWLSS |
| | | RFGTGKGADFAAMSKVYSETAFWAGRASPFRSGAEALRLIAESLKSF |
| | | CPKSFDADGILGLISGPGYKSATRNIIKAWSKRAGPVTADDLANLSA |
| | | VAAEDANKCSANTGSKGHRPWSDAILCEVENACGFTYLQPDGPAL |
| | | HSEFAVMLDHAARRVSIGHSWIKRAEAERDRFTKDALRIKEVPDPIR |
| | | VCLDRFCADRAGTSGAIDGYRIRKRAVSAWKEVITRWGQAACKTAE |
| | | DRVIAVRETQADPDIDKFGDIQLFEALAVDDAECVWRVNGEVTPQP |
| | | LIDYAAATDAEAKQKRFKVPAYRHPDPLSHPVFCDFGNSRWKIRFA |
| | | AHDAVTKLANARTSLDRREADLAKAKERLDKATAPEEQTEGKESLEE |
| | | AERHLRDARDRVAWLSSMHAFSMRLWGAGRVGDGQRLFWSCKR |
| | | LTDDMALRQNSGQAPTIAVTRADRLGRAAAGADTADSVDILGVFT |
| | | EEHWNGRLQAPRAQLDVIAAHVAKNQWDAKAKKMRDRIRWLVS |
| | | FSAKLQPVGPWIEYSATFPEAALAKPFVSRKGEYAVRHVSNDDRAG |
| | | LGKLVLSRLPGLRVLSVDLGHRYAAACAVWETVSIAQTNAACDAAG |
| | | HELPTESDLFLQLSTTDLTGKNRTTIYRRIGADMITDPKTGEKTPHPA |
| | | PWARLERQFLVKLPGEDIPARKASPAEFDAIRRLEEAFGRTRTADDP |
| | | LLVRVDELLAATVDSARLALRRHGDAARIAYAFKPSAEKLTPGGGRE |
| | | VMSPEARKAMILDALLLWHGLWHGDRWADVWASQQWDAYIKP |
| | | ELGMDLPPWSETSGEPRCQYRSKVEGLLKRVAESLAARDGAGLHLL |
| | | WAEQWRTRNAKWLGNTGHLRTLRSLLLPRGLTTSTPAAWNVGGL |
| | | SLTRIATLKSLYQLHKAYHMRPEPEDPRKNVPAKGEEELRDFGRGML |
| | | DVMERLREQRVKQLASRLAEAALGIGRMKASEGKRDRKRPRAQID |
| | | QPCHAVVIENLKNYRPEETRTRRENRQLMSWSSSKVKKYLSEACQL |
| | | NGLHLREVQASYTSRQDSRTGAPGIRCADVPVQDFFTKPWWRRQ |
| | | VSIAVGRVNQGRGDARERFLADLDAKWSAAEKSERITAPPLRIPVN |
| | | GGELFVSADLHSPAALGLQADLNAAANIGLKALLDPDWPGKWWF |
| | | VPASLDADGWRVPAAKSCAGAEWVKNWKVGQLGDSYAPNGKPL |
| | | QPTDDEGVKKAEDGVKLAKASLDDAEQALKAAKKTKRKAEIDVASA |
| | | RAQEAKKNVDDMKKALVAAKKGASAKEIINLWRDPVGVDPADFAS |
| | | GEPWRAYTVYKQRVEYHVINDVLRGRAGPRSDRP |
| 107 | Cas12 | MSITRSIKVKLIVPRDASLEARQLREGLWATHLFVNDGCHYYERLLLE |
| | | FRQRDVCVGKDDAGKDVIVPAAEWADRLRARLGRNGMVPSHIEA |
| | | ALPIFRELYENMVPSALKAKSGTGQAGRSWHSKLVSPTSRGGEASA |
| | | ARIDVLRPLLPVSGDDPAFEPAARALIEEAGDELLTSTGRCPAWVTA |
| | | YRKGPEGSAWVEKLRIQLREAVEAGDFDPPSDPQILAAGAVPAAPP |
| | | LGAGIDALRPLLPLLGGDPAFEPAARALVEDIGDELFTSTGRPPTWV |
| | | TAHPTWVRAHRKDAECLEAADDFKWVERLRQRLRDDAKAGKFEQ |
| | | PLHERLGALGALPVAKPIGAGRVVSRADLTVFERGAMELAIEHLIGW |
| | | ESAGHRARAQYVERKKRHDDLLQWIEAEAPDALLAVRAYEAARTIH |
| | | LATLGELGAAPQYTLRLREIRPWRKLREWLLQNPDATIDERRRRLAT |
| | | MQTNDPRGYGGEALAWLAAPERRALVEHPAGDVVTRIAVLNIRKSI |
| | | LDRSRLFPTCTLADPVEHPRFAKFGKPGDKNSAGYALAVDGVRREAI |
| | | IKILVPRQDGLLVPTDLRVPFAPSGQMRDLRASGLDISYERQDGRGR |
| | | QAAKLQGGNLMFDRTHFARCGAPGPEALGSVWIKVALDLSSPAAS |
| | | LAMKTATPVRTYLSTAVRGRPESTKYEKAAPPEGFRVLSVHMGLRT |
| | | AATASMLRFGAPEEGGHEVPVSGLAGETLVAFHERTVTMKLPGED |
| | | PDTRTEANRGVAKRELRGLGRGIGCLKAIRRASASATPEDRAEALVII |
| | | ETHVGQGDRHGWAPAEAVGRLDPHGDPDDWKTACAALYAAVEA |
| | | DLGVAISSWRKAARAGGATGMLGGKSLWAVDHLERSFRFLRSWDL |
| | | RARPHDGDPRRPRPGYASKLLHHIDGVKDDRVKTTADRIVQAACG |
| | | RAWIGGPTVKRGTQDVRLPGRWEQRGPRADLILLPDLTHFRFRSDR |
| | | PRAENSRLMRWAHRQLAIYVRMQAEVEGILVADTGAAFTTRFDA |
| | | WTGAPGVRCEPVTADHLRGIAKREDYWLARLLREGALKHLRIDPAS |
| | | LRVDDLVPMDHGKILVALDGVDLPGLRILDTDVNASQGLGRRYIEG |
| | | HGLAYRLPGARVPRGEGEREAAVVHIKGKRLASAMGGTVVVLRASE |
| | | GPGDITWTAEVYDRPQGARKALGLSLAAFNSIATAAVDDEGPAPEN |
| | | DDEALEEEAEEALGIATGERIVFFRDPSGAVAGGGWLEASAFWGIA |
| | | NRMVTDRLRELGRLG |
| 108 | Cas12 | MATAVDTSTTRAYTLRLSGGNNWRELLWQ.THVAVNRGAWVWG |
| | | DWLLTLRGGLPASLADGDAERRVVLALSWLSVESPASLAPQAHIVA |
| | | YGSDARDERNRKVTERFRDILRRMGIKQQQEQEWLDACLPALMAS |
| | | IREDAVWVDRSACFAEAQQCYRGLSSEWARKTLFDFLGGEDDYFKP |
| | | SAKEGASSKAKDFVQKAGRWLSRHWGAGKKSDPRDISTRLGKLAG |
| | | VDPKAIDGHTGRAALEDLLRTLGSRPAQNADAEKLYRQLKRAVGW |
| | | KGRPSKGAVALKKIRDAERVPNDLWKEIASTLREEAAVQSSQTSDH |
| | | AAVPDWRSHWPAEITGLPMPYRVDRDYIWEHGVMLDHALRRVSS |
| | | AHTWIKRAEAERRRFQQDAAKMGSIPEEARNWLDAFRERRSSSSG |
| | | ATGDYLIRERAINGWDKVVQAWETLGPNSTRDQRIAAARDVQANL |
| | | DEDEKFGDIQLFAGFGDEHVDDPERCLADDRATCVWRNSSGRADG |
| | | RILKDYVAATVAEHNQRRFKVPAYRHPDPLRHPVFVDYGKSRWSIN |
| | | YSALTAAQQRRKTTQKLAQAKTDNTRAKLQQQLASTADLRSVTLGV |
| | | WDGNRIVKISQRWRSKRFWRDLDLDHFGSHPSAAVSRADRLGRVA |
| | | ARQDPGAAVYVAKVFEQQDWNGRLQVPRRELNRLADVVYGKGA |
| | | DPDFGKLERLDPRARRLWERLSWFLTTSATVOPQGPWLDYVAAGL |
| | | PSGIQYTKSRAGYYLNYDANHGRKGRARLCLARLPGLRVLSLDLGHR |
| | | YAAACAVWQTLTIEQMTNECRQAAHPAPSNDDLFIHLRHPTHKPQ |
| | | KSGRKKGRPVTKTTIYRRIGPDKLPDGTDHPAPWARLERQFLIKLQG |
| | | EDRPARYASQKEIDEVNQFRNFVGLEPIVDRPRVDDLHSDAVRVAR |
| | | LGLRRLADAARIAFAMTAAKKPISGGHEVELTTAQRIEFLQDALLLW |
| | | QSLAASRRYRDDWAEKLWQSWVVEKLGGPQPAEIADDLPRSQRA |
| | | ASLKTARQSLRKVAEKLSDGQSPSAAELHRLWAERWQQRQTEWR |
| | | RHLRWLRRLILPRRKDHQQEDRPLQRVGGLSVKRIQTIRQLYQVLKA |
| | | FRMRPEPSDLRKNIPAPGDRSLASFGRRILNHLERLREQRIKQLASRV |
| | | VEAALGAGRISKPPGRDRRRPQQPVDRPCHAVVIENLQHYKPEDSR |
| | | LRRENRQLMDW/QARNLRKYIVEGCELHGLLFVEVSPAYTSRQDSRT |
| | | GAPGLRCEDVSRTALQEAARRMHASHSRPSNSSPGGSQTQFEREV |
| | | CRWINEFKRVEGSSSSLSARQAVLKAFLHHQASIPTSLSTILLPRRGGE |
| | | LFVSADPDSPLACGLQADLNAAANIGLKALTDPDWMGAWWFVLV |
| | | DRASGQPVEEQVQGCPIWLSCGPLSNSNPATIDPSDSPTAARRSNG |
| | | TGAKGRARANEYWWSSLSATTLPDHKAWQPTQDYWRDIEQRVV |
| | | KRLLRLLDGSEWSED |
| 109 | Cas12 | MLDKFASLYPVTKTLRFRLLPQGRTEENMQVAKVLENDLERSEAAA |
| | | VVKGLIKKYHLQFISDTLSGSTLSWQALTETLDKFKADHTATAELDSA |
| | | LAAYRCKLAELFTKSPKYKVMATPVSIIKEILKTETDPENIAALNKLNG |
| | | YTYIIFDYVSTRMLTYSADAKATSLAYRLVDENYLRFYQD1SAAAEISA |
| | | VLEEAGFDNAEVEAFIRTDYNTCLTSEGIASFNAAAGSINQFVNVLL |
| | | QQNPVLQSEPALRRHLQPLYKMLLDEAESKIIKFEDYGQLRDAVENF |
| | | RRNFQDLPQSLIDIFAGRYDYSKIYVGYKYLNEASSQIAGGYNWKLLE |
| | | NALEDFYSKPYLVNGKLPVKYKTVVNKKMNQLAYSFTELQEALDAG |
| | | DSGSSITDLFGKYAELHAAYAAADGNVFYKEYDRKSIASLKNYLDAV |
| | | NAIARFIKIFAAPEVYVKDEGFYGIVDGAADKLRDFDLLYNMVRNYIT |
| | | KKPYKKSKVALTFNSSSFGRGWDENKIYDELTTIFTYNGKYYLGVINK |
| | | NDKPDLAAAVSKDEGGYKRMVYKTFDIVKQLPRLSFTKAVKAHFAE |
| | | SDEDFIFDGPKFAKPLRVPKEIYLQSFTDNGDKLADSAKKYTKAYLD |
| | | MSGDYKGYYEAIIKRIDYTKEFLSAYKSTSIYDLAFLKPAGKAAGSLCW |
| | | TRHI |
| 110 | Cas12 | MCIRDRDLAVAALNRGDAXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXGQLNAIRNFLASDGYGAHDLVPVNYNGSNDRGAEVQVGN |
| | | QKNWTVTSSAPVVVQYNNADTTLTVQGHTDRLIVTGSGNDTITLK |
| | | DSGDDKVLLGDGNNTVVAGSGADTIVGGAGNDVLIGTNGGYGTEL |
| | | LGGAGNDVLRNAGSHGIYMDGGAGNDTFYGGTGPDTMEGGDG |
| | | NDLMYANGRGSSIDGGAGNDTIYGGPGGDTLTGGDGNDLLRSDS |
| | | AFGTKGSGNLLVGGAGNDTLWAGAGYDTLKAGSGSDTLISGTGSS |
| | | QMIGGSSGNTTFEVAYHTGNDTITGSGSGNTVYLDGRDFSDATISN |
| | | HSGVTTVSFSDGQVLKISGVQDIVFSDHDYKV |
| 111 | Cas12 | MSITRSIKVKLIVPRDASLEARQLREGLWATHLFVNDGCHYYERLLLE |
| | | FRQRDVCVGKDDAGKDVIVPAAEWADRLRARLGRNGMVPSHIEA |
| | | ALPIFRELYENMVPSALKAKSGTGQAGRSWHSKLVSPTSRGGEASA |
| | | ARIDVLRPLLPVSGDDPAFEPAARALIEEAGDELLTSTGRCPAWVTA |
| | | YRKGPEGSAWVEKLRIQLREAVEAGDFDPPSDPQILAAGAVPAAPP |
| | | LGAGIDALRPLLPLLGGDPAFEPAARALVEDIGDELFTSTGRPPTWV |
| | | TAHPTWVRAHRKDAECLEAADDFKWVERLRQRLRDDAKAGKFEQ |
| | | PLHERLGALGALPVAKPIGAGRVVSRADLTVFERGAMELAIEHLIGW |
| | | ESAGHRARAQYVERKKRHDDLLOWIEAEAPDALLAVRAYEAARTIH |
| | | LATLGELGAAPQYTLRLREIRPWRKLREWLLQNPDATIDERRRRLAT |
| | | MQTNDPRGYGGEALAWLAAPERRALVEHPAGDVVTRIAVLNIRKSI |
| | | LDRSRLFPTCTLADPVEHPRFAKFGKPGDKNSAGYALAVDGVRREAI |
| | | IKILVPRQDGLLVPTDLRVPFAPSGQMRDLRASGLDISYERQDGRGR |
| | | QAAKLQGGNLMFDRTHFARCGAPGPEALGSVWIKVALDLSSPAAS |
| | | LAMKTATPVRTYLSTAVRGRPESTKYEKAAPPEGFRVLSVHMGLRT |
| | | AATASMLRFGAPEEGGHEVPVSGLAGETLVAFHERTVTMKLPGED |
| | | PDTRTEANRGVAKRELRGLGRGIGCLKAIRRASASATPEDRAEALVII |
| | | ETHVGQGDRHGWAPAEAVGRLDPHGDPDDWKTACAALYAAVEA |
| | | DLGVAISSWRKAARAGGATGMLGGKSLWAVDHLERSFRFLRSWDL |
| | | RARPHDGDPRRPRPGYASKLLHHIDGVKDDRVKTTADRIVQAACG |
| | | RAWIGGPTVKRGTQDVRLPGRWEQRGPRADLILLPDLTHFRFRSDR |
| | | PRAENSRLMRWAHRQLAIYVRMQAEVEGILVADTGAAFTTRFDA |
| | | WTGAPGVRCEPVTADHLRGIAKREDYWLARLLREGALKHLRIDPAS |
| | | LRVDDLVPMDHGKILVALDGVDLPGLRILDTDVNASQGLGRRYIEG |
| | | HGLAYRLPGARVPRGEGEREAAVVHIKGKRLASAMGGTVVVLRASE |
| | | GPGDITWTAEVYDRPQGARKALGLSLAAFNSIATAAVDDEGPAPEN |
| | | DDEALEEEAEEALGIATGERIVFFRDPSGAVAGGGWLEASAFWGIA |
| | | NRMVTDRLRELGRLG |
| 112 | Cas12 | MAQASSTPAVSPRPRPRYREERTLVRKLLPRPGQSKQEFRENVKKLR |
| | | KAFLQFNADVSGVCQWAIQFRPRYGKPAEPTETFWKFFLEPETSLP |
| | | PNDSRSPEFRRLQAFEAAAGINGAAALDDPAFTNELRDSILAVASRP |
| | | KTKEAQRLFSRLKDYQPAHRMILAKVAAEWIESRYRRAHQNWERN |
| | | YEEWKKEKQEWEQNHPELTPEIREAFNQIFQQLEVKEKRVRICPAA |
| | | RLLQNKDNCQYAGKNKHSVLCNQFNEFKKNHLQGKAIKFFYKDAEK |
| | | YLRCGLQSLKPNVQGPFREDWNKYLRYMNLKEETLRGKNGGRLPH |
| | | CKNLGQECEFNPHTALCKQYQQQLSSRPDLVQHDELYRKWRREYW |
| | | REPRKPVFRYPSVKRHSIAKIFGENYFQADFKNSVVGLRLDSMPAGQ |
| | | YLEFAFAPWPRNYRPQPGETEISSVHLHFVGTRPRIGFRFRVPHKRS |
| | | RFDCTQEELDELRSRTFPRKAQDQKFLEAARKRLLETFPGNAEQELR |
| | | LLAVDLGTDSARAAFFIGKTFQQAFPLKIVKIEKLYEQWPNQKQAGD |
| | | RRDASSKQPRPGLSRDHVGRHLQKMRAQASEIAQKRQELTGTPAP |
| | | EIIIDQAAKKATLQPFDLRGLTVHTARMIRDWARLNARQIIQLAEE |
| | | NQVDLIVLESLRGFRPPGYENLDQEKKRRVAFFAHGRIRRKVTEKAV |
| | | ERGMRVVTVPYLASSKVCAECRKKQKDNKQWEKNKKRGLFKCEGC |
| | | GSQAQVDENAARVLGRVFWGEIELPTAIP |
| 113 | Cas12 | MNIIMENFEKFVNLYELSKTLRFELIPFSQTKVELEKDWIIEKDREIEEK |
| | | YHIIKEKLDTLH1KFVWQALEWVDLSLLEEYAELYFACKKDTKNKKLKS |
| | | KFEKLEKKIRQEITSFFDAEWNKWKEKYGFLKKWWTSFLTEKEILDVL |
| | | IDIFPENKDDFEIFKWFFTYFSNFNESRKNFYKDEWKAGQIATRAIDE |
| | | NLTTFLENIIKYKNFKKENPDFFTENEEKVFELDFYNFCLTQKWIDNY |
| | | NEIIWAKSLEEWKNTQGVNQRINLLKQKNEKSNKKNLSYPKFDILYK |
| | | QILSEKSENDFIPNIENTEELFTVIQKSIKENDKKITEIDKLFKKFFLEEN |
| | | NIDIWKVYISKQAVNTISSKYFENWSSLWWYLWENSKKKYFSLWEI |
| | | KEALEDIKEKNIFKGEYYNNKIAFENKSNFENFLAIFYYEFQTNLSLLN |
| | | WNQNNLESLQEKEFKKEEKQVDIIKKYFDSVMDLYAMSKYFFVDLK |
| | | QAKNFPKDIEFYNDFDLYFSDYEPWKVYNLVRNFLTKKEVKTDKFKL |
| | | NFSNSQFLTGWDKDKEKERFWVILRKNEKYFLAILKKNNNKIFENYR |
| | | ENNPTDFYEKMEYKQLNNVYRQIPRLGFPLQKKLDSLKWKELEEYLE |
| | | KYKNNFWYNKEIAFIKEEFDIFQKNKEKWEKFDREKLKKLIDYYKKVV |
| | | LEKYSDLYDLKKLENTDYDELVNFYDDVEKSMYSLHFTKIETEFLENLE |
| | | KNWEIYLFQIYNKDFSDYKKENTKENIHTKYFKHLFSEENLENLKIKLS |
| | | GWAEIFFRDKTHNLKQKLDKNWKKMFYWENKDKKVLEHRRYAKD |
| | | SYGFHISITLWANNWDMYKFNQFFNKNFTPKHIIGIDRWEKHLAYY |
| | | SVIDLEGNLVETDTLNIVNGINYLEKLENIEKSRMQERKSWWEIENIK |
| | | NLKDGYISAVVSKLTELIEKYQAIIVFEDLNLGFKRWREKIERQVYQKL |
| | | ELALAKKLNYLTFKNKKDCEIWGVLNGIQLVPRVKDYQDIANYKQSG |
| | | IIFYTNPAYTSTTCPECGWRKTLKFPSKITKTSILEFFKEIQMSFYWEKF |
| | | SFTYENILWKSETLFSNVKRTQWNNKSRKIEHKENITQELKSVFEKYN |
| | | INLWENISEKLQQTDMLLEDLKVLFYNFKLINNIRNSDSKLEEDIISCP |
| | | CCLFNSENWFKGAYFNGDANGAYNTARKGIIMLENIKVNPERSNLF |
| | | VRNEEWDEFLREEF |
| 114 | Cas12 | MSKLFENMTNLYSLNKTLRFELKPVGNTRELIESKDFFKNDEEKAEN |
| | | YQFMKEKMDKIHRNYIQKSLETIKMLPILDQTEQKRLKKEDIKNELKS |
| | | LRSFISAAFVSVKDLLSNKIIEWLISEASIEEKEKIKKFDKFFGYFKTYVQ |
| | | NRGNLYKAEDKAGQIAFRLIDENLPRFFKAKQIIEEIIKKTPDFVVKER |
| | | NEKQEETEKKITEYLSIFYLNSYCHYLSQSGIDLFNEIVGHINLSINLYK |
| | | QKTGVKFSLIPLLYKLPLAPRKQISRLPKQIENPEELELIVKSVLDRIDQ |
| | | KINPFINNILQTVLEENSVYDLNWIYISIKTTEQWWSIGLSGRNSLREL |
| | | GYNQSKNWEEPVLKAEKNKFPFISLGEIKSFLDAKQLAWDDIKSVF |
| | | WEGKSQSLMNWSWSNIFFHLIGKEISSLIEKYLHSRKNYQISQSKEN |
| | | QKHVLDNSLSLWRLLGWFVLMHKKTHLTPEIKESFFYDWENGLDAI |
| | | VFDENSDPIHTIYDKVRNCLTKKPYSNKDKIKVNFDCPYLLWGWDQ |
| | | NYDAFGWLIFHDWKKYFLWLIKGSWLNMEEKNKLYEWINPMNSIT |
| | | KHYDYQKPDFKNVPRLFIRSKWDTFAPMVREYWLPVNDILYLYDNE |
| | | LYKPDKKNPWKHKSYLRRLIDYFKLWLSKHKSFKHYIFKRKESDQYEN |
| | | LAEFYSDVEFSCYAIKKEKVNFDQVKALCESERLYLFEIYNKDYNQFSK |
| | | WKNKNLHTQYFEALFEERDNNLFMLSGGWSIFWRESNWKIVEKIR |
| | | SYSPKYNIEIIDKRRYTKNKLMIHIPIVLNFCRNQEWRVNDMIKSLIQS |
| | | QSNNFTILGIDRWEKHLLYYSLIRQDWTIIKTGSRNTITNKIKIVDYHK |
| | | KLDDREKKRDEAQANWEQQEQIKDLKKWYISQVINEISKMIIEHNAI |
| | | IVLEDLNGWFKRWRQKVEKSIYQQFELALAKKLNHLVFKNRWDTES |
| | | WGTMKAYQLTPLVAQFQDLSFQTWVVFYTPAWYTSTTCPCCGWR |
| | | KNIYFKYENEKQAKIELEKLNIVRENNYFSITYTAEWWNKKWKITWS |
| | | VLNKTDRILTTKWQTRLQYDRASKNTKEYDITTDFNSVFTAKFLDYKI |
| | | RLENAGSKQCRNLINSINLLLKIRNAKSGTDIDTIQCPACEFHSQWW |
| | | FQWNEFNWDANWAYNIARKGKVIIDKIVKGEKNTTVSQIEFDNEIQ |
| | | K |
| 115 | Cas12 | PAYRHPDPLLHPVFCDFGCSRWQICFDVRKNVRTTSPRRLCLTLFTG |
| | | SGMELVPFSWQSKRLARDLALEQRHRNPEASEVTRADRLGRAAAG |
| | | VSAGGAVSIKNIFNEENWNGRLQAPRSQLAAIAARVDKHGWDAK |
| | | ALRMLDRLRWLITFSPRLEPTGPWIEYALRIPDDAAAKPFLSRKNGF |
| | | AVLHRSNDDRKGLAKLILSRLPGLRVLSVDLGHRYAAACAVWETLSA |
| | | DQVREACQAAGHDGPTECDLYLHLKKDGRTVIYRRIGADTLSDGTP |
| | | HPAPWARLDRQFLIKLQGEEHKAREASNAEIWAVHQMEAALGRSV |
| | | PLIDRLVASGWGQRTEGQRARLEALKQLGWRPVAEKLDSDDEPGV |
| | | MEESPAIKPSLSIDELMSSAVRTMRLALKRHGDRARIAHYLITDEKTK |
| | | PGNVKEQLDENGRIELLQDALVLWHDLFSSKGWQDNKAKELWYV |
| | | HIATLPEYKAFQSSGEGHAGPERRGKPEEDREKLGVVAQALAPNVT |
| | | LREALHNAWKKRWEEDDARWKALLRWCKDWILPRGEAANSPAIR |
| | | KVGGLSLIRLATLTEFRRKVQVGFFARLRPDGKKAETGEKFGRKTLDA |
| | | LEELREQRVKQLASRIAEAALGIGKEHVFPTAKEPQKKCWCGSVHRK |
| | | KDPKRPRERVHSPCHAVVIENLTHYRPEETRTRRENRQLMSWSSGK |
| | | VKKYLAEACQLNGLHLREVNAAYTSRQDSRTGAPGIRCQDVPVKEF |
| | | MRSPFWREQVAQAKKKQAEGKGDARERFLCDLDEQWKNRSEDE |
| | | WKKAGPVRVPLRGGEIFVSADGTSPTAKGLHADLNAAANIGLCALT |
| | | DPDWPGRWWYVPCDPASFKPIMDKVEGSAAVQPDQALRQPAQA |
| | | QRGDAARDRKKRGKVGGRSREVVNLWRDVSSRPISPNDSWQEFT |
| | | PYWNDVOARVVNILRQSAGLTRGQGEP |
| 116 | Cas12 | MPLRSINIKMRLKRAAEGRALRQSLWLTHSVVNCAVAEIERVLLLCR |
| | | GRGYWTGDDEPVSAETVQQQALAFARETQARNGQPGIGGDAEIL |
| | | AALRGLYEAIVPSVNRDEQGRPLEGNAQAAGGFAGPMMDAESEG |
| | | FQSVFDKILEPLPSWVAKMTGGTRGWEQESVQWLKSPESERLQHA |
| | | SGSPPAWVRRLRTGEPWQEAFVQDQENKRKEVKGVPSLIQRLKKE |
| | | LGLLPLMRPPITLQFEDKRSGLTPWDRLTLRLAVAHLLSWESWNHR |
| | | AADEHGRVTERLARLETEAAPLASLIEGLREYEKIRHEELKRVAQAND |
| | | ENPFRIGARGVRGWDRVREAWLGATDDTKEGRFTSVAALQ.TKLGG |
| | | RFGDPDLFRWLAEEGREHLWGEHDPLPILARLNALSRLLRRKKDHAI |
| | | YTAPDARLHPRWTAYEAPGGGNLRNYSFEISGNDLALRLPLLRRVES |
| | | GLEEDSQRAEIALAPSGQFQSASWKGDGKPNRHLTYYSAHEQFSAE |
| | | LGGAEILYRRRHLENRKVKELEQGDIGPVWLKLVLDVQPNAPEGWF |
| | | TPRGRVVTPPTVHHFNTALVNRSKHAPDLVPGLRVLAVDLGVRTFA |
| | | ACSVFELVQGEPGNGMAFLADQERDLWARHERSFLLPLPGEAVDS |
| | | QLLAARRAAYNQLGLLRRDLGRLKGILRLSVKETAENRCGSLEELLAS |
| | | LEDEWNRGNAPAIDVAALLSAKGCLEMPQPAWEAAIAGTYQAAER |
| | | ELGNRVRDWRRTTRPRTTGEDDRRQRRGYSGGKSAWAVEYLDOV |
| | | RRLLQGWSLHGRSYAQIRRLDREKMGTFAAGLLDHINALKQDRVKT |
| | | GSDLIVQAARGYLPTGKKGWIKQYEPCRLILFEDLARYRFRTDRPRRE |
| | | NARLMRWNHRQILTETELQAEIFGLLIGTTGAGFSSRFHARSGAPGC |
| | | RTRLLTAEDLRSPTLAKQLELLAETQGIDPKCFRPGMQIPWDSGADF |
| | | VTLDSEGKLVQIHADVNAAQNLQRRFWTRFRDAYRISAVEIKQDGR |
| | | TSWYPDREGVRLRGALSTIVGGDGYARLVAADDDDGFVLEKVTRP |
| | | HWRRAAGAQAESGDDVGLDEVALEMAEAMDTDLERGEGRLVFFR |
| | | DPSGSVIRADRWYQAKAFWGQVRSRVTRALGLRRPGASS |
| 117 | Cas12 | MLDKFASLYPVTKTLRFRLLPQGRTEENMQVAKVLENDLERSEAAA |
| | | VVKGLIKKYHLQFISDTLSGSTLSWQALTETLDKFKADHTATAELDSA |
| | | LAAYRCKLAELFTKSPKYKVMATPVSIIKEILKTETDPENIAALNKLNG |
| | | YTYIIFDYVSTRMLTYSADAKATSLAYRLVDENYLRFYQD1SAAAEISA |
| | | VLEEAGFDNAEVEAFIRTDYNTCLTSEGIASFNAAAGSINQFVNVLL |
| | | QQNPVLQSEPALRRHLQPLYKMLLDEAESKIIKFEDYGQLRDAVENF |
| | | RRNFQDLPQSLIDIFAGRYDYSKIYVGYKYLNEASSQIAGGYNWKLLE |
| | | NALEDFYSKPYLVNGKLPVKYKTVVNKKMNQLAYSFTELQEALDAG |
| | | DSGSSITDLFGKYAELHAAYAAADGNVFYKEYDRKSIASLKNYLDAV |
| | | NAIARFIKIFAAPEVYVKDEGFYGIVDGAADKLRDFDLLYNMVRNYIT |
| | | KKPYKKSKVALTFNSSSFGRGWDENKIYDELTTIFTYNGKYYLGVINK |
| | | NDKPDLAAAVSKDEGGYKRMVYKTFDIVKQLPRLSFTKAVKAHFAE |
| | | SDEDFIFDGPKFAKPLRVPKEIYLQSFTDNGDKLADSAKKYTKAYLD |
| | | MSGDYKGYYEAIIKRIDYTKEFLSAYKSTSIYDLAFLKPAGKAAGSLCW |
| | | TRHI |
| 118 | Cas12 | MSRSIFSPFTNLYPIQKTLRRELKPLNENFQHDPALSALRNSEIPQRD |
| | | EQREKDYQAIKPLLDEFHNQFITESLHSLEPQDRSDFILFYQTYQKKK |
| | | KNKAEISEKELKSLDEEFESRTKALRNAIGTSFSVTAELRKSNPDYVSE |
| | | KGKPFLTQKSYKILTEAGVLWLLEKKYTSDPEKLALIRRFGNFFTYFTG |
| | | FNQNRENYYATDEKSTAVAYRAINENLLTFANNCELFEKLSVLSLSEL |
| | | EKKTFNPDSYSEYLTQSGIVFYNEMLANIRSKANLYTQEHKAKLPQP |
| | | KLLYKQIWSPRGDTIPFDLIASEAEFQETLHTMIRETDQRIPEFNKLLE |
| | | QIFEEKVDLSQIFFSKTSLNIISNRYFSSWHTLLEKGVELKLFKFKKNDE |
| | | ESFKLPAYLSLAELKELLESAPFQMAEKADADEEKHHQASLFKLQRE |
| | | NLHLEKSHSNWELLLKSMKSDFESFWTWEGEFWSYTLAKKALQSLS |
| | | ALESTNQEHKNLIKMLLDNALYAYRMLKWFKVDTSKLGFVPEGEFY |
| | | PSLDQLLQDYPLPKWYDMIRNYLTRKTYSQAKLKLNFDCSTLLNGRD |
| | | KNKEIQNLSVILRKDGKFYLAIMKKDQNKFFENSALYEGNLGTMEK |
| | | MDYKLLPWANKMLPKCLMPGSDKKKYGASDQVLELYAKGSFKKSE |
| | | KSFNLADLHTLIDFYKLALPKYEDWKVFNFQFQATENYQDISQFYRE |
| | | VEQQGYLLNWRKVNEKLIKQGIKDWSLFLFQISSKDFEGKSKTPDLQ |
| | | TLYWQQLFEFSTNVKLNGEAEIFFRPWSMKKEKKKLKVDNYDVFKH |
| | | KRYTEDKILFHVPITLWFGNNEVSPSAPSKFNQKLNQELIIPHFDDLH |
| | | VIGVDRWEKHLAFYSVVSVKTGKIVKQGTLNLLNGTDYEAKLSOKAE |
| | | NRLHARQNRDTIEKIADLKNGYISQVVNKLVELVLEYNAVIVFEDLNA |
| | | GFKRGRQKIEQSVYQKLELALAKKLNFIVKKEKAVGEPWSVTSAYQL |
| | | APQINTFWDIKWKQRGIMLYTRANYTSVTDPLTGWRKQYYFKKGS |
| | | SEEMKAQFFKSFKNLTRDAGQEAYIFDDGTWLLYSNVERRRGKRGD |
| | | HRERTQIKYDPSLELDTLFSKYQJEKSDSLFDQLKNRELPQTFWTSFFR |
| | | IIDLIMQIRNTDDEGRDIILSPIGNPQERFDSRKRYNQLPRDEKGGIIE |
| | | ESAFEYPTSWDANGAYNIARKGVMMLERIKENPEKPDLLIRDAEW |
| | | DKKITRK |
| 119 | Cas12 | MKNFQDFTNLYELSKTLRFELKPIWGTKKLIEEKNILKLDKKKRENYEK |
| | | VKPYFNKIHQEFINFALRNPNFDFSQFEEKYLNWLKDKKNKDLLKEK |
| | | ESIDKIFLEKIWKLFENSVKDFLKENGFESIVKSEDQNLKFFRRKEIFEV |
| | | LQEKYGSELETQMVNKDWEIKSIFNGWEKWLWYFDKFFNTRDNFY |
| | | KTDWTSTAIATRIIKDNLKIFLENTIIFEKVKNKKIDFSEVEKNFSVSIDT |
| | | FFEINNFNNCFLQDWIDFYNKVIWGETLENWEKLKWLNEIINKYRQ |
| | | DTGEKIPYFKKLQKQILSEKDWVFIDKIEDDGGFYEVLKNFYKNAAEK |
| | | EWFLKNIFENFYTISDKNLEKIYFNKIAFNTISHKFWSALEFERILYEEM |
| | | KKEKADWIKFEKKENKYKFPDFIQIIFIKRSLENYDSENLFWKERYYKS |
| | | EENVDWFLEKNNNNIWEQFCKILNFEFLNILKRRIIDEAWEEYEVWF |
| | | EISKNILWEKLENFELNQENKWIIKDFADYSLALYSFWKYFAVEKWR |
| | | NWDLNIDISDDFYGWEDWYIEKFYNTGYDEIVKPYNLMRNYISKKP |
| | | WEDSKKWKINFETSSLLSWWDKNLESNWSYIFQKWNKYYIWIINW |
| | | SKPAKEVLEKLYSWNGEKIKRFIYDFQKPDNKNTPRMFIRSKKDSFSP |
| | | AVGKYNLPVEDILEIYDNWLFKTENKDNSNYKESLSKLIDYFKLGFSK |
| | | HESFKHFNFVWKDSKEYENIADFYRDVEKSCYQITSEFLDFEELKKLT |
| | | FKKHLYLFQIYNKDFELDESLQKNWYNFRDEWQKNIHTKYFEALFLE |
| | | ENILRKSWAVFKLSWGWEVFFRKESIKAEKEKRKNIEVTKNRRYTEE |
| | | KYFLHFPIQVNFKNEISWNFNQEINKFLANNPDINVIWIDRWEKHLA |
| | | YFSVINQKWEILESWSFNKIENYNKNWEKLLFPEREIKEIHKDWSLID |
| | | LELVETWRKVDYVDYKLLLEYKERKRLLQRQSWKEVEQIKDLKKWYI |
| | | SALVRKIADLIIKHNAIVIFEDLNFRFKQIRGWIEKSIYQQLEKALIDKL |
| | | NFLVNKNEINLEKAGSILKAYQLTVPVDSLKEIWKQTWVIFYTEAAYT |
| | | SKIDPIKWWRPNLYLKKQNAEINKENILKFDNIIFNSKENRFEFTYDLK |
| | | KFFWKDSKFPAKTVNTVCSCVERFKWNRNLNNNKWGYIHYENLTD |
| | | WKLANKEQKEDEFSNFKELFEKYFIDINWNILEQIKNLDTKNNEKFFS |
| | | SFIDLFTLVCQIRNTNQNAKWDENDFILSPVEPFFDSRKSQNFWKSL |
| | | PKNWDENWAFNIARKGLIILNRISENPEKPDLLIFNADWDNFARNI |
| 120 | Cas12 | RPGASAALSLTPLKTLFSENFSSFMCGNWQIINDSLKTYYNENIKSKG |
| | | KAKEEKVKKAIKAIEYKSLADINQLVERYNNDELNRKAEEYISAINEKIK |
| | | DLDVNEIEYDEKINLIENETKSEEIKSKLDSIMEIMHWTKMFIIEEEIEK |
| | | DVNFYNEIEEIYDELQPLVTIYNRIRNYVTQKPYSEEKIKLNFGIPTLAN |
| | | GWSKTKEYDNNAIIMIRDGKYYLGIFNAKNKPDKKIMEGHQSEENG |
| | | DYKKMIYRLLPGPNKMLPKVFMSKTGIAEYKPSQYILECYEQNKHIKS |
| | | DKNFDIKFCRDLIDFFKTSINRHPEWSKFNFKFSETSEYEDISTFYREV |
| | | EKQGYKIEWTYISEKEIKELDENGQLYLFQIYNKDFSEKSKGKENLHT |
| | | MYLKNLFSEENLKNIVLKLNGEAEVFFRKSSIKKPIIHKKGSVLVNKTY |
| | | NENGERKSIPEEQYTEIYKYLNSIGTNELSEKSKKLMEEGKVEYYKAN |
| | | YDIVKDYRYSVDKFFIHLPMTINFKAAGFSPINNIALKNIALKDDMHII |
| | | GIDRGERNLIYVSVIDTKGNIVEQRNFNIVNGIDYKEKLKQKELDRDN |
| | | ARKNWKEIGKIKDLKEGYLSLVVHEIAKLVVKYNAIITMEDLNQGFKR |
| | | GRFKVERQVYQKFETMLINKLNYLVDKDLAVDQEGGLLRGYQLTYIP |
| | | ESLKVLGRQCGYIFYVPAAYTSKIDPTTGFVAIFNYKGMTDKDFVTSF |
| | | DSIKYDDERGLFAFEFDYENFVTHKVEMARNKWTVYTYGERIKRKFK |
| | | NGSWDTAEKVDLTYQMRSILEKYEIEYNKGQDILEQIEELDEKAQNG |
| | | ICKEIKYLVKDIVQMRNSLPDNAAEDYDAIISPVINNNGEFFDSTRGD |
| | | EDKPLDADANGAYCIALKGLYEVMQIKKNWNEETEFPRKELKIRHQ |
| | | DWLDFIQNKRYL |
| 121 | Cas12 | MQCGSLRHNSKSLDSALAYPARLTTGGNEGKTSEAHIENDSLPEITR |
| | | RPSRLARDFAPSNPFPFMKRIYQGQITGMQFHGAAGEQAVPANQ |
| | | NWEQALWDHHGLFHDAVNYYLVCLLALARPGNPVYAIREKLDAHN |
| | | GAEPDELMVWRTFRRRGCNRPGLRDSVAKYLTPGNDEPTTEECFA |
| | | AVLAGNPLGATEEGRAILNEGLAQLLGKCTGESGCRNSAKEYLPRFT |
| | | KPDFKGNFEEDTARLNRAEASKRLPFYLHDPATTPNSPALDEFGVLSI |
| | | ALPNPKRAELTGAEARDKLLEFSREWSARLPDGSADWRRLEAKITAL |
| | | GDSLAIPGYTSGSAKGENRFQLYAMFLFCFVEKSDFTLGLLRQTTRR |
| | | PVEGEEPPPRSNHSATAGDPIRIARGGRGYVFRAFTSLKQWGGDTG |
| | | GDLKWPKFDMAAFCEALKALHQVEAKAKQRAEERSKKQALLDYQR |
| | | GRIRRFKPTANSEDATPPPVLAGDPRISRLEQLLNTDLQDEYEMSEG |
| | | VAVAYGLHPRTIRGFREIRKRWISAVGNAPFSEAARATLIACVRSFQS |
| | | ENPGTVGSARLFEALAEESNWIIWREPSAAEQQSWRENADLPESAE |
| | | FALDPLQALTDERELKDEIERLSGPIRFTPADAEHSRRQFYFSDVSQID |
| | | KRNRFRPRLNEVEVELAVRPNGHWTNVWVTLQYSAPRLLRDQLPT |
| | | ADEAGGGWQQAMMAALNLRAPLKKGGEAVSFADCAALSLMPDI |
| | | SPDGEKRLLLNFPVELDGEAIANQLGRSKRWETLQFGGADDESYWL |
| | | RWPKTWIDETKVRRKAAPPKWWLSKEPFSVLGVDLGQRDAAACA |
| | | LVQVSPGNCPTGVCRHVGSADGVDWWATVRSMNMLRLPGENA |
| | | KVMRDGRFQEELSGSRGRSASIDELKEAGDICARLGFVADTILGANG |
| | | RALSFPELNDRLLFSLRSAQSRLARLQSWSCVAHADVPPARREGILR |
| | | DISEAKDDPLGLKPLATAGNLEAIASTLREVILQERTSISAQLVRVADR |
| | | ILPLRGRRWEWVARPESPSNHMLRATAPDTDPRRKLVAGQRGLSL |
| | | ARVEQLESLRQRCQSLNRALMQVPGTPSKLGRRSRGVELPDPCPDL |
| | | LDRLDALKEQRVNQTAHLILAQALGVRLRIHQVSGPQRTRSDSHGE |
| | | YERIPGREPVDFLVLENLDRYLASQGRSRSENSRLMKWCHRAILLKL |
| | | KQLCEPYGLRVLETPAAYSSKFSSRDGTAGFRAVEVTPDDLGSHRW |
| | | RKHSERLADPGASLSRDEREESTRLMAFAERLKALNQDLIARQEAAR |
| | | SSNQPFRPKWRTLLAPQQLGPIFVPAVGKPLQADINAAINIALRAIAS |
| | | PDVDDIHLRIRAARSGDKFVVRAENTRERARWGSAEAEIGLPTGAG |
| | | AKEIEERRSLLTEARVNFFFDPASVAAFDHGKVRDAKLAVTSGRGLW |
| | | GTLRREEWAIVGKINNDRLAAHGLGRPFAERVDL |
| 122 | Cas12 | MSQRDPLPSPTPRAYTLRLSTPDADNGAWRERLWKTHEVVNSGAE |
| | | AFGDWLLSLRGGLDHRLVAVKVRVGRGASKTEREPTEDERRERRIVL |
| | | ALCWLSVESKKGAPQGRVVEDPVDALKRILGQRGLSEADAEQWVF |
| | | DCRDSLSATIRDDATWVDRSAAFDETVQRLGSSLTREHAEQVIIEFF |
| | | GDIHDYLALPKNMEEDGVTSPRGGSGKEFRTQARSWLSENWGAG |
| | | QKGDKAQIVDALRKCSRCIAQEKPSTGSDLLRVLVRSLGGSPEETLRF |
| | | DELRKLVGWRTGRRSTGAFALQRTVDMDRLSESDLESLEAKLTVEID |
| | | AKSPEASRSVPQWVESMRTAVEREVGMPFRSTRDLIGEFGVMLDH |
| | | AARRVSATHSWIKRAEAERRKFEGDAAKLAQIPEEARQWLDQFCE |
| | | DRSQELNALEPYRIRRRAVEGWESVVRTWAKADCKAEGDRVKAVR |
| | | ALQPEIEKFGDAALFEALAAEDALCVWLLDGKATPRPLLDYSAATDA |
| | | QYRKRRFKVPAYRHPDALLHPVFCDFGESRWTIEFSAHRALGRRRK |
| | | AQQLVDNKTVALQKAQERLRKAKAESSRSRAEEKVRAAEHALEAAR |
| | | KKLAFLQYRRALAVCLWTGKGVESTSLRWHSKRFAKDFAIGSSPED |
| | | GASTGVPVARADRLGRASANVPRGASVTISGVFEQKEWNGRLOAP |
| | | RDALNAIAKVRDDETLPAADRSRRVQQMLSRLPWFVTFSARLVPQ |
| | | GPWLDYASEHSALGLRVDPKYWPHAEENRQRKGMARLILSRLPSLR |
| | | VLSVDLGHRYAAACAVWETLSAQEMQRICREMGSAPPSPDDLYLH |
| | | LRRADGNGKQRTTVYRRIGPDTLPDGGQHPAPWARLDRQFLIKLQ |
| | | GEDESARKASDSEIAVVKKFEADLGRPAMQRRSLRVDDLMSAAVRT |
| | | ARLALRRHGDRARIAFNLIADRRFRPGGAEEPLTDETRVDLLADTLA |
| | | TWHGLFSGGQCHDERAEEQWNEHIAPLLASSGASLSVPSDGDAVA |
| | | ATPARRRRKEVRGKLVPAAMELARRDLSQLSVLWVARWHQDDES |
| | | WRARLRWLRDWILPRGAKADSGAIRHVGGLSVTRLATIRSLWOLQ |
| | | KAYRTQPEPEDPRKNVPKKGDTSLDDFGRTILDDLEHLRENRVKQLA |
| | | SRICEAALGVGVEQPNGGAKDPKRPQERRFSPCQAVVIENLTRYRP |
| | | EETRTRRENRQLLNWSAGKVKQYLSEACELHGLHLREVSAAYTSRQ |
| | | DSRTGAPGIRCQDIPVVDFVRENGPCWGRLRSAQQSSGGTAEDQL |
| | | LLALYERWSEKERTWRDQSGNVWAMDGEGRWVARNGTOLPOG |
| | | SGLTPHPIRVPQRGGDIFVSAAPRSPAANGIQADLNAAANIGLWAL |
| | | LDPDWEGRWWRLPCSAMDLKPVKGSVNGSAVIHLDAPLKAAATK |
| | | GAAKKDVVNLWRDISVRSLTEGVWRGYDAYWNLARYRVVQTLRP |
| | | QVGIGSDE |
| 123 | Cas12 | MFKGDAFTGLYEVQKTLRFELVPIGLTQSYLENDWVIQKDKEVEENY |
| | | GKIKAYFDLIHKEFVRQSLENAWLCQLDDFYEKYIELHNSLETRKDKN |
| | | LAKQFEKVMKSLKKEFVSFFDAKWNEWKQKFSFLKKWWIDVLNEK |
| | | EVLDLMAEFYPDEKELFDKFDKFFTYFSNFKESRKNFYADDGRAWAI |
| | | ATRAIDENLITFIKNIEDFKKLNSSFREFVNDNFSEEDKQIFEIDFYNNC |
| | | LLQPWIDKYNKIVWWYSLENWEKVQWLNEKINNFKQNQNKSNSK |
| | | DLKFPRMKLLYKQILGDKEKKVYIDEIRDDKNLIDLIDNSKRRNQIKID |
| | | NANDIINDFINNNAKFELDKIYLTRQSINTISSKYFSSWDYIRWYFWT |
| | | GELQEFVSFYDLKETFWKIEYETLENIFKDCYVKGINTESQNNIVFETQ |
| | | GIYENFLNIFKFEFNQNISQISLLEWELDKIQNEDIKKNEKQVEVIKNY |
| | | FDSVMSVYKMTKYFSLEKWKKRVELDTDNNFYNDFNEYLEGFEIWK |
| | | DYNLVRNYITKKQVNTDKIKLNFDNSQFLTWWDKDKENERLGIILRR |
| | | EWKYYLWILKKWNTLNFGDYLQKEWEIFYEKMNYKQLNNVYRQLP |
| | | RLLFPLTKKLNELKWDELKKYLSKYIQNFWYNEEIAQIKIEFDIFQESKE |
| | | KWEKFDIDKLRKLIEYYKKWVLALYSDLYDLEFIKYKNYDDLSIFYSDV |
| | | EKKMYNLNFTKIDKSLIDGKVKSWELYLFQIYNKDFSESKKEWSTENI |
| | | HTKYFKLLFNEKNLQNLVVKLSWWADIFFRDKTENLKFKKDKNGQEI |
| | | LDHRRFSQDKIMFHISITLNANCWDKYWFNQYVNEYMNKERDIKII |
| | | WIDRWEKHLAYYCVIDKSWKIFNNEIWTLNELNWVNYLEKLEKIESS |
| | | RKDSRISWWEIENIKELKNGYISQVINKLTELIVKYNAIIVFEDLNIWFK |
| | | RWRQKIEKQIYQKLELALAKKLNYLTQKDKKDDEILWNLKALQLVPK |
| | | VNDYQDIWNYKQSWIMFYVRANYTSVTCPNCWLRKNLYISNSATK |
| | | ENQKKSLNSIAIKYNDWKFSFSYEIDDKSWKQKQSLNKKKFIVYSDIE |
| | | RFVYSPLEKLTKVIDVNKKLLELFRDFNLSLDINKOIQEKDLDSVFFKSL |
| | | THLFNLILQLRNSDSKDNKDYISCPSCYYHSNNWLQWFEFNWDAN |
| | | WAYNIARKGIILLDRIRKNQEKPDLYVSDIDWDNFVQSNQFPNTIIPI |
| | | QNIEKQVPLNIKI |
| 124 | Cas12 | MLSFNWVLFFFFSKMSRSIFSPFTNLYPIQKTLRRELKPLNENFQHDP |
| | | ALSALRNSEIPQRDEQREKDYQAIKPLLDEFHNQFITESLHSLEPQDR |
| | | SDFILFYQTYQKKKKNKAEISEKELKSLDEEFESRTKALRNAIGTSFSVT |
| | | AELRKSNPDYVSEKGKPFLTQKSYKILTEAGVLWLLEKKYTSDPEKLAL |
| | | IRRFGNFFTYFTGFNQNRENYYATDEKSTAVAYRAlNENLLTFANNC |
| | | ELFEKLSVLSLSELEKKTFNPDSYSEYLTQSGIVFYNEMLANIRSKANLY |
| | | TQEHKAKLPQPKLLYKQIWSPRGDTIPFDLIASEAEFQETLHTMIRET |
| | | DQRIPEFNKLLEQIFEEKVDLSQIFFSKTSLNIISNRYFSSWHTLLEKGV |
| | | ELKLFKFKKNDEESFKLPAYLSLAELKELLESAPFQMAEKADADEEKH |
| | | HQASLFKLQRENLHLEKSHSNWELLLKSMKSDFESFWTWEGEFWS |
| | | YTLAKKALQSLSALESTNQEHKNLIKMLLDNALYAYRMLKWFKVDTS |
| | | KLGFVPEGEFYPSLDQLLQDYPLPKWYDMIRNYLTRKTYSQAKLKLN |
| | | FDCSTLLNGRDKNKEIQNLSVILRKDGKFYLAIMKKDQNKFFENSALY |
| | | EGNLGTMEKMDYKLLPWANKMLPKCLMPGSDKKKYGASDQVLEL |
| | | YAKGSFKKSEKSFNLADLHTLIDFYKLALPKYEDWKVFNFQFQATEN |
| | | YQDISQFYREVEQQGYLLNWRKVNEKLIKQGIKDWSLFLFQISSKDF |
| | | EGKSKTPDLQTLYWQQLFEFSTNVKLNGEAEIFFRPWSMKKEKKKL |
| | | KVDNYDVFKHKRYTEDKILFHVPITLWFGNNEVSPSAPSKFNQKLN |
| | | QELIIPHFDDLHVIGVDRWEKHLAFYSVVSVKTGKIVKQGTLNLLNG |
| | | TDYEAKLSQKAENRLYARQNRDTIEKIADLKNGYISQVVNKLVELVLE |
| | | YNAVIVFEDLNAGFKRGRQKIEQSVYQKLELALAKKLNFIVKKEKAVG |
| | | EPWSVTSAYQLAPQINTFWDIKWKQRGIMLYTRANYTSVTDPLTG |
| | | WRKQYYFKKGSSEEMKAQFFKSFKNLTRDAGQEAYIFDDGTWLLYS |
| | | NVERRRGKRGDHRERTQIKYDPSLELDTLFSKYQIEKSDSLFDQLKNR |
| | | ELPQTFWTSFFRIIDLIMQIRNTDDEGRDIILSPIGNPQERFDSRKRYN |
| | | QLPRDEKGGIIEESAFEYPTSWDANGAYNIARKGVMMLERIKENPE |
| | | KPDLLIRDAEWDKKITRK |
| 125 | Cas12 | MLDKFASLYPVTKTLRFRLLPQGRTEENMQVAKVLENDLERSEAAA |
| | | WKGLIKKYHLQFISDTLSGSTLSWQALTETLDKFKADHTATAELDSA |
| | | LAAYRCKLAELFTKSPKYKVMATPVSIIKEILKTETDPENIAALNKLNG |
| | | YTYIIFDYVSTRMLTYSADAKATSLAYRLVDENYLRFYQD1SAAAEISA |
| | | VLEEAGFDNAEVEAFIRTDYNTCLTSEGIASFNAAAGSINQFVNVLL |
| | | QQNPVLQSEPALRRHLQPLYKMLLDEAESKIIKFEDYGQLRDAVENF |
| | | RRNFQDLPQSLIDIFAGRYDYSKIYVGYKYLNEASSQIAGGYNWKLLE |
| | | NALEDFYSKPYLVNGKLPVKYKTVVNKKMNQLAYSFTELQEALDAG |
| | | DSGSSITDLFGKYAELHAAYAAADGNVFYKEYDRKSIASLKNYLDAV |
| | | NAIARFIKIFAAPEVYVKDEGFYGIVDGAADKLRDFDLLYNMVRNYIT |
| | | KKPYKKSKVALTFNSSSFGRGWDENKIYDELTTIFTYNGKYYLGVINK |
| | | NDKPDLAAAVSKDEGGYKRMVYKTFDIVKQLPRLSFTKAVKAHFAE |
| | | SDEDFIFDGPKFAKPLRVPKEIYLQSFTDNGDKLADSAKKYTKAYLD |
| | | MSGDYKGYYEAIIKRIDYTKEFLSAYKSTSIYDLAFLKPAGKAAGSLCW |
| | | TRHI |
| 126 | Cas12 | MNRIYQGRVTKVEILNGKNADGQPQELPNWQTALWQHHELFQD |
| | | AVNYYLFCLAALASSASSPMGKLRNQLGSVWEPFGRTGRRFKGLRD |
| | | SVGPYLLPDRPTPSIEEAFAAALAGNNSQPELLQLAVDALVEDLGGD |
| | | AAIQQEGRGYLPRLCSPAYNGQFPRGANSLQKEAAKIRLPTLLHNVA |
| | | TSRNLDALAAELEFGFFANPVPNAGPIAGQEARNKLLEALAWLKAK |
| | | DTRLTHSANELEKRIGVLPDSVSFPTYSGGSINKEALKHRFFAYLLFQ |
| | | HVERSLVTFEILRDSYPTPKPSARKSSKATPDTMAQLTQFGDDPVKL |
| | | ARGTRGYVFRAFTSLPCWGAKAPSDILWSEFDIAAFKEALKTANQFR |
| | | LKTKERLDKADELAGELAWMNGEKSKFKPSEHSEQEPPAVLKGDPR |
| | | FEVLKQLFEVELAEEHYLAEGESVIYGLHPRTLRCYRELVERWNKTVQ |
| | | PGEVFTEATSKKLVASTDKFQAENKERIGSVTLFKKLLERDYWCLWQ |
| | | TPDVQTVAARQKAGFSSNIIEDYQRYLELQTDIARLKEPIRFTPADAE |
| | | QSRRLFMFSDLAGKSKHKHLPNATPYGFAVDVALAASEGGVWKET |
| | | RVRLHYSAPRLRRDGLRKGVGEDLKRTAWLQPMVAALNLSEPAPQ |
| | | DFSKCAVSLMPDKSWKGLRHLLNFPVTLDPTVVQKAIGSQARWAN |
| | | QFVAFGKGATEQKFFLRWPEEVSASQKKAGPWWDALRAFSCLSVD |
| | | LGQRDAGAFALLDVRANADWGKKPSRFIGEADGRNWRAALAAAG |
| | | LLRLPGEDALVWRDGKWQEELYGEKGRLATKEEWLETNAIFSALEQ |
| | | NSEEWIGADPKRRSFPELNSKLLVVARRGQSWLARLHRWLWMLG |
| | | DENKRERALRELLEQERQEAWRKRAEGKDLLALKQSLTAEIQRLNGL |
| | | LPEQLVFLANRCLPLRNRKWAWNQHPDKAFAEKGCHLLEMVEAPS |
| | | HLPLLAGQRGISFERIGQLEELRRRFQSLNRVLQRELGAPPKSGRELR |
| | | DDLVPDCCPDILAKLDRVKEQRVNQTAHLIVAQVLGVQLRKHQTPE |
| | | SERTERDLHGEYEIMPGRKPVDFIVLEDLSKYLSSQARGRGENVRLM |
| | | KWCHRQVTAKVKELCEPFGLPVLETPAAYSSKFCSRTGVAGFRGVE |
| | | VTLKDRQSFPWSKRLEEDSAEVAELFGWLETASAGHKTKQPRCLLA |
| | | PMSLGPLFIPVTSQAPIMQADINAAINLGLRAMAAPSVHEIHVRIRS |
| | | EAKDGQFRVRAQSKREKARWGNEPPPIKMLVETQRVALAREGGH |
| | | PNFFVDPLKAATFDRAEVEGLGLPVASGRGLWKSVRDAEWKRCREI |
| | | NLERMRRWGFIMEAPRTLQVSRKEDEDDLTF |
| 127 | Cas12 | MAVRSVKLKLLVPRDGSAESVRKRKALWATHQFVNDAAAAYAELLL |
| | | EMRQEDVCRGTDDHGKDVIEPAAHWQAKLRARLAAKQLPPVAVA |
| | | EALPLLKAFYGSRLIKSFVANDKGVAGTGNATDLNTWLSGLVDPASV |
| | | AGEKTELRKQLLAELPLCEAADADFEGAARKMLAKSDAREALLEGP |
| | | GTGVGWPAAYNANPTDSVWLDMLHKAAAKARLELADTTVSELKK |
| | | LGVFPLLQAASSNRVFGSGVLNPFERMAAAQAAAALLPWETKRHE |
| | | MRKRRDKFADQLNQWDTEFGASHATALAAIRAFEAEESERARRESL |
| | | GNEGTGYRIGGRELRDAWTLLRDWLKGHSTATAAAREDKVRELQA |
| | | KQGRSFGSHRLLSWLAKPAQQWLADHSAGDVVTRIAVRNARQRK |
| | | LDTARTLPIWTGADAVKHPRFANFDPPNNTNQPGFDLRAGTOKGR |
| | | LTLRLSLLTERADGLLLAQDHDFQLVPSRQMAEIVLHKDGKERALSW |
| | | QSQDGIGRQVGDVGGSALLFSRDHAECLLERKQITRLERGAWPAAL |
| | | PVWFKLSLDIGAEHKALLKQRFKWGVWLNSALVTRNAKDAKGVPP |
| | | PVGTRVLAVDLGLRSAATVSVWQVVDAATPVVAGKWRVPLSDTLS |
| | | AVHERSAMLALPGEHVDAGVLAARRAANEKLAGLLAATSHLSTVFK |
| | | LGRAEQGDRRRELLERLGEGDDRRARAAVATTAAERDGLRAVLGA |
| | | TQDAWAGAVAAVWRRLETDLAGAIAAYRKQQREDVQLRREARHG |
| | | PGASQLPKQAAAERLLGGKSAWQIEYKERVRKLLTRWIMRQRPGD |
| | | TAVRRLARKDLGKYCGGLLDHLTALKEDRAKTTADLIVQAARGRVR |
| | | AHKDAHGRQQDRELWLAKYAPCDLIVMEDLGRYRFATDRPPSENR |
| | | QLMQWTHREVFRLVQMQAEVEGIQVLETGAEFSSKFDARTWAPG |
| | | VRCEPITKLWVERYRNGEMPWLADKADEWRREGIELAQLVPGQLL |
| | | PTGSGEQFVAVSATGGLRVRHADLNAAQCIALRALTGHGTAFRLTA |
| | | RRLGDVFVSAKGLGKRPQGALWREFGSALPPAVVVLRPAGEVRYAL |
| | | RPFASARDAAAALGLQLGALRNVDATDAESDAEDGDLAELLAGAD |
| | | PDRATFFRDPSGDVHGGAWVQAKVFWAEVRRHVRLGLQAQGLLP |
| | | AAARSSEPRQMQLPLAGALPGDDIPL |
| 128 | Cas12 | MSTQKNPFNQFTNLYELQKTLRFELRPVPETKKLLEKGEGKNLIQMD |
| | | LEIDRLYEKEMKPMFNILHEKFINESLGLVKLDCKKLKKLENLLAEADK |
| | | LRKQIKEGRKNKNNISEVEKRLKIIIGDNSQGKNKNGEIAVLQDELRV |
| | | LIVKAFNLTADKWKKELNNKETLLPEKKGKRKIKIKKSGPKILQEENVL |
| | | AILAYFNPDKADIIKKFAGFFTYFSGFNQNRANYYTVKALATGVANR |
| | | AINRNFLIFLANRKDFARFKERLPRLAEFDNYFELENYEKYLSQ.TGIEE |
| | | YNDQIGKIKQIVNLEHNQQQKDNKFQLKGLATLEKQIGCRTKKQRE |
| | | EGGDKSAPKFLEKVGLGFQVSQDDDGEYLIWECLNYINKELAGKLKS |
| | | IKDNYQKFFADWRTGAYDLEKIWFRKEALNTISGRWFGGNNWFIIG |
| | | KALALTGVGKFDKRENTYKIPEFVSLAEIKTAFEMLENGVNYDFKKSK |
| | | KKKEGDDTDVVKYSADNLFKEEYKKKGLIKNSLFETMLAVWQSEIKR |
| | | KFEQIFDGYKLEKDDVFGRKKGEWVEPFIENFQKVSQEKFDRGVKD |
| | | ENGRSIHTEVVKNLIEEGYLRLFQLTKYHNLDKKGERDPRPFDGNFY |
| | | ATLDEFWKDNIVVVYHKALQSTLTKKPYSEDKIKLNFENGSLLGGFS |
| | | DGQERSKAGVVLKNKNKFYLGILIDRGFFRTDKANPVYDNAQNNE |
| | | WERLILTNLKFQTLAGKGFLGKHGVSYGEMGKDNPMMAVEYLQKF |
| | | IKLKYLDKYPALNEVAHKKYTIKKEFDADVKNALKDCFTMNFKPVDF |
| | | GMIRQGLTESLFYLFEIVNKDISSQAKNGKNVHTLYWEALFGDQNLK |
| | | KPILALNGGAEIFYRESQREKLEKKLDKSGKEVLDHKRYGQDKYFLHA |
| | | SITINYGQPKNIKFKEVINEKISQNADRVNIIGIDRGEKHLLYYSVVSPE |
| | | GVLLEQGSFNQIETKNKVDIKAVKAEYGERGELKKVELVPTGKKVKY |
| | | VDYQILLDYYEKKRNLARRDWQTIGKIKDLKDGYLSQTVHRIYQLILK |
| | | YNAVVAMEDLNVEFKAKRAAKVEKSVYKNFEMALAKKLNHLILKDR |
| | | RADEIGGALRAYQLTPAIPANDVGKFDKAKQWGIMFYVRANYTSTT |
| | | DPLTGWRKHKYISNSEKIDNIQKFFSPGDGIQIDYDTEKQCFKFSYDH |
| | | ELEGGAKKHWELFACDGLERFYWDNRERQIKKYNLYEEFEKLLGGL |
| | | RKEENINIQIDGVSEFRWKDLVFFWNLLNQIRNTDRSAQGDENDFL |
| | | QSPVWSEKYNCFYDSRKAPNNMPNNGDANGAFNIARKGQLILERI |
| | | KKCSDIPKFGNDNNGKNPENNYFISDADWDKFAQK |
| 129 | Cas12 | MAVKSIKVKLRLDDMPEIRAGLWKLHKEVNAGVRYYTEWLSLLRQE |
| | | NLYRRSPNGDGEQECDKTAEECKAELLERLRARQVENGHRGPAGS |
| | | DDELLQLARQLYELLVPQAIGAKGDAQQIARKFLSPLADKDAVGGL |
| | | GIAKAGNKPRWVRMREAGEPGWEEEKEKAETRKSADRTADVLRAL |
| | | ADFGLKPLMRVYTDSEMSSVEWKPLRKGQAVRTWDRDMFQQAIE |
| | | RMMSWESWNQRVGQEYAKLVEQKNRFEQKNFVGQEHLVHLVN |
| | | QLQQDMKEASPGLESKEQTAHYVTGRALRGSDKVFEKWGKLAPDA |
| | | PFDLYDAEIKNVQRRNTRRFGSHDLFAKLAEPEYQALWREDASFLTR |
| | | YAVYNSILRKLNHAKMFATFTLPDATAHPIWTRFDKLGGNLHQYTFL |
| | | FNEFGERRHAIRFHKLLKVENGVAREVDDVTVPISMSEQLDNLLPRD |
| | | PNEPIALYFRDYGAEQHFTGEFGGAKIQCRRDQLAHMHRRRGARD |
| | | VYLNVSVRVQSQSEARGERRPPYAAVFRLVGDNHRAFVHFDKLSDY |
| | | LAEHPDDGKLGSEGLLSGLRVMSVDLGLRTSASISVFRVARKDELKP |
| | | NSKGRVPFFFPIKGNDNLVAVHERSQLLKLPGETESKDLRAIREERQR |
| | | TLRQLRTQLAYLRLLVRCGSEDVGRRERSWAKLIEQPVDAANHMTP |
| | | DWREAFENELQKLKSLHGICSDKEWMDAVYESVRRVWRHMGKQ |
| | | VRDWRKDVRSGERPKIRGYAKDVVGGNSIEQIEYLERQYKFLKSWS |
| | | FFGKVSGQVIRAEKGSRFAITLREHIDHAKEDRLKKLADRIIMEALGY |
| | | VYALDERGKGKWVAKYPPCQLILLEELSEYQFNNDRPPSENNQLMQ |
| | | WSHRGVFQELINQAQVHDLLVGTMYAAFSSRFDARTGAPGIRCRR |
| | | VPARCTQEHNPEPFPWWLNKFVVEHTLDACPLRADDLIPTGEGEIF |
| | | VSPFSAEEGDFHQIHADLNAAQNLQQRLWSDFDISQIRLRCDWGE |
| | | VDGELVLIPRLTGKRTADSYSNKVFYTNTGVTYYERERGKKRRKVFA |
| | | QEKLSEEEAELLVEADEAREKSVVLMRDPSGIINRGNWTRQKEFWS |
| | | MVNQRIEGYLVKQIRSRVPLQDSACENTGDI |
| 130 | Cas12 | MSTQKNPFNQFTNLYELQKTLRFELRPVPETKKLLEKGEGKNLIQMD |
| | | LEIDRLYEKEMKPMFNILHEKFINESLGLVKLDCKKLKKLENLLAEADK |
| | | LRKQIKEGRKNKNNISEVEKRLKIIIGDNSQGKNKNGEIAVLQDELRV |
| | | LIVKAFNLTADKWKKELNNKETLLPEKKGKRKIKIKKSGPKILQEENVL |
| | | AILAYFNPDKADIIKKFAGFFTYFSGFNQNRANYYTVKALATGVANR |
| | | AINRNFLIFLANRKDFARFKERLPRLAEFDNYFELENYEKYLSQTGIEE |
| | | YNDQIGKIKQIVNLEHNQQQKDNKFQLKGLATLEKQIGCRTKKQRE |
| | | EGGDKSAPKFLEKVGLGFQVSQDDDGEYLIWECLNYINKELAGKLKS |
| | | IKDNYQKFFADWRTGAYDLEKIWFRKEALNTISGRWFGGNNWFIIG |
| | | KALALTGVGKFDKRENTYKIPEFVSLAEIKTAFEMLENGVNYDFKKSK |
| | | KKKEGDDTDVVKYSADNLFKEEYKKKGLIKNSLFETMLAVWQSEIKR |
| | | KFEQIFDGYKLEKDDVFGRKKGEWVEPFIENFQKVSQEKFDRGVKD |
| | | ENGRSIHTEVVKNLIEEGYLRLFQLTKYHNLDKKGERDPRPFDGNFY |
| | | ATLDEFWKDNIVVVYHKALQSTLTKKPYSEDKIKLNFENGSLLGGFS |
| | | DGQERSKAGVVLKNKNKFYLGILIDRGFFRTDKANPVYDNAQNNE |
| | | WERLILTNLKFQTLAGKGFLGKHGVSYGEMGKDNPMMAVEYLQKF |
| | | IKLKYLDKYPALNEVAHKKYTIKKEFDADVKNALKDCFTMNFKPVDF |
| | | GMIRQGLTESLFYLFEIVNKDISSQAKNGKNVHTLYWEALFGDQNLK |
| | | KPILALNGGAEIFYRESQREKLEKKLDKSGKEVLDHKRYGQDKYFLHA |
| | | SITINYGQPKNIKFKEVINEKISQNADRVNIIGIDRGEKHLLYYSVVSPE |
| | | GVLLEQGSFNQIETKNKVDIKAVKAEYGERGELKKVELVPTGKKVKY |
| | | VDYQILLDYYEKKRNLARRDWQTIGKIKDLKDGYLSQTVHRIYQLILK |
| | | YNAVVAMEDLNVEFKAKRAAKVEKSVYKNFEMALAKKLNHLILKDR |
| | | RADEIGGALRAYQLTPAIPANDVGKFDKAKQWGIMFYVRANYTSTT |
| | | DPLTGWRKHKYISNSEKIDNIQKFFSPGDGIQIDYDTEKQCFKFSYDH |
| | | ELEGGAKKHWELFACDGLERFYWDNRERQIKKYNLYEEFEKLLGGL |
| | | RKEENINIQIDGVSEFRWKDLVFFWNLLNQIRNTDRSAQGDENDFL |
| | | QSPVWSEKYNCFYDSRKAPNNMPNNGDANGAFNIARKGQLILERI |
| | | KKCSDIPKFGNDNNGKNPENNYFISDADWDKFAOK |
| 131 | Cas12 | MAIRSIKLKLKTHTGPEAQNLRKGIWRTHRLLNEGVAYYMKMLLLF |
| | | RQESTGERPKEELQEELICHIREQQQRNQADKNTQALPLDKALEALR |
| | | QLYELLVPSSVGQSGDAQIISRKFLSPLVDPNSEGGKGTSKAGAKPT |
| | | WQKKKEANDPTWEQDYEKWKKRREEDPTASVITTLEEYGIRPIFPLY |
| | | TNTVTDIAWLPLQSNQFVRTWDRDMLQQAIERLLSWESWNKRVQ |
| | | EEYAKLKEKMAQLNEQLEGGQEWISLLEQYEENRERELRENMTAA |
| | | NDKYRITKRQMKGWNELYELWSTFPASASHEQYKEALKRVQQRLR |
| | | GRFGDAHFFQYLMEEKNRLIWKGNPQRIHYFVARNELTKRLEEAKQ |
| | | SATMTLPNARKHPLWVRFDARGGNLQDYYLTAEADKPRSRRFVTF |
| | | SQLIWPSESGWMEKKDVEVELALSRQFYQQVKLLKNDKGKQKIEFK |
| | | DKGSGSTFNGHLGGAKLQLERGDLEKEEKNFEDGEIGSVYLNVVIDF |
| | | EPLQEVKNGRVQAPYGQVLQLIRRPNEFPKVTTYKSEQLVEWIKASP |
| | | QHSAGVESLASGFRVMSIDLGLRAAAATSIFSVEESSDKNAADFSYW |
| | | IEGTPLVAVHQRSYMLRLPGEQVEKQVMEKRDERFQLHQRVKFQI |
| | | RVLAQIMRMANKQYGDRWDELDSLKQAVEQKKSPLDQTDRTFWE |
| | | GIVCDLTKVLPRNEADWEQAVVQIHRKAEEYVGKAVQAWRKRFAA |
| | | DERKGIAGLSMWNIEELEGLRKLLISWSRRTRNPQEVNRFERGHTS |
| | | HQRLLTHIQNVKEDRLKQLSHAIVMTALGYVYDERKQEWCAEYPAC |
| | | QVILFENLSQYRSNLDRSTKENSTLMKWAHRSIPKYVHMQAEPYGI |
| | | QIGDVRAEYSSRFYAKTGTPGIRCKKVRGQDLQGRRFENLQKRLVN |
| | | EQFLTEEQVKQLRPGDIVPDDSGELFMTLTDGSGSKEVVFLQADINA |
| | | AHNLQKRFWQRYNELFKVSCRVIVRDEEEYLVPKTKSVQAKLGKGLF |
| | | VKKSDTAWKDVYVWDSQAKLKGKTTFTEESESPEQLEDFQEIIEEAE |
| | | EAKGTYRTLFRDPSGVFFPESVWYPQKDFWGEVKRKLYGKLRERFL |
| | | TKAR |
| 132 | Cas12 | MRKKLFKGYILHNKRLVYTGKAAIRSIKYPLVAPNKTALNNLSEKIIYD |
| | | YEHLFGPLNVASYARNSNRYSLVDFWIDSLRAGVIWQSKSTSLIDLIS |
| | | KLEGSKSPSEKIFEQIDFELKNKLDKEQFKDIILLNTGIRSSSNVRSLRG |
| | | RFLKCFKEEFRDTEEVIACVDKWSKDLIVEGKSILVSKQFLYWEEEFGI |
| | | KIFPHFKDNHDLPKLTFFVEPSLEFSPHLPLANCLERLKKFDISRESLLG |
| | | LDNNFSAFSNYFNELFNLLSRGEIKKIVTAVLAVSKSWENEPELEKRL |
| | | HFLSEKAKLLGYPKLTSSWADYRMIIGGKIKSWHSNYTEQLIKVREDL |
| | | KKHQIALDKLQEDLKKVVDSSLREQIEAQREALLPLLDTMLKEKDFSD |
| | | DLELYRFILSDFKSLLNGSYQRYIQTEEERKEDRDVTKKYKDLYSNLRN |
| | | IPRFFGESKKEQFNKFINKSLPTIDVGLKILEDIRNALETVSVRKPPSITE |
| | | EYVTKQLEKLSRKYKINAFNSNRFKQITEQVLRKYNNGELPKISEVFYR |
| | | YPRESHVAIRILPVKISNPRKDISYLLDKYQISPDWKNSNPGEVVDLIEI |
| | | YKLTLGWLLSCNKDFSMDFSSYDLKLFPEAASLIKNFGSCLSGYYLSK |
| | | MIFNCITSEIKGMITLYTRDKFVVRYVTQMIGSNQKFPLLCLVGEKQT |
| | | KNFSRNWGVLIEEKGDLGEEKNQEKCLIFKDKTDFAKAKEVEIFKNNI |
| | | WRIRTSKYQIQFLNRLFKKTKEWDLMNLVLSEPSLVLEEEWGVSWD |
| | | KDKLLPLLKKEKSCEERLYYSLPLNLVPATDYKEQSAEIEQRNTYLGLD |
| | | VGEFGVAYAVVRIVRDRIELLSWGFLKDPALRKIRERVQDMKKKQV |
| | | MAVFSSSSTAVARVREMAIHSLRNQIHSIALAYKAKIIYEISISNFETG |
| | | GNRMAKIYRSIKVSDVYRESGADTLVSEMIWGKKNKQMGNHISSY |
| | | ATSYTCCNCARTPFELVIDNDKEYEKGGDEFIFNVGDEKKVRGFLQK |
| | | SLLGKTIKGKEVLKSIKEYARPPIREVLLEGEDVEQLLKRRGNSYIYRCP |
| | | FCGYKTDADIQAALNIACRGYISDNAKDAVKEGERKLDYILEVRKLW |
| | | EKNGAVLRSAKFL |
| 133 | Cas12 | MSTQKNPFNQFTNLYELQKTLRFELRPVPETKKLLEKGEGKNLIQMD |
| | | LEIDRLYEKEMKPMFNILHEKFINESLGLVKLDCKKLKKLENLLAEADK |
| | | LRKQIKEGRKNKNNISEVEKRLKIIIGDNSQGKNKNGEIAVLQDELRV |
| | | LIVKAFNLTADKWKKELNNKETLLPEKKGKRKIKIKKSGPKILQEENVL |
| | | AILAYFNPDKADIIKKFAGFFTYFSGFNQNRANYYTVKALATGVANR |
| | | AINRNFLIFLANRKDFARFKERLPRLAEFDNYFELENYEKYLSQ.TGIEE |
| | | YNDQIGKIKQIVNLEHNQQQKDNKFQLKGLATLEKQIGCRTKKQRE |
| | | EGGDKSAPKFLEKVGLGFQVSQDDDGEYLIWECLNYINKELAGKLKS |
| | | IKDNYQKFFADWRTGAYDLEKIWFRKEALNTISGRWFGGNNWFIIG |
| | | KALALTGVGKFDKRENTYKIPEFVSLAEIKTAFEMLENGVNYDFKKSK |
| | | KKKEGDDTDVVKYSADNLFKEEYKKKGLIKNSLFETMLAVWQSEIKR |
| | | KFEQIFDGYKLEKDDVFGRKKGEWVEPFIENFQKVSQEKFDRGVKD |
| | | ENGRSIHTEVVKNLIEEGYLRLFQLTKYHNLDKKGERDPRPFDGNFY |
| | | ATLDEFWKDNIVVVYHKALQSTLTKKPYSEDKIKLNFENGSLLGGFS |
| | | DGQERSKAGVVLKNKNKFYLGILIDRGFFRTDKANPVYDNAQNNE |
| | | WERLILTNLKFQTLAGKGFLGKHGVSYGEMGKDNPMMAVEYLQKF |
| | | IKLKYLDKYPALNEVAHKKYTIKKEFDADVKNALKDCFTMNFKPVDF |
| | | GMIRQGLTESLFYLFEIVNKDISSQAKNGKNVHTLYWEALFGDQNLK |
| | | KPILALNGGAEIFYRESQREKLEKKLDKSGKEVLDHKRYGQDKYFLHA |
| | | SITINYGQPKNIKFKEVINEKISQNADRVNIIGIDRGEKHLLYYSVVSPE |
| | | GVLLEQGSFNQIETKNKVDIKAVKAEYGERGELKKVELVPTGKKVKY |
| | | VDYQILLDYYEKKRNLARRDWQTIGKIKDLKDGYLSQTVHRIYQLILK |
| | | YNAVVAMEDLNVEFKAKRAAKVEKSVYKNFEMALAKKLNHLILKDR |
| | | RADEIGGALRAYQLTPAIPANDVGKFDKAKQWGIMFYVRANYTSTT |
| | | DPLTGWRKHKYISNSEKIDNIQKFFSPGDGIQIDYDTEKQCFKFSYDH |
| | | ELEGGAKKHWELFACDGLERFYWDNRERQIKKYNLYEEFEKLLGGL |
| | | RKEENINIQIDGVSEFRWKDLVFFWNLLNQIRNTDRSAQGDENDFL |
| | | QSPVWSEKYNCFYDSRKAPNNMPNNGDANGAFNIARKGQLILERI |
| | | KKCSDIPKFGNDNNGKNPENNYFISDADWDKFAQK |
| 134 | Cas12 | MAQASSTPAVSPRPRPRYREERTLVRKLLPRPGQSKQEFRENVKKLR |
| | | KAFLQFNADVSGVCQWAIQFRPRYGKPAEPTETFWKFFLEPETSLP |
| | | PNDSRSPEFRRLQAFEAAAGINGAAALDDPAFTNELRDSILAVASRP |
| | | KTKEAQRLFSRLKDYQPAHRMILAKVAAEWIESRYRRAHQNWERN |
| | | YEEWKKEKQEWEQNHPELTPEIREAFNQIFQQLEVKEKRVRICPAA |
| | | RLLQNKDNCQYAGKNKHSVLCNQFNEFKKNHLQGKAIKFFYKDAEK |
| | | YLRCGLQSLKPNVQGPFREDWNKYLRYMNLKEETLRGKNGGRLPH |
| | | CKNLGQECEFNPHTALCKQYQQQLSSRPDLVQHDELYRKWRREYW |
| | | REPRKPVFRYPSVKRHSIAKIFGENYFQADFKNSVVGLRLDSMPAGQ |
| | | YLEFAFAPWPRNYRPQPGETEISSVHLHFVGTRPRIGFRFRVPHKRS |
| | | RFDCTQEELDELRSRTFPRKAQDQKFLEAARKRLLETFPGNAEQELR |
| | | LLAVDLGTDSARAAFFIGKTFQQAFPLKIVKIEKLYEQWPNQKQAGD |
| | | RRDASSKQPRPGLSRDHVGRHLQKMRAQASEIAQKRQELTGTPAP |
| | | ElIIDQAAKKATLQPFDLRGLTVHTARMIRDWARLNARQIIQLAEE |
| | | NQVDLIVLESLRGFRPPGYENLDQEKKRRVAFFAHGRIRRKVTEKAV |
| | | ERGMRVVTVPYLASSKVCAECRKKQKDNKQWEKNKKRGLFKCEGC |
| | | GSQAQVDENAARVLGRVFWGEIELPTAIP |
| 135 | Cas12 | MAVRSVKLKLLVPRDGSAESVRKRKALWATHQFVNDAAAAYAELLL |
| | | EMRQEDVCRGTDDHGKDVIEPAAHWQAKLRARLAAKQLPPVAVA |
| | | EALPLLKAFYGSRLIKSFVANDKGVAGTGNATDLNTWLSGLVDPASV |
| | | AGEKTELRKQLLAELPLCEAADADFEGAARKMLAKSDAREALLEGP |
| | | GTGVGWPAAYNANPTDSVWLDMLHKAAAKARLELADTTVSELKK |
| | | LGVFPLLQAASSNRVFGSGVLNPFERMAAAQAAAALLPWETKRHE |
| | | MRKRRDKFADQLNQWDTEFGASHATALAAIRAFEAEESERARRESL |
| | | GNEGTGYRIGGRELRDAWTLLRDWLKGHSTATAAAREDKVRELQA |
| | | KQGRSFGSHRLLSWLAKPAQQWLADHSAGDVVTRIAVRNARQRK |
| | | LDTARTLPIWTGADAVKHPRFANFDPPNNTNQPGFDLRAGTOKGR |
| | | LTLRLSLLTERADGLLLAQDHDFQLVPSRQMAEIVLHKDGKERALSW |
| | | QSQDGIGRQVGDVGGSALLFSRDHAECLLERKQITRLERGAWPAAL |
| | | PVWFKLSLDIGAEHKALLKQRFKWGVWLNSALVTRNAKDAKGVPP |
| | | PVGTRVLAVDLGLRSAATVSVWQVVDAATPVVAGKWRVPLSDTLS |
| | | AVHERSAMLALPGEHVDAGVLAARRAANEKLAGLLAATSHLSTVFK |
| | | LGRAEQGDRRRELLERLGEGDDRRARAAVATTAAERDGLRAVLGA |
| | | TQDAWAGAVAAVWRRLETDLAGAIAAYRKQQREDVQLRREARHG |
| | | PGASQLPKQAAAERLLGGKSAWQIEYKERVRKLLTRWIMRQRPGD |
| | | TAVRRLARKDLGKYCGGLLDHLTALKEDRAKTTADLIVQAARGRVR |
| | | AHKDAHGRQQDRELWLAKYAPCDLIVMEDLGRYRFATDRPPSENR |
| | | QLMQWTHREVFRLVQMQAEVEGIQVLETGAEFSSKFDARTWAPG |
| | | VRCEPITKLWVERYRNGEMPWLADKADEWRREGIELAQLVPGQLL |
| | | PTGSGEQFVAVSATGGLRVRHADLNAAQCIALRALTGHGTAFRLTA |
| | | RRLGDVFVSAKGLGKRPQGALWREFGSALPPAVVVLRPAGEVRYAL |
| | | RPFASARDAAAALGLQLGALRNVDATDAESDAEDGDLAELLAGAD |
| | | PDRATFFRDPSGDVHGGAWVQAKVFWAEVRRHVRLGLQAQGLLP |
| | | AAARSSEPRQMQLPLAGALPGDDIPL |
| 136 | Cas12 | MNRIYQGRVSKIEIKDSEGNFRNVPVGSPDTCPLWRHHRIFQDAVN |
| | | YYLVALGALAGTGSENAFVGLGSKDRVIHDLYSRLFDSWERFPRDM |
| | | HGASSLRDSLRRTLPGLSERASLQDAFDAILSGNEANARERVLSLLSLI |
| | | QDLGGDIQKGSKRYFPFFCEPATKATFPRARVGLLKVEGKDFVPRLL |
| | | WSSDLEIAPDQVVEQLKFEYFANPNESVQPIEGNEARVRLIEALDNP |
| | | QLGIELPIEILSDLRKRVHLIETDIRIPRYFFGGAGAELRKFRLDLFLIAA |
| | | YVTPDPSILRALRNSFKEPSASKSSKKKDETEEVENLLRSLGDDPLILA |
| | | RGERGFVFPSFTSLPTWVGANAQKPIWRDFDIAAFAEALKSLNQFT |
| | | AKTEEREEKLKKAEETLHYMLGISDAIPRSSDSETEEQAPSRPGKDPR |
| | | WPLVAQLEKELGENLSEGTWQLSRSAMRGLRDIIGLWRKHPGASV |
| | | VTLQKDVKTYQADEKHKREIGSVQLFLLLCEERYHALWQTETDDER |
| | | GDESEENDDPARILSDAIEVHQIRREVERFREPIRLTPAEPVFSRRLF |
| | | MFSDLTDKLAKVKFGETTEENSEVKSQFVEAAIALKEGENLKEARVRI |
| | | TFSAPRLHRDELLGGAESRWLQPITAALGFSNPAPSVKFDSAVALM |
| | | PDHMDDGRIRHLLNFPVNFDSAWLHQSIGKADLWKSQFNGTKDK |
| | | NLHLHWAGTARDTTRKNTWWENRTIIENGFTVLSNDLGQRSAGA |
| | | WALLKVTCSRPDTKHPVRSIGHDGTREWFATVLATGIHRLPGEDQR |
| | | ILKNGKWATEQSGKKGRNATFSEYEAACVLAKNLGCESVENWLGM |
| | | SGEKSYPALNDQLVKIANRRITRLGTYHRWSCFSPEKFEDPARRANV |
| | | IGGQLAELSAYQDENVTVSADILKSGDFEGFRHRAGAAFEALRTELE |
| | | VHLVNLANLTAPLRQKVWSWQKRPDSSGYGDLLMVDLDDCHPKIR |
| | | GQRGLSMARLEQLEGLRRLFLRYNRSLDRSPGIPAKFGREDVGRTSG |
| | | EPCQALLVKIDRMKEQRVNQTAHLILAQALGVRLCPHRIEENERKSR |
| | | DLHGEYEKIPGREPVDFIVIEDLSRYLSSQGRAPSENSRLMKWAHRA |
| | | VRDKLKMLAEEPFGIPVVETVPAYSSRFHALNGQAGSRLHELHELEA |
| | | YQQQSLINLAAKTDFQNRDRSKAAGELFEQFQALAKLNERRRAEGK |
| | | KVPRTLYYPKSGGPLFLASRDGDTIHADVNAAINLGLRAIAAPACIDI |
| | | HRRLRATKEKEVYRPRVGNAREKSAFSKDDIIQPSGAPSKKFASSSSP |
| | | NFFYEPEDLKQANGEPLFDRAMFGEYSLVSGVSLWSMVNNAIYIRC |
| | | VELNRTRLHGKDPDDQIPM |
| 137 | Cas12 | MADDLSTQRAYTLRLQGTDPEDQSWRDALWMTHEAVNAGGRAF |
| | | GDWLLTLRGGIAHELADTPVKGKKDITDELRKKRRILLALSWLSVESR |
| | | RGAPDKFIVAGGEEPAGSRNEKVLQALKEILKRRGLSAEESESWMS |
| | | DCRASLSAAIRDDAVWVNRSAAFDDAQVRIGASLTREDIWDMLDP |
| | | FFGSREAYLTPAKKKKEDEDSSEGTGEEKAKDLVQKAGQWLSSRFG |
| | | TGKGANFDAMAEVYSKISEWAGTAQEGVSGKEGIKNLADALAAFS |
| | | PVSQNLEGVLKLISGPGYKSATRNLLGELDSLPVVSRDHLSALHEKAA |
| | | EDTVKCKESTGTKGRRPYADAILNDVEKRCGFTYLTDSDNRSVSILDT |
| | | SEFPSDYKWGTARHSEFAVILDHAARRISVAHSWIKLAEAERDRCEE |
| | | DAAKVYDLPDKVKEWLDTFCSNRSDISGAQGEGYRIRRKAIEGWKE |
| | | WASWGRSSCITAEDRVAAARALQDDPEIDKFGDIQLFEILAQDEAL |
| | | CVWHKDGDVAKSPDAQMLIDYVLASDAESKKRRFKVPAYRHPDAL |
| | | LHPIFCDFGNSRWDITYDIHGARGKKKAKRGSKKEEAMPRGVAMK |
| | | LWTGSDVLSVSLRWQSKKLAADLALDQEAEEVTDTAAVSRADRLG |
| | | RAAAGIDRGAGVTIAGLFEEAHWNGRLQAPRQQLEAIAAVRDNQK |
| | | LSSEERERRIAFMKDRIRWLVTFSAKLRPQGPWHSYAPTQGLQSDP |
| | | KYWPHSEINKKRKGQAKLILSRLPGLRILSVDLGHRFAAACAVWET |
| | | MSSEAIQEACRLANHQLPAPADLYLHLKRTVQKNLIDGEKTVEESTV |
| | | YRRIGADRLPDGTAHPAPWARLDRQFLIKLQGEEKVREASNEEVW |
| | | QVHLMESALGLSFPLIDRLVYAGWGGTEKQAARLEALREKGWKPT |
| | | GTPADQDEEGGGYKPSLAVDELMFSAVRTLRLALKYHGDRARIAFA |
| | | LTADYKPMPGDTRYYFSEAKDRSSGADAAEREAKHKDYLLDMLLL |
| | | WHDLAFSRKWRDEEAKELWNLHIAALPGYQAPAAPIQEEAGQGRK |
| | | KAREEARAKMTPAAEALLADGTLREKLHGLWKERWEKDDAQWKK |
| | | HLRWMKDGILPRGGRAATPSIRYVGGLSLTRLATLTEFRRKVQVGFY |
| | | TRLFPSGEKREIKEAFGQTALDALERLREQRVKQLASRIAEAALGAGR |
| | | VSRTALKQDPKRPEARVDAACHAVIIENLEHYRPEETRTRRENRGLM |
| | | NWASSKVKKYLSEACQLHGLFLREVPAGYTSRQDSRTGAPGMRCQ |
| | | DVTVKTFLNSPFWQKQCVQAQKNKSTARDRFLCALKEAVAQGGM |
| | | EEEKKMGPIRVPVPGGEVFVSADAASPAAKGLQADLNAAANIGLRA |
| | | LLDPDWPGKWWYVPCDRKTAYPAKEKVEGSAAVDVKQALPFVLPE |
| | | EKENKGKTKGGKKGKGEVMNLWRDVSAEPLMTGQWLDYTAYRK |
| | | EVENRVIQVLTAQLKARNPLRFGNLGDEEEIPY |
| 138 | Cas12 | MAIRSIKLKLKTHTGPEAQNLRKGIWRTHRLLNEGVAYYMKMLLLF |
| | | RQESTGERPKEELQEELICHIREQQQRNQADKNTQALPLDKALEALR |
| | | QLYELLVPSSVGQSGDAQIISRKFLSPLVDPNSEGGKGTSKAGAKPT |
| | | WQKKKEANDPTWEQDYEKWKKRREEDPTASVITTLEEYGIRPIFPLY |
| | | TNTVTDIAWLPLQSNQFVRTWDRDMLQQAIERLLSWESWNKRVQ |
| | | EEYAKLKEKMAQLNEQLEGGQEWISLLEQYEENRERELRENMTAA |
| | | NDKYRITKRQMKGWNELYELWSTFPASASHEQYKEALKRVQQRLR |
| | | GRFGDAHFFQYLMEEKNRLIWKGNPQRIHYFVARNELTKRLEEAKQ |
| | | SATMTLPNARKHPLWVRFDARGGNLQDYYLTAEADKPRSRRFVTF |
| | | SQLIWPSESGWMEKKDVEVELALSRQFYQQVKLLKNDKGKQKIEFK |
| | | DKGSGSTFNGHLGGAKLQLERGDLEKEEKNFEDGEIGSVYLNVVIDF |
| | | EPLQEVKNGRVQAPYGQVLQLIRRPNEFPKVTTYKSEQLVEWIKASP |
| | | QHSAGVESLASGFRVMSIDLGLRAAAATSIFSVEESSDKNAADFSYW |
| | | lEGTPLVAVHQRSYMLRLPGEQVEKQVMEKRDERFQLHQRVKFQI |
| | | RVLAQIMRMANKQYGDRWDELDSLKQAVEQKKSPLDQTDRTFWE |
| | | GIVCDLTKVLPRNEADWEQAVVQIHRKAEEYVGKAVQAWRKRFAA |
| | | DERKGIAGLSMWNIEELEGLRKLLISWSRRTRNPQEVNRFERGHTS |
| | | HQRLLTHIQNVKEDRLKQLSHAIVMTALGYVYDERKQEWCAEYPAC |
| | | QVILFENLSQYRSNLDRSTKENSTLMKWAHRSIPKYVHMQAEPYGI |
| | | QIGDVRAEYSSRFYAKTGTPGIRCKKVRGQDLQGRRFENLQKRLVN |
| | | EQFLTEEQVKQLRPGDIVPDDSGELFMTLTDGSGSKEVVFLQADINA |
| | | AHNLQKRFWQRYNELFKVSCRVIVRDEEEYLVPKTKSVQAKLGKGLF |
| | | VKKSDTAWKDVYVWDSQAKLKGKTTFTEESESPEQLEDFQEIIEEAE |
| | | EAKGTYRTLFRDPSGVFFPESVWYPQKDFWGEVKRKLYGKLRERFL |
| | | TKAR |
| 139 | Cas12 | MPVRSINLKIVISRNTQGEKSRQSIWTTHAAVNDAVRYYEEQLLIMR |
| | | GLGYHISDKDVVSKESIQQERLSRIRRAQLENGLPEPLGTDAELNSLV |
| | | RKFYEFIVPSSVKEDGNAQQANGFLSPLTDPISIGYLSIFEKLGTIPDW |
| | | VGQLKAGDPQAVENAKKWSATSAGIKRLSETGAPPKWKKLFLTGD |
| | | PSWPQSFSEDIDKKIKEIEGAPKVICQLMEMGVLPLFPAYFADKLEGS |
| | | DGSLSRWDRLAFRLAVGHMLSWESWCIKSAEDHFERKRRVESFSEK |
| | | HTTPSLIICFETLEKYQKERQEKELGQNRSLPMQRPFRITRRQIRGWE |
| | | DLRDKWLKNTTRTYDSLKSIASKEQTKKGGRFGDPHLFLWLAKPEN |
| | | HAVWDADEDALSIFAKMNAMRGLLERSRETAYMTLPDPIEHPRSIQ |
| | | WEAEGGSNFKNYVITHSPVEGLHVQLPLLCKSESGKLIDQTFEFPLAP |
| | | SDQFKVAQISKTKSEVTITHQSVLDEEYRSKVGAADLLMDWPYLKN |
| | | RRFESVEHGDIGPVFLKLSLDIERILPDGWTPKRPQAISHFSSASGNS |
| | | KHKLSVVSGLRVLSVDLGIRSFGACSVFELSEHKPTSGMSFEIEGLNL |
| | | WANHERSFMLNLPDEDVGNKGRQLQKTKDAELRAMRRVLGRYRK |
| | | IYALAGIDPEDRKDILELLCQDQDIFEFERTIYKGLVTSTSVSQPLWEG |
| | | KIKESLKALRNAFGRKVREWRRANRLNSNLKYAGKTMWAIQHLED |
| | | TRRFLHSWSHLGRFSGEIRRADRVKRGVFATRLLQHLDSVKRDRLKT |
| | | GADLLVQSARGFLRDNQGNWKKSYAPCQVILFEDLSRYLMQTDRP |
| | | RRENSQLMKWSHRSIPLEVAMQGELYGIHVCDTSAAFSSRYHARLA |
| | | TPGIRCHALRKEDLSNQFLIESLQKENPDIDFGICKAGDLIPRGGGEIF |
| | | VSCDGNGGISRIHADINAAQNLQRRFWLRHGEAIRIPARKITLKGDEI |
| | | WVPRSIGKRLQGAMSGCGYLIPTGHESGSCRWERITASKWESISRSS |
| | | VAQKEEVNEDLLDIALLEEEALELSNEYTTFFRDPSGITLPSDLWFPM |
| | | KTFWGMTRAKIKSAIKQ |
| 140 | Cas12 | MTVKSIKVKLRLDNMPEIRAGLWKLHTEVNAGVRYYTEWLSLLRQE |
| | | NLYRRSPNGDGEQECYKTAEECKVELLERLRARQVENGHRDPAGSD |
| | | DELLQLARQLYELLVPQAIGAKGDAQQIARKFLSPLADKDAVGGLGI |
| | | AKAGNKPRWVRMRDAGEPGWEEEKAKAEARKSTDRTADVLRALA |
| | | DFGLKPLMRVYTDSDMSSVQWKPLRKGQAVRTWDRDMFQQAIE |
| | | RMMSWESWNQRVGEAYAKLVEQKSRFEQKNFVGQEHLVQLVNQ |
| | | LQQDMKEASHGLESKEQTAHYLTGRALRGSDKVFEKWEKLDPDAP |
| | | FDLYDTEIKNVQRRNTRRFGSHDLFAKLAEPKYQALWREDASFLTRY |
| | | AAYNSILRKLNHAKMFATFTLPDATAHPIWTRFDKLGGNLHQYTFLF |
| | | NEFGEGRHAIRFQKLLTIEHGVAKEVDDVTVPISMSAQLDDLLPGES |
| | | NEPTELSFRDHGTDQHFTGEFGGAKIQYRRDQLDHVHRRRGARDV |
| | | YLNLSVRVQSQSEARGERRPPYAAVFRLVGDTHRAFAHFDKLSNYL |
| | | AEHPDDGKLGSEGLLSGLRVMSVDLGLRTSASISIFRVARKDELKPNS |
| | | EGRVPFFFPIKGNDNLVAVHERSQLLKLPGETESKDLRAIREERQRIL |
| | | RQLRTQLAYLRLLVRCGSEDVGRRERSWAKLIEQSVDAANHMTPD |
| | | WREAFEGELQKLKSLYGICGDREWTEAVYESVRRVWRHMGKQVR |
| | | DWRKDVRSGERPKIRGYQKDVVGGNSIEQIEYLERQYKFLKSWSFF |
| | | GKVSGQVIRAEKGSRFATTLREHIDHAKEDRLKKLADRIIMEALGYVY |
| | | ALDAERGKGTWVAKYPPCQLILLEELSEYRFNNDRPPSENNQLMQ |
| | | WSHRGVFQELLNQAQVHDLLVGTMYAAFSSRFDARTGAPGIRCRR |
| | | VPARCAREQNPEPFPWWLNKFVAEHKLDGCPLRADDLIPTGEGEFF |
| | | VSPFSAEEGDFHQIHADLNAAQNLQRRLWSDFDISQIRLRCDWGE |
| | | VDGEPVLIPRLTGKRTADSYGNKVFYTNTGVTYYERERGKKRRKAFA |
| | | QEELSEEEAELLVEADEAREKSVVLMRDPSGIINRGDWTRQKEFWS |
| | | MVNQRIEGYLVKQIRSRVCLPESACENTGDI |
| 141 | Cas12 | MKNFQDFTNLYELSKTLRFELKPIGGTKKLIEEKNILKLDKKKRENYEK |
| | | VKPYFNKIHQEFINFALRNPNFDFSQFEEKYLNWLKDKKNKDLLKEK |
| | | ESIDKIFLEKIGKLFENSVKDFLKENGFESIVKEEDQNLKFFRRKEIFEVL |
| | | QEKYGSELETQMVNKDGEIKSIFNGWEKWLGYFDKFFNTRDNFYKT |
| | | DGTSTAIATRIIKDNLKIFLENIVAFGKIKNKKIDFSEVEKNFSVSIDTFF |
| | | EINNFNNCFLQDGIDFYNKVIGGETLENGEKLKGLNEIINKYRQDTGE |
| | | KIPYFKKLQKQILSEKDGVFIDKIEDDGGFYEVLKNFYKNAAEKEGFLK |
| | | NIFENFYTISDKNLEKIYFNKIAFNTISHKFGSALEFERILYEEMKKEKA |
| | | DGIKFEKKENKYKFPDFIQIIFIKRSLENYDSENLFWKERYYKSEENVD |
| | | GFLEKNNNNLWGQFCKILNFEFLNILKRRIIDEAGEEYEVGFEISKNIL |
| | | GEKLENFELNQENKGIIKDFADYSLALYSFGKYFAVEKGRNWDLNIDI |
| | | SDDFYGGEDGYIEKFYNTGYDEIVKPYNLMRNYISKKPWEDNKKWK |
| | | INFETSSLLSGWDKNLESNGSYIFQKGNKYYLGIINGSKPAKEILEKLYS |
| | | GDGEKIKRFIYDFQKPDNKNTPRMFIRSKKDSFSPAVEKYNLPINDIL |
| | | EIYDNGLFKTENKGNPNYKESLRKLIDYFKLGFSRHESFKHFNFVWK |
| | | DSKSYENIADFYRDVEKSCYKIDFEFLNFEELKKLTFEKHLYLFQIYNKD |
| | | FELDESLQEKGYNFKGEGQKNIHTKYFEALFLEENISRKSGAVFKLSG |
| | | GGEVFFRKKSIKAKKEKRNSVEVIKNKRYTECKYFLHFPIQVNFKEEIS |
| | | GNFNQEINKFLANNPDINVIGIDRGEKHLAYFSVINQKGEILESGSFN |
| | | KIENYNKNGEKLLFPEREIKEIHKDGSLIDLELVETGRKVDYVDYKLLLE |
| | | YKERKRLLQRQSWKEVEQIKDLKKGYISALVRKIADLIIKHNAIVIFEDL |
| | | NFRFKQIRGGIEKSIYQQLEKALIDKLNFLVNKNEINLEKAGSILKAYQ |
| | | LTVPVDSLKEIGKQTGVIFYTEAAYTSKIDPITGWRPNLYLKKNNSKIN |
| | | KENILKFDNIVFNSKENRFEFTYDLKKFFGKDSKFPAKTVNTVCSCVE |
| | | RFKWNRNLNNNKGGYIHYENLTDGKLANKEQKEDEFSNFKELFEKY |
| | | FIDINGNILEQIKNLDTKNNEKFFSSFIDLFTLVCQIRNTNQNAKGDE |
| | | NDFILSPVEPFFDSRKSQNFGKSLPKNGDENGAFNIARKGLIILNRISE |
| | | NPEKPDLLIFNADWDNFARNI |
| 142 | Cas12 | MAQAEAPRRLRAYKFALDPTEAQLREFEQHAGSARWAYNHANAIL |
| | | SRYSDTLRNRWNAWIAQHHGLSREQLYALPDRERTAIQAAARAAV |
| | | KAENAQLAAELRIIDDHRKRVTHKGKPSVEPGEQPAEDAPERAYQL |
| | | WRERVELARLHAEDPQAYRAERKRILDEIRPLVNATKRKLIEQGAYR |
| | | PTAMDISTLWREIRDLPPDEGGSPWWPEVSIYAFTSGFAHAETAW |
| | | KNYLESLAGRRAGRPVGKPRFKKKRRSRRSFTLYGSVKLVTYRRIQVP |
| | | SIGSVRLHGSAKRLHRALERRGGIIKSITISQGGHRWYASVLVDELDIT |
| | | PGRETQRGPSRRQRDRGAVGVDLGVHHLVALSDPNEKTLDNPRHL |
| | | RKARKRLLKAQRAMSRRRGPDKRTGQEPSRRWVKARNRVARLHH |
| | | ELAVRRAGHLHEITKRLATSYELVAIEDLNVAGMTRSARGTIDQPGR |
| | | GVRAKAGLNRSILDTSPAEFRROLOYKASWYGATVAVIDRWAPTSR |
| | | TCSSCGAVKAKLSLAERTFFCEHCGMELDRDINAARNILAFAQSAYP |
| | | GEGKALNACGGSVSPGSQSVVQAGADEAGRPARKPRRSSRGSDPP |
| | | ATPTTRA |
| 143 | Cas12 | MKRIAKFRHDKPVKREAWSKGYRVHKNRIINKVTRSIKYPLVVKDE |
| | | WKKRLIDDAAHDYRWLVGPINYSDWCRDPNQYSILEFWIDFLCVG |
| | | GVFQSSHSNICRLAIQLSGGSVFEQEWKDLSPFVRANLIQGIKPAEFI |
| | | GFLTAEFRSSSNPKNFISKFFEGSNEDLESLTNEFASIVDFIKAKDISLLR |
| | | KSLPSCKKIAPNLWEKAVGSHSTNELLKLLTKYTRVMLVAEPSHSDR |
| | | VFSQTVLQSNDQDDPELTGPLPSHKVGKASYLFIPEFIREVNLDKISKL |
| | | DLSAKSKLAVEQVKKLSELTSDFKQIENQSEAYFGLSTSFNELSNFLGI |
| | | LIRTLRNAPEAILKDQIALCAPLDKDILKITLDWLCDRAQALPENPRFE |
| | | TNWAEYRSYLGGKIKSWFSNYENFFEIPQAASSQQNNNREKKLGNR |
| | | SAIRALNLKKEAFEKARETFKGDKGTLEKIDLAYRLLGSISPEVLQCDE |
| | | GLKLYQQFNDELLVLNETINQKFQDAKRDIKAKKEKESFEKLQRNLSS |
| | | PLPRIPEFFGERAKKGYQKARVSPKLARHLLECLNDWLARFAKVEES |
| | | AFSEKEFQRILDWLRTSDFLPVFIRKSKDPPSWLRYIARVATGKYYFW |
| | | VSEYSRKRVQIIDKPIAQNPLKELISWFLLNKDAFSRDNELFKGLSSK |
| | | MVTLARIMAGILRDRGEGLKELQAMTSKLDNIGLLHPSFSVPVTDSL |
| | | KDAAFYRAFFSELEGLLNIGRSRLIIERITLQSQQSKNKKTRRPLMPEP |
| | | FINEDKEVFLAFPKFETKNKVKGTRVVYNSPDEVNWLLSPIRSSKGQL |
| | | SFMFRCLSEDAKIMTTSGGCSYIVEFKKLLEAQEEVLSIHDCDIIPRAF |
| | | VSIPFTLERESEETKPDWKPNRFMGVDIGEYAVAYCVIEKGTDSIEIL |
| | | DCGIVRNGAHRVLKEKVDRLKRRQRSMTFGAMDTSIAAARESLVG |
| | | NYRNRLHAIALKHGAKLVYEYEVSAFESGGNRIKKVYETLKKSDCTGE |
| | | TEADKNARKHIWGETNAVGDQIGAGWTSQTCAKCGRSFGADLKA |
| | | GNFGVAVPVPEKVEDSKGHYAYHEFPFEDGLKVRGFLKPNKIISDQK |
| | | ELAKAVHAYMRPPLVALGKRKLPKNARYRRGNSSLFRCPFSDCGFT |
| | | ADADIQAAYNIAVKQLYKPKKGYPKERKWQDFVILKPKEPSKLFDKQ |
| | | FYRPN |
| 144 | Cas12 | MAQAEAPRRLRAYKFALDPTEAQLREFEQHAGSARWAYNHANAIL |
| | | SRYSDTLRNRWNAWIAQHHGLSREQLYALPDRERTAIQAAARAAV |
| | | KAENAQLAAELRIIDDHRKRVTHKGKPSVEPGEQPAEDAPERAYQL |
| | | WRERVELARLHAEDPQAYRAERKRILDEIRPLVNATKRKLIEQGAYR |
| | | PTAMDISTLWREIRDLPPDEGGSPWWPEVSIYAFTSGFAHAETAW |
| | | KNYLESLAGRRAGRPVGKPRFKKKRRSRRSFTLYGSVKLVTYRRIQVP |
| | | SIGSVRLHGSAKRLHRALERRGGIIKSITISQGGHRWYASVLVDELDIT |
| | | PGRETQRGPSRRQRDRGAVGVDLGVHHLVALSDPNEKTLDNPRHL |
| | | RKARKRLLKAQRAMSRRRGPDKRTGQEPSRRWVKARNRVARLHH |
| | | ELAVRRAGHLHEITKRLATSYELVAIEDLNVAGMTRSARGTIDQPGR |
| | | GVRAKAGLNRSILDTSPAEFRROLOYKASWYGATVAVIDRWAPTSR |
| | | TCSSCGAVKAKLSLAERTFFCEHCGMELDRDINAARNILAFAQSAYP |
| | | GEGKALNACGGSVSPGSQSVVQAGADEAGRPARKPRRSSRGSDPP |
| | | ATPTTRA |
| 145 | Cas12 | MAIRSIKLKLKTHTGPEAQNLRKGIWRTHRLLNEGVAYYMKMLLLF |
| | | RQESTGERPKEELQEELICHIREQQQRNQADKNTQALPLDKALEALR |
| | | QLYELLVPSSVGQSGDAQIISRKFLSPLVDPNSEGGKGTSKAGAKPT |
| | | WQKKKEANDPTWEQDYEKWKKRREEDPTASVITTLEEYGIRPIFPLY |
| | | TNTVTDIAWLPLQSNQFVRTWDRDMLQQAIERLLSWESWNKRVO |
| | | EEYAKLKEKMAQLNEQLEGGQEWISLLEQYEENRERELRENMTAA |
| | | NDKYRITKRQMKGWNELYELWSTFPASASHEQYKEALKRVQQRLR |
| | | GRFGDAHFFQYLMEEKNRLIWKGNPQRIHYFVARNELTKRLEEAKQ |
| | | SATMTLPNARKHPLWVRFDARGGNLQDYYLTAEADKPRSRRFVTF |
| | | SQLIWPSESGWMEKKDVEVELALSRQFYQQVKLLKNDKGKQKIEFK |
| | | DKGSGSTFNGHLGGAKLQLERGDLEKEEKNFEDGEIGSVYLNVVIDF |
| | | EPLQEVKNGRVQAPYGQVLQLIRRPNEFPKVTTYKSEQLVEWIKASP |
| | | QHSAGVESLASGFRVMSIDLGLRAAAATSIFSVEESSDKNAADFSYW |
| | | IEGTPLVAVHQRSYMLRLPGEQVEKQVMEKRDERFQLHQRVKFQI |
| | | RVLAQIMRMANKQYGDRWDELDSLKQAVEQKKSPLDQTDRTFWE |
| | | GIVCDLTKVLPRNEADWEQAVVQIHRKAEEYVGKAVQAWRKRFAA |
| | | DERKGIAGLSMWNIEELEGLRKLLISWSRRTRNPQEVNRFERGHTS |
| | | HQRLLTHIQNVKEDRLKQLSHAIVMTALGYVYDERKQEWCAEYPAC |
| | | QVILFENLSQYRSNLDRSTKENSTLMKWAHRSIPKYVHMQAEPYGI |
| | | QIGDVRAEYSSRFYAKTGTPGIRCKKVRGQDLQGRRFENLOKRLVN |
| | | EQFLTEEQVKQLRPGDIVPDDSGELFMTLTDGSGSKEVVFLQADINA |
| | | AHNLQKRFWQRYNELFKVSCRVIVRDEEEYLVPKTKSVQAKLGKGLF |
| | | VKKSDTAWKDVYVWDSQAKLKGKTTFTEESESPEQLEDFQEIIEEAE |
| | | EAKGTYRTLFRDPSGVFFPESVWYPQKDFWGEVKRKLYGKLRERFL |
| | | TKAR |
| 146 | Cas12 | MVVRSIKLKMKTNSGTDSIYLRKALWRTHRLINEGIVYYMSLLTLYR |
| | | QDTLGDRTKEEIQSELIKKIREQQRNNGLSEELGSDQEILSLLRHLYELI |
| | | IPSINGESGDANQLGNKFLYPLVDPNSQSGKGTSNAGRKSKWKRM |
| | | KEEGNPDWEVEFKKDEERKANDPTIKVFDHLKKYSLLPLFPLFTNNQ |
| | | KDIEWLPMGKRQSVRKWDKDMFIQAIERLLSWESWNRRLGAERE |
| | | KLEEKIENFYKEHLSGGQIWIEKIREFERVRDRELGETSFSSNDGYLITS |
| | | RQIRGWDRVYEKWSKISESASKEELWRVVAEQQSKMREGFGDPKV |
| | | FSFLAELENMDIWRKHPERIYHIATYNGLLKKLSHTKAQATFTLPDAV |
| | | KHPLWIRYEAQGGTNLNLFKLEETTKKNCKVILSKIIWPTEDGWFEK |
| | | ENVEVDLAPSKQFYRQIKLQDHIKGKQEISFSDYSSGISLKGVLGGSRI |
| | | QFDRKYIENHQELLPSGDIGPVFFNLVIDLLPIQETRNGRLKSPIGKTL |
| | | KVVSSEFPKVIDYKPKELTEWINVSSVTGKVGVESITEGMRVMSIDL |
| | | GQRTSASVSIFEVVKELPKDKEKMLYYNIKDTELFALHKRSFLLNLPGE |
| | | EVTKRNRQKRKDRRKKLLFIRSQIRMLASVLKLETKNTPDERKKAINK |
| | | LVEIVDSYEWTESEKEIWNSEFEYLTNKAVFKQEIWRESLIKSHHRM |
| | | EPHVGQLVSEWRKSLNEGRRNLAGITMWNIEELEDTRRLLISWSKR |
| | | SRTPGEANRINNDEPFGAKLLEHIQNVKDDRLKQLANLIVMTALGYK |
| | | YDKEEKSRDKRWKEKYPACQVILFENLNRYLFSLDRSKRENSKLMK |
| | | WAHRSIPRTVWMQGEMFGLQVGDVRSEYSSRFHAKSGAPGIRCH |
| | | SLNDEDLKEESFKLKHLIETGFISEEEISSLKKGDIVPWPGGELFVTLSK |
| | | PYKKGKDIELTVIHADINAAQNLQKRFWQQNSEVYRIPCQLEKAGD |
| | | DEFFIPKSQTEIVKKYFGKGRFVKINDKKEVYNWEESEKMKIKTESTIT |
| | | LQDLEGFEDIFQTLELAQEQQKKYSTLFRDPSGYFFNEKNWRPQKEF |
| | | WSIVNNIIRSSLKKKILKNKVEV |
| 147 | Cas12 | MSNPNIPNISPNITLTRDDVVNLLMSSIAMEELGLAHIINAEGEKIQF |
| | | ALGTLQGASGPPATLQQVLEVNQSTQAMLDTIFRQEMMLDSKLKT |
| | | ATNIPTLRGPTGPVGPTGAPGGVISINGQTGVVTLDASNGVMPFM |
| | | REQSTSSLDDYKDPGIYXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXGATGATGATGATGPQEPRALREPRAPQARREPRGLLEPQVL |
| | | RGPQEPRALREPRAPRALREPRALRALRALQELRAPRALRGLQEPRV |
| | | LQGPRERQVRPEPRVLQGPRERQVRQGLQGLLEPRAKRERQVRPE |
| | | PREPQERQAPLVQQALLEPQALLGQAL |
| 148 | Cas12 | MNRIYQGRVSKVEIPNPGDKGKPWQPLPNWQDILWQHHELFQD |
| | | AVNYYLLALLSLARDSANPATPIRKRMDDPASEHQIWTSFRRRGQM |
| | | RSGMRDSVAKYLCPVKTEPTLDECFAAVLEGNKENPDVLDLALQELL |
| | | DECDGDGAIQQEGRSMLPRFCSPSYKGDFPQSAAAKDKKSAKEWL |
| | | PTFLHSAENASNLKLVRQRLKFELFANRDLTGRILDAVESRKRLTEML |
| | | GWLTERNPSLAAERERLQKIIAALPDTFNLPAWRGGSVNKEALKQR |
| | | FYAWLIFEHVESSRITFDILRDSFPTPKEKRKSVSETIQSEKPSLELSDD |
| | | PIKLARGTRGYVFRAFTSLPCWGADNANLLAWKEFDVAAFKEALKA |
| | | LHQVESKSEERNKERERLLQRHSVMRGSIKWKPSPESEEKEPDVLAG |
| | | DPRIERLEKLLKTELASEYEMSEGQTVEYGLQPRTIRGFRDLRKEWN |
| | | KIVKPGEPFTEQKKGNLVTALRSYQIENPNVIGSVRLYEALLEKGNWL |
| | | VWQEADSATVEKWAKQKFASDPLEALTEERQLLRDIERLKQPIRFTP |
| | | ADAVHSRRQFYLAEKGDLSVKNRFDPQNQ.TLQVPIAIKNDDHWKQ |
| | | QFVKIHFSAPRAVRDQLIDETMEESKEARWQQPMMEALGLSLKLT |
| | | KNNAEVSLSECTAVSLMPEELASGDRRILLNFPITLETAPMVKALGKA |
| | | SLWDGQFAAYGDDNFYLRWPKDKWPAEKESNAWYRRLTEFSLLSV |
| | | DLGQRDAGAFAVIGATTGKPTRPASRFIGEANGQSWHASVRATGV |
| | | LRLPGEDAHVFRSGQWQEELYGERGRSADATEWTEAKRICETLGLK |
| | | FDEILGGDPQWFSFPELNDRLLFAVRRAQTRLARLQSWSWMIADE |
| | | TRRENIREAILAADDDELSLKAAAEKSLWPVLAEKLASEVNRLRNLIP |
| | | QQLVLLANRILPLRGRRWEWIKRDDTSGCHVLRETNPGTDTTSKKV |
| | | CGQRGLSMKRLEQLDELRRRFQSLNRALMQTPGQTAGLGKSKRGI |
| | | ELPDPCPDLLDKTEQLREQRVNQTAHLILAQALGVRLRTPQKGDAL |
| | | REQNNVHGEYETFRLPVDFIVLEDLSRYLSSQGRGRNENSRLMKWC |
| | | HRAILLKLKQLCESYGLKILETNAAYSSRFCSRTGVAGFRAVELTPDAR |
| | | KEFRWRKHLNRLEKAASGEIKLDREARAESEHVKRLFDMLDQLNAG |
| | | RKEAGKPLRTLLAPIAGGQIFIPMQGHATQADINAAINLGLRAIAAP |
| | | DCHAIHVRIRTERKDKTLRVRTGSNREKVRWEGKNPEIQMNKAADL |
| | | ASLTGDRQPNFFPDPSAIACYDRAKIEGVQLPFASGRGLWGTMNIE |
| | | LQWTRVNQLNNDRAEKKWNLTRSLPEQSSEEDYIPM |
| 149 | Cas12 | MAVKSIKVKLMLGHLPEIREGLWHLHEAVNLGVRYYTEWLALLRQG |
| | | NLYRRGKDGAQECYMTAEQCRQELLVRLRDRQKRNGHTGDPGTD |
| | | EELLGVARRLYELLVPQSVGKKGQAQMLASGFLSPLADPKSEGGKG |
| | | TSKSGRKPAWMGMKEAGDSRWVEAKARYEANKAKDPTKQVIASL |
| | | EMYGLRPLFDVFTETYKTIRWMPLGKHQGVRAWDRDMFQQSLER |
| | | LMSWESWNERVGAEFARLVDRRDRFREKHFTGQEHLVALAQRLE |
| | | QEMKEASPGFESKSSQAHRITKRALRGADGIIDDWLKLSEGEPVDRF |
| | | DEILRKRQAQNPRRFGSHDLFLKLAEPVFQPLWREDPSFLSRWASY |
| | | NEVLNKLEDAKQFATFTLPSPCSNPVWARFENAEGTNIFKYDFLFDH |
| | | FGKGRHGVRFQRMIVMRDGVPTEVEGIVVPIAPSRQLDALAPNDA |
| | | ASPIDVFVGDPAAPGAFRGQFGGAKIQYRRSALVRKGRREEKAYLC |
| | | GFRLPSQRRTGTPADDAGEVFLNLSLRVESQSEQAGRRNPPYAAVF |
| | | HISDQTRRVIVRYGEIERYLAEHPDTGIPGSRGLTSGLRVMSVDLGLR |
| | | TSAAISVFRVAHRDELTPDAHGRQPFFFPIHGMDHLVALHERSHLIR |
| | | LPGETESKKVRSIREQRLDRLNRLRSQMASLRLLVRTGVLDEQKRDR |
| | | NWERLQSSMERGGERMPSDWWDLFQAQVRYLAQHRDASGEAW |
| | | GRMVQAAVRTLWRQLAKQVRDWRKEVRRNADKVKIRGIARDVP |
| | | GGHSLAQLDYLERQYRFLRSWSAFSVQAGQVVRAERDSRFAVALRE |
| | | HIDNGKKDRLKKLADRILMEALGYVYVTDGRRAGQWQAVYPPCQL |
| | | VLLEELSEYRFSNDRPPSENSQLMVWSHRGVLEELIHQAQVHDVLV |
| | | GTIPAAFSSRFDARTGAPGIRCRRVPSIPLKDAPSIPIWLSHYLKQTER |
| | | DAAALRPGELIPTGDGEFLVTPAGRGASGVRVVHADINAAHNLQRR |
| | | LWENFDLSDIRVRCDRREGKDGTVVLIPRLTNQRVKERYSGVIFTSE |
| | | DGVSFTVGDAKTRRRSSASQGEGDDLSDEEQELLAEADDARERSVV |
| | | LFRDPSGFVNGGRWTAQRAFWGMVHNRIETLLAERFSVSGAAEKV |
| | | RG |
| 150 | Cas12 | MGLVDTAGGFLIPAALDPAlLLSGDGSTNPIRQVXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXPSRCRCLAFGRSSSAIRRPAASSSEGVKSL |
| | | RSVFSERSALARSTTSAIVWHTCTGMRSHPSSRAASSRRWPNTSRLS |
| | | GVTPIGCSKRQGDSPCGCCCGAHLVVAQRCVAFCCHDRGVTEKLLY |
| | | SSQVVGAAVGAGGVSVPQGVHGRVTG |
| 151 | Cas12 | MRRQHHGGQNARDWRRKVAAAALRQKESVFTYKFGLSVNDGDF |
| | | DFDAAARTYDITEGIERGSLIGLVCAVHLSGFRLFSKVAETRQFLNRS |
| | | RYPENEFAQALAAHTEIENPSVTVQSIESVFVTPPRKQDGVARLWSA |
| | | DELAKRLFQTWNNRSPREGERNHPELLLAQGIARAVTKAFSGWKEL |
| | | ADNAVHALTCADNYLATLGNRFPKLSDLPPLTAGSTQTGTLAFDPES |
| | | PFLNMTGNEDIWLHQVVAVCAGRLKRYMPEIDPSSRKFASRLTDSI |
| | | VSSQNNGLSWLFGNGLRFLRQSSIAQIAETLSVSQNEHRRVEQLKEF |
| | | ADAIPVNPFFATDGYAEFRGSVGGKISSWVSNYWKRICELTVLHSQ |
| | | PPDITIPEGLLASENATLFSGQHTAAAGLVALSARLPSQVRDAGKALF |
| | | VLSGDGVPRADDIATVEDVAGELAELTGQLAMLDNRIQQEIERAQD |
| | | ANDEGRVGSLASLRPNPTKELKEPPKLNRISGGTADAAGELARLETSL |
| | | NDLIRARREHFYRLAEWTGNTASLDPLPALAERERKALTDRGMDPT |
| | | LAEADEYALRRLLHRIAGMARRLSPNEAKRVRETMTPLFLKKREANL |
| | | YFHNRAGALYRHPFSNSRHQPYSIDLNRARATDWLAWLEERAREM |
| | | LGLLGSGAPANHEYLRDLLSIETFVFTTRLSGLPAQVPGYLAKPKSDLT |
| | | NIPPLLAAQLDVDEVSRDVALRAFNLFNSAINGLSFRAFRDSFIVRTK |
| | | FLRLGHDELFYVPKARAWKPPADYRSAKGKISKGLALPAVKRNEAGS |
| | | ILPRETTQGLSRAKFPEGSHALLSQAPHDWFVELDLRHDKMPQLAG |
| | | LPVKMNADGLKGWRARRRPTFRLAGPPSFKTWLDRALTSTAVKLG |
| | | DYTLILDQSFKQSLRVEDGEVRLSAEPAGIKAEIAVPVIDARPFPETEA |
| | | EALFDNIIGIDLGERRIGYAVFSLPALLKSGNPTRVKPTVVGSVAIPAF |
| | | RRLMAAVRRHRGSRQPNQKVSQTYSTALQQFRENVVGDVCNRID |
| | | TLCERYRAFPVLESSVANFETGANQLKLIYGTVLRRYTFSNVDAHKSA |
| | | RSAYWYSANRWQHPYLFVREWNKAQRTFTGSAKPLAIYPGVTIHP |
| | | AGTSQICHRCGRNALRALRNMPDRTIRVGKDGLIVLADSTIRLLERA |
| | | DYSDRELKTFKRRKQRPPLNMPVPEGARPRDQLERVLRRNMRQQP |
| | | QSEMSPDTTQARFTCVYTDCGFEGHADENAAVNIGRRFLERIDIEAS |
| | | SRT |
| 152 | Cas12 | MQEIKRINKIRRRLVKDSNTKKAGKTGPMKTLLVRVMTPDLRERLE |
| | | NLRKKPENIPQPISNTSRANLNKLLTDYTEMKKAILHVYWEEFQKDP |
| | | VGLMSRVAQPAPKNIDQRKLIPVKDGNERLTSSGFACSQCCQPLYV |
| | | YKLEQVNDKGKPHTNYFGRCNVSEHERLILLSPHKPEANDELVTYSL |
| | | GKFGQRALDFYSIHVTRESNHPVKPLEQIGGNSCASGPVGKALSDA |
| | | CMGAVASFLTKYQDIILEHQKVIKKNEKRLANLKDIASANGLAFPKIT |
| | | LPPQPHTKEGIEAYNNVVAQIVIWVNLNLWQKLKIGRDEAKPLQRL |
| | | KGFPSFPLVERQANEVDWWDMVCNVKKLINEKKEDGKVFWQNLA |
| | | GYKRQEALLPYLSSEEDRKKGKKFARYQFGDLLLHLEKKHGEDWGK |
| | | VYDEAWERIDKKVEGLSKHIKLEEERRSEDAQSKAALTDWLRAKASF |
| | | VIEGLKEADKDEFCRCELKLOKWYGDLRGKPFAIEAENSILDISGFSK |
| | | QYNCAFIWQKDGVKKLNLYLIINYFKGGKLRFKKIKPEAFEANRFYTV |
| | | INKKSGEIVPMEVNFNFDDPNLIILPLAFGKRQGREFIWNDLLSLETG |
| | | SLKLANGRVIEKTLYNRRTRQDEPALFVALTFERREVLDSSNIKPMNL |
| | | IGIDRGENIPAVIALTDPEGCPLSRFKDSLGNPTHILRIGESYKEKQRTI |
| | | QAAKEVEQRRAGGYSRKYASKAKNLADDMVRNTARDLLYYAVTQD |
| | | AMLIFENLSRGFGRQGKRTFMAERQYTRMEDWLTAKLAYEGLPSK |
| | | TYLSKTLAQYTSKTCSNCGFTITSADYDRVLEKLKKTATGWMTTING |
| | | KELKVEGQITYYNRYKRQNVVKDLSVELDRLSEESVNNDISSWTKGR |
| | | SGEALSLLKKRFSHRPVQEKFVCLNCGFETHADEQAALNIARSWLFL |
| | | RSQEYKKYQTNKTTGNTDKRAFVETWQSFYRKKLKEVWKPAV |
| 153 | Cas12 | MGKQSTNSSGLKATSGAPSIEKPVTTQRAYTLRLRGIEGDKTWRDSL |
| | | WATHEAINLGAKAFGDWLLTLRGGLEHTLADATVAGGKGKPARPP |
| | | SADERRDRRVLLALSWLTVEDERGAPKAPGLIVAYGDDCKSAKDSQ |
| | | DGRDRKVEDALRDILIKRSVAPGAVESWVNDCVVSLKARIRDDAVW |
| | | VNRSAAFDALRASWRGLTCPNARTVLEQFFGPVADWITLPARAAD |
| | | DGEGDTGGPTAAGSSNDTEFKLVARSFLSENFGTGLKSSKSDISDAL |
| | | MQATRSLAALRGGSPGSAVWSHLCTEFGLLAADDEARAKALRVRIG |
| | | WTSGRRSKGRLALATAASKASLAQVDIELLIKKLSEEAQEKLAGSTKL |
| | | PLPWVNDLMAALERSIGFGFVTERNLIDEFGVMLDHAARRVSIACS |
| | | WIKLAELERRQFEEDAKKLDQVRTQHADAAAFLDTLGCRRGSESGS |
| | | AGGASVVIRKRAVLGWNEVVGDWARQGCKSVQERIDAARLLQGE |
| | | LEKFGDIRLFEELAANDAQVVWQNAEGETDPTILARYSAGSVAKAN |
| | | QQRFKVPAYRHPDPLRHPVFGDFGKSRWKIDFAVHESDRAQSGGK |
| | | RVRSLDAAWQSDVRNMRMALWTGKRVEMVPLRWSSKRLSKDLG |
| | | ISDLSLKDPTVVTRGDRLGRAAVGPGDPVRVASVFAEDNWNGRLQ |
| | | APRVELNRVARLTQSGNLVQARRLRDRLSWLVSFSPKLAPSGPFIDY |
| | | AAAHGIEPHKKSGEYWPNSAMNKERKGHHSKLIYSRLPSLRVLSVDL |
| | | GHRFAAACAVWESMSAAQMRLAASKGKVVRGGIAEGALFIHIEST |
| | | VADGTVRTTVYRRIGEDALPDGSPHPAPWAKLERQFLIKLQGEETPS |
| | | RMAALDELVMIADWERALGYQPVTASSTKQSNGVAGLMGRTVRL |
| | | FMHASRRHFDRARIAHNLTAEHRTKPGGVPESLTEETRIELLIDTLAL |
| | | WHGLFAGDRWRDPRAQKEWETSGLPSLKLPGRGDDDETAFGGPG |
| | | RKAAMVVYRNELKPHAERMAHSDLSNLSKCWTDRWAQDDREWT |
| | | AKSGRLRSLRRWITPRGLRPVSGDSAQLIERKAAAALRARHVGGLS |
| | | MQRINTLTDLYRLLKSFKNRPEPSNLRKNIPEKGDARLIGFNQRLLDV |
| | | RDRLREQRVKQLASRIVEAALGIGRIKIRRVAAGSPRPTLRVDAACHA |
| | | VVTENLSNYQPAELQTRRENRQLMAWASSKVHKYLAESCQLHGLH |
| | | LREVQPNYTSRQCSRTGAAGLRGvAVSATDLLTNPWWMRDVAQA |
| | | KARIEKALKNGRNGAVADQLLVSAEAWALRIPERDRHDRKADESVK |
| | | AFHAERVILPRKGGDLFVAESARCNGKGHVPALQADLNAAANVGIR |
| | | ALLDPDWPGKWWWVPCVGGTYSPSPEKTSGAKVLEHIDQLPHEM |
| | | VDPPQVAPKSSAPQPKSSRAAKVKAKEFRAIENRWRTCSSELLSQGK |
| | | WIPSGEYWSKVEAAVCLVLNDQIT |
| 154 | Cas12 | MAAFQRSYTMNLKPATSEQDKFILWNRLFLTHWSVNEGAKIFGELF |
| | | LNLRGGLSPELDIFDLDKVKDDKKKKAFVMGRRRLLALGWLSVEDN |
| | | LSAGEHPFRIREIPVGRNMGKSQASTLLTEILKNKGIKDEAVIKEWID |
| | | DCTPSLIANIREDAVWINRASSFNSITPCPTKDEVWIVLSGLLGLRFLD |
| | | LSLEEVKGKETEYLYDLGEKETQSKSDPSKKARELFGNLFTQNPVLMK |
| | | NSRDKKDTFAKEFYLAFKEFKDYEKLKEKIESWRKEKEFPLIENPVAEK |
| | | YPPEVTFTGSPCTVSKRYRKLLVSLELWPSSQDENGNIPKTEKTEDKT |
| | | HNQVLLDYLLKACNEGNKGTQKHTPVWANNLKAELELKMNEIIRIG |
| | | ESSSTELQRLMIKMAARRISQTLSWIKINEQTKHDAYQKKNKAFKLL |
| | | SEIDKNGEACKWLENYELFRTDDSGGEEYHISLRAISCWKQILEEWQ |
| | | KNDSPKALREKVKEVQAEEEKFGDARLFEDLADDNARSVWLLPDG |
| | | NKTPDILNWWCEYRTAEIDESRFKIPCYCHPHPFKHPVYVEYGKSNP |
| | | KVIFAMKNNKVKKGHIEHGWNPQNPRSIALSLFNNGNRESSLVPFI |
| | | WESKRLWKDLGGEATQIGDIPRSDRMGLSGKRESVKPKAPFQKEV |
| | | WNARLQSDRRTLEKLEKYWNPESMKWIDDGKFLIQSKWFITFGPD |
| | | METAEGPWKLYLKEKYVDNKILGNRSKENQKRGYRAKKLLSGYPAG |
| | | MRILSVDLGHRYAASCAVWETITKKQITEELAYQPDNNSLFEHSCKTI |
| | | DKKIKNTVYRRIGEDSIDAPWAKLEKQFTIKLQGEDKSCYLLRSDEKE |
| | | LFRSILSKLSCLNNDTGHNILEMIENLLRIVKAKIYRQGILARISYSMTA |
| | | QYKPGKGGQKSPLSDEDKIHYLSENLAAWSAIMGNQEWNEDVISD |
| | | WYKTYISHLVSGPKPKEGNRKSDRDKIIEYFLPAARKLYDDNETRIKIH |
| | | DLFKELWDENNKQLSAVLKEIKKIILPKGIRYFDKNTDNPSKWKNNQ |
| | | SKLKQITHRGGLSMQRIVAIEEYYKLAKAYKNHPEPDDLTKNIPLPGD |
| | | NSSAGFNQRIRDTLERMKEQRVKQIASRIVESALGLGIEGYKKRPLTP |
| | | ENKPCQAIVIEDLSHYRPDELQTRRENRRLMQWSSSKVKKYLKEACE |
| | | MHDVRLVEISPEYTSRQDSRTGAAGLRCIDINIREFLKDSSRWQNKI |
| | | NTIQKKPANKKSNLDQYLIELNESLGNKYKDKVIPSDNFVRIPRKGGD |
| | | VFVSSSKESPVSKGIQADLNAAANIGLKALLDPDWAGAWWYILIEV |
| | | KSNHVIPYGEKYKGSECLRAWKFSGLENQVMKNNMNLWRDLOSQ |
| | | FSGEDKWMSYKEYNELTEKRVINILRERAGLELIKE |
| 155 | Cas12 | MEDYSGFVNIYSIQKTLRFELKPVGKTLEHIEKKGFLKKDKIRAEDYKA |
| | | VKKIIDKYHRAYIEEVFDSVLHQKKKKDKTRFSTQFIKEIKEFSELYYKT |
| | | EKNIPDKERLEALSEKLRKMLVGAFKGEFSEEVAEKYKNLFSKELIRNE |
| | | IEKFCETDEERKQVSNFKSFTTYFTGFHSNRQNIYSDEKKSTAIGYRII |
| | | HQNLPKFLDNLKIIESIQRRFKDFPWSDLKKNLKKIDKNIKLTEYFSID |
| | | GFVNVLNQKGIDAYNTILGGKSEESGEKIQGLNEYINLYRQKNNIDR |
| | | KNLPNVKILFKQILGDRETKSFIPEAFPDDQSVLNSITEFAKYLKLDKKK |
| | | KSIIAELKKFLSSFNRYELDGIYLANDNSLASISTFLFDDWSFIKKSVSFK |
| | | YDESVGDPKKKIKSPLKYEKEKEKWLKQKYYTISFLNDAIESYSKSQDE |
| | | KRVKIRLEAYFAEFKSKDDAKKQFDLLERIEEAYAIVEPLLGAEYPRDR |
| | | NLKADKKEVGKIKDFLDSIKSLQFFLKPLLSAEIFDEKDLGFYNQLEGY |
| | | YEEIDSIGHLYNKVRNYLTGKIYSKEKFKLNFENSTLLKGWDENREVA |
| | | NLCVIFREDQKYYLGVMDKENNTILSDIPKVKPNELFYEKMVYKLIPT |
| | | PHMQLPRIIFSSDNLSIYNPSKSILKIREAKSFKEGKNFKLKDCHKFIDF |
| | | YKESISKNEDWSRFDFKFSKTSSYENISEFYREVERQGYNLDFKKVSKF |
| | | YIDSLVEDGKLYLFQIYNKDFSIFSKGKPNLHTIYFRSLFSKENLKDVCL |
| | | KLNGEAEMFFRKKSINYDEKKKREGHHPELFEKLKYPILKDKRYSEDK |
| | | FQFHLPISLNFKSKERLNFNLKVNEFLKRNKDINIIGIDRGERNLLYLV |
| | | MINQKGEILKQTLLDSMQSGKGRPEINYKEKLQEKEIERDKARKSW |
| | | GTVENIKELKEGYLSIVIHQISKLMVENNAIVVLEDLNIGFKRGRQKVE |
| | | RQVYQKFEKMLIDKLNFLVFKENKPTEPGGVLKAYQLTDEFQSFEKL |
| | | SKQTGFLFYVPSWNTSKIDPRTGFIDFLHPAYENIEKAKQWINKFDSI |
| | | RFNSKMDWFEFTADTRKFSENLMLGKNRVWVICTTNVERYFTSKT |
| | | ANSSIQYNSIQITEKLKELFVDIPFSNGQDLKPEILRKNDAVFFKSLLFYI |
| | | KTTLSLRQNNGKKGEEEKDFILSPVVDSKGRFFNSLEASDDEPKDAD |
| | | ANGAYHIALKGLMNLLVLNETKEENLSRPKWKIKNKDWLEFVWER |
| | | NR |
| 156 | Cas12 | MKRIAKFRHDKPVKREAWSKGYRVHKNRIINKVTRSIKYPLVVKDE |
| | | WKKRLIDDAAHDYRWLVGPINYSDWCRDPNQYSILEFWIDFLCVG |
| | | GVFQSSHSNICRLAIQLSGGSVFEQEWKDLSPFVRANLIQGIKPAEFI |
| | | GFLTAEFRSSSNPKNFISKFFEGSNEDLESLTNEFASIVDFIKAKDISLLR |
| | | KSLPSCKKIAPNLWEKAVGSHSTNELLKLLTKYTRVMLVAEPSHSDR |
| | | VFSQTVLQSNDQDDPELTGPLPSHKVGKASYLFIPEFIREVNLDKISKL |
| | | DLSAKSKLAVEQVKKLSELTSDFKQIENQSEAYFGLSTSFNELSNFLGI |
| | | LIRTLRNAPEAILKDQIALCAPLDKDILKITLDWLCDRAQALPENPRFE |
| | | TNWAEYRSYLGGKIKSWFSNYENFFEIPQAASSQQNNNREKKLGNR |
| | | SAIRALNLKKEAFEKARETFKGDKGTLEKIDLAYRLLGSISPEVLQCDE |
| | | GLKLYQQFNDELLVLNETINQKFQDAKRDIKAKKEKESFEKLQRNLSS |
| | | PLPRIPEFFGERAKKGYQKARVSPKLARHLLECLNDWLARFAKVEES |
| | | AFSEKEFQRILDWLRTSDFLPVFIRKSKDPPSWLRYIARVATGKYYFW |
| | | VSEYSRKRVQJIDKPIAQNPLKELISWFLLNKDAFSRDNELFKGLSSK |
| | | MVTLARIMAGILRDRGEGLKELQAMTSKLDNIGLLHPSFSVPVTDSL |
| | | KDAAFYRAFFSELEGLLNIGRSRLIIERITLQSQQSKNKKTRRPLMPEP |
| | | FINEDKEVFLAFPKFETKNKVKGTRVVYNSPDEVNWLLSPIRSSKGQL |
| | | SFMFRCLSEDAKIMTTSGGCSYIVEFKKLLEAQEEVLSIHDCDIIPRAF |
| | | VSIPFTLERESEETKPDWKPNRFMGVDIGEYAVAYCVIEKGTDSIEIL |
| | | DCGIVRNGAHRVLKEKVDRLKRRQRSMTFGAMDTSIAAARESLVG |
| | | NYRNRLHAIALKHGAKLVYEYEVSAFESGGNRIKKVYETLKKSDCTGE |
| | | TEADKNARKHIWGETNAVGDQIGAGWTSQTCAKCGRSFGADLKA |
| | | GNFGVAVPVPEKVEDSKGHYAYHEFPFEDGLKVRGFLKPNKIISDQK |
| | | ELAKAVHAYMRPPLVALGKRKLPKNARYRRGNSSLFRCPFSDCGFT |
| | | ADADIQAAYNIAVKQLYKPKKGYPKERKWQDFVILKPKEPSKLFDKQ |
| | | FYRPN |
| 157 | Cas12 | MAIRSIKLKLKTHTGPEAQNLRKGIWRTHRLLNEGVAYYMKMLLLF |
| | | RQESTGERPKEELQEELICHIREQQQRNQADKNTQALPLDKALEALR |
| | | QLYELLVPSSVGQSGDAQIISRKFLSPLVDPNSEGGKGTSKAGAKPT |
| | | WQKKKEANDPTWEQDYEKWKKRREEDPTASVITTLEEYGIRPIFPLY |
| | | TNTVTDIAWLPLQSNQFVRTWDRDMLQQAIERLLSWESWNKRVQ |
| | | EEYAKLKEKMAQLNEQLEGGQEWISLLEQYEENRERELRENMTAA |
| | | NDKYRITKRQMKGWNELYELWSTFPASASHEQYKEALKRVQQRLR |
| | | GRFGDAHFFQYLMEEKNRLIWKGNPQRIHYFVARNELTKRLEEAKQ |
| | | SATMTLPNARKHPLWVRFDARGGNLQDYYLTAEADKPRSRRFVTF |
| | | SQLIWPSESGWMEKKDVEVELALSRQFYQQVKLLKNDKGKQKIEFK |
| | | DKGSGSTFNGHLGGAKLQLERGDLEKEEKNFEDGEIGSVYLNVVIDF |
| | | EPLQEVKNGRVQAPYGQVLQLIRRPNEFPKVTTYKSEQLVEWIKASP |
| | | QHSAGVESLASGFRVMSIDLGLRAAAATSIFSVEESSDKNAADFSYW |
| | | IEGTPLVAVHQRSYMLRLPGEQVEKQVMEKRDERFQLHQRVKFQI |
| | | RVLAQIMRMANKQYGDRWDELDSLKQAVEQKKSPLDQ.TDRTFWE |
| | | GIVCDLTKVLPRNEADWEQAVVQIHRKAEEYVGKAVQAWRKRFAA |
| | | DERKGIAGLSMWNIEELEGLRKLLISWSRRTRNPQEVNRFERGHTS |
| | | HQRLLTHIQNVKEDRLKQLSHAIVMTALGYVYDERKQEWCAEYPAC |
| | | QVILFENLSQYRSNLDRSTKENSTLMKWAHRSIPKYVHMQAEPYGI |
| | | QIGDVRAEYSSRFYAKTGTPGIRCKKVRGQDLQGRRFENLQKRLVN |
| | | EQFLTEEQVKQLRPGDIVPDDSGELFMTLTDGSGSKEVVFLQADINA |
| | | AHNLQKRFWQRYNELFKVSCRVIVRDEEEYLVPKTKSVQAKLGKGLF |
| | | VKKSDTAWKDVYVWDSQAKLKGKTTFTEESESPEQLEDFQEIIEEAE |
| | | EAKGTYRTLFRDPSGVFFPESVWYPQKDFWGEVKRKLYGKLRERFL |
| | | TKAR |
| 158 | Cas12 | MTHAKKIPFPVLKRSTLRKARQRIAAGSITAGERPFNSTVTRVVPVK |
| | | DPVSDQVWAVAREAAMTLRGFGQGSLFDMLIHLHADGFRLFPSG |
| | | REREAFFLKDLFDPTEFDDGARRAFGDVMPGFTANSLREILGAPARK |
| | | CGKVTSVEILLPRLSKGLGVKKSAAPPEVLSSLAAALCEAFPTWSLLTA |
| | | VDGGVGKVIDDVLRTHGSRLPSLEKAWSTNLPEVPKGLGVPTLAFD |
| | | DQAPAQSEQTPTGRFAGVVARYLAETFASNPEATAGDASKAVQAK |
| | | VTTPNGNALSWLFAVGRRAMCSTTLDELAIGLNITSPRGRHALSSLK |
| | | ERMMALPALSVLGERAYPDSRATLQGTVDSLIANYVNRLFELSSSAT |
| | | SIAQTKLILPAAIQGDTAVFDGMPFSAEDVGALFEQLPSEIAKLEHAV |
| | | KVLVGKERTSTLGYQKAVDDVDEFGVWASSVDAVIGQINARLKTLE |
| | | RAQEPLGKLMGDGKLKRLVNIHEPEGPAVEIIPVLDQELQDVLTSCR |
| | | TAFADLEARYPMTVAKAQRHAEAEVRNALELASRKEGGLSLASADV |
| | | PALAKRKILEPIISIARRSSPAMATAVLTECLRQKLIVKGTGSERSLRGY |
| | | VLSGEQVIYAHPLSRRRSIVRLDREGLQNFDALEFLDALQKDATQRT |
| | | NVRESLIVEMARQSLLLSALPDRIEIGAISWQTPSQNQHAPWANLR |
| | | PVNGTVGRSETIKSFTAVFHSRISGLLYRLNRQKFMEKYDLRCFIGSTL |
| | | LFSPKNADWAPPPQYRHGRFSALLARSDFPWEGAEGTHANAVRLA |
| | | KFLIDETRNATDLQQAIAAKALLAQLPHDWVVCCDFDGAPSYEGAF |
| | | VSAGEVSAWAKRSGYLLTPPRHFAGAFLEGFKSTKISPHGLTFERML |
| | | ERDGDSVIETGRRVTAAFPITQEVAPAAQPWKPRHLAGLDLGEAGL |
| | | GVCLKNLDNGHEQTLLLKTRKTRLLAHSAEHYRRKDQPRQVFRKQY |
| | | NQSSENAIKAAIGEVCGLIDNLIARYDAVPVFESQAAAARGSNRMV |
| | | ARVYAGVLQRYTYVVGNGAADATRTSHWLGANRWSYSFGADVIP |
| | | KVRDLSPEVLRSIKKPENVFRDALGFPGVLANAWRTSMICSVCGTDP |
| | | IGALEEAIAANQISFVTDNEGEGSLDLGDGRKVTLRVEVPTSSALTKR |
| | | EASRRKRRAPWEAKVGTVWTLTRKSHRDDLLTTIRRSLRRPSSTFQG |
| | | STTKQWEFHCPCCGQIQQADVNAASNLVRRYFVRASDNARARQH |
| | | WADDSKRLAFIASMGPDRSAREEKVS |
| 159 | Cas12 | MNRIYQGRVTKVEKLKNGKSPDDREELKDWQTALWRHHELFQDA |
| | | VSYYTLALAAMAEGLPDKHPINVLRKRMEEAWEEFPRKTVTPAKNL |
| | | RDSVRPWLGLSESASFGDALKKILPPAPENKEVRALAVALLAEKARTL |
| | | KPQKTSASYWGRFCDDLKKKPNWDYSEEELARKTGSGDWVAGLW |
| | | SEDALNKIDELAKSLKLSSLVKCVPDGQINPEGARNLVKEALDHLEGV |
| | | SNGTKKEKNDPGPAKKTNNWLRQHASDVRNFIHKNKNQFSSLPNG |
| | | RLITERARGGGININKTYAGVLFKAFPCPFTFDYVRAAVPEPKVKKVD |
| | | QEKKSEQSATWTELEKRILRIGDDPIELARKNNKPIFKAFTALEKWSD |
| | | QNSKSCWSDFDKCAFEEALKTLNQFNQKTEEREKRRSEAEAELKYM |
| | | MDENPEWKPKKETEGDDVREVPILKGDPRYEKLVKLFGDLDEEGSE |
| | | HATGKIYGPSRASLRGFGKLRNEWVDLFTKANDNPREQDLQKAVT |
| | | GFQREHKLDMGYTAFFLKLCERDYWDIWRDDTEVEVKKIREKRWV |
| | | KSVVYAAADTRELAEELERLQEPVRYTPAEPQFSRRLFMFSDIKGKQ |
| | | GAKHIREGLVEVSLAVKDQSGKYGTCRVRLHYSAPRLIRDHLSDGSS |
| | | SMWLQPMMAALGLSSDARGCFTRDSKGNVKEPAVALMSDFVGR |
| | | KRELRMLLNFPVDLDISKLEENIGKKARWEKQMNTAYEKNKLKQRF |
| | | HLIWPGMELKETQEPGQFWWDNPTIQKEGMYCLAIDLSQRRAAD |
| | | YALLHAGVNRDSKTFVELGQAGGQSWFTKLCAAGSLRLPGEDTEVI |
| | | REGKRQIELSGKKGRNATQSEYDQAIALAKQLLHNENSAELESAARD |
| | | WLGDNAKRFSFPEQNDKLIDLYYGALSRYKTWLRWSWRLTEQHKE |
| | | LWDKTLDEIRKVPYFASWGELAGNGTNEATVQQLQKLIADAAVDL |
| | | RNFLEKALLHIAYRALPLRENTWRWIENGKDGKGKPLHLLVSDGQS |
| | | PAEIPWLRGQRGLSIARIEQLENFRRAVLSLNRLLRHEIGTKPEFGSST |
| | | CGESLPDPCPDLTDKIVRLKEERVNQTAHLIIAQSLGVRLKGHSLFTEE |
| | | REKADMHGEHEVIPGRSPVDFVVLEDLSRYTTDKSRSRSENSRLMK |
| | | WCHRKINEKVKLLAEPFGIPVIEVFASYSSKFDARTGAPGFRAVEVTS |
| | | EDRPFWRKTIEKQSVAREVFDCLDNLVGKGLNGIHLVLPQNGGPLFI |
| | | AAVKEDQPLPAIRQADINAAVNIGLRAIAGPSCYHAHPKVRLIKGES |
| | | GTDKGKWLPRKGKEANKRENAQFGNVDLDLEVKFNRLDIDSDVLK |
| | | GDNTNLFHDPLNIACYGFATIQNLQHPFLAHASAVFSRQKGAVARL |
| | | QWEVCRAINSRRLEAWQKKAEKAAVKR |
| 160 | Cas12 | MSHELAEQPSPPKNKEPTCEESDAIRKNRRILLALSWLSVEDDCSAP |
| | | TGTFRVASGKDSEAERKNKVLTAFRSILTARRMRSQDVESWIADCA |
| | | ASLSAKIREDAVWINRSACFDQRALDLKVLSREYAKAAVMSFFGPLD |
| | | EYFKLPDEADDTKPAVGGDGPDFRTLARQWVSTNFGTGKKSDSEAI |
| | | AQNLRKLADANI.APFSGKPKAALIAHLSVELDGSTADIDGLCRAIGW |
| | | NTGRPSKGRVAIERLPDPPTETSIQTMQQKFREEAEAKASSKGLRQ.V |
| | | PEWMPAFQKSIERDCGMPFKLGEGRDHIGEFSVMLDHAARRVSIG |
| | | HSWIKRAEAERRRFEADAQRLNHIPAAAKDWLDQFVQFRSGSSGA |
| | | AAAGGEYRIRRRAIEGWDEIIKRWKRAACKSPEDRVAAAREVQADP |
| | | EIEKFGDIQLFEALAADDAECVWRGDGNGTPDPLKDYVAATDALDK |
| | | MRRFKVPAYRHPDPLAHPVFGDFGNSRGDIRFAVHEAAKATRGTK |
| | | RIAKDQKEWIRERHGLRMGLWDGQSVRTADLRWSSKRLVDDLALR |
| | | NHVTTRRTGPVSRADRLGRAAAGLGADEAACVAGLFELPDWNGRL |
| | | QAPRAQLDAIAACVAANGGKWDDKARKLRDRIEWLVSFSAKLECC |
| | | GPFMEYASQNGIQPNGKGEYYPHAERNKGRTGHAKLILSRLPGLRV |
| | | LAVDLGHRFAAACAVWEALSKIAFDAETKGREVVSGGRAADDLYC |
| | | HTRHLDCAGKARTTIYRRIGPDKLPDGSDHPAPWARLDRQFLIKLO |
| | | GEERPARAAGPAETAAVQQIETDLGRARGQEDLPPRPVDSLMREA |
| | | VRTIRIALRRHGDAARIAYAFKPGAKRLKPGGGAQDHTPETHADAIL |
| | | EALLRWHELATGARWRDPWAETQWKDWVQPHISATLPELANDA |
| | | DRWERKRHRAALEQVLRPVAQMLIQRPTDALHQVWSKHWADED |
| | | LKWPSRLRWLRNWLLPRGPRARSGAARNVGGLSLLRIATLRELYQT |
| | | QKAYAMRPEPDDPRKRIAGRNDDRYDELGRSVLQVIERLREQRVK |
| | | QLASRIVEAALGVGRAKPTRGRQRPQSRVDVPCHAVIIESLRNYRPD |
| | | ELQTRRENRAIMNWSAGKVRKYLEEACQLHGLHLREVMPNYTSRE |
| | | DSRTGLPGVRCVDVPVDPKLGKPKAYWWNSVLSTARKKSIGDAAS |
| | | HDKQGDATSRFIVELAGCLDRLKADGKPLPKTVRVPRIGGDLFVAAP |
| | | PTSCTAPAHQPHPACDGARALQADLNAAANIGLRALLDPDFPAKW |
| | | WYVPCIDDQRGLALPRADKVLGSACFPGDPATFGSLLKTRTAAGPA |
| | | VDGQAAPDRKPRTGTHRPGSAKSRSLGDGKATTNYWSDRSARDLR |
| | | PADEGGHWQPTNVYWNWVRKRALLGLYSFNGLSPPSDDRP |
| 161 | Cas12 | MAIRSIKLKLKTNTGPEAQNLRKGIWRTHRLLNEGVAYYMNMLLLF |
| | | RQESTDKKTKQEIHEELIRHIRAQQQRNHADEKTQALPLEKALEALR |
| | | KLYELLVPSSVGQSGDSQIISRKFLSPLVDPNSEGGKGTSKAGAKPA |
| | | WQKKKEANDPTWKQDYEKWKKRREEDPTASVITTLEEYGIRPLFPL |
| | | YTNTVAEIAWLPLKSGQFVRTWDRDMFQQAIEGMLSWESWNRR |
| | | VQEEYAKLEGKMAQLNEQLEGGEEWIRLLEQYEEKREQELRENMT |
| | | AANDKFRITKRQMKGWKELYEVWSTFLPSASQEQYKEAIKRVQQR |
| | | LRGKFGDFHFFQYLSEEENRLIWKGNPQRIHYFVARNELTKKLEKAK |
| | | QSARRTLPDANKHPLWVRYDARGGNLQDYYLTAESDKPRSRRFVTF |
| | | SQLIWPSESGWLEKKDVQAELALSRQFYQQVTFLKNDKGKQEIEFK |
| | | DKGSGTTFSGHLGGAKLQLERSVLENKERKFEEGEIGKAYLNVAIDFK |
| | | PLQEVKNGRVQAPYGQVLQLIRLPNAFPKVRTYKSEELVEWIKASPQ |
| | | HLSGVESLASGFRVMSIDLGLRAAAATSIFSVEESSDKNATKLAYWIE |
| | | GTPLVAVHQRSYMLRLPGEQVEQHVWEKRDERGDQHKRVRFQIR |
| | | RLAEIIRLANKQYGDRWDELNRLDEAVSKEKSPLDQADRTFWEGIVS |
| | | DLTTALPLNDADWTEAVVOIHRKAELYVGKWQAWRKRFNADERK |
| | | GIAGLSMWSIEELDGLRKLLISWSRRTRNPQEVNRFEPEHTGHKRLL |
| | | THIQNVKKDRLKQVSHAIVMTALGYIYDEKKQKWCAKYPACQVILF |
| | | ENLSQYRSNLDRSAKENSTLMKWAHRSIPKYVHMQAEPYGIQIGD |
| | | VRAEYSSRYYAKTGTPGIRCKKLREHDVKGWRLDHLKKRLVNEQFLT |
| | | EAQVEQLKAGDIIPDDSGELFMTMTDGSGGKEIVFLQADINAAQNL |
| | | QKRFWQRNSELFKVSCRVIVRDEAEYLVPQAKKVQEKLGKGVFVKK |
| | | SDTAWKEVYVWDSQAKLKGKTTFTEESESPEQLEDLQEMIEEAEEA |
| | | KGTYRTLFRDPSGVFFPDFVWNTPKDFWGEVKRKLYGKLRERLLTK |
| | | VR |
| 162 | Cas12 | MNRIYQGRVTKVEVPDGKDEKGNIKWKKLENWSDILWQHHMLF |
| | | QDAVNYYTLALAAISGSAVGSDEKSIILREWAVQVQNIWEKAKKKA |
| | | TVFEGPQKRLTSILGLEQNASFDIAAKHILRTSEAKPEQRASALIRLLEE |
| | | IDKKNHNVVCGERLPFFCPRNIQSKRSPTSKAVSSVQEQKRQEEVRR |
| | | FHNMQPEEVVKNAVTLDISLFKSSPKIVFLEDPKKARAELLKQFDNA |
| | | CKKHKELVGIKKAFTESIDKHGSSLKVPAPGSKPSGLYPSAIVFKYFPV |
| | | DITKTVFLKATEKLAMGKDREVTNDPIADARVNDKPHFDYFTNIALI |
| | | REKEKNRAAWFEFDLAAFIEAIMSPHRFYQDTQKRKEAARKLEEKIK |
| | | AIEGKGGQFKESDSEDDDVDSLPGFEGDTRIDLLRKLVTDTLGWLGE |
| | | SETPDNNEGKKTEYSISERTLRIFPDIQKQWSELAEKGETTEGKLLEVL |
| | | KHEQTEHQSDFGSATLYQHLAKPEFHPIWLKSGTEEWHAENPLKA |
| | | WLNYKELQYELTDKKRPIHFTPAHPVYSPRYFDFPKKSETEEKEVSKN |
| | | THSLTTSLASEHIKNSLQFTAGLIRKTNVGKKAIKARFSYSAPRLRRDC |
| | | LRSENNENLYKAPWLQPMMRALGIDEEKADRQNFANTRITLMAK |
| | | GLDDIQLGFPVEANSQELQKEVSNGISWKGQFNWGGIASLSALRW |
| | | PHEKKPKNPPEQPWWGIDSFSCLAVDLGQRYAGAFARLDVSTIEKK |
| | | GKSRFIGEACDKKWYAKVSRMGLLRLPGEDVKVWRDASKIDKENG |
| | | FAFRKELFGEKGRSATPLEAEETAELIKLFGANEKDVMPDNWSKELS |
| | | FPEQNDKLLIVARRAQAAVSRLHRWAWFFDEAKRSDDAIREILESD |
| | | DTDLKQKVNKNEIEKVKETIISLLKVKQELLPTLLTRLANRVLPLRGRS |
| | | WEWKKHHQKNDGFILDQTGKAMPNVLIRGQRGLSMDRIEQITELR |
| | | KRFQALNQSLRRQIGKKAPAKRDDSIPDCCPDLLEKLDHMKEQRVN |
| | | QTAHMILAEALGLKLAEPPKDKKELNETCDMHGAYAKVDNPVSFIVI |
| | | EDLSRYRSSQGRSPRENSRLMKWCHRAVRDKLKEMCEVFFPLCERR |
| | | KAGSAWVSLPPLLETPAAYSSRFCSRSGVAGFRAVEVIPGFELKYPW |
| | | SWLKDKKDKAGNLAKEALNIRTVSEQLKAFNQDKPEKPRTLLVPIAG |
| | | GPIFVPISEVGLSSFGLKPQVVQADINAAINLGLRAISDPRIWEIHPRL |
| | | RTEKRDGRLFAREKRKYGEEKVEVQPSKNEKAKKVKDDRKPNYFAD |
| | | FSGKVDWGFGNIKNESGLTLVSGKALWWTINQLQWERCFDINKRH |
| | | IEDWSNKQKQ |
| 163 | Cas12 | MGEENNFSQFTGLYELSKTLRFELKPVWETEKLLIENQVFPKDKIVYE |
| | | SYKKIRPYLDKLHLQFIEESLSSVKLDFNEIEKKFLEWDKEKDKTTKNKL |
| | | KEEIFWKNKKWWLNSNLRKDMVSYFNLNWKKWINNFENKTIVNE |
| | | KWKEEKIKIKWEWYKILLSDSNLNILSDFFTQEKEWDNVLIEKYIENP |
| | | NFKENDWEKKFILEKQNLFKSFKWFTTYFTNFNHSRENFYKDDWKS |
| | | WRIATRIIDENLIFFLKNKKAFDEKYKNNSEITVKFNEKLLNFWEKLED |
| | | FFSLDFYNRCYTWKQIEYYNQLIWELNSVVNHEKQAKFSEYTQNKK |
| | | VSENNKFNKYDFPIFKELYKQILSEKATEQKFIEINDFKELKQNLQELIE |
| | | KNKQKNKFAINLTKSLIEKIDEYDFEKIYISKLSLNTISSKFFWSNKWFFI |
| | | QENLEKNIWKKNKNWKIDLPDFIKLSDIKTALENFNKILFEDKENKEN |
| | | IFKEEFDNITENNIFIKFMKIYENEFINLIEWKKNNKWDYEIIWYNKSL |
| | | LDLEKNILNISDFSDKKEEKEKQIEYIKKYLDSSLNIYRVMKYFALEKGK |
| | | ESVEWEFETDDIFYNEFKKFYIDNEIISYYNEFRNYLTKKPYSSNKLKLN |
| | | FENWTLIDGWDKNKEPDNYWTILRKNWKYFLALQIKWKNNIFYKK |
| | | KWSWVIDIEEAYKIDLWDEFYEKMDYKFLPDPKKMLPKVIFAKSNV |
| | | KLFNPSKEILQIKENETFKTWDKFKVNDFYKIVDFYKENIIKYPDWKIF |
| | | NFKFSDTKTYNNLSDFYKEIELWSYDLNFRKVSKKYILKSIEEKNIYLFEI |
| | | YNKDFADWKTWSENLHTMYFKWLFEENNLNNIVLKLNWQAEIFR |
| | | REASLKEKEVNRAKENKEKSHNIIENARYTKDKLFFHCPIKLNFAKHN |
| | | EKINQEILKYISDNKEINIIWIDRWEKHLAYYSVINRDWNIIKDKNWN |
| | | LVKWSLNIVWNNQNYHDKLETREKERQDARWSWKTIWNIKDLKQ |
| | | GYISQVVHKLAELVIEHNAIIVFEDLNSWFKRWRQKIERQVYQKLEK |
| | | ALIEKLNYLTFKDKNFWENWHYLKAYQLTAPFETFEKVWKQTWVIF |
| | | YTDPSYTSSTCPACWFRKDLYLKYSNLKNALWDIEKIDSIIFDWKRFIF |
| | | EYNNKKVFTDRDRKRNISSEESWIKWWKTIDNNITEFLEILFKKSNID |
| | | YTSWNNLILDIQEINEKELYKWVFDNFNAILNIRNSTIKDNSRTWDFI |
| | | CCPACDFDSRKENKIWIENWDDNWAFNIARKWIIILNKIDSYLNEK |
| | | WSLDKILWWDMIVKQIDWDDFTHKK |
| 164 | Cas12 | MAQASSTPAVSPRPRPRYREERTLVRKLLPRPGQSKQEFRENVKKLR |
| | | KAFLQFNADVSGVCQWAIQFRPRYGKPAEPTETFWKFFLEPETSLP |
| | | PNDSRSPEFRRLQAFEAAAGINGAAALDDPAFTNELRDSILAVASRP |
| | | KTKEAQRLFSRLKDYQPAHRMILAKVAAEWIESRYRRAHQNWERN |
| | | YEEWKKEKQEWEQNHPELTPEIREAFNQIFQQLEVKEKRVRICPAA |
| | | RLLQNKDNCQYAGKNKHSVLCNQFNEFKKNHLQGKAIKFFYKDAEK |
| | | YLRCGLQSLKPNVQGPFREDWNKYLRYMNLKEETLRGKNGGRLPH |
| | | CKNLGQECEFNPHTALCKQYQQQLSSRPDLVQHDELYRKWRREYW |
| | | REPRKPVFRYPSVKRHSIAKIFGENYFQADFKNSVVGLRLDSMPAGQ |
| | | YLEFAFAPWPRNYRPQPGETEISSVHLHFVGTRPRIGFRFRVPHKRS |
| | | RFDCTQEELDELRSRTFPRKAQDQKFLEAARKRLLETFPGNAEQELR |
| | | LLAVDLGTDSARAAFFIGKTFQQAFPLKIVKIEKLYEQWPNQKQAGD |
| | | RRDASSKQPRPGLSRDHVGRHLQKMRAQASEIAQKRQELTGTPAP |
| | | EIIIDQAAKKATLQPFDLRGLTVHTARMIRDWARLNARQIIQLAEE |
| | | NQVDLIVLESLRGFRPPGYENLDQEKKRRVAFFAHGRIRRKVTEKAV |
| | | ERGMRVVTVPYLASSKVCAECRKKQKDNKQWEKNKKRGLFKCEGC |
| | | GSQAQVDENAARVLGRVFWGEIELPTAIP |
| 165 | Cas12 | MGEENNFSQFTGLYELSKTLRFELKPVWETEKLLIENQVFPKDKIVYE |
| | | SYKKIRPYLDKLHLQFIEESLSSVKLDFNEIEKKFLEWDKEKDKTTKNKL |
| | | KEEIFWKNKKWWLNSNLRKDMVSYFNLNWKKWINNFENKTIVNE |
| | | KWKEEKIKIKWEWYKILLSDSNLNILSDFFTQEKEWDNVLIEKYIENP |
| | | NFKENDWEKKFILEKQNLFKSFKWFTTYFTNFNHSRENFYKDDWKS |
| | | WRIATRIIDENLIFFLKNKKAFDEKYKNNSEITVKFNEKLLNFWEKLED |
| | | FFSLDFYNRCYTWKQJEYYNQLIWELNSVVNHEKQAKFSEYTQNKK |
| | | VSENNKFNKYDFPIFKELYKQILSEKATEQKFIEINDFKELKQNLQELIE |
| | | KNKQKNKFAINLTKSLIEKIDEYDFEKIYISKLSLNTISSKFFWSNKWFFI |
| | | QENLEKNIWKKNKNWKIDLPDFIKLSDIKTALENFNKILFEDKENKEN |
| | | IFKEEFDNITENNIFIKFMKIYENEFINLIEWKKNNKWDYEIIWYNKSL |
| | | LDLEKNILNISDFSDKKEEKEKQIEYIKKYLDSSLNIYRVMKYFALEKGK |
| | | ESVEWEFETDDIFYNEFKKFYIDNEIISYYNEFRNYLTKKPYSSNKLKLN |
| | | FENWTLIDGWDKNKEPDNYWTILRKNWKYFLALQIKWKNNIFYKK |
| | | KWSWVIDIEEAYKIDLWDEFYEKMDYKFLPDPKKMLPKVIFAKSNV |
| | | KLFNPSKEILQIKENETFKTWDKFKVNDFYKIVDFYKENIIKYPDWKIF |
| | | NFKFSDTKTYNNLSDFYKEIELWSYDLNFRKVSKKYILKSIEEKNIYLFEI |
| | | YNKDFADWKTWSENLHTMYFKWLFEENNLNNIVLKLNWQAEIFR |
| | | REASLKEKEVNRAKENKEKSHNIIENARYTKDKLFFHCPIKLNFAKHN |
| | | EKINQEILKYISDNKEINIIWIDRWEKHLAYYSVINRDWNIIKDKNWN |
| | | LVKWSLNIVWNNQNYHDKLETREKERQDARWSWKTIWNIKDLKQ |
| | | GYISQVVHKLAELVIEHNAIIVFEDLNSWFKRWRQKIERQVYQKLEK |
| | | ALIEKLNYLTFKDKNFWENWHYLKAYQLTAPFETFEKVWKQTWVIF |
| | | YTDPSYTSSTCPACWFRKDLYLKYSNLKNALWDIEKIDSIIFDWKRFIF |
| | | EYNNKKVFTDRDRKRNISSEESWIKWWKTIDNNITEFLEILFKKSNID |
| | | YTSWNNLILDIQEINEKELYKWVFDNFNAILNIRNSTIKDNSRTWDFI |
| | | CCPACDFDSRKENKIWIENWDDNWAFNIARKWIIILNKIDSYLNEK |
| | | WSLDKILWWDMIVKQIDWDDFTHKK |
| 166 | Cas12 | MATAINYPTTQRAYTLRLRGIDPQDQSWRDALWATHEAVNRGAK |
| | | VFGEWLLTLRGGLDHQLADAPVKVRGGTTRLPSDEERRDRRVLLAL |
| | | SWLSVEDAHGAPPDASLIVAKGTDSADCRARKLADALIAILQARSVA |
| | | ASEIGDPSKPPEDQPGTWLGDCMGSLSAAIRDDAVWVNRSKAFDA |
| | | ATQSCPSLTRDEIWDFLEPFFASPDAYLKPERAESDEGDSTSAATEDK |
| | | AKDLVQKAGGWLSKRMGAGGGANFQDLARAYQAIAQWASSAQP |
| | | GQSAQQAVGSLAGYLSQHGFSPTANDATGVLAVISGPGYKSATRN |
| | | HITAIATSPEITPQDLSKLQELATKDKAGCSSKIGGKGPRPYATMILO |
| | | QVEAACGFTYLQSDGPARHREFSVMLDHAARRVNVAHSWIKNAE |
| | | AERRQFESDARRIKKVPQDALNWLRGYCEERGGASGSLEGYRIRRR |
| | | AIDGWDQVVIRWSRSDCQSADDRIAAARQLQDDPEIDKFGDIQLFE |
| | | ALAAEEALCVWKPDGNPTAQPLKDFVAATEADAKKKRFKVPAYRH |
| | | PDPLRHPVFTDFGNSRWGIEYSAHRAPAKCDELGQQVDRLTOAVA |
| | | EAQRNLDGATAAQRASRESKLAEAQSKLVAAQTEFAAINDPHRVEL |
| | | KLWNGQAVAAIPMRWSSKRLIADLSLRRATQPSSDQRIGVTRADRL |
| | | GRAAGNADDGRPVTITGLFQQDHWNGRLQAPRAQLDAIAKHVDK |
| | | HGWDAKARRQIARIRWVVSFSAELSQQGPWFEFCHRFGEDAPAR |
| | | PFVSRHGEYAVKHRDNDQRKGHAKLILSRLPGLRVLAVDLGHRYAA |
| | | ACAVWEAISSDQMRQACAAANAPAPHPLAMYIHLKSTTAKGKPTT |
| | | TIYRRIGPDKLPDGTPHPAPWARLDRQFLIKLPGEDRPARAASPDEIK |
| | | AVEDFEDSVGRVRTAVDPPRKRGVDLLMHDAVRTARLALARHGRR |
| | | ARIAFQLISQVRILPGGRPQTLDDAGRRDLLNDTLADWYALATDSR |
| | | WTDAAARQLWNERLAALNGGFTIDPPADASQPEAERTRAQRRQA |
| | | EQELRHRLASLVEALFCNPTLCQQLHQAWTDRWNADDQQWRSRL |
| | | KWLSRWLLPRGGSRRDGSRRHVGGLSLTRISTLIDFRRKVQVGYFTR |
| | | LRPDGSRAEIGPQFGQSTLDAIQRLKDQRIKQLTSRIVEAALGIGVEQ |
| | | DRIWDAAKRKWRTVKRPREPRYHVDDQGVQQRDPRFQACHAVVI |
| | | EDLSHYRPEETRTRRENRATMDWKSAETRKRLADHCQLYGLHLRD |
| | | VNPQYTSRQDSRTGAPGCRCVDVSVADFLTKPAWRKQVAQARGK |
| | | VASNRGDARDRLLVELDHQLTTANSLRGEMDSLRIPVNGGEVFVSA |
| | | DPRSPLAAGIQADLNAAANIGLRALMDPDFLGTWWYVPCDPSTKK |
| | | PHIEKVKGSILATVGALQATSEEAAAPPRRGRGGTRSAAPREVINLW |
| | | RDPSAVRIQDATAGEVWDVTPVYWSIVKDRVVDVLRQRNTKSGD |
| 167 | Cas12 | MAVRSVKLKLLVPRDGSAESVRKRKALWATHQFVNDAAAAYAELLL |
| | | EMRQEDVCRGTDDHGKDVIEPAAHWQAKLRARLAAKQLPPVAVA |
| | | EALPLLKAFYGSRLIKSFVANDKGVAGTGNATDLNTWLSGLVDPASV |
| | | AGEKTELRKQLLAELPLCEAADADFEGAARKMLAKSDAREALLEGP |
| | | GTGVGWPAAYNANPTDSVWLDMLHKAAAKARLELADTTVSELKK |
| | | LGVFPLLQAASSNRVFGSGVLNPFERMAAAQAAAALLPWETKRHE |
| | | MRKRRDKFADQLNQWDTEFGASHATALAAIRAFEAEESERARRESL |
| | | GNEGTGYRIGGRELRDAWTLLRDWLKGHSTATAAAREDKVRELQA |
| | | KQGRSFGSHRLLSWLAKPAQQWLADHSAGDVVTRIAVRNARQRK |
| | | LDTARTLPIWTGADAVKHPRFANFDPPNNTNQPGFDLRAGTQKGR |
| | | LTLRLSLLTERADGLLLAQDHDFQLVPSRQMAEIVLHKDGKERALSW |
| | | QSQDGIGRQVGDVGGSALLFSRDHAECLLERKQITRLERGAWPAAL |
| | | PVWFKLSLDIGAEHKALLKQRFKWGVWLNSALVTRNAKDAKGVPP |
| | | PVGTRVLAVDLGLRSAATVSVWQVVDAATPVVAGKWRVPLSDTLS |
| | | AVHERSAMLALPGEHVDAGVLAARRAANEKLAGLLAATSHLSTVFK |
| | | LGRAEQGDRRRELLERLGEGDDRRARAAVATTAAERDGLRAVLGA |
| | | TQDAWAGAVAAVWRRLETDLAGAIAAYRKQQREDVQLRREARHG |
| | | PGASQLPKQAAAERLLGGKSAWQIEYKERVRKLLTRWIMRQRPGD |
| | | TAVRRLARKDLGKYCGGLLDHLTALKEDRAKTTADLIVQAARGRVR |
| | | AHKDAHGRQQDRELWLAKYAPCDLIVMEDLGRYRFATDRPPSENR |
| | | QLMQWTHREVFRLVQMQAEVEGIQVLETGAEFSSKFDARTWAPG |
| | | VRCEPITKLWVERYRNGEMPWLADKADEWRREGIELAQLVPGQLL |
| | | PTGSGEQFVAVSATGGLRVRHADLNAAQCIALRALTGHGTAFRLTA |
| | | RRLGDVFVSAKGLGKRPQGALWREFGSALPPAVVVLRPAGEVRYAL |
| | | RPFASARDAAAALGLQLGALRNVDATDAESDAEDGDLAELLAGAD |
| | | PDRATFFRDPSGDVHGGAWVQAKVFWAEVRRHVRLGLQAQGLLP |
| | | AAARSSEPRQMQLPLAGALPGDDIPL |
| 168 | Cas12 | MAAFQRSYTMNLKPATSEQDKFILWNRLFLTHWSVNEGAKIFGELF |
| | | LNLRGGLSPELGIFDLNKDKDDRKKKALVMGRRRLLALGWLSVEDN |
| | | LSAGDHPFRIREIPVGRNMEIKQATTLLTEILKNKGVKDEAVIKEWID |
| | | DCTPSLIANIREDAVWINRAKSFYSMNPCPTKDEVWKILSYVLNTSFL |
| | | DLSLNDSSERDNTKNKKGTKENEKDVSNKSKELYGWLFTKNPNKM |
| | | REAGENKDKFINNFRENFNTFTDYANLKVEIELWRKNNISNTLLITQK |
| | | AKYPPEVKEANHPSKFSVGYRKLLVHLELWPSSKDENGDIPKGIEGK |
| | | DKSHNQILLDYLLEVCNEGNKTTKKVIVPAWADGIKTELESKASIKVG |
| | | DSTSSVLQRLM1KMAARRISQTLSWIKINEQVRHDAYQKKNKAFKLL |
| | | CEIDKNGEACKWLENYELFRRDDSGGEEYHISARAISCWKQILEEW |
| | | QKNDSSKALREKVKVVQAAEDKFGDARLFEDLADDNARSVWLLPD |
| | | GNKTPDILNWWCEYRTADIDESRFKIPCYCHPHPFKHPVYVEYGKS |
| | | NPQVIFSLKHDKARKNRIDNGWNPKNPRILALLLLDIVRQKSTLAPF |
| | | VWESKRLWKDLGGDATVTYKIPRSDRMGLSSIGNIDYARPEVPFLKE |
| | | KWNARLQSDRRTLEKLEKYWNPESMKWIDDGKFLIQSKWFITFGP |
| | | DMETAEGPWKLYLKENINDNNYLGNRSKENQKRGYRAKKLLSGYP |
| | | AGMRILSVDLGHRYAASCAIWETITKKQITEELAYQPDKNSVFEHSC |
| | | KTIDKKIKNTVYRRIGDDSIDAPWAKLEKQFTIKLQGEDKSCYLLRSDE |
| | | KELFRSILSKLSCLNNDTGHNILEMIENLLRIVKAKIYRQGILARISYSM |
| | | TAQYKPGKGGQKSPLSDEDKIHYLSENLAAWSALMGNQEWNEDGI |
| | | SDWYKKYISHLVSGPKPKEGNRKSDRDKIIEYFLPAARKLYDDNETRI |
| | | NIHDLFKELWDENNKQLSAVLKEIKKIILPKGIRYFDKNNDSSSRWKN |
| | | NQSKLKQITHRGGLSLRRIVAIEGYYKLAKAYKNHPEPDNLTKNIPLP |
| | | GDNSSAGFNQRIRDTLERMKEQRVKQIASRIVESALGLGIEGYKKRP |
| | | LTPESKPCQAIVIEDLSHYRPDELQTRRENRRLMQWSSSKVKKYLSE |
| | | ACEMHDVLLVEISPEYTSRQDSRTGVAGLRCIDINIREFLKDSSSWQ |
| | | NKIKTIQMKPTNKKSNLDQYLIELNESLGERYKDKVIPSDKFVRIPRKG |
| | | GDIFVSSSKESPVSKGIQADLNAAANIGLKALLDPDWAGAWWYILIE |
| | | AKSNHVIPYGKKYKGAECLRDFKFSGLENQVMKNNMNLWRDLOS |
| | | QFSSEDKWMSYKEYNELTEKRVINILRERAGLELIEE |
| '169 | Cas12 | MPTRTINLKMVLGRKDDTAELRRALWTTHEHVNLAVAEVERVLLRC |
| | | RGRSYWTLDRRGDPVHVPESQVAEDALAMAREAQRRNGWPVVG |
| | | EDEEILLALRYLYEQIVPSCLLDDLGKPLKGDAQKIGTNYAGPLFDSDT |
| | | CRRDEGKDVACCGPFHEVAGKYLGALPEWATPISKQEFDGKDASHL |
| | | RFKATGGDDAFFRVSIEKANAWYEDPANQDALKNKAYNKDDWKK |
| | | EKDKGISSWAVKYIQKQLQLGQDPRTEVRRKLWLELGLLPLFIPVFD |
| | | KTMVGNLWNRLAVRLALAHLLSWESWNHRAVQDQALARAKRDE |
| | | LAALFLGMEDGFAGLREYELRRNESIKQHAFEPVDRPYVVSGRALRS |
| | | WTRVREEWLRHGDTQESRKNICNRLQDRLRGKFGDPDVFHWLAE |
| | | DGQEALWKERDCVTSFSLLNDADGLLEKRKGYALMTFADARLHPR |
| | | WAMYEAPGGSNLRTYQIRKTENGLWADVVLLSPRNESAAVEEKTF |
| | | NVRLAPSGQLSNVSFDQIQKGSKMVGRCRYQSANQQFEGLLGGAE |
| | | ILFDRKRIANEQHGATDLASKPGHVWFKLTLDVRPQAPQGWLDGK |
| | | GRPALPPEAKHFKTALSNKSKFADQVRPGLRVLSVDLGVRSFAACSV |
| | | FELVRGGPDQGTYFPAADGRTVDDPEKLWAKHERSFKITLPGENPS |
| | | RKEEIARRAAMEELRSLNGDIRRLKAILRLSVLQEDDPRTEHLRLFME |
| | | AIVDDPAKSALNAELFKGFGDDRFRSTPDLWKQHCHFFHDKAEKVV |
| | | AERFSRWRTETRPKSSSWQDWRERRGYAGGKSYWAVTYLEAVRG |
| | | LILRWNMRGRTYGEVNRQDKKQFGTVASALLHHINQLKEDRIKTGA |
| | | DMIIQAARGFVPRKNGAGWVQVHEPCRLILFEDLARYRFRTDRSRR |
| | | ENSRLMRWSHREIVNEVGMQGELYGLHVDTTEAGFSSRYLASSGA |
| | | PGVRCRHLVEEDFHDGLPGMHLVGELDWLLPKDKDRTANEARRLL |
| | | GGMVRPGMLVPWDGGELFATLNAASQLHVIHADINAAQNLQRRF |
| | | WGRCGEAIRIVCNQLSVDGSTRYEMAKAPKARLLGALQQLKNGDA |
| | | PFHLTSIPNSQKPENSYVMTPTNAGKKYRAGPGEKSSGEEDELALDI |
| | | VEQAEELAQGRKTFFRDPSGVFFAPDRWLPSEIYWSRIRRRIWQVT |
| | | LERNSSGRQERAEMDEMPY |
| 170 | Cas12 | MVTRALNLKLVVPRRPGELTKAEALWSTHDIVNRATSYYESQLLLCR |
| | | QQDYQTRELTVSAGDQAPDLDALIANARDRNRYRGLEKPQVVREK |
| | | LRNLYEAIVPPAIGKTGTAQAVGAFVSPLLDADSRGFTEIFDKIEALPN |
| | | WVDGVRAEEPDALEAAADWLKSPQGKERLRPTGAPPTWIKLAKKK |
| | | DAGWAAAFVADIDKKLKEVEGTPTLMQELRALGVMPLFPSFFASRI |
| | | AGHKGAVSTWDRLALRLAVAHLLSWESWVELAAKEHAARVAKLEK |
| | | FRDDNILGEIADAVEALRLYEKERTEELQQKAQLDAEEVRTTSRTIRG |
| | | WVDLREKWLKTDASPDALISLVAAEQKRKSGKFGDPQLFRWLAKPE |
| | | NHFVWNKPDFDPPSLFASLRMIEGLVERSKETAWMTLPDARLHPR |
| | | SSQWEPHGGGNLKTFRLEQGEGGSLSVTLPLLRKSGDDSYVEEEHA |
| | | FSLAGSKQIPNASLDVRRNKYCLSYRTPTGEEAEAVVGSADLLLDWY |
| | | FLQQRSEHRPEEGDIGPAFLKLALDITPIDPVWGEREKTPAIHHFKTA |
| | | SGKNTRHADGVAPGFRMLAVDLGIRTLATCSVFELKATAPAGRLSFP |
| | | IAHLDLHAVHERSFTLTLDGEDPDRDAERWRENKSAELRRLRMGLT |
| | | RYRNIRNMREDAPDEREVLLEDLQEKVQEHGWAFEEPLLRELAKHK |
| | | DTPEPIWEAELTKALAQFRSDFGVIVGEWRRSNRARSTDSHAGKSM |
| | | WAIDHLTNSRRFLMSWSLLSKPGQIRRLDRDKQGVFAKHLLDHLEG |
| | | LKADRLKTGSDLIVQAARGFRRDKRGNWHKAYKPCHGILFEDLSRY |
| | | RMRTDRPRRENSQLMKWAHRAVPKEVGMQAEVYGIRVEDTGAA |
| | | FSSRFHAASHTPGIRMHPICQKDLENEWLLDEIEKQNSGVKRRELKL |
| | | GQLVQLNGGELFACVTASGVKTLHADINAAQNLQRRFFTRHGDAF |
| | | RIVARKVLVDEEEVWVPRSLGKRLLGALGSHGKLVPTGHESGSCRFE |
| | | EITTRAWSKLSGEKLSDDRVGNEEDQIIASIEEEALERTGEVVVFFRD |
| | | PSGQVLPRDLWYPSKTFWSIVKSTTLSKLKAAP |
| 171 | Cas12 | MEDKQFLERYKEFIGLNSLSKTLRNSLIPVGSTLKHIQEYGILEEDSLRA |
| | | QKREELKGIMDDYYRNYIEMHLRDVHDIDWNELFEALTEVKKNQT |
| | | DDAKKCLEKIQEKKRKEIYQYLSDDAVFSEMFKEKMISGILPDFIRCN |
| | | EEYSEEEKEEKLKTVALFHRFTSSFNDFFLNRKNVFTKEAIATAIGYRV |
| | | VHENAEIFLENMVAFQNIQKSAESQISIIERKNEHYFMEWKLSHIFTA |
| | | DYYMMLMTQKAIEHYNEMCGVVNQHMKEYCQKEKKNWNLYRM |
| | | KRLHKQILSNASTSFKIPEKYENDAEVYESVNSFLQNVMEKTVMERI |
| | | AVLKNNTDNFDLSKIYITAPYYEKISNYLCGSWNTIADCLTHYYEQQI |
| | | AGKGARKDQKVKAAVKADKWKSLSEIEQLLKEYARAEEVKRKPEEYI |
| | | AEIENIVSLKEVHLLEYHPEVNLIENEKYATEIKDVLDNYMELFHWMK |
| | | WFYIEEAVEKEVNFYGELDDLYEEIRDIVPLYNKVRNYVTQKPYSDTK |
| | | IKLNFGTPTLANGWSKSKEYDYNAILLQKDGKYYMGIFNPVQKPEKE |
| | | IIEGHSHPLEGNEYKKMVYYYLPSANKMLPKVLLSKKGMEIYQPSEYI |
| | | INGYKERRHIKSEEKFDLQFCHDLIDYFKSGIERNPDWKVFGFHFSDT |
| | | DTYQDISGFYREVEDQGYKIDWTYIKEADIDRLNEEGKLYLFQIYNKD |
| | | FSEKSTGRENLHTMYLKNLFSEENIREQVLKLNGEAEIFFRKSSVKKPII |
| | | HKKGTMLVNRTYMEEMHGESVKKNIPEKEYQEIYNYMNHRWKGE |
| | | LSAEAKEYLKKAVCHETKKDIVKDYRYSVDKFFIHLPITINYRASGKEA |
| | | LNSVAQRYIAHQNDMHVIGIDRGERNLIYVSVINMQGEIIEQKSFNV |
| | | VNKYNYKEKLKEREQNRDEARKNWKEIGQIKDLKEGYLSGVIHEIAK |
| | | MMIKYHAIVAMEDLNYGFKRGRFKVERQVYQKFENMLIQKLNYLV |
| | | FKDRSADEDGGVLRGYQLAYIPDSVKKLGRQCGMIFYVPAAFTSKID |
| | | PATGFVDIFNHKAYTTDQAKREFILSFDEICYDVERQLFRFTFDYANF |
| | | ATHNVTLARNNWTIYTNGTRTQKEFVNRRVRDKKEVFDPTEKMLK |
| | | LLELEGVEYQSGANLLPKLEKISDPHLFHELQRIVRFTVQLRNSKNEE |
| | | NDVDYDHVISPVLNEEGKFFDSSKYENKEEKKESLLPVDADANGAYC |
| | | IALKGLYIMQAIQKNWSEEKALSPDVLRLNNNDWFDYIQNKRYR |
| 172 | Cas12 | MAQASSTPAVSPRPRPRYREERTLVRKLLPRPGQSKQEFRENVKKLR |
| | | KAFLQFNADVSGVCQWAIQFRPRYGKPAEPTETFWKFFLEPETSLP |
| | | PNDSRSPEFRRLQAFEAAAGINGAAALDDPAFTNELRDSILAVASRP |
| | | KTKEAQRLFSRLKDYQPAHRMILAKVAAEWIESRYRRAHQNWERN |
| | | YEEWKKEKQEWEQNHPELTPEIREAFNQIFQQLEVKEKRVRICPAA |
| | | RLLQNKDNCQYAGKNKHSVLCNQFNEFKKNHLQGKAIKFFYKDAEK |
| | | YLRCGLQSLKPNVQGPFREDWNKYLRYMNLKEETLRGKNGGRLPH |
| | | CKNLGQECEFNPHTALCKQYQQQLSSRPDLVQHDELYRKWRREYW |
| | | REPRKPVFRYPSVKRHSIAKIFGENYFQADFKNSVVGLRLDSMPAGQ |
| | | YLEFAFAPWPRNYRPQPGETEISSVHLHFVGTRPRIGFRFRVPHKRS |
| | | RFDCTQEELDELRSRTFPRKAQDQKFLEAARKRLLETFPGNAEQELR |
| | | LLAVDLGTDSARAAFFIGKTFQQAFPLKIVKIEKLYEQWPNOKQAGD |
| | | RRDASSKQPRPGLSRDHVGRHLQKMRAQASEIAQKRQELTGTPAP |
| | | ElIIDQAAKKATLQPFDLRGLTVHTARMIRDWARLNARQIIQLAEE |
| | | NQVDLIVLESLRGFRPPGYENLDQEKKRRVAFFAHGRIRRKVTEKAV |
| | | ERGMRVVTVPYLASSKVCAECRKKQKDNKQWEKNKKRGLFKCEGC |
| | | GSQAQVDENAARVLGRVFWGEIELPTAIP |
| 173 | Cas12 | MAVKSIKVKLRLSECPDILAGMWQLHRATNAGVRYYTEWVSLMR |
| | | QEILYSRGPDGGQQCYMTAEDCQRELLRRLRNRQLHNGRQDQPG |
| | | TDADLLAISRRLYEILVLQSIGKRGDAQQIASSFLSPLVDPNSKGGRGE |
| | | AKSGRKPAWQKMRDQGDPRWVAAREKYEQRKAVDPSKEILNSLD |
| | | ALGLRPLFAVFTETYRSGVDWKPLGKSQGVRTWDRDMFQQALERL |
| | | MSWESWNRRVGEEYARLFQQKMKFEQEHFAEQSHLVKLARALEA |
| | | DMRAASQGFEAKRGTAHQITRRALRGADRVFEIWKSIPEEALFSOY |
| | | DEVIRQVQAEKRRDFGSHDLFAKLAEPKYQPLWRADETFLTRYALY |
| | | NGVLRDLEKARQFATFTLPDACVNPIWTRFESSQGSNLHKYEFLFDH |
| | | LGPGRHAVRFQRLLVVESEGAKERDSVVVPVAPSGQLDKLVLREEE |
| | | KSSVALHLHDTARPDGFMAEWAGAKLQYERSTLARKARRDKQGM |
| | | RSWRRQPSMLMSAAQMLEDAKQAGDVYLNISVRVKSPSEVRGOR |
| | | RPPYAALFRIDDKQRRVTVNYNKLSAYLEEHPDKQIPGAPGLLSGLR |
| | | VMSVDLGLRTSASISVFRVAKKEEVEALGDGRPPHYYPIHGTDDLVA |
| | | VHERSHLIQMPGETETKQLRKLREERQAVLRPLFAQLALLRLLVRCG |
| | | AADERIRTRSWQRLTKQGREFTKRLTPSWREALELELTRLEAYCGRV |
| | | PDDEWSRIVDRTVIALWRRMGKQVRDWRKQVKSGAKVKVKGYQL |
| | | DVVGGNSLAQIDYLEQQYKFLRRWSFFARASGLVVRADRESHFAVA |
| | | LRQHIENAKRDRLKKLADRILMEALGYVYEASGPREGQWTAQHPPC |
| | | QLIILEELSAYRFSDDRPPSENSKLMAWGHRGILEELVNQAQVHDVL |
| | | VGTVYAAFSSRFDARTGAPGVRCRRVPARFVGATVDDSLPLWLTEF |
| | | LDKHRLDKNLLRPDDVIPTGEGEFLVSPCGEEAARVRQVHADINAA |
| | | QNLQRRLWQNFDITELRLRCDVKMGGEGTVLVPRVNNARAKQLF |
| | | GKKVLVSQDGVTFFERSQTGGKPHSEKQTDLTDKELELIAEADEARA |
| | | KSVVLFRDPSGHIGKGHWIRQREFWSLVKQRIESHTAERIRVRGVGS |
| | | SLD |
| 174 | Cas12 | MSNPNIPNISPNITLTRDDVVNLLMSSIAMEELGLAHIINAEGEKIQF |
| | | ALGTLQGASGPPATLQQVLEVNQSTQAMLDTIFRQEMMLDSKLKT |
| | | ATNIPTLRGPTGPVGPTGAPGGVISINGQTGVVTLDASNGVMPFM |
| | | REQSTSSLDDYKDPGIYXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXGATGATGATGATGPQEPRALREPRAPQARREPRGLLEPQVL |
| | | RGPQEPRALREPRAPRALREPRALRALRALQELRAPRALRGLQEPRV |
| | | LQGPRERQVRPEPRVLQGPRERQVRQGLQGLLEPRAKRERQVRPE |
| | | PREPQERQAPLVQQALLEPQALLGQAL |
| 175 | Cas12 | MIKKSNFWQFTGLYELSKTLRFELKPVWETLNILEKDWVSQKDKEVE |
| | | ENYNKIKVFFDSLHREFVKQSLENWYLELLENFYNSYIELNKNIENKK |
| | | NKSLQKLFEKSSKELKKELVSFFEWKWNDWKQKYSFLKKWWIDVL |
| | | NEKEVLDLMWEFYPKEKELFKKFDKFFTYFSNFKESRKNFYADDWR |
| | | AWAIATRVIDENLITFIKNIEDFKKFKNNFSDFIENGFSDWKIKINWFT |
| | | LEEKQVFDLDFYNNCLLQDWIDNYNKILWWFSEENWNKIQWINEK |
| | | INLFKQNQNKTNSKDVKFPRFKLLYKQILSEKEKLIFVDEIENDEKLINF |
| | | IKESKNNNLIKVEKAIEITENFIKNNETFELDKIYLSKISINTISNKFFSSW |
| | | DYILKEGFDKWEIKEFISFEDLKNAFGKIKYENLEDIFKSNYITDYIAIN |
| | | WEDLYKNFLNIFLYEFKQNINEINFYNSELEKLFLEKFEKTETQVQIIKN |
| | | YFDSVLSLYKMTKYFALEKGKKKIEDLETDNNFYNDFFVYYEDFEIWK |
| | | DYNLVRNFITKKQVKTDKFKLNFENSQFLTGWDKDKEKERLWIILKK |
| | | DEKYYLWILKNNKIFNSYNYESWDFYEKMSYKQLNNVYRQLPRFAF |
| | | SKAKREVYWITPELEQIKEEFDIFQKNKEKWEKFDIEKLKKLINCYKK |
| | | WFIKTYENEFDLEKIKNTDYLDLATFYDEIEQKTYKIDFNKISENFINAK |
| | | VNSWELYLFQIYNKDFSETKKAWSKENIHTKYFKLLFDEKNLEKLVIKL |
| | | SWWAEMFFREKTEKLKTKLDKSWKEVLEHRRYSKDKIMLHLSITLN |
| | | ANKWDSFWFNKMVNEYLNKNEDIKIIWIDRGEKHLAYYSVIDKNW |
| | | KIEEIDTLNIIKSSDWKITNYLEKLEKIESSRKDSRVSWWEIENIKELKN |
| | | GYISQVVNKLAELIIKYNAIIVFEDLNIWFKRWRQKIEKQIYQKLELAL |
| | | AKKLNYLTQKDKNDNEVLWNLKALQLVPKVNDYQDIANYKQSGIM |
| | | FYTRANYTSTTCPCCWFRKNIYISNSDTKEKQKKDFEKIDIKFDGEKFI |
| | | FSYEIIQDKKAKQKSNKTNFSVNSNFSRFKYNSKKMLVEEVNLNLEL |
| | | QNLFKDIDLKWDINKQILEKDSYFYKSLTYYFNLILQLRNSDSKNDIDY |
| | | ITCPSCNYHSKDWFQGLEYNADANWAYNIARKWIIMLDRIEKNFEK |
| | | PDLYVSDIDWDNFTQK |
| 176 | Cas12 | MIKKSNFWQFTGLYELSKTLRFELKPVWETLNILEKDWVSQKDKEVE |
| | | ENYNKIKVFFDSLHREFVKQSLENWYLELLENFYNSYIELNKNIENKK |
| | | NKSLQKLFEKSSKELKKELVSFFEWKWNDWKQKYSFLKKWWIDVL |
| | | NEKEVLDLMWEFYPKEKELFKKFDKFFTYFSNFKESRKNFYADDWR |
| | | AWAIATRVIDENLITFIKNIEDFKKFKNNFSDFIENGFSDWKIKINWFT |
| | | LEEKQVFDLDFYNNCLLQDWIDNYNKILWWFSEENWNKIQWINEK |
| | | INLFKQNQNKTNSKDVKFPRFKLLYKQILSEKEKLIFVDEIENDEKLINF |
| | | IKESKNNNLIKVEKAIEITENFIKNNETFELDKIYLSKISINTISNKFFSSW |
| | | DYILKEGFDKWEIKEFISFEDLKNAFGKIKYENLEDIFKSNYITDYIAIN |
| | | WEDLYKNFLNIFLYEFKQNINEINFYNSELEKLFLEKFEKTETQVQIIKN |
| | | YFDSVLSLYKMTKYFALEKGKKKIEDLETDNNFYNDFFVYYEDFEIWK |
| | | DYNLVRNFITKKQVKTDKFKLNFENSQFLTGWDKDKEKERLWIILKK |
| | | DEKYYLWILKNNKIFNSYNYESWDFYEKMSYKQLNNVYRQLPRFAF |
| | | SKAKREVYWITPELEQIKEEFDIFQKNKEKWEKFDIEKLKKLINCYKK |
| | | WFIKTYENEFDLEKIKNTDYLDLATFYDEIEQKTYKIDFNKISENFINAK |
| | | VNSWELYLFQIYNKDFSETKKAWSKENIHTKYFKLLFDEKNLEKLVIKL |
| | | SWWAEMFFREKTEKLKTKLDKSWKEVLEHRRYSKDKIMLHLSITLN |
| | | ANKWDSFWFNKMVNEYLNKNEDIKIIWIDRGEKHLAYYSVIDKNW |
| | | KIEEIDTLNIIKSSDWKITNYLEKLEKIESSRKDSRVSWWEIENIKELKN |
| | | GYISQVVNKLAELIIKYNAIIVFEDLNIWFKRWRQKIEKQIYQKLELAL |
| | | AKKLNYLTQKDKNDNEVLWNLKALQLVPKVNDYQDIANYKQSGIM |
| | | FYTRANYTSTTCPCCWFRKNIYISNSDTKEKQKKDFEKIDIKFDGEKFI |
| | | FSYEIIQDKKAKQKSNKTNFSVNSNFSRFKYNSKKMLVEEVNLNLEL |
| | | QNLFKDIDLKWDINKQILEKDSYFYKSLTYYFNLILQLRNSDSKNDIDY |
| | | ITCPSCNYHSKDWFQGLEYNADANWAYNIARKWIIMLDRIEKNFEK |
| | | PDLYVSDIDWDNFTQK |
| 177 | Cas12 | MATRSFILKIEPNEEVKKGLWKTHEVLNHGIAYYMNILKLIRQEAIYE |
| | | HHEQDPKNPKKVSKAEIQAELWDFVLKMQKCNSFTHEVDKDVVFN |
| | | ILRELYEELVPSSVEKKGEANQLSNKFLYPLVDPNSQSGKGTASSGRK |
| | | PRWYNLKIAGDPSWEEEKKKWEEDKKKDPLAKILGKLAEYGLIPLFIP |
| | | FTDSNEPIVKEIKWMEKSRNQSVRRLDKDMFIQALERFLSWESWNL |
| | | KVKEEYEKVEKEHKTLEERIKEDIQAFKSLEQYEKERQEQLLRDTLNT |
| | | NEYRLSKRGLRGWREIIQKWLKMDENEPSEKYLEVFKDYQRKHPRE |
| | | AGDYSVYEFLSKKENHFIWRNHPEYPYLYATFCEIDKKKKDAKQQAT |
| | | FTLADPINHPLWVRFEERSGSNLNKYRILTEQLHTEKLKKKLTVQLDR |
| | | LIYPTESGGWEEKGKVDIVLLPSRQFYNQIFLDIEEKGKHAFTYKDESI |
| | | KFPLKGTLGGARVQFDRDHLRRYPHKVESGNVGRIYFNMTVNIEPT |
| | | ESPVSKSLKIHRDDFPKFVNFKPKELTEWIKDSKGKKLKSGIESLEIGLR |
| | | VMSIDLGQRQAAAASIFEVVDQKPDIEGKLFFPIKGTELYAVHRASE |
| | | NIKLPGETLVKSREVLRKAREDNLKLMNQKLNFLRNVLHFQQFEDIT |
| | | EREKRVTKWISRQENSDVPLVYQDELIQIRELMYKPYKDWVAFLKQL |
| | | HKRLEVEIGKEVKHWRKSLSDGRKGLYGISLKNIDEIDRTRKFLLRWS |
| | | LRPTEPGEVRRLEPGQRFAIDQLNHLNALKEDRLKKMANTIIMHAL |
| | | GYCYDVRKKKWQAKNPACQIILFEDLSNYNPYEERSRFENSKLMKW |
| | | SRREIPRQVALQGEIYGLQVGEVGAQFSSRFHAKTGSPGIRCSVVTK |
| | | EKLQDNRFFKNLQREGRLTLDKIAVLKEGDLYPDKGGEKFISLSKDRK |
| | | LVTTHADINAAQNLQKRFWTRTHGFYKVYCKAYQVDGQTVYIPESK |
| | | DQKQKIIEEFGEGYFILKDGVYEWGNAGKLKIKKGSSKQSSSELVDSD |
| | | ILKDSFDLASELKGEKLMLYRDPSGNVFPSDKWMAAGVFFGKLERIL |
| | | ISKLTNQYSISTIEDDSSKQSM |
| 178 | Cas12 | MAESMYNQQNINQKSTETPTEEDALGIKNPLLTPTTLGQNFLSLQSI |
| | | SPLGSRSINLINWSLISPNQTLLQNFQDNENWQDSSISEFRPFSKNSL |
| | | TENSPIIEPQSDKTLPSSVPIQLSSELPIQQPPETSFIDSESPIPQPPETP |
| | | SSDSESPIGQTENIFPFQIDQKTRRNSVIQNSKSSISHFFQKKSEFPRE |
| | | KISQENVNKLTKKSAENQDISTTEESVINSIETNDLQTINPTDIPNPEIP |
| | | VSTLQKQPDSTIDNIPQTESVSTINPTDIPXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | XXXXXXXXXXXXXXXXXXXXXXXXXXXXXXXXXXXXXXXXXXXXXXXX |
| | | xXXXXxxxxxxxxxxXXXXXXXXXXXXXXXSKNNRIPQLITFLKLNQY |
| | | QQLILQIFLTQKFSFQLSKNNRIPPLITFLKLNQYQQLIPRVLLHQKFLF |
| | | QLFKNNRIPPLITFPKLNQYQQLIPRIFLTQKFPFQISRNNRIPQLITFP |
| | | KLNOYQ |
| 179 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKOK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIOKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYOIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 180 | Cas13 | MARDNFLNTIKLLADKMKLGAISGFGKDGNEVNDINKLFGDKNTYA |
| | | NIENVVEFYFPWIKALEGRFSLDKGDRNLNDMKMFYKSVLTAFFTA |
| | | VDSLRNKYTHYSHKDLNIREIKIECTLGGKDYCIGLLNALDCIYDSAVN |
| | | LLKLRFMVGEDEVAHLRRCKAVNKKVVVRTEKDGFYYRLSDNGGVT |
| | | EKGVIFIASMFLNRKYGFLFLKQLEGFKRSDEKRYRLTLEAFLAFSNIK |
| | | PVDRLKSDKLDRASLGLDMLNELTKIPKELSETLSVDCLYKYLASDGE |
| | | DDLRSRIRYQDRFVPLALEFISQSDEFKDFRFYTYVGNYVYKGYIKRLI |
| | | DGTDKERYLSDRLCGFYKSVNDASSDAIAQKYGVEIKDSNEPDYMLP |
| | | DSFRPHVLRATPHFVINNNNIGIKICGNDCLPIVNGKGVESPEPDYW |
| | | LSIYELPAMLFYAYLREKNGKRFKDYKSIRELIEGVEKKADEKNDRDK |
| | | GALMARHIDKEIIWTQTKLDEVKRLEEKKVAAYGKKGRVVLKSGRM |
| | | ADLLAHDMVRLQPATKGSDKITGVNFQALQVSLAYFKRDILADVFS |
| | | RAMLTTGNHRHPFLYRIDVSHCSSLRDFYVAYLGERRKYFEDVAKKI |
| | | AKNKLNTPCHILRRLQREGSGEEAGKDVKPKFLPRGIFTDSIKNCLEQ |
| | | SKLNIYIRNARNDVKPAINAAYLILMYYKEIEKGEFQGFYGEKRRYDIL |
| | | EEGKPLDLAERKKALASIKPAKIDVSEANMPMSKEEHLMRKRYHAV |
| | | CNNESAIRMYQVQDILLLLMAKDIFKKALSEGVMSKKIGLENLNGIF |
| | | DAPVNFVKNFDNIKLTATGIKIKNYGKVCRLGTDFKFNSLIKAFHKVY |
| | | SKSVEMDYSDYLKEEEEFEKYRLNMVKLCREVERGITEDLHLSLDGKS |
| | | HLSFNDDVIKPYNDKYNVFNGDDLTFFINARNMFMHGDYKYECVK |
| | | YVVSEHFKGSLNDVSFAKETYGHFCNLLESMRKKTGLRIDI |
| 181 | Cas13 | MKKKISLKEQRNTKKAENKLKYQKAQAERAAAAQQ.TAAGAESEEN |
| | | PCFDVVKDTKRKALNPLHVEIEAPSAKKSSVKANGLKSLLLTDGKTV |
| | | MTSFGRGSEANVEKRFDETGTKTFDRDPELFSAKPLETGYRIQRFNA |
| | | SPKDAGLAYRPAGVRPDQIGAKAALEKRYFGKETPGDNIHVQIAYQI |
| | | QDIEKLLAVYISNIIYAVNNVTGVSAMKDSKGRPVDLLGDYGILGEEG |
| | | LTKRLQRIPEQADEEAKALQAFLCSERLSYFGKEFCLVRNSPKQPDKE |
| | | EKRQYKLMRVLCLLGELRQFLVHGKKKEKEFAWLYRLDRQLSQEYR |
| | | KLLGEFYDAQVDKVNKSFLTNSTVNLEVLFRALKTGTDPERKTVTQE |
| | | YYQFTVRKEDGNLGFSLKTLREILLSAYKHEVRDKEYDSIRHKLYQLFS |
| | | FALYHYYKTGVGAERREAFVAKLRAVMTAEAKQRAYADEAAEIWN |
| | | DEGSGIRAAFLEILEAVDFGSAVKGIKARSSVAGDKRFAEWLEEVRIR |
| | | PEGVSCFTKLMYLLTRFLDGKEINELLTGLINKLENIQSFLDVMQQEH |
| | | AETGLSDAFSFFEYSGEIAAELRMTRSFARMAAADPEAKRFMVVDG |
| | | AKLLGFNPKDTESEDEGIIRAIYGDACAEYLQFSEEEKEAFYVQEGLY |
| | | GKEREKFSPYAYFHTDTSLRNFIAKNVVESARFRYVIRYVSPEIARKYA |
| | | RQEALVRFALHRVPLLQLRRYYQSCCGPKKDPDAAECVDFLAGVVN |
| | | RVDFANFTDVRTGDSSKSEQEKKQKYQAIVGLYLTVVYWIVKNLVN |
| | | VNSRYVMAFHILERDTVLLEGKRLFVGGMKAEDPFLLTDGYVSRQD |
| | | AYVRKRIGENKRANRHGLNCVLENRNALGSDPASTDAAASLIWSYR |
| | | NAAAHLTAVAAAQEYVSELREIHSYFEVYHYAMQRYLKSGAEFAELV |
| | | SKNGPASGKIAAWANAVDRCHSFCKDWLWLLNVPFAYNPARYKN |
| | | LSIANLFDKNEAAPVTEDASEQKEDE |
| 182 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKOKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIOKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 183 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 184 | Cas13 | CILDFFTQDKAIAEYQLGVEFLQKNLPVIRYLYLPTSHKRFENVPKNQ |
| | | LISEQRNYFKNSLKVLKNLIRDYRNFYTHHFHKPIPVFPETYKLLDDLF |
| | | LAVANDVKKHRMKTDASKQLLKKGLIEELAQLEKLKLEDLKKLKREG |
| | | KKVNLNDKEAITNAILNDSFSHLLPKENTISKYYSAVPTEDIDTENGVT |
| | | ISESGIIFLLGLFLTKKQSEDLRSRVKGFKAKLIVNPENPINKKNNSLKY |
| | | MATHWVFGYLGFKGLKNRFTTTFTKDTLLAQIVDELSKVPDELYQVL |
| | | PEELKNEFLEDMNEYLKEENSESLDKATVIHPVIRKRYENKFAYFALR |
| | | FLDEFVDFPTLRFQLHLGNYVHDKREKPIEGTKYVTERIVKEKIKAFAK |
| | | LSEAAQLKQKYFEEKENHQSIGLQLYPNPSYNFVGNNIPIHLNLNEH |
| | | FFPKEVKIVAGRLKKRNSSYKSDHPEEYKVRTDNKIKPDAILQDLGKP |
| | | EKLAPVAMLSLNELPALLHLVLTKKTPEEIEIIIAQKIAERYNVLTNYKA |
| | | GDDISKGQITKNLLKAKQKKEVNLDKLQLAIEKEIAVTNDKLQTIALHI |
| | | KERNDPKQKRKYVFTNKEIGLQVTWLANDLKRFMPKGSRQNWRG |
| | | QHHSQLQKSLAFYDIQPKEPLSLLEEVWDFKNEAYLWNNGIRRSFD |
| | | KRDFISFYTSYLNNRKETFQRFKDQLNGIRSNKKILDKFIKQQHLWNL |
| | | FHKRLYVIDTIEEQVEKLLVKPMQFPKGVFDHKPTYIKGKSIQENPEC |
| | | FADWYVAWNQHTDYQKFYSWDRDYKSAYLSGEQEKTEKRFIRVQ |
| | | GSKINKVKQQDVLLAKMASIIFNELYLPEDAEHLDLNLSDIYKTQTER |
| | | KAEIEAALIQSHKTTGDNSANIIKSTSAWTLTVPYCSKNIYEPQVKLKE |
| | | LGKFKKFIASQKVQTLFEYKPQKIWNKTELEEVLELKANSYEVIRRDYL |
| | | LKSIQEFEKYMIKKLPTLIDTNEHPNFNKYLTTFLKSLELVSEEDAKWLI |
| | | SKKDFDTTPIDELKKQSKIMEKAFLLVMIRNKFSHNQLPRKIYYDEIYK |
| | | NVPNAVSINFNELFLEYTNQTILEFK |
| 185 | Cas13 | NRRDMEASPGRLARYIVLKTLYERAFPRWLEAREAETLNGWIGRAA |
| | | DRATVAARHINKDENAAARMAGLVRLADGKGIADFTDRLAAETAS |
| | | EYRVQRGYDSDPAVARKQAKHIEDLRCDVVGQAFEAYLADVARKL |
| | | AWTMNDPPGGPLPDEKKASLETAAPPAEDAGAEAEEDWLARLYFL |
| | | LHMLPVEEVGGLRHQLRKWSVLEREPDSVLEREPDADVEAIERILGL |
| | | YLDMHDAKFEGGDGVAGAEALTDLFTSPAQFRRVCPEDGAGNGG |
| | | HVPWRGLREMLRFGGGEPRLMWAFRKWPIGDGMVDGLNALEAT |
| | | VAEAHKQREDLHAKWAGKKGFSRKDKDEYRAALETVVVHRHLAAH |
| | | VQLVNHARLHRLAMAVLARLADYAGLWERDLYFTTLALIRLENGKP |
| | | EDVFRSRELEWLREGRIVDALKELKKNADGSEPAVGTALQRLFGKGI |
| | | LTGGGTVSVRRDLLHFNMLQRKANEPFNLTTAVNDTRKLMAYDRK |
| | | LKNAVSRSIIELLAREGLDLAWEMKDHQLAGAVLKTRQAVHLGGAK |
| | | VGGGPITEDLHGPEFTAMAAALFGGEARAATDAAEAATGRPDRRE |
| | | RKSGGASPRPARPAKTLPAPTPRLLPAAGERVEGVLVEEKTRKGGW |
| | | KAAVEIGGPRIVGDIFNSGEVPSDAEPGLEAEFVVRVANPANASFM |
| | | WLSPDVEERLKKAAAPRRGGRPAGRQR |
| 186 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLOPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKOKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIOKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 187 | Cas13 | MGAIENKHIFAAYANLAIDGLIKTLNFIAKKLDTQKQLSSWDIKFMTL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 188 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 189 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 190 | Cas13 | MEEANRYIYGAYFNMARDNFLNTIKLLADKMKLGATSGFGKDGNE |
| | | VNDINELFGDKNTYANIENVVEFYFPWIKALEGRFSLDKGDRNLND |
| | | MKMFYKSVLTAFFTAVDSLRNKYTHYSHKDLNIREIKIECTLGGKDYC |
| | | IGLLNALDCIYDSAVNLLKLRFMAGEYEVAHLRRCKAVNKKVVVRTE |
| | | KDGFYYRLSDNGGVTEKGVIFIASMFLNRKYGFLFLKQLEGFKRSDEK |
| | | RYRLTLETFLAFSNIKPVDRLKSDKLDRASLGLDMLNELTKIPRELSETL |
| | | SVDCLYKYLTSDGEDDLRSRIRYQDRFVPLALEFISQSDEFKDFRFYTY |
| | | VGNYVYKGYIKRLIDGTDKERYLSDRLCGFYKSVNDASSDAIAQKYG |
| | | VEIKDSNEPDYMLPDSFRPHVLRATPHFVINNNNIGIKICGNDCLPIV |
| | | NGKGVESPEPDYWLSIYELPAMLFYAYLREKNGKLLKDYKSIRELIED |
| | | VEKKADEKNDRDKGALMARHIDKEIIWTQTKLDEVKRLEEKKVAAY |
| | | GKKGRVVLKSGRMADLLAHDMVRLQPATKGSDKITGANFQALQVS |
| | | LAYFKRDILADVFSRAMLTTGNHRHPFLYRIDVSHCSSLRDFYVAYLG |
| | | ERRKYFEDVAKKIAKNKLNTPCHILRRLQREGSGEEAGKDVKPKFLPR |
| | | GIFTGSIKSCLEKSALNINIRNARNDVKPAINAAYLILMYYKEIEKGEFQ |
| | | GFYGEKRRYDILEEGKPLDLDERKKALASIKPAKIDVSEANMPMSKEE |
| | | HLMRKRYHAVCNNESAIRMYQVQDILLLLMAKDIFKKALSEGVMSK |
| | | KIGLENLNGIFDAPVNFVKNFDNIKLTATGIKIKDYGKVCRLGTDFKF |
| | | NSLVKAFHKVYSKSVEMDYSDYLKEEEEFEKYRLNMVKLCREVERGI |
| | | TEDLHLSLDGKSHLGFNDDVIKPYNDKYNVFNGGDLTFFINARNMF |
| | | MHGDYKYECVKYVVSEHFKGSLNDVSFAKETYGHFCNLLESMRKKT |
| | | GLRIDI |
| 191 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKOK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIOKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAOPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 192 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 193 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEIEHLRRYTRKKGRVVLKTEDDHFYYTLVNNNDLSEKGYA |
| | | FFISMFLERKYSYLFLKKLSGFKRGDSLQYRLTLEVFTALSTKPPVERLR |
| | | TTKDTKQDRALDILNELSRIPIELYQTLEPKYREMYNETLQPTDAEDP |
| | | YGLPDRSRIRFRSRFEAFALHFLDKQADFKEIGFYTYLGNYFHNGYOK |
| | | TRVDRETKDRYINFQLAGFCKNIQDISAKKLSEALNVKSIDISTDSIPDI |
| | | NSFEPYLVQSTPHYIVNGNNIGIKVLPEGKDTYPTIDEKGAKMPIADF |
| | | WLSKYELPAMLFYTYLRNNNIHKSHCPLSVKDIIERSIHKSTKOKHPE |
| | | ERSELMLRRVMKAIFWTDSKLNEVERIKSQKSAFGKRQHEILKAGRI |
| | | AETLVRDMLWLQPSKNNGRDKVTEPNFQAIQVSLAYFGIRRNDLTE |
| | | IFTRAGLINSSNPHPFLAQIGTNYTSLIEFYIAYLKERKVYFSRIQKKILO |
| | | GKLNIQCHPLRDLQREPNKPQDKEEAIFLPRGLFNEAIINCLKKSKLK |
| | | QLIESPTREKSPALNVSYLIQNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDLSNDSSA |
| 194 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 195 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFETFALHFLDKQPDFKEIGFYTYLGNYFHNGYQ |
| | | KTRVDRETKDRYINFQLAGFCKNIQDISAKKLSEALNVKSIDISTDSIP |
| | | DINSFEPYLVQSMPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAEMLVRDMLWLQPSKNNGRDKVTEPNFQAIQVSLAYFGIRRN |
| | | DLTEIFTRAGLINSSNPHPFLAQIGTNYTSLIEFYIAYLKERKVYFSRIQK |
| | | KILQGKLNIQCHPLRDLQREPNKPQEKEEAIFLPRGLFNEAIINCLKKS |
| | | KLKQLIESPTREKSPALNVSYLIQNYFRAYFEDQSQEFYAQPRNYRLF |
| | | DKLSPNKGKSKSYLSLEQRIKKMEELRPSKIPVAEANKLLEKEDRLYRK |
| | | NYNEICDNESIIRLYQIQDILLFMMTKEYLPSDLYNRINKYKLENVKGI |
| | | LNERVSYLIDLNPLKIQGEDIKIKDYGKLFYIHHDTRISSLNKVLSKVKR |
| | | NNSISSSVKIQPYENYKRECLDFEEAQIQIIPIIHSFEIAMVSMFPDLKK |
| | | ATPGNYYDFNELITEYEKRTKQKIDSSFLIKTRNMFLHDKYEAECIKEIS |
| | | DDFVYAKKIIAEFKMKIENIKLEDLSNDSSA |
| 196 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 197 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEIEHLRRYTRKKGRVVLKTEDDHFYYTLVNNNDLSEKGYA |
| | | FFISMFLERKYSYLFLKKLSGFKRGDSLQYRLTLEVFTALSTKPPVERLR |
| | | TTKDTKQDRALDILNELSRIPIELYQTLEPKYREMYNETLOPTDAEDP |
| | | YGLPDRSRIRFRSRFEAFALHFLDKQADFKEIGFYTYLGNYFHNGYQK |
| | | TRVDRETKDRYINFQLAGFCKNIQDISAKKLSEALNVKSIDISTDSIPDI |
| | | NSFEPYLVQSTPHYIVNGNNIGIKVLPEGKDTYPTIDEKGAKMPIADF |
| | | WLSKYELPAMLFYTYLRNNNIHKSHCPLSVKDIIERSIHKSTKQKHPE |
| | | ERSELMLRRVMKAIFWTDSKLNEVERIKSQKSAFGKRQHEILKAGRI |
| | | AETLVRDMLWLQPSKNNGRDKVTEPNFQAIQVSLAYFGIRRNDLTE |
| | | IFTRAGLINSSNPHPFLAQIGTNYTSLIEFYIAYLKERKVYFSRIQKKILQ |
| | | GKLNIQCHPLRDLQREPNKPQDKEEAIFLPRGLFNEAIINCLKKSKLK |
| | | QLIESPTREKSPALNVSYLIQNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDLSNDSSA |
| 198 | Cas13 | MGAIENKHIFAAYANLAIDGLIKTLNFIAKKLDTQKQLSSWDIKFMTL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIOKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 199 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDK |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDLSNDSSA |
| 200 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSKKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNETLQPTDAED |
| | | PYGLPDRSRIRFRSRFEAFALHFLDKQADFKEIGFYTYLGNYFHNGYO |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKOKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIOKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 201 | Cas13 | MKHVFAAYFSDARLNIMASLNDVREKSGLKRYKNEAENVQNFEGIF |
| | | PKNIASDIRDKRITLLRIRFPFIETIVDPGRGKENPASKYGELSWLFQLV |
| | | NDMRNVFIHSTGSEEEIDYQHHKKIFNALRKVYDCGLRTVKSRFQLE |
| | | NDTTMPLLRCDNRGRPKPFNKFSLALCTSPERNGNKKQSDVLHDFG |
| | | RVLLCSLFLEKRQISGLVSHFWDKNGYGTDWNDSEQTIIRELLYVNRI |
| | | RLPSQRLRTDSTLTSVTLDTISELARCPRPLFELLDADGQENFRVGRS |
| | | PKNPQNRNDDPSYLLLRGHQSRFIPLAMRHLDFDSKCKLRFAVDLG |
| | | QYYHSVRLKPAESFIDGNPGIRRLGQKIIAFGRLKDFEDAEKPEIWKK |
| | | LEENGQKFAEEEEELLKRASITGSPEELKPYIIKTFPHYHLYRDKIGFCID |
| | | WEEKKDQKVKYPNLGVRGEDSKSTDSGGIKKRENRQLSRHQFWISP |
| | | NKIIDLAFYHYLQTEIKQEITASVKTGRKKCPYPSVENILKEYYEGMW |
| | | LIKELKEQGPLPPWTEIPQISERRAECHRWINKEIIKAENFTISLADLPK |
| | | AIRRHLKVLDSRETSVSDIIKRTKTMIEETCKKQKEIEYLLKYPKKRGKK |
| | | GFRPIKHGNIADFLTEDLLRFQPKDSSKKNGGKLTSKNYQILQKAIAY |
| | | YDKPYCIVDLLKKSGLLEGEFKHPFLCKIITEENHEQYSTLIDFYQKYLE |
| | | ERKAFLEGFIDTFVAGSSIPGWLRLRKPSTFESWLDQQLDEDEKLCQ |
| | | PLPVPKSLFYQMLLKMTAEKLDLTPEMLCKKGTQRFIRDSQEVEVKP |
| | | SVSWFIRQYMEHRDDTAQEMYMFKRRHELFDFFHDKKKTTKKLHE |
| | | EKTCCLSEKVRQEELESVHKGIEKLKKLLRTYKRSEKKIRHFSTMDMV |
| | | LYLLAKKNFEKLILCDETSGPDWSLKTLETKILSTKIKYELNVPGTDRTI |
| | | VHRACAIKKTGELRLLVRDRRLPSLLDYYPKTEKSIDQEEIKVELADYS |
| | | CKRIEAMKLISDLEQKIEESCNRGFCKPVPDKLKKIFGAKKHGTLLYAL |
| | | YHRFHEEKDKSVDEQGFNKDCFAHARQIRNAFAHNEYPVSEQFNQI |
| | | VRQVNETSQPHNHNSPVNHPKIADQLVKELDKLYKPWRNFLKRIAG |
| 202 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 203 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 204 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 205 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 206 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 207 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSKKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKOKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 208 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMKHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | KIPIELYQTLEPKYREMYNETLOPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKQLIESPTREKSPALNVSYLIQ |
| | | NYFRTYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRINSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDLVYAKKIIAEFKMKIENIKLE |
| | | DLSNDSSA |
| 209 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKOK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 210 | Cas13 | MGAIENKHIFAAYANLAIDGLIKTLNFIAKKLDTOKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKOKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIOKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYOIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 211 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYO |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKOKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIOKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 212 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPRGYDIPSSLNCIYDSAINIIKE |
| | | RFQAEEKEMEHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | RIPIELYQTLEPKYREMYNETLOPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKHLIESPTREKSPALNVSYLIH |
| | | NYFRAYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRISSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDFVYAKKIIAEFKMKIENIKLE |
| | | DLSNDSSA |
| 213 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNATLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 214 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFETFALHFLDKQPDFKEIGFYTYLGNYFHNGYQ |
| | | KTRVDRETKDRYINFQLAGFCKNIQDISAKKLSEALNVKSIDISTDSIP |
| | | DINSFEPYLVQSMPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAEMLVRDMLWLQPSKNNGRDKVTEPNFQAIQVSLAYFGIRRN |
| | | DLTEIFTRAGLINSSNPHPFLAQIGTNYTSLIEFYIAYLKERKVYFSRIQK |
| | | KILQGKLNIQCHPLRDLQREPNKPQEKEEAIFLPRGLFNEAIINCLKKS |
| | | KLKQLIESPTREKSPALNVSYLIQNYFRAYFEDQSQEFYAQPRNYRLF |
| | | DKLSPNKGKSKSYLSLEQRIKKMEELRPSKIPVAEANKLLEKEDRLYRK |
| | | NYNEICDNESIIRLYQIQDILLFMMTKEYLPSDLYNRINKYKLENVKGI |
| | | LNERVSYLIDLNPLKIQGEDIKIKDYGKLFYIHHDTRISSLNKVLSKVKR |
| | | NNSISSSVKIQPYENYKRECLDFEEAQIQIIPIIHSFEIAMVSMFPDLKK |
| | | ATPGNYYDFNELITEYEKRTKOKIDSSFLIKTRNMFLHDKYEAECIKEIS |
| | | DDFVYAKKIIAEFKMKIENIKLEDLSNDSSA |
| 215 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 216 | Cas13 | MGKLYGYKRWYEIEQNNGEKGSFKRKIRVKRVYDEESKSYVVRRDE |
| | | VEDTELIKNKNFIINLKDYKTRNSNIQKFYDKFHVGNILFKLKASVKKG |
| | | KKTFYKDLEKAETVLTNVEILEQ.TIKLANNSKINRKKLEKELTQLNINEK |
| | | SEEVTIDNIKIYLRQGEKFDKKENINAIKWRKLTSEELTIKVEIYRECQSI |
| | | NSNLYSLLDYVLSNEEYDDRYYLEEVENKLLIFGTGNKNKNGRNYYYF |
| | | DYVLKSLSKIKGLIKKDDRNLNYLMFLFNIQKTSENKEHFINKIFNYFK |
| | | YEVRIEKEDIVEFLVGELEYYDLIKRIEKKPSENQNQTNLEKTYILLDKH |
| | | EKLKETIDTKNEIVQKLTIELKNNNLRNRIEIILHKYKILELVDKLNKNIK |
| | | NGKINTELYGIYKEHYGQCIEYINFNELALEEKELYKIIYRYLKGRIEKLL |
| | | QNRNKIKIGELRIEDIFIFQKLLEKIEIRVKQYLLEHILYLGKLKHHNIDE |
| | | VNTIRFVEEHANEELSLELITLFSATNVELNRLLKVKGENGSYEKDYDF |
| | | FSAKPDKGKIKIKDTNSVMKFELLKKLKFINSEATETNQEAIDFLKEAY |
| | | NLRNNILHGKNEEIIEDNKKNLSKSYKNINELIEELRPSDNEICKSLNLD |
| | | IIFKGNRKINDINAKLFGNNREKIYLPSFSKLVPEIKNIIESYDKNGTFN |
| | | DERIKKIVLNGAIYVNKILYLKECSNKDGEFIKNLKEELNKDSKEKSKYV |
| | | SIEELYKKSQISASKGNKKAIYKYQRKIIEIYLKYLKENYVEILDFSKLNLN |
| | | IEQIENDIKNRKNSENKVLIESIKQKVFPENDFEYIISIFALLNDNIFINKI |
| | | RNRFFSTDTWLKNNRYSNIIKILDEVISVNLLRTELLNTSIDIEEIKDDV |
| | | LTEDIENIIPEIREEILQRTKKDFKTLLGNNAISKEGLSNEDIDKIRNIED |
| | | AKINLDFVNNEIKVSLKEYGSLPPNGVLSRNTSKYYNNEIAKKIDQISIL |
| | | TFTKKSIGTISDEKFRNIYWQERKESDESKKIFVYNKNILYLVTKHSFEK |
| | | LYKNFLEEELNNLKLEDTKYLRDLDLRREKNLKVDNTLKEINEKVRGY |
| | | SKEYKKKFIENLKNNDEYFGKVVSGRFKNYQEFKEIYDEVSEYKKIRD |
| | | VVNFNPLNKVYNYLIEINWKLAIQMARAERDLHYIVNGLNELKLIEL |
| | | NQGQNDGISRAYPKYKLNKEKNKKELRLEECHYNFDIDNYKKFEKIC |
| | | EKLGIDLSENGELQQENETNIRNYISHFYILRKPFVDISISEAIKRVSKLL |
| | | SYRTRYNNSTYSSVFEVFKKDVELNYDFLKKKIELNGKTYDEVVQRKK |
| | | ISCLELESYLDYKPIIKKILF |
| 217 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 218 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 219 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTHTAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 220 | Cas13 | MGKLYGYKRWYEIEQNNGEKGSFKRKIRVKRVYDEESKSYVVRRDE |
| | | VEDTELIKNKNFIINLKDYKTRNSNIQKFYDKFHVGNILFKLKASVKKG |
| | | KKTFYKDLEKAETVLTNVEILEQTIKLANNSKINRKKLEKELTQLNINEK |
| | | SEEVTIDNIKIYLRQGEKFDKKENINAIKWRKLTSEELTIKVEIYRECQSI |
| | | NSNLYSLLDYVLSNEEYDDRYYLEEVENKLLIFGTGNKNKNGRNYYYF |
| | | DYVLKSLSKIKGLIKKDDRNLNYLMFLFNIQKTSENKEHFINKIFNYFK |
| | | YEVRIEKEDIVEFLVGELEYYDLIKRIEKKPSENQNQTNLEKTYILLDKH |
| | | EKLKETIDTKNEIVQKLTIELKNNNLRNRIEIILHKYKILELVDKLNKNIK |
| | | NGKINTELYGIYKEHYGQCIEYINFNELALEEKELYKIIYRYLKGRIEKLL |
| | | QNRNKIKIGELRIEDIFIFQKLLEKIEIRVKQYLLEHILYLGKLKHHNIDE |
| | | VNTIRFVEEHANEELSLELITLFSATNVELNRLLKVKGENGSYEKDYDF |
| | | FSAKPDKGKIKIKDTNSVMKFELLKKLKFINSEATETNQEAIDFLKEAY |
| | | NLRNNILHGKNEEIIEDNKKNLSKSYKNINELIEELRPSDNEICKSLNLD |
| | | IIFKGNRKINDINAKLFGNNREKIYLPSFSKLVPEIKNIIESYDKNGTFN |
| | | DERIKKIVLNGAIYVNKILYLKECSNKDGEFIKNLKEELNKDSKEKSKYV |
| | | SIEELYKKSQISASKGNKKAIYKYQRKIIEIYLKYLKENYVEILDFSKLNLN |
| | | IEQIENDIKNRKNSENKVLIESIKQKVFPENDFEYIISIFALLNDNIFINKI |
| | | RNRFFSTDTWLKNNRYSNIIKILDEVISVNLLRTELLNTSIDIEEIKDDV |
| | | LTEDIENIIPEIREEILQRTKKDFKTLLGNNAISKEGLSNEDIDKIRNIED |
| | | AKINLDFVNNEIKVSLKEYGSLPPNGVLSRNTSKYYNNEIAKKIDQISIL |
| | | TFTKKSIGTISDEKFRNIYWQERKESDESKKIFVYNKNILYLVTKHSFEK |
| | | LYKNFLEEELNNLKLEDTKYLRDLDLRREKNLKVDNTLKEINEKVRGY |
| | | SKEYKKKFIENLKNNDEYFGKVVSGRFKNYQEFKEIYDEVSEYKKIRD |
| | | VVNFNPLNKVYNYLIEINWKLAIQMARAERDLHYIVNGLNELKLIEL |
| | | NQGQNDGISRAYPKYKLNKEKNKKELRLEECHYNFDIDNYKKFEKIC |
| | | EKLGIDLSENGELQQENETNIRNYISHFYILRKPFVDISISEAIKRVSKLL |
| | | SYRTRYNNSTYSSVFEVFKKDVELNYDFLKKKIELNGKTYDEVVQRKK |
| | | ISCLELESYLDYKPIIKKILF |
| 221 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEIEHLRRYTRKKGRVVLKTEDDHFYYTLVNNNDLSEKGYA |
| | | FFISMFLERKYSYLFLKKLSGFKRGDSLQYRLTLEVFTALSTKPPVERLR |
| | | TTKDTKQDRALDILNELSRIPIELYQTLEPKYREMYNETLQPTDAEDP |
| | | YGLPDRSRIRFRSRFEAFALHFLDKQADFKEIGFYTYLGNYFHNGYOK |
| | | TRVDRETKDRYINFQLAGFCKNIQDISAKKLSEALNVKSIDISTDSIPDI |
| | | NSFEPYLVQSTPHYIVNGNNIGIKVLPEGKDTYPTIDEKGAKMPIADF |
| | | WLSKYELPAMLFYTYLRNNNIHKSHCPLSVKDIIERSIHKSTKOKHPE |
| | | ERSELMLRRVMKAIFWTDSKLNEVERIKSQKSAFGKRQHEILKAGRI |
| | | AETLVRDMLWLQPSKNNGRDKVTEPNFQAIQVSLAYFGIRRNDLTE |
| | | IFTRAGLINSSNPHPFLAQIGTNYTSLIEFYIAYLKERKVYFSRIQKKILO |
| | | GKLNIQCHPLRDLQREPNKPQDKEEAIFLPRGLFNEAIINCLKKSKLK |
| | | QLIESPTREKSPALNVSYLIQNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFRIKTRNMFLHDKYEAECIKEISD |
| | | DFVYAKKIIAEFKMKIENIKLEDLSNDSSA |
| 222 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 223 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYIRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNETLOPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKOKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIOKKI |
| | | LQGKLNIQCHPLRDLQREPNKPQEKEEAIFLPRGLFNEAIINCLKKSKL |
| | | KQLIESPTREKSPVLNVSYLIQNYFRAYFEDQSQEFYAQPRNYRLFDK |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 224 | Cas13 | MGAIENKHIFAAYANLAIDGLIKTLNFIAKKLDTQKQLSSWDIKFMTL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMKHLRNYTLVNNNGLSEKGYAFFISKFLERKYSYLFLKKL |
| | | SGFKRGDSLQYRLTLEVFTALSTKPPVERLRTTKDTKQDRALDILNELS |
| | | KIPIELYQTLEPKYREMYNETLQPTDAEDPYGLPDRSRIRFRSRFEAFA |
| | | LHFLDKQADFKEIGFYTYLGNYFHNGYQKTRVDRETKDRYINFQLAG |
| | | FCKNIQDISAKKLSEALNVKSIDISTDSIPDINSFEPYLVQSTPHYIVNG |
| | | NNIGIKVLPEGKDTYPTIDEKGAKMPIADFWLSKYELPAMLFYTYLR |
| | | NNNIHKSHCPLSVKDIIERSIHKSTKQKHPEERSELMLRRVMKAIFWT |
| | | DSKLNEVERIKSQKSAFGKRQHEILKAGRIAETLVRDMLWLQPSKNN |
| | | GRDKVTEPNFQAIQVSLAYFGIRRNDLTEIFTRAGLINSSNPHPFLAQ |
| | | IGTNYTSLIEFYIAYLKERKVYFSRIQKKILQGKLNIQCHPLRDLQREPN |
| | | KPQEKEEAIFLPRGLFNEAIINCLKKSKLKQLIESPTREKSPALNVSYLIQ |
| | | NYFRTYFEDQSQEFYAQPRNYRLFDKLSPNKGKSKSYLSLEQRIKKM |
| | | EELRPSKIPVAEANKLLEKEDRLYRKNYNEICDNESIIRLYQIQDILLFM |
| | | MTKEYLPSDLYNRINKYKLENVKGILNERVSYLIDLNPLKIQGEDIKIK |
| | | DYGKLFYIHHDTRINSLNKVLSKVKRNNSISSSVKIQPYENYKRECLDF |
| | | EEAQIQIIPIIHSFEIAMVSMFPDLKKATPGNYYDFNELITEYEKRTKQ |
| | | KIDSSFLIKTRNMFLHDKYEAECIKEISDDLVYAKKIIAEFKMKIENIKLE |
| | | DLSNDSSA |
| 225 | Cas13 | MGAIKNKHIFAAYANLAIDGLIKTLNFIAKKLDTQKQLSSWDIKHVIT |
| | | LIDSIFDQNPQNNLEQVVEGYLPWIKPIIEMKTPKKGERQSDKLCIEY |
| | | KTIITAFASLLNDVRNYYTHYYHDPICIYPGGYDIPSSLNCIYDSAINIIK |
| | | ERFQAEEKEMEHLRRYTRKKGRVVLKTEDDHFYYTLANNNDLSEKG |
| | | YAFFISMFLERKYSYLFLKKLSGFKRGDSLQYRLTLEVFTALSTKPPVER |
| | | LRTTKDTKQDRALDILNELSKIPIELYQTLEPKYREMYNETLQPTDAE |
| | | DPYGLPDRSRIRFRSRFEAFALHFLDKQADFKEIGFYTYLGNYFHNGY |
| | | QKTRVDRETKDRYINFQLAGFCKNIQDISAKKLSEALNVKSIDISTDSI |
| | | PDINSFEPYLVQSTPHYIVNGNNIGIKVLPEGKDTYPTIDEKGAKMPI |
| | | ADFWLSKYELPAMLFYTYLRNNNIHKSHCPLSVKDIIERSIHKSTKQK |
| | | HPEERSELMLRRVMKAIFWTDSKLNEVERIKSQKSAFGKRQHEILKA |
| | | GRIAETLVRDMLWLQPSKNNGRDKVTEPNFQAIQVSLAYFGIRRND |
| | | LTEIFTRAGLINSSNPHPFLAQIGTNYTSLIEFYIAYLKERKVYFSRIQKK |
| | | ILQGKLNIQCHPLRDLQREPNKPQDKEEAIFLPRGLFNEAIINCLKKSK |
| | | LKQLIESPTREKSPALNVSYLIQNYFRTYFEDQSQEFYAQPRNYRLFD |
| | | KLSPNKGKSKSYLSLEQRIKKMEELRPSKIPVAEANKLLEKEDRLYRKN |
| | | YNEICDNESIIRLYQIQDILLFMMTKEYLPSDLYNRINKYKLENVKGIL |
| | | NERVSYLIDLNPLKIQGEDIKIKDYGKLFYIHHDTRISSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIGSSFLIKTRNMFLHDKYEAECIKEIS |
| | | DDFVYAKKIIAEFKMKIENIKLEDLSNDSSA |
| 226 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 227 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKQKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIQKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 228 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSKIPIELYQTLEPKYREMYNETLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKOKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIOKKI |
| | | LQGKLNIQCHPLRDLQREPNKPQEKEEAIFLPRGLFNEAIINCLKKSKL |
| | | KHLIESPTREKSPALNVSYLIQNYFRAYFEDQSQEFYAQPRNYRLFDN |
| | | LSPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDFSNDSSA |
| 229 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 230 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSOI |
| 231 | Cas13 | MDTPNFSERIPVSLQSHPYYFAHYLNMARHNAYVILEYVNRELIKPG |
| | | KNLDEDNLIQSTVLKDGYFDRKPDELSHRNRLLVQHFPFLREAENEG |
| | | ARTCNPVSYKLKTALAALNQWRNNASHYPLNQNHEKDFDLQPFFS |
| | | FAIEACKKRMREVFQPDDFYLLETNEKQFYTLHNENGFTEKGLYCFIC |
| | | FFLEKKYAFQFLAGIKGFKNTTDNKFRATLETFTEHCCRLPKPKLDSS |
| | | DIKLDMLGELSRCPAPLFDLLDIEERKKFIREPEEVKPDESGDREEVQ |
| | | QVLMKRYDDRFPYFALRYFEEKNLLKGISFHIHIGRWIKSEHTKKIMG |
| | | AERDRRLLKDIRTFGELKEFSPEHAPDYWLRDGITPDDVDQFSPQYR |
| | | IVGNRIGIKLNYNGHNRWSVPDKEINVKPDAIISTYEFLNLFLYEHLY |
| | | QKKLTGLSPAEFIQDYLDRFNNFLSEFKAGHIRPVGDFSLEKRRGQG |
| | | DEPDLTARRKSLQKELDRFVLKGKDLPDKIREYLLGYKQKSEKKQAK |
| | | WILGGMIKETVYWRNKAEQSPEKMRSGDMAQQLARDIIFLTPPHT |
| | | VKEHKQKLNSLEYDVLQYALAYFSSNREKLYSFFKEHQLTVKGDRAH |
| | | PFLYKIRLDECQGILDFFIVYMQQKEKWLGWLDRNLKSPRLNEEEFF |
| | | NTYSYFIKTDTKRAIEMDYESCPNYLPRGIFNEPIAKALQKAGVKIKDE |
| | | DNASYALSVYSNGKTQPFYNKERYYNKGIFRMEELPEKLQPKELLGKI |
| | | QWTIKSSGKDTEEFRSLQNLKNRILNTEKEIRYVQSTDRALWIMVAD |
| | | LFPETFELRPDDLECIGHDLSDDLLSRPYQMKEKVYNYTITDYLPIKRY |
| | | GEFRRFLKDRRLENLLTYFEEGVPLHREALVAELEAYDLQRKNLLEIIY |
| | | RFEKLVFDRHRHELTFSGEGENQYVNHWDYLDFVARKYGLSAEVKE |
| | | LNSERFTELRNKMLHNQIPYQLWIKEAIAAREENTVCGRIMGMIGEI |
| | | YERMTTEIEKQMQV |
| 232 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 233 | Cas13 | MENKTSLGNNIYYNPFKPQDKSYFAGYFNAAMENTDSVFRELGKRL |
| | | KGKEYTSENFFDAIFKENISLVEYERYVKLLSDYFPMARLLDKKEVPIK |
| | | ERKENFKKNFKGIIKAVRDLRNFYTHKEHGEVEITDEIFGVLDEMLKS |
| | | TVLTVKKKKVKTDKTKEILKKSIEKQLDILCQKKLEYLRDTARKIEEKRR |
| | | NQRERGEKELVAPFKYSDKRDDLIAAIYNDAFDVYIDKKKDSLKESSK |
| | | AKYNTKSDPQQEEGDLKIPISKNGVVFLLSLFLTKQEIHAFKSKIAGFK |
| | | ATVIDEATVSEATVSHGKNSICFMATHEIFSHLAYKKLKRKVRTAEIN |
| | | YGEAENAEQLSVYAKETLMMQMLDELSKVPDVVYQNLSEDVQKTF |
| | | IEDWNEYLKENNGDVGTMEEEQVIHPVIRKRYEDKFNYFAIRFLDEF |
| | | AQFPTLRFQVHLGNYLHDSRPKENLISDRRIKEKITVFGRLSELEHKKA |
| | | LFIKNTETNEDREHYWEIFPNPNYDFPKENISVNDKDFPIAGSILDRE |
| | | KQPVAGKIGIKVKLLNQQYVSEVDKAVKAHQLKQRKASKPSIQNIIEE |
| | | IVPINESNPKEAIVFGGQPTAYLSMNDIHSILYEFFDKWEKKKEKLEK |
| | | KGEKELRKEIGKELEKKIVGKIQAQIQQIIDKDTNAKILKPYQDGNSTA |
| | | IDKEKLIKDLKQEQNILOKLKDEQTVREKEYNDFIAYQDKNREINKVR |
| | | DRNHKQYLKDNLKRKYPEAPARKEVLYYREKGKVAVWLANDIKRF |
| | | MPTDFKNEWKGEQHSLLOKSLAYYEQCKEELKNLLPEKVFQHLPFK |
| | | LGGYFQQKYLYQFYTCYLDKRLEYISGLVQQAENFKSENKVFKKVEN |
| | | ECFKFLKKQNYTHKELDARVQSILGYPIFLERGFMDEKPTIIKGKTFKG |
| | | NEALFADWFRYYKEYQNFQTFYDTENYPLVELEKKQADRKRKTKIYQ |
| | | QKKNDVFTLLMAKHIFKSVFKQDSIDQFSLEDLYQSREERLGNQERA |
| | | RQTGERNTNYIWNKTVDLKLCDGKITVENVKLKNVGDFIKYEYDQR |
| | | VQAFLKYEENIEWQAFLIKESKEEENYPYVVEREIEQYEKVRREELLKE |
| | | VHLIEEYILEKVKDKEILKKGDNQNFKYYILNGLLKQLKNEDVESYKVF |
| | | NLNTEPEDVNINQLKQEATDLEQKAFVLTYIRNKFAHNQLPKKEFW |
| | | DYCQEKYGKIEKEKTYAEYFAEVFKKEKEALIK |
| 234 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSOI |
| 235 | Cas13 | MTEQSERPYNGTYYTLEDKHFWAAFLNLARHNAYITLTHIDRQLAYS |
| | | KADITNDQDVLSFKALWKNLDNDLERKSRLRSLILKHFSFLEGAAYG |
| | | KKLFESKSSGNKSSKNKELTKKEKEELQANALSLDNLKSILFDFLQKLK |
| | | DFRNYYSHYRHSGSSELPLFDGNMLQRLYNVFDVSVQRVKRDHEH |
| | | NDKVDPHYHFNHLVRKGKKDRYGHNDNPSFKHHFVDSEGMVTEA |
| | | GLLFFVSLFLEKRDAIWMQKKIRGFKGGTGPYEQMTNEVFCRSRISL |
| | | PKLKLESLRTDDWMLLDMLNELVRCPKPLYDRLREKDRACFRVPVDI |
| | | LPDEDDTDGGGEDPFKNTLVRHQDRFPYFALRYFDLKKVFTSLRFHI |
| | | DLGTYHFAIYKKMIGEQPEDRHLTRNLYGFGRIQDFAEEHRPEEWK |
| | | RLVRDLDYLETGDKPYISQTTPHYHIEKGKIGLRFVPEGQHLWPSPEV |
| | | GTTRTGRSKCAQDKRLTAEAFLSVHELMPMMFYYFLLREKYSEEVS |
| | | AEKVQGRIKRVIEDVYAIYDAFARDEINTLKELDTCLADKGIRRGHLP |
| | | KQMITILSQERKDMKEKIRKKLQEMIADTDHRLDMLDRQTDRKIRI |
| | | GRKNAGLPKSGVIADWLVRDMMRFQPVAKDASGKPLNNSKANST |
| | | EYRMLQRALALFGGEKERLTPYFRQMNLTGGNNPHPFLHETRWES |
| | | HTNILSFYRSYLRARKAFLERIGRSDRVENCPFLLLKEPKTDRQTLVAG |
| | | WKDEFHLPRGIFTEAVRDCLIEMGYDEVGSYREVGFMAKAVPLYFE |
| | | RACEDRVQPFYDSPFNVGNSLKPKKGRFLSKEDRAEEWERGMERF |
| | | RDLEAWSHSAARRIKDAFAGIEYASPGNKKKIEQLLRDLSLWEAFES |
| | | KLKVRADKINLAKLKKEILEAQEHPYHDFKSWQKFERELRLVKNQDII |
| | | TWMMCRDLMEENKVEGLDTGTLYLKDIRPNVQEQGSLNVLNRVK |
| | | PMRLPVVVYRADSRGHVHKEAPLATVYIEERNTKLLKQGNFKSFVK |
| | | DRRLNGLFSFVDTGGLAMEQYPISKLRVEYELAKYQTARVCVFELTL |
| | | RLEESLLSRYPHLPDESFREMLESWSDPLLAKWPELHGKVRLLIAVR |
| | | NAFSHNQYPMYDEAVFSSIRKYDPSSPDAIEERMGLNIAHRLSEEVK |
| | | QAKETVERIIQA |
| 236 | Cas13 | MEKPLPPNVYTLKHKFFWGAFLNIARHNAFITICHINEQLGLTTPPN |
| | | DDKIADVVCGTWNNILNNDHDLLKKSQLTELILKHFPFLAAMCYHP |
| | | PKKEGKKKGSQKEQQKEKENEAQSQAEALNPSELIKALKTLVKQLRT |
| | | LRNYYSHYKHKKPDAEKDIFKHLYKAFDASLRMVKEDYKAHFTVNLT |
| | | QDFAHLNRKGKNKQDNPKFDRYRFEKDGFFTESGLLFFTNLFLDKR |
| | | DAYWMLKKVSGFKASHKQSEKMTTEVFCRSRILLPKLRLESRYDHN |
| | | QMLLDMLSELSRCPKLLYEKLSEKDKKHFQVEADGFLDEIEEEQNPF |
| | | KDALIRHQDRFPYFALRYLDLNESFKSIRFQVDLGTYHYCIYDKKIGDE |
| | | QEKRHLTRTLLSFGRLQDFTEINRPQEWKALTKDLDYKETSKQPFISK |
| | | TTPHYHITDNKIGFRLGTSKELYPSLEVKDGANRIAKYPYNSDFVAHA |
| | | FISVHELLPLMFYQHLTGKSEDLLKETVRHIQRIYKDFEEERINTIEDLE |
| | | KANQGRLPLGAFPKQMLGLLQNKQPDLSEKAKIKIEKLIAETKLLSHR |
| | | LNTKLKSSPKLGKRREKLIKTGDWLVKDFMRFQPVAYDVQNQPIESS |
| | | KANSTEFQLIQRALALYGGEKNRLEGYFKQTNLIGNTNPHPFLNKFN |
| | | WKACRNLVDFYQQYLEQREKFLEAIKNQPWEPYQYCLLLKIPKENR |
| | | KNLVKGWEQGGISLPRGLFTEAIRETLSEDLTLSKPIRKEIKKHGRVGF |
| | | ISRAITLYFRERYQDDHQSFYNLPYELEAKASTPKPPLPKKREYVLRAE |
| | | HYEYWQQNKPQSPTELORLELHTSDRWKDYLLYKRWQHLEKKLRL |
| | | YRNQDVMLWLMTLELTKNHFKELKLNYHQLKLENLAVNVQEADAK |
| | | LNPLNQTLPMVLPVKVYPATAFGEVQYQETPIRTVYIREEQTKALKM |
| | | GNFKALVKDRRLNGLFSFIKEENDTQKHPISQLRLRRELEIYQSLRVD |
| | | AFKETLSLEEKLLNKHASLSSLENEFRTLLEEWKKKYAASSMVTDEHI |
| | | AFIASVRNAFCHNQYPFYKETLHAPILLFTVAQPTTEEKDGLGIAEALL |
| | | RVLREYCEIVKSQI |
| 237 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 238 | Cas13 | MENKTSLGNNIYYNPFKPQDKSYFAGYFNAAMENTDSVFRELGKRL |
| | | KGKEYTSENFFDAIFKENISLVEYERYVKLLSDYFPMARLLDKKEVPIK |
| | | ERKENFKKNFKGIIKAVRDLRNFYTHKEHGEVEITDEIFGVLDEMLKS |
| | | TVLTVKKKKVKTDKTKEILKKSIEKQLDILCOKKLEYLRDTARKIEEKRR |
| | | NQRERGEKELVAPFKYSDKRDDLIAAIYNDAFDVYIDKKKDSLKESSK |
| | | AKYNTKSDPQQEEGDLKIPISKNGVVFLLSLFLTKQEIHAFKSKIAGFK |
| | | ATVIDEATVSEATVSHGKNSICFMATHEIFSHLAYKKLKRKVRTAEIN |
| | | YGEAENAEQLSVYAKETLMMQMLDELSKVPDVVYQNLSEDVQKTF |
| | | IEDW/NEYLKENNGDVGTMEEEQVIHPVIRKRYEDKFNYFAIRFLDEF |
| | | AQFPTLRFQVHLGNYLHDSRPKENLISDRRIKEKITVFGRLSELEHKKA |
| | | LFIKNTETNEDREHYWEIFPNPNYDFPKENISVNDKDFPIAGSILDRE |
| | | KQPVAGKIGIKVKLLNQQYVSEVDKAVKAHQLKQRKASKPSIQNIIEE |
| | | IVPINESNPKEAIVFGGQPTAYLSMNDIHSILYEFFDKWEKKKEKLEK |
| | | KGEKELRKEIGKELEKKIVGKIQAQIQQIIDKDTNAKILKPYQDGNSTA |
| | | IDKEKLIKDLKQEQNILQKLKDEQTVREKEYNDFIAYQDKNREINKVR |
| | | DRNHKQYLKDNLKRKYPEAPARKEVLYYREKGKVAVWLANDIKRF |
| | | MPTDFKNEWKGEQHSLLQKSLAYYEQCKEELKNLLPEKVFQHLPFK |
| | | LGGYFQQKYLYQFYTCYLDKRLEYISGLVQQAENFKSENKVFKKVEN |
| | | ECFKFLKKQNYTHKELDARVQSILGYPIFLERGFMDEKPTIIKGKTFKG |
| | | NEALFADWFRYYKEYQNFQTFYDTENYPLVELEKKQADRKRKTKIYQ |
| | | QKKNDVFTLLMAKHIFKSVFKQDSIDQFSLEDLYQSREERLGNQERA |
| | | RQTGERNTNYIWNKTVDLKLCDGKITVENVKLKNVGDFIKYEYDOR |
| | | VQAFLKYEENIEWQAFLIKESKEEENYPYVVEREIEQYEKVRREELLKE |
| | | VHLIEEYILEKVKDKEILKKGDNQNFKYYILNGLLKQLKNEDVESYKVF |
| | | NLNTEPEDVNINQLKQEATDLEQKAFVLTYIRNKFAHNQLPKKEFW |
| | | DYCQEKYGKIEKEKTYAEYFAEVFKKEKEALIK |
| 239 | Cas13 | MFFSFHNAQRVIFKHLYKAFDASLRMVKEDYKAHFTVNLTRDFAHL |
| | | NRKGKNKQDNPDFNRYRFEKDGFFTESGLLFFTNLFLDKRDAYWM |
| | | LKKVSGFKASHKQREKMTTEVFCRSRILLPKLRLESRYDHNQMLLD |
| | | MLSELSRCPKLLYEKLSEENKKHFQVEADGFLDEIEEEQNPFKDTLIR |
| | | HQDRFPYFALRYLDLNESFKSIRFQVDLGTYHYCIYDKKIGDEQEKRH |
| | | LTRTLLSFGRLQDFTEINRPQEWKALTKDLDYKETSNQPFISKTTPHY |
| | | HITDNKIGFRLGTSKELYPSLEIKDGANRIAKYPYNSGFVAHAFISVHE |
| | | LLPLMFYQHLTGKSEDLLKETVRHIQRIYKDFEEERINTIEDLEKANQG |
| | | RLPLGAFPKQMLGLLQNKQPDLSEKAKIKIEKLIAETKLLSHRLNTKLK |
| | | SSPKLGKRREKLIKTGVLADWLVKDFMRFQPVAYDAQNQPIKSSKA |
| | | NSTEFWFIRRALALYGGEKNRLEGYFKQTNLIGNTNPHPFLNKFNW |
| | | KACRNLVDFYQQYLEQREKFLEAIKNQPWEPYQYCLLLKIPKENRKN |
| | | LVKGWEQGGISLPRGLFTEAIRETLSEDLMLSKPIRKEIKKHGRVGFIS |
| | | RAITLYFKEKYQDKHQSFYNLSYKLEAKAPLLKREEHYEYWQQNKPQ |
| | | SPTESQRLELHTSDRWKDYLLYKRWQHLEKKLRLYRNQDVMLWLM |
| | | TLELTKNHFKELNLNYHQLKLENLAVNVQEADAKLNPLNQTLPMVL |
| | | PVKVYPATAFGEVQYHKTPIRTVYIREEHTKALKMGNFKALVKDRRL |
| | | NGLFSFIKEENDTQKHPISQLRLRRELEIYQSLRVDAFKETLSLEEKLLN |
| | | KHTSLSSLENEFRALLEEWKKEYAASSMVTDEHIAFIASVRNAFCHN |
| | | QYPFYKEALHAPIPLFTVAQPTTEEKDGLGIAEALLKVLREYCEIVKSQI |
| 240 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSOI |
| 241 | Cas13 | MEKPLPPNVYTLKHKFFWGAFLNIARHNAFITICHINEQLGLTTPPN |
| | | DDKIADVVCGTWNNILNNDHDLLKKSQLTELILKHFPFLAAMCYHP |
| | | PKKEGKKKGSQKEQQKEKENEAQSQAEALNPSELIKALKTLVKQLRT |
| | | LRNYYSHYKHKKPDAEKDIFKHLYKAFDASLRMVKEDYKAHFTVNLT |
| | | QDFAHLNRKGKNKQDNPKFDRYRFEKDGFFTESGLLFFTNLFLDKR |
| | | DAYWMLKKVSGFKASHKQSEKMTTEVFCRSRILLPKLRLESRYDHN |
| | | QMLLDMLSELSRCPKLLYEKLSEKDKKHFQVEADGFLDEIEEEQNPF |
| | | KDALIRHQDRFPYFALRYLDLNESFKSIRFQVDLGTYHYCIYDKKIGDE |
| | | QEKRHLTRTLLSFGRLQDFTEINRPQEWKALTKDLDYKETSKQPFISK |
| | | TTPHYHITDNKIGFRLGTSKELYPSLEVKDGANRIAKYPYNSDFVAHA |
| | | FISVHELLPLMFYQHLTGKSEDLLKETVRHIQRIYKDFEEERINTIEDLE |
| | | KANQGRLPLGAFPKQMLGLLQNKQPDLSEKAKIKIEKLIAETKLLSHR |
| | | LNTKLKSSPKLGKRREKLIKTGVLADWLVKDFMRFQPVAYDVQNQP |
| | | IESSKANSTEFQLIQRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLTLSKPIRKEIKKHGRV |
| | | GFISRAITLYFRERYQDDHQSFYNLPYELEAKASTPKPPLPKKREYVLR |
| | | AEHYEYWQQNKPQSPTELQRLELHTSDRWKDYLLYKRWQHLEKKL |
| | | RLYRNQDVMLWLMTLELTKNHFKELKLNYHQLKLENLAVNVQEAD |
| | | AKLNPLNQTLPMVLPVKVYPATAFGEVQYQETPIRTVYIREEQTKAL |
| | | KMGNFKALVKDRRLNGLFSFIKEENDTQKHPISQLRLRRELEIYQSLR |
| | | VDAFKETLSLEEKLLNKHASLSSLENEFRTLLEEWKKKYAASSMVTDE |
| | | HIAFIASVRNAFCHNQYPFYKETLHAPILLFTVAQPTTEEKDGLGIAEA |
| | | LLRVLREYCEIVKSQI |
| 242 | Cas13 | MEKPLLPNVYTLKHKFFWGAFLNIARHNAFITICHINEQLGLKTPSN |
| | | DDKIVDWCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSOI |
| 243 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSOI |
| 244 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQO |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 245 | Cas13 | MFFSFHNAQRVIFKHLYKAFDASLRMVKEDYKAHFTVNLTRDFAHL |
| | | NRKGKNKQDNPDFNRYRFEKDGFFTESGLLFFTNLFLDKRDAYWM |
| | | LKKVSGFKASHKQREKMTTEVFCRSRILLPKLRLESRYDHNQMLLD |
| | | MLSELSRCPKLLYEKLSEENKKHFQVEADGFLDEIEEEQNPFKDTLIR |
| | | HQDRFPYFALRYLDLNESFKSIRFQVDLGTYHYCIYDKKIGDEQEKRH |
| | | LTRTLLSFGRLQDFTEINRPQEWKALTKDLDYKETSNQPFISKTTPHY |
| | | HITDNKIGFRLGTSKELYPSLEIKDGANRIAKYPYNSGFVAHAFISVHE |
| | | LLPLMFYQHLTGKSEDLLKETVRHIQRIYKDFEEERINTIEDLEKANQG |
| | | RLPLGAFPKQMLGLLQNKQPDLSEKAKIKIEKLIAETKLLSHRLNTKLK |
| | | SSPKLGKRREKLIKTGVLADWLVKDFMRFQPVAYDAQNQPIKSSKA |
| | | NSTEFWFIRRALALYGGEKNRLEGYFKQ.TNLIGNTNPHPFLNKFNW |
| | | KACRNLVDFYQQYLEQREKFLEAIKNQPWEPYQYCLLLKIPKENRKN |
| | | LVKGWEQGGISLPRGLFTEAIRETLSEDLMLSKPIRKEIKKHGRVGFIS |
| | | RAITLYFKEKYQDKHQSFYNLSYKLEAKAPLLKREEHYEYWQQNKPQ |
| | | SPTESQRLELHTSDRWKDYLLYKRWQHLEKKLRLYRNQDVMLWLM |
| | | TLELTKNHFKELNLNYHQLKLENLAVNVQEADAKLNPLNQTLPMVL |
| | | PVKVYPATAFGEVQYHKTPIRTVYIREEHTKALKMGNFKALVKDRRL |
| | | NGLFSFIKEENDTQKHPISQLRLRRELEIYQSLRVDAFKETLSLEEKLLN |
| | | KHTSLSSLENEFRALLEEWKKEYAASSMVTDEHIAFIASVRNAFCHN |
| | | QYPFYKEALHAPIPLFTVAQPTTEEKDGLGIAEALLKVLREYCEIVKSQI |
| 246 | Cas13 | MDTPNFSERIPVSLQSHPYYFAHYLNMARHNAYVILEYVNRELIKPG |
| | | KNLDEDNLIQSTVLKDGYFDRKPDELSHRNRLLVQHFPFLREAENEG |
| | | ARTCNPVSYKLKTALAALNQWRNNASHYPLNQNHEKDFDLQPFFS |
| | | FAIEACKKRMREVFQPDDFYLLETNEKQFYTLHNENGFTEKGLYCFIC |
| | | FFLEKKYAFQFLAGIKGFKNTTDNKFRATLETFTEHCCRLPKPKLDSS |
| | | DIKLDMLGELSRCPAPLFDLLDIEERKKFIREPEEVKPDESGDREEVQ |
| | | QVLMKRYDDRFPYFALRYFEEKNLLKGISFHIHIGRWIKSEHTKKIMG |
| | | AERDRRLLKDIRTFGELKEFSPEHAPDYWLRDGITPDDVDQFSPQYR |
| | | IVGNRIGIKLNYNGHNRWSVPDKEINVKPDAIISTYEFLNLFLYEHLY |
| | | QKKLTGLSPAEFIQDYLDRFNNFLSEFKAGHIRPVGDFSLEKRRGQG |
| | | DEPDLTARRKSLQKELDRFVLKGKDLPDKIREYLLGYKQKSEKKQAK |
| | | WILGGMIKETVYWRNKAEQSPEKMRSGDMAQQLARDIIFLTPPHT |
| | | VKEHKQKLNSLEYDVLQYALAYFSSNREKLYSFFKEHQLTVKGDRAH |
| | | PFLYKIRLDECQGILDFFIVYMQQKEKWLGWLDRNLKSPRLNEEEFF |
| | | NTYSYFIKTDTKRAIEMDYESCPNYLPRGIFNEPIAKALQKAGVKIKDE |
| | | DNASYALSVYSNGKTQPFYNKERYYNKGIFRMEELPEKLQPKELLGKI |
| | | QWTIKSSGKDTEEFRSLQNLKNRILNTEKEIRYVQSTDRALWIMVAD |
| | | LFPETFELRPDDLECIGHDLSDDLLSRPYQMKEKVYNYTITDYLPIKRY |
| | | GEFRRFLKDRRLENLLTYFEEGVPLHREALVAELEAYDLQRKNLLEIIY |
| | | RFEKLVFDRHRHELTFSGEGENQYVNHWDYLDFVARKYGLSAEVKE |
| | | LNSERFTELRNKMLHNQIPYQLWIKEAIAAREENTVCGRIMGMIGEI |
| | | YERMTTEIEKOMOV |
| 247 | Cas13 | MTEQNEKPYNGTYYTLEDKHFWAAFLNLARHNAYITLTHIDRQLAY |
| | | SKADITNDEDILFFKGQWKNLDNDLERKARLRSLILKHFFFLEGAAYG |
| | | KKLFESQSSGNKSSKKKELTKKEKEELQANALSLDNLKSILFDFLQKLK |
| | | DFRNYYSHYRHPESSELPLFDGNMLQRLYNVFDVSVQRVKRDHEH |
| | | NDKVDPHRHFNHLVRKGKKDKYGNNDNPFFKHHFVDREGKVTEA |
| | | GLLFFVSLFLEKRDAIWMQKKIRGFKGGTETYQQMTNEVFCRSRISL |
| | | PKLKLESLRTDDWMLLDMLNELVRCPKSLYDRLREEDRARFRVPVDI |
| | | LSDEDDTDGAEEDPFKNTLVRHQDRFPYFALRYFDLKKVFTSLRFHID |
| | | LGTYHFAIYKKNIGEQPEDRHLTRNLYGFGRIQDFAEEHRPEEWKRL |
| | | VRDLDYFETGDKPYITQTTPHYHIEKGKIGLRFVPEGQHLWPSPEVG |
| | | ATRTGRSKYAQDKRFTAEAFLSVHELMPMMFYYFLLREKYSEEASAE |
| | | RVQGRIKRVIEDVYAVYDAFARDEINTRDELDACLADKGIRRGHLPR |
| | | QMIAILSQKHKDMEEKVRKKLQEMIADTDHRLDMLDRQTDRKIRIG |
| | | RKNAGLPKSGVIADWLVRDMMRFQPVAKDTSGKPLNNSKANSTE |
| | | YRMLQRALALFGGEKERLTPYFRQMNLTGGNNPHPFLHETRWESH |
| | | TNILSFYRSYLKARKAFLQSIGRSDRVENHRFLLLKEPKTDRQTLVAG |
| | | WKGEFHLPRGIFTEAVRDCLIEMGLDEVRSYKEVGFMAKAVPLYFE |
| | | RASKDRVQPFYDYPFNVGNSLKPKKGRFLSKEKRAEEWESGKERFR |
| | | DLEAWSHSAARRIEDAFAGIENASRENKKKIEQLLQDLSLWETFESKL |
| | | KVKADKINIAKLKKEILEAKEHPYLDFKSWQKFERELRLVKNQDIITW |
| | | MMCRDLMEENKVEGLDTGTLYLKDIRTDVQEQGSLNVLNHVKPM |
| | | RLPVVVYRADSRGHVHKEQAPLATVYIEERDTKLLKQGNFKSFVKDR |
| | | RLNGLFSFVDTGGLAMEQYPISKLRVEYELAKYQTARVCAFEQTLELE |
| | | ESLLTRYPHLPDKNFRKMLESWSDPLLAKWPELHEKVRLLIAVRNAF |
| | | SHNQYPMYDEAVFSPIRKYDPSSPDAIEERMRLNIAHRLSEEVKQAK |
| | | ETVERIIQA |
| 248 | Cas13 | MDTPNFSERIPVSLQSHPYYFAHYLNMARHNAYVILEYVNRELIKPG |
| | | KNLDEDNLIQSTVLKDGYFDRKPDELSHRNRLLVQHFPFLREAENEG |
| | | ARTCNPVSYKLKTALAALNQWRNNASHYPLNQNHEKDFDLQPFFS |
| | | FAIEACKKRMREVFQPDDFYLLETNEKQFYTLHNENGFTEKGLYCFIC |
| | | FFLEKKYAFQFLAGIKGFKNTTDNKFRATLETFTEHCCRLPKPKLDSS |
| | | DIKLDMLGELSRCPAPLFDLLDIEERKKFIREPEEVKPDESGDREEVQ |
| | | QVLMKRYDDRFPYFALRYFEEKNLLKGISFHIHIGRWIKSEHTKKIMG |
| | | AERDRRLLKDIRTFGELKEFSPEHAPDYWLRDGITPDDVDQFSPQYR |
| | | IVGNRIGIKLNYNGHNRWSVPDKEINVKPDAIISTYEFLNLFLYEHLY |
| | | QKKLTGLSPAEFIQDYLDRFNNFLSEFKAGHIRPVGDFSLEKRRGQG |
| | | DEPDLTARRKSLQKELDRFVLKGKDLPDKIREYLLGYKQKSEKKQAK |
| | | WILGGMIKETVYWRNKAEQSPEKMRSGDMAQQLARDIIFLTPPHT |
| | | VKEHKQKLNSLEYDVLQYALAYFSSNREKLYSFFKEHQLTVKGDRAH |
| | | PFLYKIRLDECQGILDFFIVYMQQKEKWLGWLDRNLKSPRLNEEEFF |
| | | NTYSYFIKTDTKRAIEMDYESCPNYLPRGIFNEPIAKALQKAGVKIKDE |
| | | DNASYALSVYSNGKTQPFYNKERYYNKGIFRMEELPEKLQPKELLGKI |
| | | QWTIKSSGKDTEEFRSLQNLKNRILNTEKEIRYVQSTDRALWIMVAD |
| | | LFPETFELRPDDLECIGHDLSDDLLSRPYQMKEKVYNYTITDYLPIKRY |
| | | GEFRRFLKDRRLENLLTYFEEGVPLHREALVAELEAYDLQRKNLLEIIY |
| | | RFEKLVFDRHRHELTFSGEGENQYVNHWDYLDFVARKYGLSAEVKE |
| | | LNSERFTELRNKMLHNQIPYQLWIKEAIAAREENTVCGRIMGMIGEI |
| | | YERMTTEIEKOMOV |
| 249 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEMEHLRRYTRKKGRVVLKTEDDHFYYTLANNNDLSEKGY |
| | | AFFISMFLERKYSYLFLKKLSGFKRGDSLQYRLTLEVFTALSTKPPVERL |
| | | RTTKDTKQDRALDILNELSRIPIELYQTLEPKYREMYNATLQPTDAED |
| | | PYGLPDRSRIRFRSRFEAFALHFLDKQADFKEIGFYTYLGNYFHNGYQ |
| | | KTRVDRETKDRYINFQLAGFCKNIQDISAKKLSEALNVKSIDISTDSIP |
| | | DINSFEPYLVQSTPHYIVNGNNIGIKVLPEGKDTYPTIDEKGAKMPIA |
| | | DFWLSKYELPAMLFYTYLRNNNIHKSHCPLSVKDIIERSIHKSTKOKH |
| | | PEERSELMLRRVMKAIFWTDSKLNEVERIKSQKSAFGKRQHEILKAG |
| | | RIAETLVRDMLWLQPSKNNGRDKVTEPNFQAIQVSLAYFGIRRNDL |
| | | TEIFTRAGLINSSNPHPFLAQIGTNYTSLIEFYIAYLKERKVYFSRIOKKI |
| | | LQGKLNIQCHPLRDLQREPNKPQEKEEAIFLPRGLFNEAIINCLKKSKL |
| | | KQLIESPTREKSPALNVSYLILNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRINSLNKVLSKVKRN |
| | | NSISSSVKIQPYENYKRECLDFEEAQIQIIPIIHSFEIAMVSMFPDLKKA |
| | | TPGNYYDFNELITEYEKRTKQKIDSSFLIKTRNMFLHDKYEAECIKEIS |
| | | DDFVYAKKIIAEFKMKIENIKLEDFSNDSSA |
| 250 | Cas13 | MEKPLPPNVYTLKHKFFWGAFLNIARHNAFITICHINEQLGLTTPPN |
| | | DDKIADVVCGTWNNILNNDHDLLKKSQLTELILKHFPFLAAMCYHP |
| | | PKKEGKKKGSQKEQQKEKENEAQSQAEALNPSELIKALKTLVKQLRT |
| | | LRNYYSHYKHKKPDAEKDIFKHLYKAFDASLRMVKEDYKAHFTVNLT |
| | | QDFAHLNRKGKNKQDNPKFDRYRFEKDGFFTESGLLFFTNLFLDKR |
| | | DAYWMLKKVSGFKASHKQSEKMTTEVFCRSRILLPKLRLESRYDHN |
| | | QMLLDMLSELSRCPKLLYEKLSEKDKKHFQVEADGFLDEIEEEQNPF |
| | | KDALIRHQDRFPYFALRYLDLNESFKSIRFQVDLGTYHYCIYDKKIGDE |
| | | QEKRHLTRTLLSFGRLQDFTEINRPQEWKALTKDLDYKETSKQPFISK |
| | | TTPHYHITDNKIGFRLGTSKELYPSLEVKDGANRIAKYPYNSDFVAHA |
| | | FISVHELLPLMFYQHLTGKSEDLLKETVRHIQRIYKDFEEERINTIEDLE |
| | | KANQGRLPLGAFPKQMLGLLQNKQPDLSEKAKIKIEKLIAETKLLSHR |
| | | LNTKLKSSPKLGKRREKLIKTGVLADWLVKDFMRFQPVAYDVQNQP |
| | | IESSKANSTEFQLIQRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLTLSKPIRKEIKKHGRV |
| | | GFISRAITLYFRERYQDDHQSFYNLPYELEAKASTPKPPLPKKREYVLR |
| | | AEHYEYWQQNKPQSPTELQRLELHTSDRWKDYLLYKRWQHLEKKL |
| | | RLYRNQDVMLWLMTLELTKNHFKELKLNYHQLKLENLAVNVQEAD |
| | | AKLNPLNQTLPMVLPVKVYPATAFGEVQYQETPIRTVYIREEQTKAL |
| | | KMGNFKALVKDRRLNGLFSFIKEENDTQKHPISQLRLRRELEIYQSLR |
| | | VDAFKETLSLEEKLLNKHASLSSLENEFRTLLEEWKKKYAASSMVTDE |
| | | HIAFIASVRNAFCHNQYPFYKETLHAPILLFTVAQPTTEEKDGLGIAEA |
| | | LLRVLREYCEIVKSOI |
| 251 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQO |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSOI |
| 252 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 253 | Cas13 | MFFSFHNAQRVIFKHLYKAFDASLRMVKEDYKAHFTVNLTRDFAHL |
| | | NRKGKNKQDNPDFNRYRFEKDGFFTESGLLFFTNLFLDKRDAYWM |
| | | LKKVSGFKASHKQREKMTTEVFCRSRILLPKLRLESRYDHNQMLLD |
| | | MLSELSRCPKLLYEKLSEENKKHFQVEADGFLDEIEEEQNPFKDTLIR |
| | | HQDRFPYFALRYLDLNESFKSIRFQVDLGTYHYCIYDKKIGDEQEKRH |
| | | LTRTLLSFGRLQDFTEINRPQEWKALTKDLDYKETSNQPFISKTTPHY |
| | | HITDNKIGFRLGTSKELYPSLEIKDGANRIAKYPYNSGFVAHAFISVHE |
| | | LLPLMFYQHLTGKSEDLLKETVRHIQRIYKDFEEERINTIEDLEKANQG |
| | | RLPLGAFPKQMLGLLQNKQPDLSEKAKIKIEKLIAETKLLSHRLNTKLK |
| | | SSPKLGKRREKLIKTGVLADWLVKDFMRFQPVAYDAQNQPIKSSKA |
| | | NSTEFWFIRRALALYGGEKNRLEGYFKQ.TNLIGNTNPHPFLNKFNW |
| | | KACRNLVDFYQQYLEQREKFLEAIKNQPWEPYQYCLLLKIPKENRKN |
| | | LVKGWEQGGISLPRGLFTEAIRETLSEDLMLSKPIRKEIKKHGRVGFIS |
| | | RAITLYFKEKYQDKHQSFYNLSYKLEAKAPLLKREEHYEYWQQNKPQ |
| | | SPTESQRLELHTSDRWKDYLLYKRWQHLEKKLRLYRNQDVMLWLM |
| | | TLELTKNHFKELNLNYHQLKLENLAVNVQEADAKLNPLNQTLPMVL |
| | | PVKVYPATAFGEVQYHKTPIRTVYIREEHTKALKMGNFKALVKDRRL |
| | | NGLFSFIKEENDTQKHPISQLRLRRELEIYQSLRVDAFKETLSLEEKLLN |
| | | KHTSLSSLENEFRALLEEWKKEYAASSMVTDEHIAFIASVRNAFCHN |
| | | QYPFYKEALHAPIPLFTVAQPTTEEKDGLGIAEALLKVLREYCEIVKSQI |
| 254 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 255 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 256 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQO |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSOI |
| 257 | Cas13 | MDTPNFSERIPVSLQSHPYYFAHYLNMARHNAYVILEYVNRELIKPG |
| | | KNLDEDNLIQSTVLKDGYFDRKPDELSHRNRLLVQHFPFLREAENEG |
| | | ARTCNPVSYKLKTALAALNQWRNNASHYPLNQNHEKDFDLQPFFS |
| | | FAIEACKKRMREVFQPDDFYLLETNEKQFYTLHNENGFTEKGLYCFIC |
| | | FFLEKKYAFQFLAGIKGFKNTTDNKFRATLETFTEHCCRLPKPKLDSS |
| | | DIKLDMLGELSRCPAPLFDLLDIEERKKFIREPEEVKPDESGDREEVQ |
| | | QVLMKRYDDRFPYFALRYFEEKNLLKGISFHIHIGRWIKSEHTKKIMG |
| | | AERDRRLLKDIRTFGELKEFSPEHAPDYWLRDGITPDDVDQFSPQYR |
| | | IVGNRIGIKLNYNGHNRWSVPDKEINVKPDAIISTYEFLNLFLYEHLY |
| | | QKKLTGLSPAEFIQDYLDRFNNFLSEFKAGHIRPVGDFSLEKRRGQG |
| | | DEPDLTARRKSLQKELDRFVLKGKDLPDKIREYLLGYKQKSEKKQAK |
| | | WILGGMIKETVYWRNKAEQSPEKMRSGDMAQQLARDIIFLTPPHT |
| | | VKEHKQKLNSLEYDVLQYALAYFSSNREKLYSFFKEHQLTVKGDRAH |
| | | PFLYKIRLDECQGILDFFIVYMQQKEKWLGWLDRNLKSPRLNEEEFF |
| | | NTYSYFIKTDTKRAIEMDYESCPNYLPRGIFNEPIAKALQKAGVKIKDE |
| | | DNASYALSVYSNGKTQPFYNKERYYNKGIFRMEELPEKLQPKELLGKI |
| | | QWTIKSSGKDTEEFRSLQNLKNRILNTEKEIRYVQSTDRALWIMVAD |
| | | LFPETFELRPDDLECIGHDLSDDLLSRPYQMKEKVYNYTITDYLPIKRY |
| | | GEFRRFLKDRRLENLLTYFEEGVPLHREALVAELEAYDLQRKNLLEIIY |
| | | RFEKLVFDRHRHELTFSGEGENQYVNHWDYLDFVARKYGLSAEVKE |
| | | LNSERFTELRNKMLHNQIPYQLWIKEAIAAREENTVCGRIMGMIGEI |
| | | YERMTTEIEKOMOV |
| 258 | Cas13 | MFFSFHNAQRVIFKHLYKAFDASLRMVKEDYKAHFTVNLTRDFAHL |
| | | NRKGKNKQDNPDFNRYRFEKDGFFTESGLLFFTNLFLDKRDAYWM |
| | | LKKVSGFKASHKQREKMTTEVFCRSRILLPKLRLESRYDHNQMLLD |
| | | MLSELSRCPKLLYEKLSEENKKHFQVEADGFLDEIEEEQNPFKDTLIR |
| | | HQDRFPYFALRYLDLNESFKSIRFQVDLGTYHYCIYDKKIGDEQEKRH |
| | | LTRTLLSFGRLQDFTEINRPQEWKALTKDLDYKETSNQPFISKTTPHY |
| | | HITDNKIGFRLGTSKELYPSLEIKDGANRIAKYPYNSGFVAHAFISVHE |
| | | LLPLMFYQHLTGKSEDLLKETVRHIQRIYKDFEEERINTIEDLEKANQG |
| | | RLPLGAFPKQMLGLLQNKQPDLSEKAKIKIEKLIAETKLLSHRLNTKLK |
| | | SSPKLGKRREKLIKTGVLADWLVKDFMRFQPVAYDAQNQPIKSSKA |
| | | NSTEFWFIRRALALYGGEKNRLEGYFKQTNLIGNTNPHPFLNKFNW |
| | | KACRNLVDFYQQYLEQREKFLEAIKNQPWEPYQYCLLLKIPKENRKN |
| | | LVKGWEQGGISLPRGLFTEAIRETLSEDLMLSKPIRKEIKKHGRVGFIS |
| | | RAITLYFKEKYQDKHQSFYNLSYKLEAKAPLLKREEHYEYWQQNKPQ |
| | | SPTESQRLELHTSDRWKDYLLYKRWQHLEKKLRLYRNQDVMLWLM |
| | | TLELTKNHFKELNLNYHQLKLENLAVNVQEADAKLNPLNQTLPMVL |
| | | PVKVYPATAFGEVQYHKTPIRTVYIREEHTKALKMGNFKALVKDRRL |
| | | NGLFSFIKEENDTQKHPISQLRLRRELEIYQSLRVDAFKETLSLEEKLLN |
| | | KHTSLSSLENEFRALLEEWKKEYAASSMVTDEHIAFIASVRNAFCHN |
| | | QYPFYKEALHAPIPLFTVAQPTTEEKDGLGIAEALLKVLREYCEIVKSQI |
| 259 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ. |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQO |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 260 | Cas13 | MTNTPKRRTLHRHPSYFGAFLNIARHNAFMIMEHLSTKYDMEDKN |
| | | TLDEAQLPNAKLFGCLKKRYGKPDVTEGVSRDLRRYFPFLNYPLFLHL |
| | | EKQQNAEQAATYDINPEDIEFTLKGFFRLLNQMRNNYSHYISNTDY |
| | | GKFDKLPVQDIYEAAIFRLLDRGKHTKRFDVFESKHTRHLESNNSEYR |
| | | PRSLANSPDHENTVAFVTCLFLERKYAFPFLSRLDCFRSTNDAAEGD |
| | | PLIRKASHECYTMFCCRLPQPKLESSDILLDMVNELGRCPSALYNLLS |
| | | EEDQARFHIKREEITGFEEDPDEELEQEIVLKRHSDRFPYFALRYFDDT |
| | | EAFQTLRFDVYLGRWRTKPVYKKRIYGQERDRVLTQSIRTFTRLSRLL |
| | | PIYENVKHDAVRQNEEDGKLVNPDVTSQFHKSWIQIESDDRAFLSD |
| | | RIEHFSPHYNFGDQVIGLKFINPDRYAAIQNVFPKLPGEEKKDKDAKL |
| | | VNETADAIISTHEIRSLFLYHYLSKKPISAGDERRFIQVDTETFIKQYIDT |
| | | IKLFFEDIKSGELQPIADPPNYQKNEPLPYVRGDKEKTQEERAQYRER |
| | | QKEIKERRKELNTLLQNRYGLSIQYIPSRLREYLLGYKKVPYEKLALQKL |
| | | RAQRKEVKKRIKDIEKMRTPRVGEQATWLAEDIVFLTPPKMHTPER |
| | | KTTKHPQKLNNDQFRIMQSSLAYFSVNKKAIKKFFQKETGIGLSNRE |
| | | TSHPFLYRIDVGRCRGILDFYTGYLKYKMDWLDDAIKKVDNRKHGK |
| | | KEAKKYEKYLPSSIQHKTPLELDYTRLPVYLPRGLFKKAIVKALAAHAD |
| | | FQVEPEEDNVIFCLDQLLDGDTQDFYNWQRYYRSALTEKETDNQLV |
| | | LAHPYAEQILGTIKTLEGKQKNNKLGNKAKQKIKDELIDLKRAKRRLL |
| | | DREQYLRAVQAEDRALWLMIQERQKQKAEHEEIAFDQLDLKNITKI |
| | | LTESIDARLRIPDTKVDITDKLPLRRYGDLRRVAKDRRLVNLASYYHV |
| | | AGLSEIPYDLVKKELEEYDRRRVAFFEHVYQFEKEVYDRYAAELRNEN |
| | | PKGESTYFSHWEYVAVAVKHSADTHFNELFKEKVMQLRNKFHHNE |
| | | FPYFDWLLPEVEKASAALYADRVFDVAEGYYQKMRKLMRQ |
| 261 | Cas13 | MTNTPKRRTLHRHPSYFGAFLNIARHNAFMIMEHLSTKYDMEDKN |
| | | TLDEAQLPNAKLFGCLKKRYGKPDVTEGVSRDLRRYFPFLNYPLFLHL |
| | | EKQQNAEQAATYDINPEDIEFTLKGFFRLLNQMRNNYSHYISNTDY |
| | | GKFDKLPVQDIYEAAIFRLLDRGKHTKRFDVFESKHTRHLESNNSEYR |
| | | PRSLANSPDHENTVAFVTCLFLERKYAFPFLSRLDCFRSTNDAAEGD |
| | | PLIRKASHECYTMFCCRLPQPKLESSDILLDMVNELGRCPSALYNLLS |
| | | EEDQARFHIKREEITGFEEDPDEELEQEIVLKRHSDRFPYFALRYFDDT |
| | | EAFQTLRFDVYLGRWRTKPVYKKRIYGQERDRVLTQSIRTFTRLSRLL |
| | | PIYENVKHDAVRQNEEDGKLVNPDVTSQFHKSWIQIESDDRAFLSD |
| | | RIEHFSPHYNFGDQVIGLKFINPDRYAAIQNVFPKLPGEEKKDKDAKL |
| | | VNETADAIISTHEIRSLFLYHYLSKKPISAGDERRFIQVDTETFIKQYIDT |
| | | IKLFFEDIKSGELQPIADPPNYQKNEPLPYVRGDKEKTQEERAQYRER |
| | | QKEIKERRKELNTLLQNRYGLSIQYIPSRLREYLLGYKKVPYEKLALOKL |
| | | RAQRKEVKKRIKDIEKMRTPRVGEQATWLAEDIVFLTPPKMHTPER |
| | | KTTKHPQKLNNDQFRIMQSSLAYFSVNKKAIKKFFQKETGIGLSNRE |
| | | TSHPFLYRIDVGRCRGILDFYTGYLKYKMDWLDDAIKKVDNRKHGK |
| | | KEAKKYEKYLPSSIQHKTPLELDYTRLPVYLPRGLFKKAIVKALAAHAD |
| | | FQVEPEEDNVIFCLDQLLDGDTQDFYNWQRYYRSALTEKETDNQLV |
| | | LAHPYAEQILGTIKTLEGKQKNNKLGNKAKQKIKDELIDLKRAKRRLL |
| | | DREQYLRAVQAEDRALWLMIQERQKQKAEHEEIAFDQLDLKNITKI |
| | | LTESIDARLRIPDTKVDITDKLPLRRYGDLRRVAKDRRLVNLASYYHV |
| | | AGLSEIPYDLVKKELEEYDRRRVAFFEHVYQFEKEVYDRYAAELRNEN |
| | | PKGESTYFSHWEYVAVAVKHSADTHFNELFKEKVMQLRNKFHHNE |
| | | FPYFDWLLPEVEKASAALYADRVFDVAEGYYQKMRKLMRQ |
| 262 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 263 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 264 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQO |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSOI |
| 265 | Cas13 | MESIKNSQKSTGKTLQKDPPYFGLYLNMALLNVRKVENHIRKWLGD |
| | | VALLPEKSGFHSLLTTDNLSSAKWTRFYYKSRKFLPFLEMFDSDKKSY |
| | | ENRRETTECLDTIDRQKISSLLKEVYGKLQDIRNAFSHYHIDDQSVKH |
| | | TALIISSEMHRFIENAYSFALQKTRARFTGVFVETDFLQAEEKGDNKK |
| | | FFAIGGNEGIKLKDNALIFLICLFLDREEAFKFLSRATGFKSTKEKGFLA |
| | | VRETFCALCCRQPHERLLSVNPREALLMDMLNELNRCPDILFEMLD |
| | | EKDQKSFLPLLGEEEQAHILENSLNDELCEAIDDPFEMIASLSKRVRYK |
| | | NRFPYLMLRYIEEKNLLPFIRFRIDLGCLELASYPKKMGEENNYERSVT |
| | | DHAMAFGRLTDFHNEDAVLQQITKGITDEVRFSLYAPRYAIYNNKIG |
| | | FVRTGGSDKISFPTLKKKGGEGHCVAYTLQNTKSFGFISIYDLRKILLLS |
| | | FLDKDKAKNIVSGLLEQCEKHWKDLSENLFDAIRTELQKEFPVPLIRY |
| | | TLPRSKGGKLVSSKLADKQEKYESEFERRKEKLTEILSEKDFDLSQIPRR |
| | | MIDEWLNVLPTSREKKLKGYVETLKLDCRERLRVFEKREKGEHPVPP |
| | | RIGEMATDLAKDIIRMVIDQGVKQRITSAYYSEIQRCLAQYAGDDNR |
| | | RHLDSIIRELRLKDTKNGHPFLGKVLRPGLGHTEKLYQRYFEEKKEWL |
| | | EATFYPAASPKRVPRFVNPPTGKQKELPLIIRNLMKERPEWRDWKQ. |
| | | RKNSHPIDLPSQLFENEICRLLKDKIGKEPSGKLKWNEMFKLYWDKE |
| | | FPNGMQRFYRCKRRVEVFDKVVEYEYSEEGGNYKKYYEALIDEVVR |
| | | QKISSSKEKSKLQVEDLTLSVRRVFKRAINEKEYQLRLLCEDDRLLFMA |
| | | VRDLYDWKEAQLDLDKIDNMLGEPVSVSQVIQLEGGQPDAVIKAE |
| | | CKLKDVSKLMRYCYDGRVKGLMPYFANHEATQEQVEMELRHYED |
| | | HRRRVFNWVFALEKSVLKNEKLRRFYEESQGGCEHRRCIDALRKASL |
| | | VSEEEYEFLVHIRNKSAHNQFPDLEIGKLPPNVTSGFCECIWSKYKAII |
| | | CRIIPFIDPERRFFGKLLEOK |
| 266 | Cas13 | MEKPLPPNVYTLKHKFFWGAFLNIARHNAFITICHINEQLGLTTPPN |
| | | DDKIADVVCGTWNNILNNDHDLLKKSQLTELILKHFPFLAAMCYHP |
| | | PKKEGKKKGSQKEQQKEKEKEKENEAQSQAEALNPSELIKVLKTIVK |
| | | QLRTLRNYYSHHSHKKPDTEKDIFKHLYKAFDASLRMVKEDYKAHFT |
| | | VNLTRDFAHLNRKGKNKQDNPDFNRYRFEKDGFFTESGLLFFTNLFL |
| | | DKRDAYWMLKKVSGFKASHKQREKMTTEVFCRSHILLPKLRLESRY |
| | | DHNQMLLDMLSELSRCPKLLYEKLSEENKKHFQVEADGFLDEIEEEQ |
| | | NPFKDTLIRHQDRFPYFALRYLDLNESFKSIRFQVDLGTYHYCIYDKKI |
| | | GDEQEKRHLTRTLLSFGRLQDFTEINRPQEWKALTKDLDYKETSNQP |
| | | FISKTTPHYHITDNKIGFRLGTSKELYPSLEIKDGANRIAKYPYNSGFVA |
| | | HAFISVHELLPLMFYQHLTGKSEDLLKETVRHIQRIYKDFEEERINTIE |
| | | DLEKANQGRLPLGAFPKQMLGLLQNKQPDLSEKAKIKIEKLIAETKLL |
| | | SHRLNTKLKSSPKLGKRREKLIKTGVLADWLVKDFMRFQPVAYDAQ |
| | | NQPIKSSKANSTEFWFIRRALALYGGEKNRLEGYFKQTNLIGNTNPH |
| | | PFLNKFNWKACRNLVDFYQQYLEQREKFLEAIKNQPWEPYQYCLLL |
| | | KIPKENRKNLVKGWEQGGISLPRGLFTEAIRETLSEDLMLSKPIRKEIK |
| | | KHGRVGFISRAITLYFKEKYQDKHQSFYNLSYKLEAKAPLLKREEHYEY |
| | | WQQNKPQSPTESQRLELHTSDRWKDYLLYKRWQHLEKKLRLYRNQ |
| | | DIMLWLMTLELTKNHFKELNLNYHQLKLENLAVNVQEADAKLNPL |
| | | NQTLPMVLPVKVYPATAFGEVQYQETPIRTVYIREEHTKALKMGNF |
| | | KALVKDRRLNGLFSFIKEENDTQKHPISQLRLRRELEIYQSLRVDAFKE |
| | | TLDLEEKLLKKHTSLSSLENKFRILLEEWKKEYAASSMVTDEHIAFIAS |
| | | VRNAFCHNQYPFYEEALHAPIPLFTVAQQTTEEKDGLGIAEALLRVL |
| | | REYCEIVKSQI |
| 267 | Cas13 | MFFSFHNAQRVIFKHLYKAFDASLRMVKEDYKAHFTVNLTRDFAHL |
| | | NRKGKNKQDNPDFNRYRFEKDGFFTESGLLFFTNLFLDKRDAYWM |
| | | LKKVSGFKASHKQREKMTTEVFCRSRILLPKLRLESRYDHNQMLLD |
| | | MLSELSRCPKLLYEKLSEENKKHFQVEADGFLDEIEEEQNPFKDTLIR |
| | | HQDRFPYFALRYLDLNESFKSIRFQVDLGTYHYCIYDKKIGDEQEKRH |
| | | LTRTLLSFGRLQDFTEINRPQEWKALTKDLDYKETSNQPFISKTTPHY |
| | | HITDNKIGFRLGTSKELYPSLEIKDGANRIAKYPYNSGFVAHAFISVHE |
| | | LLPLMFYQHLTGKSEDLLKETVRHIQRIYKDFEEERINTIEDLEKANQG |
| | | RLPLGAFPKQMLGLLQNKQPDLSEKAKIKIEKLIAETKLLSHRLNTKLK |
| | | SSPKLGKRREKLIKTGVLADWLVKDFMRFQPVAYDAQNQPIKSSKA |
| | | NSTEFWFIRRALALYGGEKNRLEGYFKQTNLIGNTNPHPFLNKFNW |
| | | KACRNLVDFYQQYLEQREKFLEAIKNQPWEPYQYCLLLKIPKENRKN |
| | | LVKGWEQGGISLPRGLFTEAIRETLSEDLMLSKPIRKEIKKHGRVGFIS |
| | | RAITLYFKEKYQDKHQSFYNLSYKLEAKAPLLKREEHYEYWQQNKPQ |
| | | SPTESQRLELHTSDRWKDYLLYKRWQHLEKKLRLYRNQDVMLWLM |
| | | TLELTKNHFKELNLNYHQLKLENLAVNVQEADAKLNPLNQTLPMVL |
| | | PVKVYPATAFGEVQYHKTPIRTVYIREEHTKALKMGNFKALVKDRRL |
| | | NGLFSFIKEENDTQKHPISQLRLRRELEIYQSLRVDAFKETLSLEEKLLN |
| | | KHTSLSSLENEFRALLEEWKKEYAASSMVTDEHIAFIASVRNAFCHN |
| | | QYPFYKEALHAPIPLFTVAQPTTEEKDGLGIAEALLKVLREYCEIVKSQI |
| 268 | Cas13 | MEKPLPPNVYTLKHKFFWGAFLNIARHNAFITICHINEQLGLTTPPN |
| | | DDKIADVVCGTWNNILNNDHDLLKKSQLTELILKHFPFLAAMCYHP |
| | | PKKEGKKKGSQKEQQKEKEKEKENEAQSQAEALNPSELIKVLKTIVK |
| | | QLRTLRNYYSHHSHKKPDTEKDIFKHLYKAFDASLRMVKEDYKAHFT |
| | | VNLTRDFAHLNRKGKNKQDNPDFNRYRFEKDGFFTESGLLFFTNLFL |
| | | DKRDAYWMLKKVSGFKASHKQREKMTTEVFCRSHILLPKLRLESRY |
| | | DHNQMLLDMLSELSRCPKLLYEKLSEENKKHFQVEADGFLDEIEEEQ |
| | | NPFKDTLIRHQDRFPYFALRYLDLNESFKSIRFQVDLGTYHYCIYDKKI |
| | | GDEQEKRHLTRTLLSFGRLQDFTEINRPQEWKALTKDLDYKETSNQP |
| | | FISKTTPHYHITDNKIGFRLGTSKELYPSLEIKDGANRIAKYPYNSGFVA |
| | | HAFISVHELLPLMFYQHLTGKSEDLLKETVRHIQRIYKDFEEERINTIE |
| | | DLEKANQGRLPLGAFPKQMLGLLQNKQPDLSEKAKIKIEKLIAETKLL |
| | | SHRLNTKLKSSPKLGKRREKLIKTGVLADWLVKDFMRFQPVAYDAQ |
| | | NQPIKSSKANSTEFWFIRRALALYGGEKNRLEGYFKQTNLIGNTNPH |
| | | PFLNKFNWKACRNLVDFYQQYLEQREKFLEAIKNQPWEPYQYCLLL |
| | | KIPKENRKNLVKGWEQGGISLPRGLFTEAIRETLSEDLMLSKPIRKEIK |
| | | KHGRVGFISRAITLYFKEKYQDKHQSFYNLSYKLEAKAPLLKREEHYEY |
| | | WQQNKPQSPTESQRLELHTSDRWKDYLLYKRWQHLEKKLRLYRNQ |
| | | DIMLWLMTLELTKNHFKELNLNYHQLKLENLAVNVQEADAKLNPL |
| | | NQTLPMVLPVKVYPATAFGEVQYQETPIRTVYIREEHTKALKMGNF |
| | | KALVKDRRLNGLFSFIKEENDTQKHPISQLRLRRELEIYQSLRVDAFKE |
| | | TLDLEEKLLKKHTSLSSLENKFRILLEEWKKEYAASSMVTDEHIAFIAS |
| | | VRNAFCHNQYPFYEEALHAPIPLFTVAQQTTEEKDGLGIAEALLRVL |
| | | REYCEIVKSQI |
| 269 | Cas13 | MGAIENKHIFAAYANLAIDGLIKTLNFIAKKLDTQKQLSSWDIKHVITL |
| | | IDSIFDQNPQNNLEQVVEGYLPWIKPIIEMKTPKKGERQSDKLCIEYK |
| | | TIITAFASLLNDVRNYYTHYYHDPICIYPGGYDIPSSLNCIYDSAINIIKE |
| | | RFQAEEKEIEHLRRYTRKKGRVVLKTEDDHFYYTLVNNNDLSEKGYA |
| | | FFISMFLERKYSYLFLKKLSGFKRGDSLQYRLTLEVFTALSTKPPVERLR |
| | | TTKDTKQDRALDILNELSRIPIELYQTLEPKYREMYNETLQPTDAEDP |
| | | YGLPDRSRIRFRSRFEAFALHFLDKQADFKEIGFYTYLGNYFHNGYOK |
| | | TRVDRETKDRYINFQLAGFCKNIQDISAKKLSEALNVKSIDISTDSIPDI |
| | | NSFEPYLVQSTPHYIVNGNNIGIKVLPEGKDTYPTIDEKGAKMPIADF |
| | | WLSKYELPAMLFYTYLRNNNIHKSHCPLSVKDIIERSIHKSTKOKHPE |
| | | ERSELMLRRVMKAIFWTDSKLNEVERIKSQKSAFGKRQHEILKAGRI |
| | | AETLVRDMLWLQPSKNNGRDKVTEPNFQAIQVSLAYFGIRRNDLTE |
| | | IFTRAGLINSSNPHPFLAQIGTNYTSLIEFYIAYLKERKVYFSRIQKKILO |
| | | GKLNIQCHPLRDLQREPNKPQDKEEAIFLPRGLFNEAIINCLKKSKLK |
| | | QLIESPTREKSPALNVSYLIQNYFRTYFEDQSQEFYAQPRNYRLFDKL |
| | | SPNKGKSKSYLSLEQRIKKMEELRPSKIPVAEANKLLEKEDRLYRKNY |
| | | NEICDNESIIRLYQIQDILLFMMTKEYLPSDLYNRINKYKLENVKGILN |
| | | ERVSYLIDLNPLKIQGEDIKIKDYGKLFYIHHDTRISSLNKVLSKVKRNN |
| | | SISSSVKIQPYENYKRECLDFEEAQIQIIPIIHSFEIAMVSMFPDLKKAT |
| | | PGNYYDFNELITEYEKRTKQKIDSSFLIKTRNMFLHDKYEAECIKEISD |
| | | DFVYAKKIIAEFKMKIENIKLEDLSNDSSA |
| 270 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKDWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSOI |
| 271 | Cas13 | MEKPLPPNVYTLKHKFFWGAFLNIARHNAFITICHINEQLGLTTPPN |
| | | DDKIADVVCGTWNNILNNDHDLLKKSQLTELILKHFPFLAAMCYHP |
| | | PKKEGKKKGSQKEQQKEKENEAQSQAEALNPSELIKALKTLVKQLRT |
| | | LRNYYSHYKHKKPDAEKDIFKHLYKAFDASLRMVKEDYKAHFTVNLT |
| | | QDFAHLNRKGKNKQDNPKFDRYRFEKDGFFTESGLLFFTNLFLDKR |
| | | DAYWMLKKVSGFKASHKQSEKMTTEVFCRSRILLPKLRLESRYDHN |
| | | QMLLDMLSELSRCPKLLYEKLSEKDKKHFQVEADGFLDEIEEEQNPF |
| | | KDALIRHQDRFPYFALRYLDLNESFKSIRFQVDLGTYHYCIYDKKIGDE |
| | | QEKRHLTRTLLSFGRLQDFTEINRPQEWKALTKDLDYKETSKQPFISK |
| | | TTPHYHITDNKIGFRLGTSKELYPSLEVKDGANRIAKYPYNSDFVAHA |
| | | FISVHELLPLMFYQHLTGKSEDLLKETVRHIQRIYKDFEEERINTIEDLE |
| | | KANQGRLPLGAFPKQMLGLLQNKQPDLSEKAKIKIEKLIAETKLLSHR |
| | | LNTKLKSSPKLGKRREKLIKTGVLADWLVKDFMRFQPVAYDVQNQP |
| | | IESSKANSTEFQLIQRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLTLSKPIRKEIKKHGRV |
| | | GFISRAITLYFRERYQDDHQSFYNLPYELEAKASTPKPPLPKKREYVLR |
| | | AEHYEYWQQNKPQSPTELQRLELHTSDRWKDYLLYKRWQHLEKKL |
| | | RLYRNQDVMLWLMTLELTKNHFKELKLNYHQLKLENLAVNVQEAD |
| | | AKLNPLNQTLPMVLPVKVYPATAFGEVQYQETPIRTVYIREEQTKAL |
| | | KMGNFKALVKDRRLNGLFSFIKEENDTQKHPISQLRLRRELEIYQSLR |
| | | VDAFKETLSLEEKLLNKHASLSSLENEFRTLLEEWKKKYAASSMVTDE |
| | | HIAFIASVRNAFCHNQYPFYKETLHAPILLFTVAQPTTEEKDGLGIAEA |
| | | LLRVLREYCEIVKSQI |
| 272 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQTNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQO |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSQI |
| 273 | Cas13 | MEKPLLPNVYTLKHKFFWGAFLNIARHNAFITICHINEQLGLKTPSN |
| | | DDKIVDVVCETWNNILNNDHDLLKKSQLTELILKHFPFLTAMCYHPP |
| | | KKEGKKKGHQKEQQKEKESEAQSQAEALNPSKLIEALEILVNQLHSL |
| | | RNYYSHYKHKKPDAEKDIFKHLYKAFDASLRMVKEDYKAHFTVNLTR |
| | | DFAHLNRKGKNKQDNPDFNRYRFEKDGFFTESGLLFFTNLFLDKRD |
| | | AYWMLKKVSGFKASHKQREKMTTEVFCRSRILLPKLRLESRYDHNQ |
| | | MLLDMLSELSRCPKLLYEKLSEENKKHFQVEADGFLDEIEEEQNPFK |
| | | DTLIRHQDRFPYFALRYLDLNESFKSIRFQVDLGTYHYCIYDKKIGDEQ |
| | | EKRHLTRTLLSFGRLQDFTEINRPQEWKALTKDLDYKETSNQPFISKT |
| | | TPHYHITDNKIGFRLGTSKELYPSLEIKDGANRIAKYPYNSGFVAHAFI |
| | | SVHELLPLMFYQHLTGKSEDLLKETVRHIQRIYKDFEEERINTIEDLEK |
| | | ANQGRLPLGAFPKQMLGLLQNKQPDLSEKAKIKIEKLIAETKLLSHRL |
| | | NTKLKSSPKLGKRREKLIKTGVLADWLVKDFMRFQPVAYDAQNQPI |
| | | KSSKANSTEFWFIRRALALYGGEKNRLEGYFKQ.TNLIGNTNPHPFLN |
| | | KFNWKACRNLVDFYQQYLEQREKFLEAIKNQPWEPYQYCLLLKIPKE |
| | | NRKNLVKGWEQGGISLPRGLFTEAIRETLSEDLMLSKPIRKEIKKHGR |
| | | VGFISRAITLYFKEKYQDKHQSFYNLSYKLEAKAPLLKREEHYEYWQQ |
| | | NKPQSPTESQRLELHTSDRWKDYLLYKRWQHLEKKLRLYRNQDVM |
| | | LWLMTLELTKNHFKELNLNYHQLKLENLAVNVQEADAKLNPLNQTL |
| | | PMVLPVKVYPATAFGEVQYHKTPIRTVYIREEHTKALKMGNFKALVK |
| | | DRRLNGLFSFIKEENDTQKHPISQLRLRRELEIYQSLRVDAFKETLSLEE |
| | | KLLNKHTSLSSLENEFRALLEEWKKEYAASSMVTDEHIAFIASVRNAF |
| | | CHNQYPFYKEALHAPIPLFTVAQPTTEEKDGLGIAEALLKVLREYCEIV |
| | | KSOI |
| 274 | Cas12a | MKSLAQFQNLYALQKTLRFELKPEGHTRETFNRWLEEIEKEQASENE |
| | | NIVYQDLLRAKKYEKIKIILDEYHKDFIEQALAYANLTELEKYEELYRKS |
| | | NRTSEEEEEFENTKESLRKQIANIFIKNPNKTVQERWKFLFSKKLIQNE |
| | | LIVWVKGNYELLSEKLKNEFPDESSIISTIEDFKYFTTYFRNYHENRKNL |
| | | YSNEDKFSTIAHRLIHENLPKFIDNIAIYQKAKAVLNINEVEKELGLPED |
| | | TLDKIFSLDFFSKALTQKGIDQYNYFLGGKTENEVKKIKGLNEFINLYN |
| | | QQQQDKNQRLPFLKVLYKLPLFERTSTSFRFEPIENDRDLIERIGKFYY |
| | | NDLKQYRDDSQGDTTDILSGINTLLRHVHDYRDGLYVNGGITLTOIS |
| | | QKIFGSWSYINNALAYFYDTYIDASGVDHQGERKPKKQKOIQEKTK |
| | | WLKQKQFPVILVEKALSEYKSIETNEDLKTRISDTTLCDFFKRCGNDD |
| | | NGQDLFDRIEARLREKNEEGYSLEDLLKKEFTTERKLMQDKTKTLLIK |
| | | NFLDVIQGDKDDITAGLLHFVKCLIPRTEISEKNELFYSGMEKYLNILS |
| | | EVTPLYNKARNYLTQKPYSIEKVKLNFENSTLLDGWDENEESDNSCV |
| | | LLRKRGYYYLGIMNKKHNMIFDRKIYPKATEGEAYYEKMIYKLLPGAY |
| | | KMLPKVFFSEKNIDYFKPSEEILRIRNTASYSKNGQPQEGYQKASFSIE |
| | | DCRKYIDFFKKCIANHWDWQKFNFNFSPTEYYQSIDEFYREIERQGY |
| | | KIDFVKIPESYINQLIKENKLYLFKIYNKDFSEKKKSKGKDNLHTLYWK |
| | | MLFDEKNLKDVVLKLNGEAEVFFRQKSILYNEEIWNKGHHYSELKDR |
| | | FSYPIISNKRYAEDKFFLHVPITLNFKADGINNVNNMVNEFIKDNRDI |
| | | HIIGIDRGERHLLYVSVINQKGDIVEQCSLNEIVTEYNGKIFKKNYHEE |
| | | LDNLEKERDRARKDWQTIANIKELKEGYLSHVIHKISKLILKYNAIVV |
| | | MEDLNSGFKRGRQKVEKQVYQNFEKQLIEKLNYLVLKESNVDEPGG |
| | | VLRAYQLANKFETFKKLGKQSGIIFYVPAAYTSAIDPVTGYIQYLYPLK |
| | | QADSVEKARKFYSQFKRISYNPHKQWFEFSFDYNDFNIIYHGKSSWT |
| | | ICTTNTERYMWNRLLNNGHGGEELVYVTNELELLFGEYNIIYGDGKD |
| | | IKQQITDVQDIDVDRTAKQFYKRINELLNLTLKLRHNNGKKGADEED |
| | | YILSPVEPYFDSRFESRKPSMQQTLPINADANGAFNIARKGLLLLERL |
| | | NQLGVEEFEKTKKSNNKKTQWLPHELWVEYAQNHTRK |
| 275 | Cas12a | MQDKTGWSSFTNKYSLSKTLRFELKPVGNTQKMLEDDGVFQKDRE |
| | | RQENYKKVKPFMDKLHREFIKEALNNLKLEGLTEYFEIFKKFRKDKNN |
| | | KELKNAEKKLRQIIGRCYTETAQIWVEKYKEFGFKKKNIGFLFEEGVFE |
| | | LMKLKYGNDEASQIEKNGEVLSIFDGWKGFLGYFKKFFETRNNFYKD |
| | | DGTSTAVSTRIINENLKIYLDNLIKYNKIKDKVDFKEADILQENKLNLS |
| | | DFFNVESYAKYSLQKGIDYYNEILGGKTLKNGTKLKGLNEVINEYKQK |
| | | NKSGELSKFKMLKKQILGEGEDRTLFEEIENEDELKDVLKDFFYNADP |
| | | KITLFKTLLEDFFSNTEKYKDELDKIYFNTVAINGILHRWVDDSGVFQ |
| | | KYLFEVLKSNKLVKSNHYDKKEDSYKFPDFISFEHIKVALENCERDGLK |
| | | DKFWKEKYYTKECLTENGLANLWQEFLEIYKCEFKKLYDYKTDDNDC |
| | | YLQYRDNYKKYILDANFNPKEKSAKDIIKDYLDSVLSIYQLAKYFALEK |
| | | KKVWTTDYETGDFYYEYIKFYEDTYEQJIKPYNLVRNYLTRKPINTAKK |
| | | WKLNFDNAYLASGWDKDKEVSNLTVILRRDEQYYLAIMKKGKNKIF |
| | | EKKFSCGEFEKMEYKQIAEASSDIHNLVLMNDGSCRRCIKMHDKRK |
| | | YWPLDISIIKEKKSYAKENFVRRDFERFVNYMKKCSLLYWKEYDLKFS |
| | | DTSTYKNINDFTNEIASQGYKLSFSAIPESYINEKNNNGELYLFQIYNK |
| | | DFGIKTEGNKNLHTMYWESIFSEENRFRNFIVKLNGKAEIFYRPKSEQ |
| | | VEKEQRNFTREIIKNRRYTENKIYFHCPITLNRISRENVKKFNNGINNY |
| | | lATNPNINILGVDRGEKHLVYYAlVDQDGKLIDAEDATGSFNTIGSTD |
| | | YHRLLEEKAKDREKERKDWDLIRGIKDLKKGYISLVVRKIADLAIKYNA |
| | | IIIFEDLNTRFKQIRGGMEKSVYQQLEKALINKLSFLVNKGEKDPEQA |
| | | GHLLKAYQLAAPFQTFDKMGRQTGIIFYTQASYTSKIDPITGWRPNL |
| | | YLKYRNIDDSKESIKKFKSILFNKEKNRFEFTYDLKDFVDFEEDKIPEKT |
| | | EWTLCSSVERHKWNRHMNNNKGGYEVYKDLTENFYKLFDENNIS |
| | | MNKDIVDQVESISNGNFFRQFIYLFNLVCQIRNTDEKAEDVDKRDFI |
| | | LSPVEPFFDSRRAKDFKAYGDNLPKNGDENGAYNIARKGVLIIKKIKE |
| | | YYNQNGSCDKLGWGDLSISHKEWDDFATNN |
| 276 | Cas12a | MDSYEQFTKLYPIQKTIRFELKPQGRTKEHFDNSNFLEKDRERDDNY |
| | | KILKEVIDDYHREFIDECLSNIQLNWDDLKKFSEEYRRSKEKKNNRDS |
| | | ESEQKRMSTTSETRAINKKNLEAEQKRMRGEIVSAFKKDDRFKHLFS |
| | | EKLFSILLKNQIYEKGTLEEIEAFDCFNKFSGYFKSFHENRKNMYSDED |
| | | KETAISYRIINENFPKLLDNFEKYQYVCREYPEQIREAESTLAEAGCYIK |
| | | MDEIFSIDNFNNVMMQGGKESGISRYNLAIGGIVQGTGEKPKGLNE |
| | | FLNLAYQNEPNGRKKIRMEPLYKQILSKEESFSYRLEAFTDDSQLLSAI |
| | | RSFFDIVEKDKNGNIFDRAVNLMSSFSNYDTSKIYIRKAYLNQVSKEIF |
| | | GYRGKSDSKPAKTADESLNKSGGWEKLGQMLRDYKADSIGDRNLE |
| | | KTCKKVDKWLDSDEFTLSDILGAISLAGSNETFEAYVSEICVARRNIDK |
| | | EKEKEKNINVEKISGDTESIQIIKALLDSVQEFFHLLSPFQLHPNTPHD |
| | | WTFYAEFNDIYDKLSAITPLYNQARNHLTKKNLDTSKIKLNFNNPTLA |
| | | NGWDVNKEYENTAVILIRDGKYYLGIMNPKNKRKIKFDEGSGAGPF |
| | | YQKMVYKLLPGPYRMLPKVFFAKKNIDYYNPSQEIREGYKAGKHKK |
| | | GKEFDKGFCHKLIDFFKESIQKNENWKVFDFKFSPTESYDDISEFYQE |
| | | VEKQGYRMYFVNIPSDTIDRYVEGGDMFLFQIYNKDFAKGAKGNK |
| | | DMHTLYWNAVFSEENLQKGVMKLSGEAELFYRKKSDIKDPPHREG |
| | | EILVNRTYIDRTHVSGVMGEQNTVKESRIPVPDEIHKNLFDYYNHGR |
| | | ELTKEEKEYCDKVGSFKAYYGIVKDRRYLENKMYFHVPLTLNFKAIGE |
| | | KRINKMAIEKFLTDENACIIGIDRGERNLLYYSIIDRNGKIIDQKSLNVI |
| | | DGFDYHEKLSQRQTEREVARQSWNSIGKIKDLKEGYLAKAVHEISK |
| | | MAIKYNAIVVLEDLHFGFKKGRLKVEKQIYQKFEEMLINKLNYLVFKD |
| | | VSDSSDAGGVLNAYQLTAPLESFSKLGKQSGILFYVPAAFTSVIDPTT |
| | | GFVDLFNSSSITSTQKKKEFLQRFESIVYSARDGGIFAFTFDYRNFSKIA |
| | | TDHRNMWTVYTHGERIRYVRDEKCYKTTDPTKRIKEALSGIEYDDGS |
| | | DIRDKITQSGDNNLINTVYHSFMDTIKMRNKDGRIDYIISPVKNRNG |
| | | EFFRSDYKHRDFPVDADANGAYHIALKGELLMRMIGKTYDSNSDK |
| | | MPKLEHKDWFEFMQTRGDO |
| 277 | Cas12b | MCVSRLPWFNITLTGKLNRQRLNQMCVSRLPWFCTPKGQLAATPK |
| | | TVVAQQENAMLAIIRDVHEAAPADLKTVAQRLEPGYFVTQFPKQQ |
| | | MTGDEARAEAERLFAACQKKFKELAEYEDGYRQCLDALGPNLSLPR |
| | | LGRKPKGAYPYAVVFKLMPTNATWECFKRVTASLYKRAQKGVVSP |
| | | VSADSIADVRTNDEPLFEYFTNLALVRPPGNKDRAVWFEFDLAAFIE |
| | | AIKSPHQFFQDTIKREQAVAQIKAKLDAMDGQGRAASGEEDALPGF |
| | | EGDDRITLLRELVTDTLGYLAEADASTSPGGKIEYSIQERTVRGFAEVK |
| | | RRWRDLVEKGKATEDALLKVLAEEQTEHRDDFGSATLYRELAKPKF |
| | | QPIWRDPGTQPWHADDPLRAWLEYRELGRELEDKQRPIRFTPVHP |
| | | VHSPRFFIFPKKKGGGRFGTVHEPGQLRVMAGIVAQTQHGWEPVP |
| | | VRITYAAPRLRRDQLRDDVETDLESRPWLQPMMQALGLPEPDTAD |
| | | FSNCRVTLQPSAPDDIQLTFPVDVSADKLTTAIGKAARWAKQFNLFP |
| | | DGDNFYNASLRWPHEKKPSKPPVPWHEALDNFSVLAADLGQRCA |
| | | GAFARLEVRANDDFAGKPSRFIGETPGKKWRAALVAAGMLRLPGE |
| | | EQTVWRPGATGPNFHTELSGSRGRMARPHEADDTADLLRAFDCPE |
| | | ESLMPADWRTSLSFPEQNDKLLVAARRYQSRLARLHRWCWFLTDE |
| | | KKRQTALDEIREAEDMPAADDPQLTDKLRALLLQKQAALPGLLVRL |
| | | ANRILPLRGRSWQWETHPDKADCHLLTQTGPALPDVWIRGQRGLS |
| | | MQRIEQIEELRRRFQSLNQMQRREIGGKPPIRRDDSIPDCCPDLLDK |
| | | LDQIKEQRANQAAHMILAEALGLRLAPPPADKRQLRASRDVHGOY |
| | | VKSREPVDFIVIEDLSRYRSSQGRAPRENSRLMKWCHRAVRDKLREL |
| | | CEPFGIPVVETPAAYSSRFCSRSGVAGFRAVEVGPGFDREFPWMML |
| | | KDREDEGEPVRQLILQVATLNQGRDGKPPRTLLAPLAGGPIFVPIVD |
| | | KLNGADIQPALAQADINAAINLGLRAIADPRLWSIHPRCRTQRQGD |
| | | QMLTREKRKFGETGQPLAVHRADGVKPDDTRNPNFFADISGSLPA |
| | | WESATLDGQHLLSGRCLRSEIKKRQWQRCAEINDRRMNRWMKGE |
| 278 | Cas12b | MTELQTQRAYTLRLKGIDEKDQSWRDALWKTHEAVNKGAKVFGD |
| | | WLLTLRGGLDHTLADAEIPGEKGKPDRAPTQEERKHRRILLALSWLS |
| | | VESERGAPEEFIVATGKEPAATRNDKVIAALKDILRGRNLTEEKISEW |
| | | TEVCTPSLSAAIREDAVWVNRSRAFDEAVKRIGSSLTREEVWDMLE |
| | | CFFGSRNAYLAPVKISEDESSDGEQEEKAKDLVQKAGQWLSSRFGT |
| | | GEGADFAKMAAVYAKIAAWAGNAQAGTTGNEVINNLATALREFT |
| | | PKSNDLKGVLDLISGPGYKSATRNLLKQIANTKTVTREDISKLQETAG |
| | | EDSEECATKTGSKGKRAYADAILKDVESVCGFTYRIDKDGQPVSVAD |
| | | YSKYDEDYKWGSSRHKEFAVMLDHAARRVSLAHTWIKRAEAERRK |
| | | FEEDSKKIMQVPQAAKDWLDAYCAQRSEASGALEPYRIRKRAIQG |
| | | WKEIIASWNKPDCKTAEDRIAAARQLQDDPEIEKFGDIQLFEALAED |
| | | DAQCVWKKEDGTLDPEILINYTLASEAMFKKQHFKVPSYRHPDAFL |
| | | YPVFCDFGNSRWELDFSIREAATKLKEIEAKIEKQRQEVHKVQQALE |
| | | KCENDEKRPKMEERLKEAQKKLQESQNYGEYLHSNNKITMVLFDGT |
| | | FVKKHIFAWQSKRLTKDLALYQEPSADPKNVVSRADRLGRAVASVG |
| | | INDAVKVAGLFEQENWNGRLQAPRQQLEAIAQYVEKHGWDNKAE |
| | | KMRASIKWFITFSAKLQSKGPWNEFARKHGLKEDPHYWPHAEKNE |
| | | NRTAHSRLILSRLPGLRVLSVDLGHRYAAACAVWEALGSEAFKKDIE |
| | | GKRIIRGDTDENALYCHTEHEANGKKHITIYRRIGADTLPDGAHHPA |
| | | PWARLDRQFLIKLQGEDEQAREASNEEIWKVHQLENTLGRRTPLID |
| | | RLIAGGWGYTEKQKARLEVLTNLGWCPTNKTDNQEEGDEEETAILS |
| | | KPSLLVDDLMFSAVRTLRLALKRHGDRARIAHYLITDEKTKPGGVKEK |
| | | LDKNGRVELLLDALGLWHDLFSSPGWHDEKAKQLWNAYIAGLLPE |
| | | GELQQAKSVTTSAALGGQQKKEKKEKLRAVAEALYLNSDLCHSLNE |
| | | VWRKRWEEDDKQWRIYIRWFKDWIMPRGANAKSPAIRHVGGLSL |
| | | TRLATLTEFRRKVQVGFFTRLHPDGTKTETREDFGQKTLDTLEHLRE |
| | | QRVKQLASRIVEAALGIGSEDKRHWDGKKRPRQRIADPRFVPCHAV |
| | | VIENLTHYRPEETRTRRENRQIMEWASSKVKKYLSEICQLHGLHLRE |
| | | VSAAYTSHQDSRTGAPGIRCQDVSLIEFMKSPFWRKQVAQAEKKQ |
| | | KEGKGDAVERYLCELNQKWKGASEEEWRKAGFVRIPLRGGEIFVSA |
| | | AGHDSPAAKGIHADLNAAANIGLRALLDPDWSGKWWYVPCNSST |
| | | MCPARDKVTGSAAVNPGQPLQVSAQLESDDAAKDTKKRKKKGDG |
| | | KSKEIINLWRDISSYPLEDTRGGTWSNKTVYWNRVQSNVVHILQNQ |
| | | MKG |
| 279 | Cas12b | MPETTQRAYTLRLQGHDPKDASWREALWKTHEAVNRGAKAFGD |
| | | WLLTLRGGLDHSLADEGAPGQTPTEEQRKQRRILLALSWLSVESEN |
| | | GAPQEYIVPHDRDNESGARQNWKTREALREILKNRGCRDDEIESW |
| | | CHDCEPSLTSAIRKDAVWVNRSKAFDNAVQSIPNFSREEIWDLLGCF |
| | | FVSSQAYLAPLESPKDDKPDASKKDSSKDLIQSAGQWLSRRFGRGK |
| | | GLNFARLAETYEAIARWASVANPGDTNDLIADLAKTLNAETPELDGI |
| | | LKVVSGPGHKSKTRNLLRSLSAVNHITKDTLQRLKDTANEDAKKAKL |
| | | KKGEKGHRAYAYKVLEAVEDACGFTYLQEGDRAKHCEFAVMLDHA |
| | | ARRVSSLHTWIKRAEAERRRFEIDTKKKDQLPPSVKEWLDTYCQKRS |
| | | KETGAVEPYRIRRGAIEGWKEIVEAWSKAGTTTAEDRKHEARRLPD |
| | | NPHIDKSGDIKLFEDLALEDALPVWHANGDPNNPPDPQLLIDYVEG |
| | | SEAEFKKRAFKVPTYCHPDPLVHPVFCDYGCSRWNVSFAIQPVKKQ |
| | | KLSSEEKLPAKGLLLDLLHGTAIRPVALRWQSKRFARDLALNTTDSSD |
| | | KPNEVTRADRFGCALAKCPSSQKIRIRGLFEEKYWNGRLQAPRPELT |
| | | ALAKRVAKYGWDKKARKLRNSLNWFITFSANLRPSGPWEEYTKYAE |
| | | KAFSSNASAKPSVSRGGFWVVHASPNKRGKMAQLRLCRLPELRVLS |
| | | VDLGHRYAAACAVWETLSKSAFEQEIHERKILRGGTGPNDLFCHTQ. |
| | | HDTNGQSKVTIYRRIGADTLPNGTPHPAPWARLDRQFLIKLPGEERE |
| | | ARKASPTELANVEKLEKELGLKTSENRVKRIDDLMSDTLRTVRQALR |
| | | RHSLRARIAFNLATLRDQSDGDEESQSKQKRDTRWNNTVKIWHSLL |
| | | ESNEWTDDWAKALWDELGPLSDPQKADDAEWLKLAAEKFYTRW |
| | | QEDEQTWRERLRWLRRWILPRGSQAASQKGSIRHVGGLSLTRLATI |
| | | KTLYQVLKAYHMRLKPDNSRKNIPAEGDEALQNFGQKILDDLEHMR |
| | | EQRVKQLASRIVEAALGLGRMKQVTIGKDPKRPREPVDQSCHAVVI |
| | | ENLTHYRPEKRQTRRENRQLMDWSAAKVKKYLKECCQLHGLHLVE |
| | | VSASYTSRQDSRTGAPGIRCQEVPLTDFLKKNFWREQVKQAKQRLS |
| | | EGKANARDRYLCQLNERWGNAPAPVTQTAIRLRIPLNGGELFVSAD |
| | | QNSPASKGIQADLNAAANIGLRAITDPDWPGAWWYVPCEANTFK |
| | | PVKDKVAGSAAIDSNVSLKKDSPNSEKPASDRKSRTSKSMINLWCD |
| | | TSSKSLSEKDQWQESAPYWEDVAARTINILQASLACSTTNSQ |
| 280 | Cas12a | MKSLAQFQNLYALQKTLRFELKPEGHTRETFNRWLEEIEKEQASENE |
| | | NIVYQDLLRAKKYEKIKIILDEYHKDFIEQALAYANLTELEKYEELYRKS |
| | | NRTSEEEEEFENTKESLRKQIANIFIKNPNKTVQERWKFLFSKKLIQNE |
| | | LIVWVKGNYELLSEKLKNEFPDESSIISTIEDFKYFTTYFRNYHENRKNL |
| | | YSNEDKFSTIAHRLIHENLPKFIDNIAIYQKAKAVLNINEVEKELGLPED |
| | | TLDKIFSLDFFSKALTQKGIDQYNYFLGGKTENEVKKIKGLNEFINLYN |
| | | QQQQDKNQRLPFLKVLYKLPLFERTSTSFRFEPIENDRDLIERIGKFYY |
| | | NDLKQYRDDSQGDTTDILSGINTLLRHVHDYRDGLYVNGGITLTQIS |
| | | QKIFGSWSYINNALAYFYDTYIDASGVDHQGERKPKKQKQIQEKTK |
| | | WLKQKQFPVILVEKALSEYKSIETNEDLKTRISDTTLCDFFKRCGNDD |
| | | NGQDLFDRIEARLREKNEEGYSLEDLLKKEFTTERKLMQDKTKTLLIK |
| | | NFLDVIQGDKDDITAGLLHFVKCLIPRTEISEKNELFYSGMEKYLNILS |
| | | EVTPLYNKARNYLTQKPYSIEKVKLNFENSTLLDGWDENEESDNSCV |
| | | LLRKRGYYYLGIMNKKHNMIFDRKIYPKATEGEAYYEKMIYKLLPGAY |
| | | KMLPKVFFSEKNIDYFKPSEEILRIRNTASYSKNGQPQEGYQKASFSIE |
| | | DCRKYIDFFKKCIANHWDWQKFNFNFSPTEYYQSIDEFYREIERQGY |
| | | KIDFVKIPESYINQLIKENKLYLFKIYNKDFSEKKKSKGKDNLHTLYWK |
| | | MLFDEKNLKDVVLKLNGEAEVFFRQKSILYNEEIWNKGHHYSELKDR |
| | | FSYPIISNKRYAEDKFFLHVPITLNFKADGINNVNNMVNEFIKDNRDI |
| | | HIIGIDRGERHLLYVSVINQKGDIVEQCSLNEIVTEYNGKIFKKNYHEE |
| | | LDNLEKERDRARKDWQTIANIKELKEGYLSHVIHKISKLILKYNAIVV |
| | | MEDLNSGFKRGRQKVEKQVYQNFEKQLIEKLNYLVLKESNVDEPGG |
| | | VLRAYQLANKFETFKKLGKQSGIIFYVPAAYTSAIDPVTGYIQYLYPLK |
| | | QADSVEKARKFYSQFKRISYNPHKQWFEFSFDYNDFNIIYHGKSSWT |
| | | ICTTNTERYMWNRLLNNGHGGEELVYVTNELELLFGEYNIIYGDGKD |
| | | IKQQITDVQDIDVDRTAKQFYKRINELLNLTLKLRHNNGKKGADEED |
| | | YILSPVEPYFDSRFESRKPSMQQTLPINADANGAFNIARKGLLLLERL |
| | | NQLGVEEFEKTKKSNNKKTQWLPHELWVEYAQNHTRK |
| 281 | Cas12b | MAYQNGKEQPTVTNQRAYTLRLSGTNDQDSIWRNRLWHTHEAV |
| | | NKGAKTFGDWLLTMRGGLCHTLAEADVPGKGNKPARHPTPQEIRS |
| | | RRVVLALSWLSVESQHGAPERHLVSHDLDIATGERKNWKTVEALRE |
| | | ILHGRCLCKELIDEWANDCRDSLSATIREDAVWVNRSKAFDLAAKKI |
| | | GASLTREELWDFLQPFFANKHGYLQMDTVAGVTNGDSETDAEEAK |
| | | EDSSEEKAKDLSQKAGQWLSSRFGTGTGADFSRFSKVYEVLAARCG |
| | | SVAVGVSGVEAIRILAGTLADFSPGSNDIEGMLGLMSGPGYKSATR |
| | | NILQKINTLQTVSQQDLDRLREASEKDALQSKQKVGGKGSRPYANAI |
| | | LQDVEAACGICYAGTGESPARHWQYAVILDHAARRVSMAHSWIKR |
| | | AEEQRSKFEIEKDKLDHVPKDALAWLDAFCARRSSESGASDAYRIRR |
| | | SAVDGWKQVVAAWAALPPKPENQGSELLSDAESARIQAARELQDT |
| | | VEKFGDIQLFEALSLTGAKCVWQPDGRPDAQPLLDYVAGTDAISKK |
| | | QRFKVPAYRHPDALLHPVFCDFGNSRWNINYAIHRAPEKLTPAQQL |
| | | LEKKKAEIDKAELTLAKAGDAAKQANISEKINGLRAAFIQQQEKVAW |
| | | LNSRHAMTMSLWDGTHIEDTPLIWQSKRFGSDIGQPVEAQPLPVS |
| | | RADRFGRAVALAQDNVPVIPSGLFDLSDWNGRLQAPRRQLEAIAAl |
| | | RDSAKLSVNEKQQLVAKRIQSIRWLLTFSAKLQSHGPFIAYAAQHGF |
| | | DWRYGAHGPENKSRQGLAKLILCRLPGLRILSVDLGHRYAAACAVW |
| | | ETLNAGQIQKACLDAGKEAPGPCTLYLHLKQIANGKEKKTIFRRIAAD |
| | | TLPDGSPHPAPWARLDRQFLIKLQGEDRDARLATSEEIAAVEQMEN |
| | | ELGVVRQLKRKGRELLVDELMSDALRTLRLGLRRHGVRARIAFNLTA |
| | | NKRIRPGGKEEVLDQEGRVLLLTETLLAWYELYTAERWTDEPAREL |
| | | WNRHIQPLLGATILQNTVNQEDTPSAAKRRKLREETSGKLKHVAEEl |
| | | AKNDSLCRQLHVLWSAQWQTEDVIWRTRLRMMRRWLLPRGVKR |
| | | NAQLRISIRDVGGLSLTRIASFKSLYQVQKAYQMRPHPEDPRLNIPER |
| | | GDSRLENFGQRVLDAMERMRENRVKQLASRIAEAALGIGGETGISS |
| | | KDGSQKKRPTERSSDPRFAPCHAVVIEDLTHYRPDETQTRRENRQL |
| | | MSWSSSKVKKYLGEACELNGLYLREVSPAYTSRQDSRTGAPGLRCN |
| | | DVTVVEFNNSPFWRKQVGAAEKNQKEGNKGDARERYLLSIEEGIRG |
| | | AANDRDIFRIPVKGGEIFVSACITDGGNNAKKNAPPGLQADLNAAA |
| | | NIGLRAIFDPDWEGRWWYIPCDAATLCPDAKKFIGCKAVDPTKPLR |
| | | VVAEEGAISASGIGSKKSGRKKNAATDGTRIVNLWRDPSGAPIHRD |
| | | VLRSPEWQDYAGYWNEVQHRVIRNLKTCYEQTSQQEDPFVSQDA |
| | | DKPF |
| 282 | Cas12b | MKRLAETALADKVKCETNSRPKGERAYANSILHDVESACGFTYRVD |
| | | KGEQPVPVSDYSHYANDYRWGPANHSEFAVMLDHAARRVSLAHT |
| | | WIKRAEAERRQFEENAKKIDKVPKVAREWLDSLCAERSIVLGALEPY |
| | | RIRRRAVDGWKHVVAAWSKSDCKTAQDRITAARLLQEDPEIDKFG |
| | | DIQLFEALAEDHAVCVWQRDGEAGKTSDPQLLIDYALAAEAEFKKR |
| | | HFKVPAYRHPEAFWHPVFCDFGQSRWKICFDVHKNRQSRRQRAC |
| | | ANRISRKICFDVHKKRQTLRLSLEVWTGSKMLDMPLCWQCKRLAR |
| | | DLALGQDHKKDRSCQVTRADRLGRAVSNVARNQEVQILGLFEQEY |
| | | WNGRLQAPRPQLEALGRYIEKHGWDAKAQKSCRAIRWMISFSPRL |
| | | QPAGPWGKFAEKLQLNPNPKYWPHAEDNKDRGSRSKLILCRLPGL |
| | | RVLSVDLGHRYAAACAVWEAVDAEQVKEACQAAGHREPNENDLY |
| | | LHLKKRTTKQKKGSQGVVEETTIYRRIGADTLPDCTPHPAPWARLDR |
| | | QFLIRLQGEEDEARAASNEEVWAVHKLEAELGRTIPLIDRLLGAGW |
| | | GQTEKQKARLKALRELGWTPANKCQAFNSTDETELRRPSLAVDEL |
| | | MLDAVGTLRLALKRHGDRARIARYLITDERTKPGGVKEKLDENGRIE |
| | | LLQDALIIWHGLFSSPRWRDDAAKQLWNEHIAKLVGEQNLVEVSED |
| | | ASGSERRTKQKQNREKLREAAKALVDDVALRQALHDMWKRRWEE |
| | | EDREWRRRLRWFKDWVLPRREQARKAYSRPAETGSSSHPKRRARY |
| | | AAIRRVGGLSLTRLATLTEFRRKVQVGFFTRLKPDGTKAEAKEGFGQ |
| | | STLDALEHLRAQRVKQLASRIVEAALGVGRIRRFPGVKNPKRPDTPV |
| | | DKPCHAIVIENLTHYRPEETRTRRENRQLMTWSSSKIKKYLAEACQLY |
| | | GLHLREVTAAYTSRQDSRTGAPGLRCQDVPVKEFMRSLFWRKEVA |
| | | QAEKKLTAGKGSSYERLLCELNQRWKDNSPGDGKRAELLRLPHKGG |
| | | EIFVSAAPDSPAARGLQADLNAAANIGLRALTDPDWPGKWWHVP |
| | | CNAVTFRPVEDKVKGSAAVKLDQSLRQVAHPQSKDPGAKKSKEIVN |
| | | LWCDISSLPLEHREWKLDWEPYPAYWNNVQCRVIRVLQGKV |
| 283 | Cas12b | MANAKVKTTTRSYTLSLNAPSDTTDRSPLWHRIFRTHYAICCGAREF |
| | | GKLLLDLRGGLPTSLAQLGEGIAENDRRQTQRGTRRILALGWLSVED |
| | | LDHARNDPHRVQDTAPGSPLDQDLAEKILRKILITKGIKSEEEQSNWI |
| | | SDCLPALTANIRPDAVWVNRAESFAQWQRGTQPGAQPPTPEEAQ |
| | | QILFSLCGESLVTLTLPEQPAAAGQKQPDQETSPDPEEQTDRPPAAP |
| | | SADDEMDPSNASRGIFGDLFGENAEGKRSRSQGKDNFACAVRDFL |
| | | CANPTPSADAITEFREKQKPREPNPPGPEKYPPEVSTSGAPTAVAKR |
| | | YRKLLVCAGLWPKSADEDGSSRNSAKTKFADPKEPQKTEIQINALDLI |
| | | DACNQAAPADDSGTSPKAGRVFAPAWASNIAEKVASATQMPANA |
| | | KSLNEFKRLMFALAARRFSQTQSWTRRNEAERHMAAARQDAAVA |
| | | RLREIDPDHKAQDWLRGYEQRRADQSGSNGEFRITRRMIGEAEAV |
| | | FKAWAGTNSAAERELKTVAVQTTAEKFGDAALYSEIARNTAAEAV |
| | | WRSGSAPEILDQWVKLRKAQSDQQRTRVPRFCHPNAFRHPTWCE |
| | | FGESSKPGVWYAWNPKSKPRKPEVGGEGDGTRRLWVLLPDFNSGI |
| | | GQAVPLRWRSKRLSKDLGEALQPSDAPIPRADRVSIAAAGLNLEGA |
| | | NGVPARYRPSLPFSENTKGWNARLQANRTALLHLESKWDAEAAT |
| | | WRDGGRSLLALKWFTTFSPELAMSEGPGRAIHPKLGWNSEPHSDL |
| | | NRAQKRGGNAKLILSRLPGLRVLSVDLGHRYAAACAVWETLTTEQ |
| | | MNAACQAKNHTQPAESDMYVHLAHPTERVVKSGRKKGQNLIQTT |
| | | VYRRIAADTLPDGTPHPAPWGRLDRQFLIKLQGEQRPTRAASKNEA |
| | | DLANALFHRLGLRSDADSENKSRAVDKLMARTVRVATLGLKRHARR |
| | | AKIAYALDPNTKAIPGMGGSSAAFTPGDEPHIHLLTDALFDWQSLAT |
| | | DAKWDDAHARSLWNHHIATLPGGFHLENPTPRDESAHEPSRQRQ |
| | | RSGDDALRATLKPIAEKLSKADRQEVHAAWKKYWGDSDGQSAIVP |
| | | KVLQGQRGPEKTTPSASASGWHGKIRWITDWIMGKYLEGCTGHA |
| | | WKHDVGGLSVSRITTMKSLYQLHKAFAMRATPEKPRGAPEKGESN |
| | | LGAAQGILTAMESMRQQRVKQLASRIAEAALGAGIERRSDNGREL |
| | | QRPRERVDDPRFAACHAVVVEDLTNYRPDEMQTRRENROLMQW |
| | | ASSKVKKYLSEACQLHGLYLRGVPAGYTSRQDSRTGAPGVRCGDIPV |
| | | EELMAAPRWRRQILTAEKTRRENNTGTARDRYILTLDEKYRLLTAEQ |
| | | RKKTPPARIPVKGGDLFVSADPDSPAASGIQADLNAAANIGLKALID |
| | | PDWPGRWWYIPCDATTHKPSPERTRGSAAVDCDVPLGPDSTGTPE |
| | | DRDAKPKKNQRNSKIAGRGQSAIINLWRDPTHLPIKENPSAWCESK |
| | | KYWNQVEHNVVKVIESKGQKLTQTAEAATGESASSPPIAPTDVPW |

Those skilled in the art will appreciate that, given that their amino acid sequences are known, these enzymes can readily be produced (e.g., through culturing of source organisms and/or by recombinant expression/purification, as, for example, may be contracted from any of a variety of commercial sources). Produced enzymes can then be assessed for direct and/or collateral cleavage at varying temperature(s) and/or for other evidence of stability and/or functionality at relevant temperature(s).

### Example 4: Thermostability of Cas13

This example confirms and further demonstrates the thermostability of Cas13 enzymes. This example provides certain thermostable Cas13 candidates for use in improved collateral activity assays as described herein. The thermostability of TccCas13a and ThpCas13a was tested. Briefly, varying ranges of labeled RNA target was incubated with TccCas13a or ThpCas13a; Rnase Inhibitor; T7 RNA Polymerase, MgCl2 and a cRNA (either in forward or reverse complement orientation). FIG. 3A demonstrates the significant activity of TccCas13a at 65° C. The data of FIG. 3A also suggests the structure of the cRNA can influence the activity of thermostable enzymes. Further, FIG. 3B demonstrates that TccCas13a is active across a wide range of temperatures including the range 40° C. to 65° C.

Additional assays were performed with TccCas13a with or without some components to identify parameters contributing to background within the assays. FIG. 3C demonstrates that presence of the complex of the Cas enzyme and a cRNA contributes to background in the assay.

### Example 5: Exemplary Discovery and Screening for Thermostable Cas Enzymes

The present example demonstrates an exemplary method of discovery and screening thermostable Cas enzyme candidates (e.g., Cas12 and Cas13 enzymes) (FIG. 4). Novel Cas12 and Cas13 enzymes were discovered using a custom-built in silico pipeline. In brief, publicly available microbial genomes and metagenome databases were first filtered on the basis of environmental metadata such as sample collection temperature and sequencing read quality. CRISPR repeats were subsequently identified in the filtered genomic datasets using published repeat annotation methods. Next, all coding sequences were annotated in genomes that had CRISPR repeats using published open-reading-frame (ORF) discovery methods, and these ORFs were subsequently classified using a Hidden Markov Model (HMIM) that was pre-trained on known Cas12 and Cas13 enzymes. An enzyme was annotated as a putative candidate if and only if its direct-repeats were conserved (>95%) and the predicted enzyme had a domain topology consistent with previously discovered enzymes.

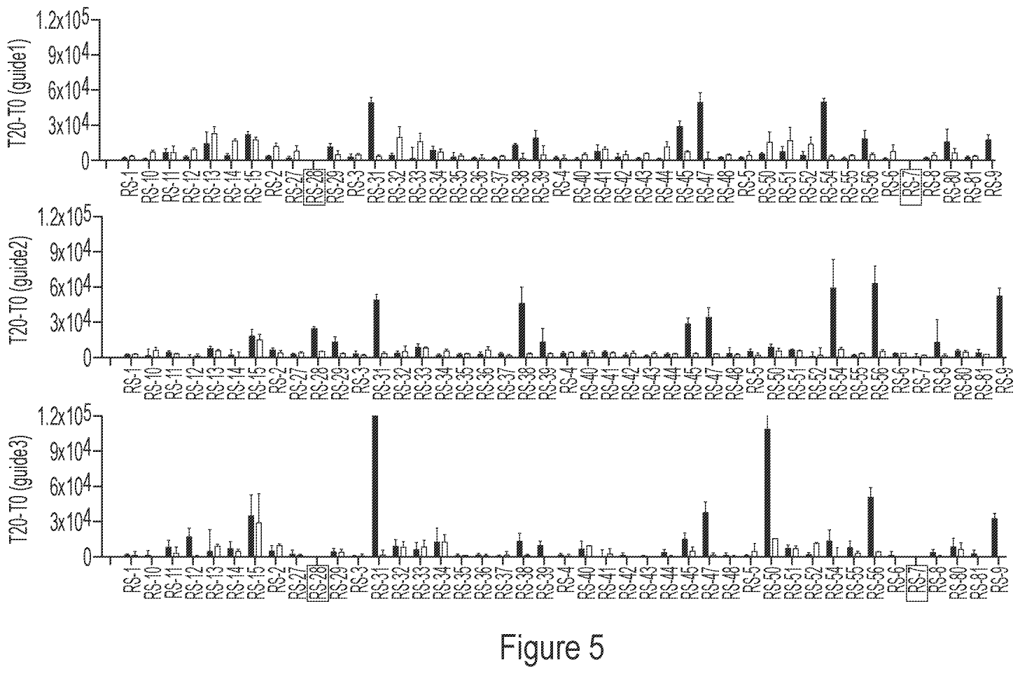

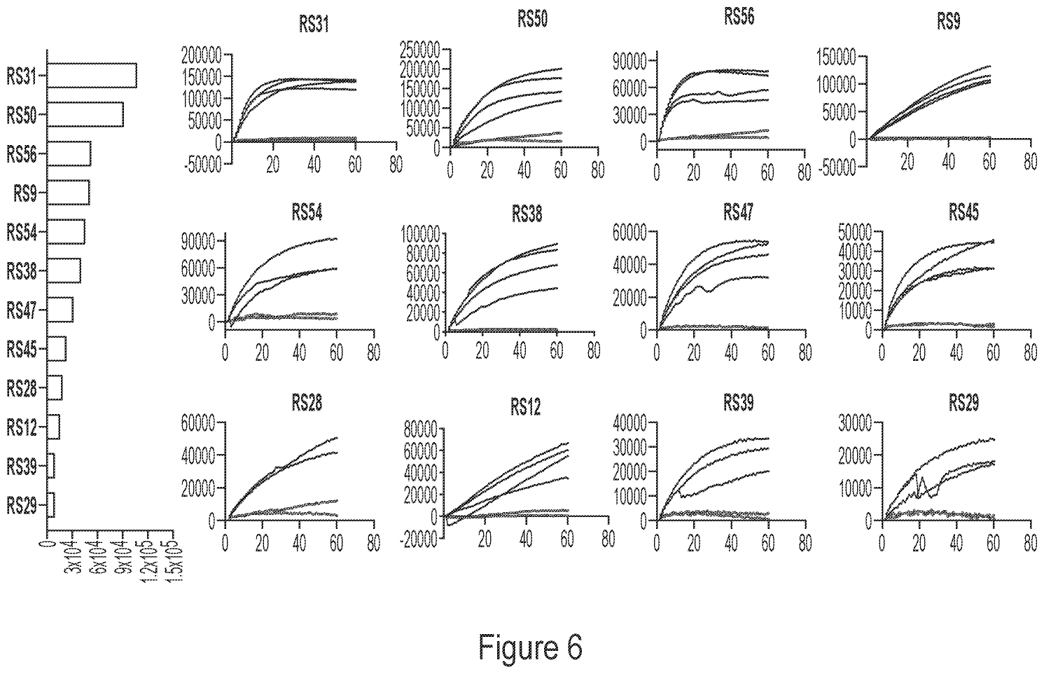

Candidate enzymes were expressed by in vitro protein synthesis (e.g. PURExpress in vitro Protein Synthesis Kit by New England BioLabs) according to manufacturer's instructions. An initial pool of Cas12a candidate enzymes were assessed by endpoint (FIG. 5) and kinetic analyses (FIG. 6) using no template as a negative control for activity at 52° C. Each candidate was tested with three different guide/target pairs at 52° C. (FIG. 5).

A subset of candidates (e.g., 12 candidates) that demonstrated highest activity among the 44 initial candidates at 52° C. were selected in combination with their most efficient guide and target for further assessment at higher temperatures (e.g., 58° C., 60° C., 62° C.).

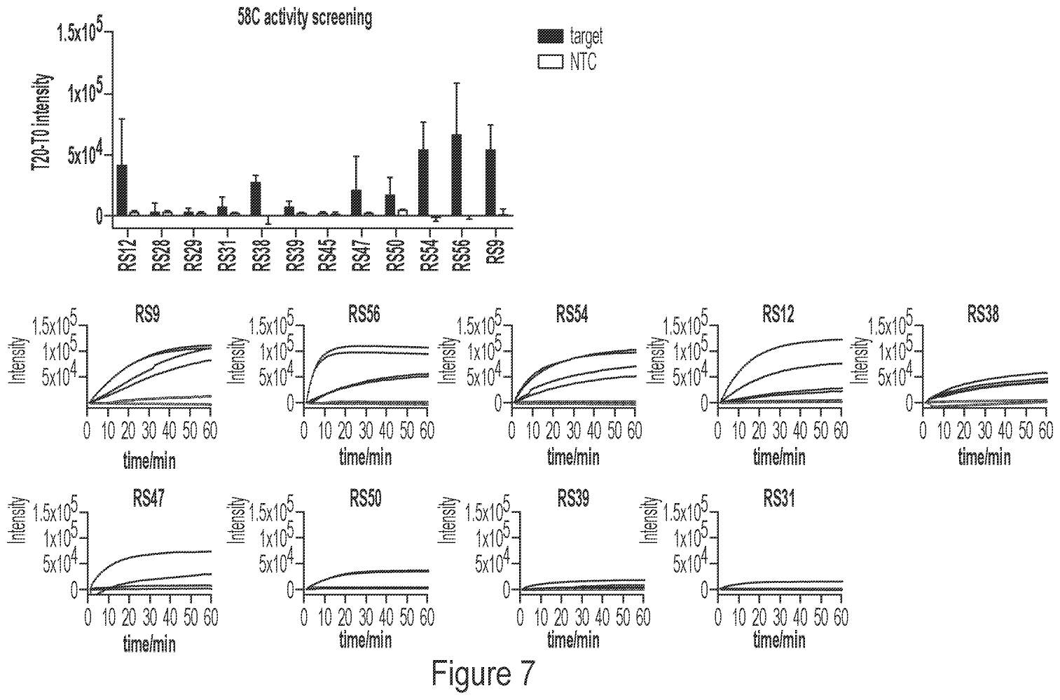

Both endpoint and kinetic analyses indicated a subset of candidate enzymes (e.g., 9 of 12 candidate enzymes) that displayed some activity at 58° C. Of the nine with some activity, five were classified as high activity (RS9, RS12, RS38, RS54, and RS56), and the remaining four were classified as lower activity (RS31, RS39, RS47 and RS50) (FIG. 7).

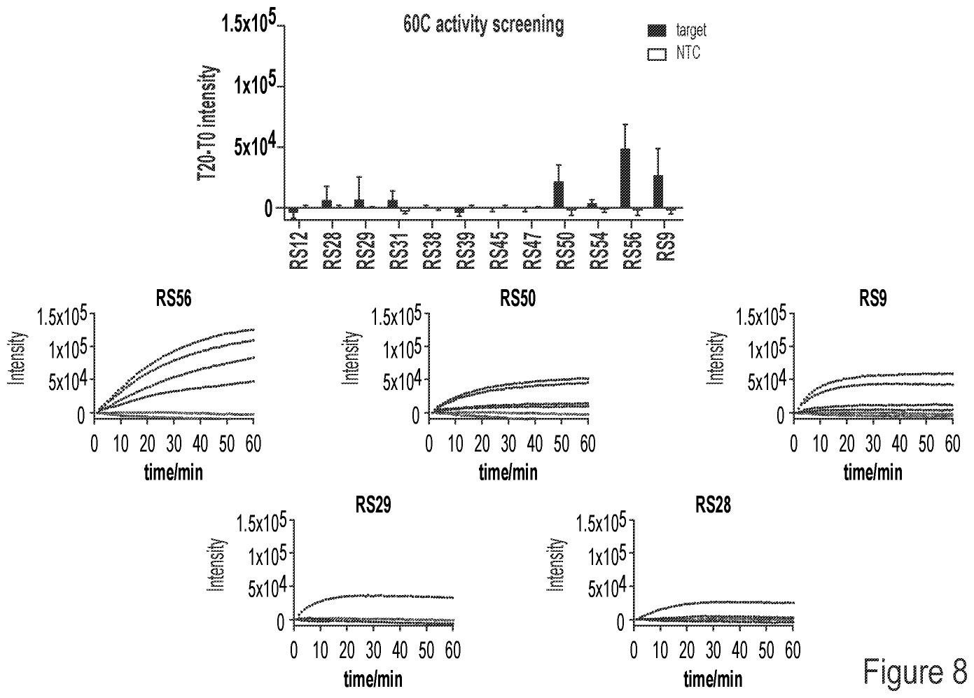

Both end point and kinetic analyses indicated a subset (e.g., 5 of 12 candidate enzymes) that displayed some activity at 60° C. Of the five with some activity, three were classified as high activity (RS50, RS56, and RS9), and the remaining two were classified as lower activity (RS28 and RS29) (FIG. 8).

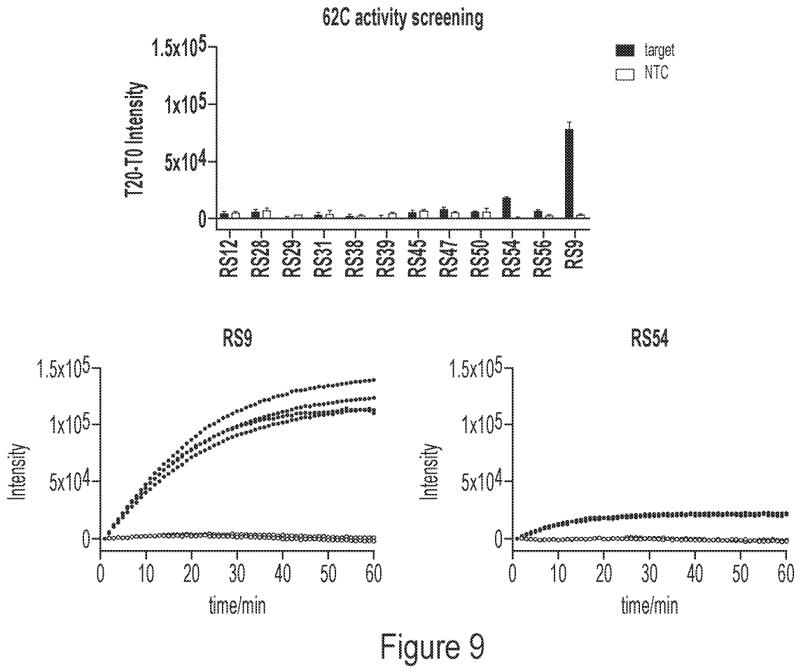

Both end point and kinetic analyses indicated a subset (e.g., 2 of 12 candidate enzymes) that displayed some activity at 62° C. (RS9 and RS54) (FIG. 9).

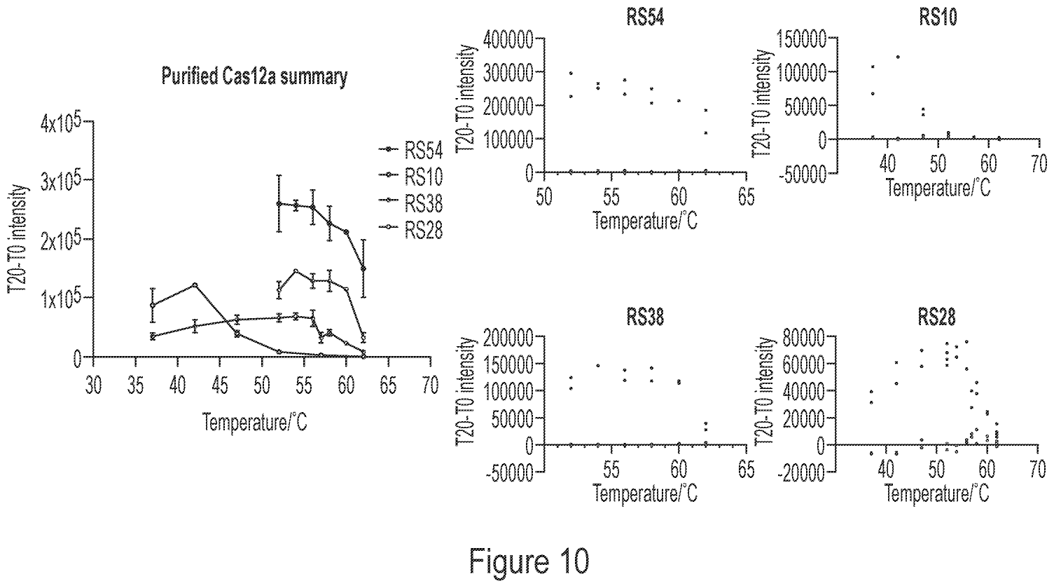

While the refined list of 12 candidate enzymes were assessed at varying temperature, four priority candidate enzymes (RS10, RS28, RS38, and RS54) were purified and assessed for activity at varying temperatures (e.g., approximately 35° C. to approximately 65° C.). RS54 showed activity at LAMP temperatures (e.g., 61° C.) (FIG. 10).

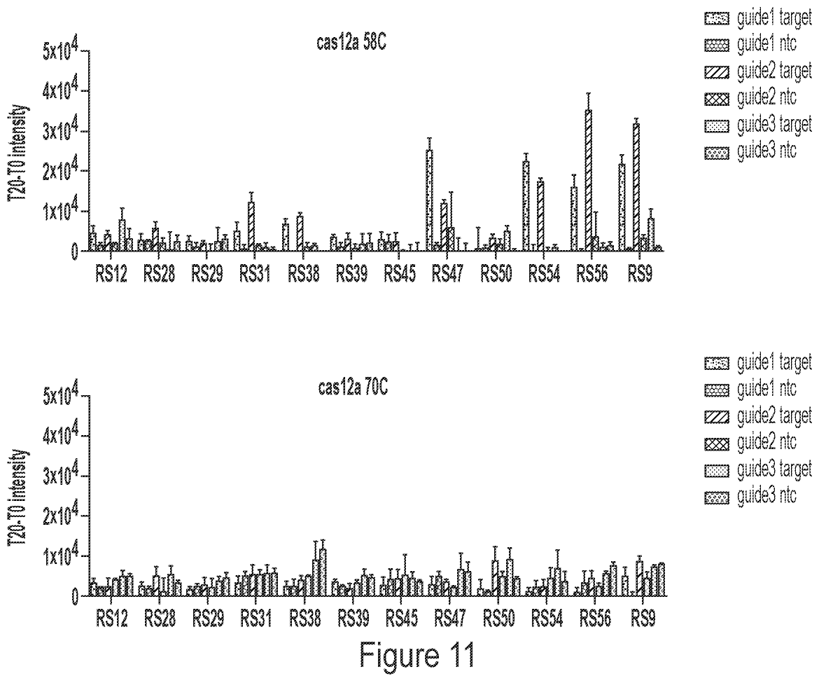

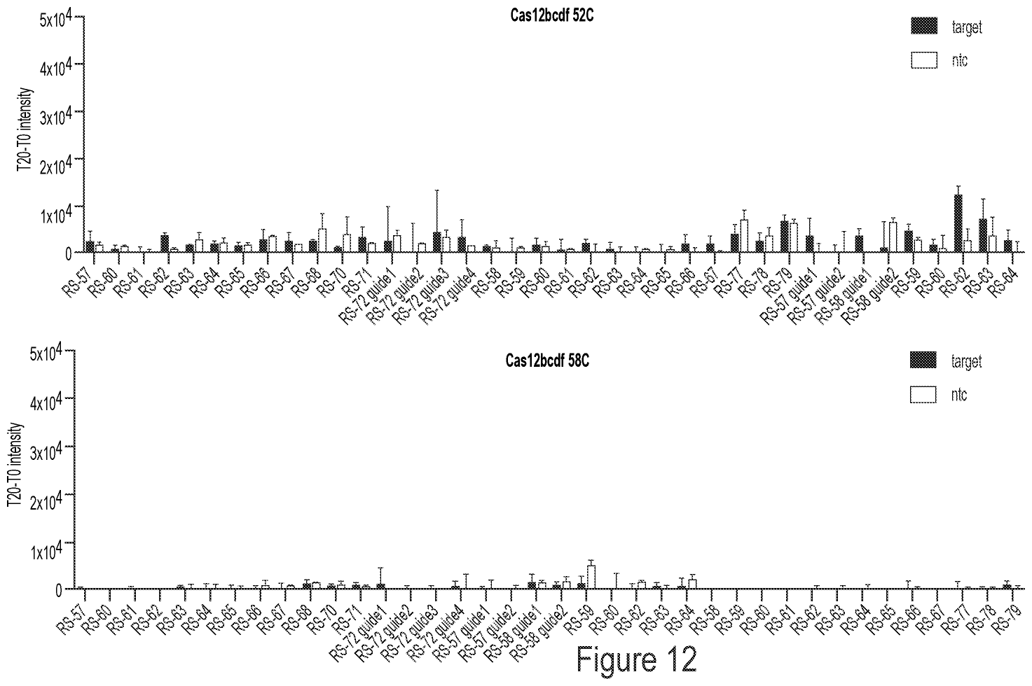

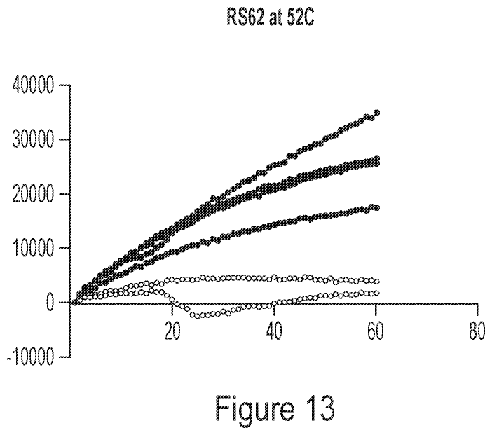

A subset of Cas12a candidates were further examined using three different guide and target sets compared to no template control at both 58° C. and 70° C. (FIG. 11). Cas12bcdf was also assessed with all guide/target pairs at both 52 and 58° C. (FIG. 12). Kinetic analysis were also performed for Cas12a candidate, RS62, which showed activity at 52° C. (FIG. 13).

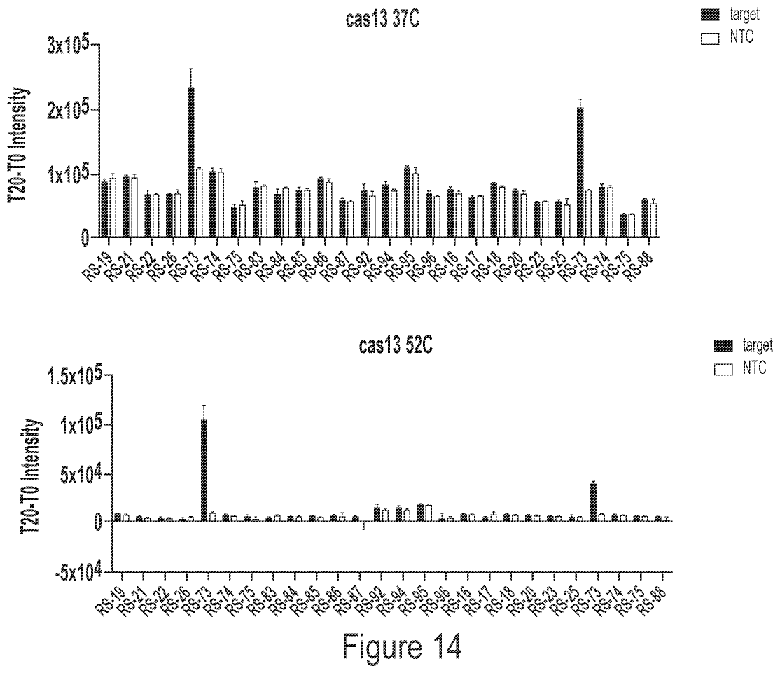

An initial pool of Cas13 candidate enzymes were assessed by endpoint analysis (FIG. 14) using no template as a negative control for activity at both 37° C. and 52° C. A single Cas13 candidate enzyme (RS73) was identified that displayed thermostability.

### Example 6: Exemplary Characterization of a Thermostable Cas12a Enzyme

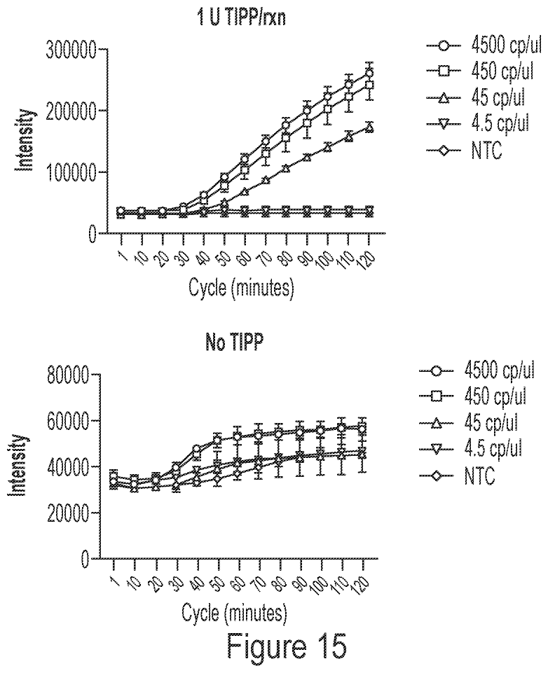

The present example demonstrates characterization of an exemplary thermostable Cas12a enzyme, RS9. To determine whether RS9 required use of Thermostable Inorganic Pyrophosphatase (TIPP) for amplification of a target nucleic acid, real time detection of ORF1ab amplification was completed over a range of starting concentrations of ORF1ab (4.5 copies/4 to 4,500 copies/4) in the presence or absence of TIPP. An exemplary reaction included 30 ng/ul RS9, 112.5 XL-213 (ORF1ab guide), 1× HKFB (ORF1ab) primer set, 1× wsLAMP mix, 125 nM DNase Alert, with or without 1 U Thermostable Inorganic Pyrophosphatase (TIPP) with indicated viral RNA template concentration present. The exemplary reaction was incubated at 58° C. for 120 minutes on QS5 with detection in VIC channel. Real-time reactions that did not contain TIPP resulted in no statistically significant amplification of ORF1ab compared to the no template control regardless of the starting concentration of template. Real-time reactions that did contain TIPP, with the exception of that with a starting concentration of template at 4.5 copies/4, demonstrated significant amplification of ORF1ab compared to the no template control, suggesting RS9 requires use of TIPP for amplification (FIG. 15).

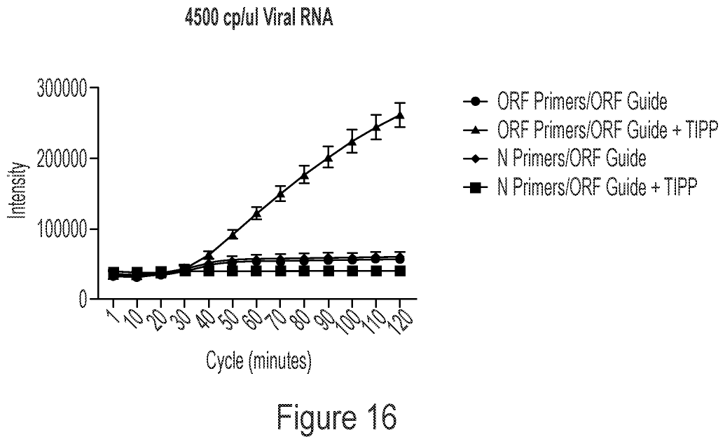

Specificity of RS9 was also assessed using real-time analysis. Amplification was conducted with a starting concentration of 4,500 copies/μL ORF1ab template and either primers and guides specific to ORF1ab or non-targeting primers with an ORF1ab guide. Each reaction condition was also conducted in the presence or absence of TIPP. An exemplary reaction included 30 ng/ul RS9, 112.5 XL-213 (ORF1ab guide), 1×HKFB (ORF1ab) or CFB (N) primer set, 1× wsLAMP mix, 125 nM DNase Alert, with or without 1 U Thermostable Inorganic Pyrophosphatase (TIPP), with 4,500 copies/μl viral RNA present. An exemplary reaction was incubated at 58° C. for 120 minutes on QS5 and detected in VIC channel. No amplification was detected for reactions containing non-targeting primers and/or no TIPP. Robust amplification was detected in reactions containing ORF1ab primers, an ORF1ab guide, and TIPP, indicating RS9 is specific for its target and requires TIPP for amplification (FIG. 16).

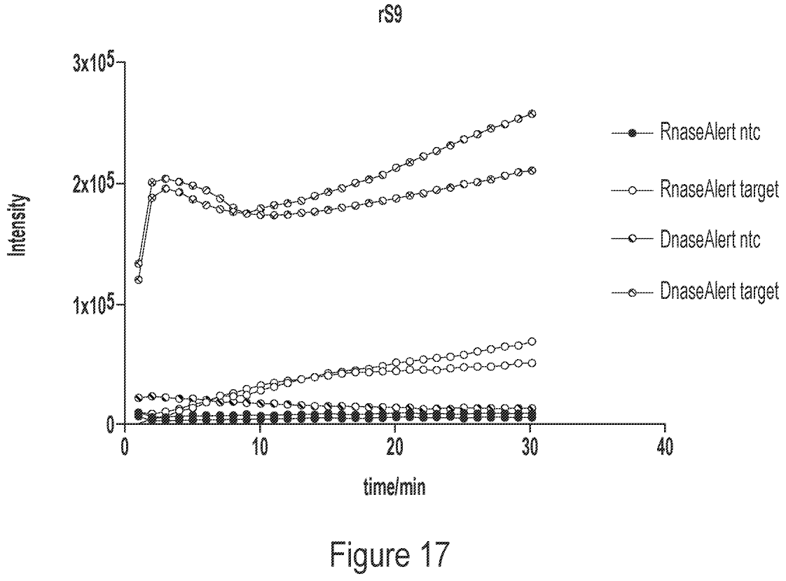

RS9 collateral cleavage activity was assessed using 100 nM of single stranded DNA target and DNaseAlert or RNaseAlert as reporters. A no target condition was utilized as a negative control. RS9 was unable to cleave DNaseAlert, resulting in intensity measurements significantly above no target control conditions. RS9 was able to cleave RNaseAlert, resulting in measured intensity similar to that of the no target control conditions, indicating RS9 has RNA-specific collateral cleavage activity (FIG. 17).

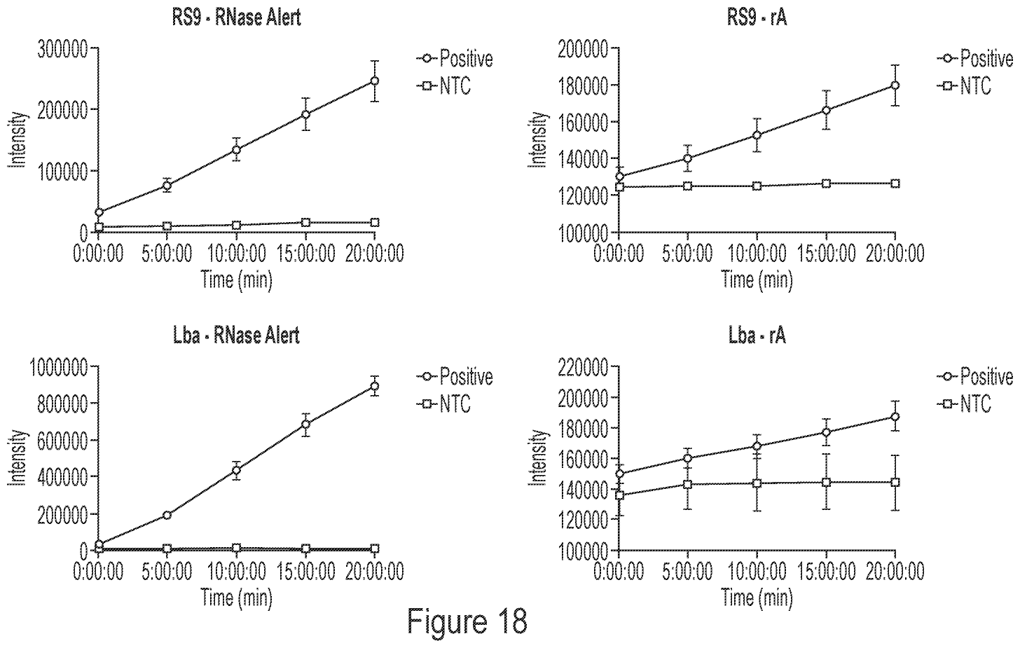

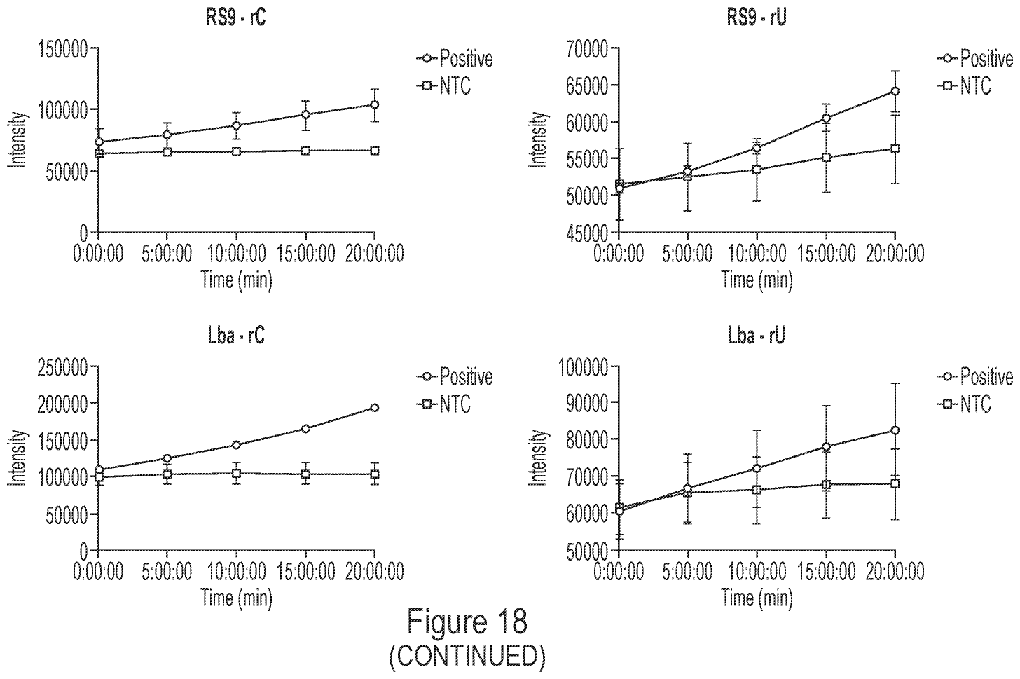

RS9 collateral cleavage activity was further evaluated alongside known Cas12a, LbaCas12a, using ORF LAMP product as target and either RNaseAlert, PolyrA, PolyrC, or PolyrU reporters. A no target condition was utilized as a negative control. Both RS9 and LbaCas12a were able to cleave RNaseAlert more efficiently than either PolyrA, PolyrC, or PolyrU (FIG. 18).

### Example 7: Exemplary Characterization of a Thermostable Cas13a

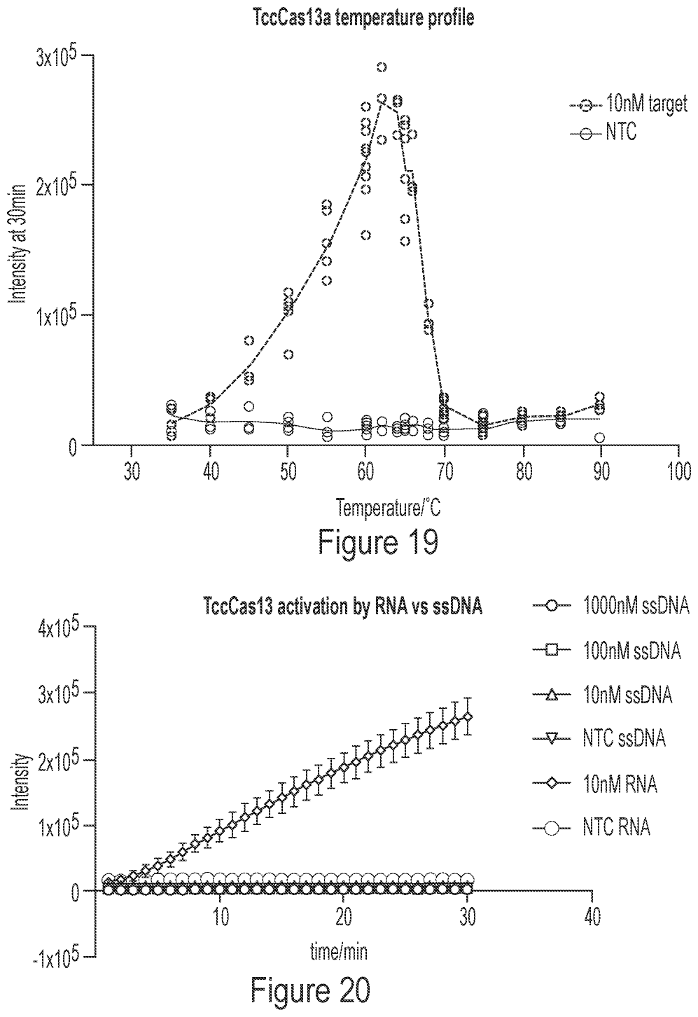

The present example demonstrates characterization of an exemplary thermostable Cas13a enzyme, TccCas13a. To determine optimal temperature for TccCas13a activity, a Cas reaction was conducted over a range of temperatures using 10 nM target and RNaseAlert as reporter. A no target condition was utilized as a negative control. Temperature profiles suggest TccCas13a shows highest activity at approximately 62° C. (FIG. 19).

To determine whether TccCas13a could be activated by ssDNA in addition to RNA, a Cas reaction was completed at 62° C. with RNaseAlert as a reporter. Different targets were utilized at different concentrations (e.g., 10 nM, 100 nM, or 1,000 nM ssDNA or 10 nM RNA). A no target condition was utilized as a negative control. Results indicated that TccCas13a can be activated by RNA, but cannot be activated by ssDNA even at the highest concentrations of ssDNA template at 62° C. (FIG. 20).

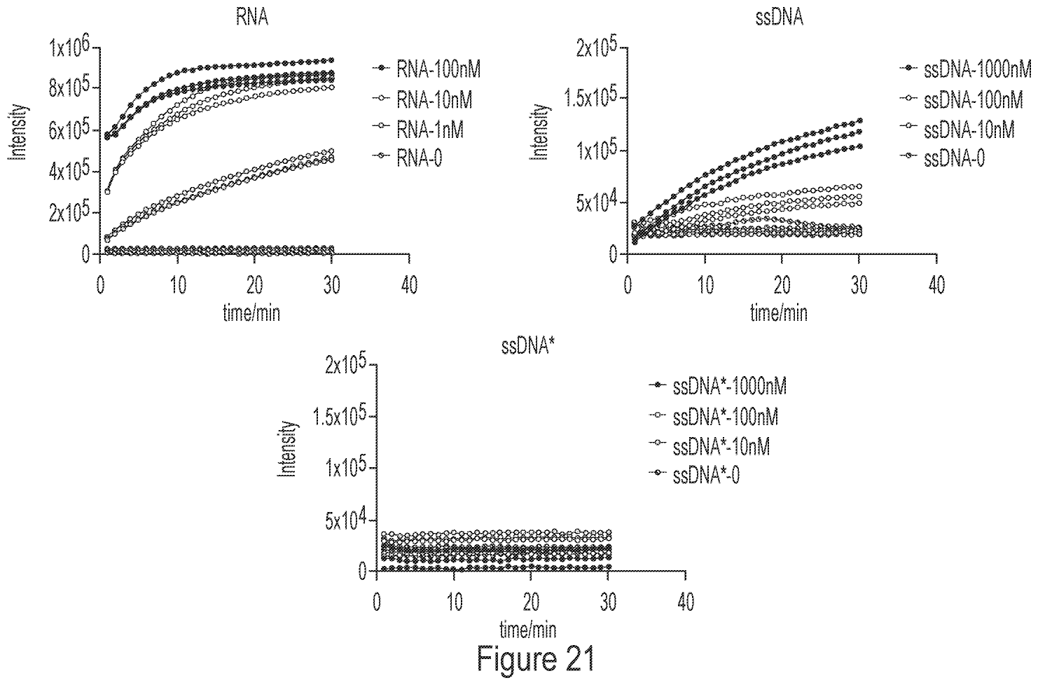

TccCas13a activation by ssDNA was also assessed at 58° C. TccCas13a was activated at 58° C. in the presence of 1 nM, 10 nM, and 100 nM RNA target compared to no target control, while TccCas13a activation by ssDNA at 58° C. was only detected when 100 nM or 1,000 nM of ssDNA target was utilized. 10 nM of ssDNA target at 58° C. showed no difference compared to no target control, suggesting TccCas13a activation requires a higher concentration of ssDNA than RNA, similar to that observed for LwaCas13 ssDNA activation. Interestingly, TccCas13a was not activated by ssDNA* at any concentration (10 nM, 100 nM, or 1,000 nM) compared to control (no target) (FIG. 21).

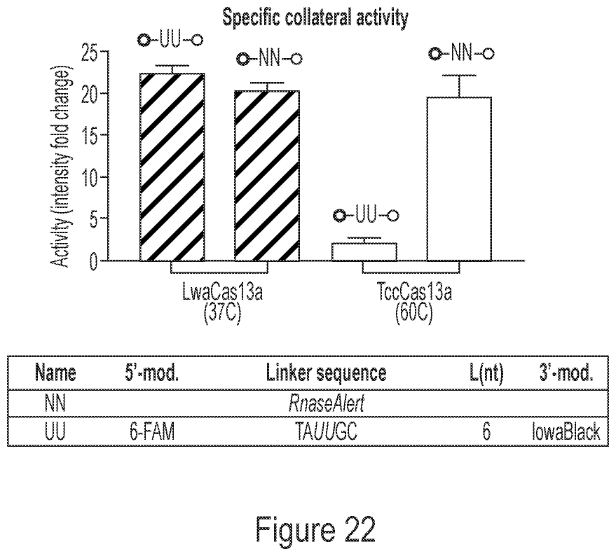

To determine whether TccCas13a showed different specific collateral activity to that of LwaCas13, a Cas reaction was done with two different reporters, RNaseAlert which contains multiple different bases (“NN”) and a “UU”-specific reporter with only two “UU” bases and a DNA backbone. Reactions were conducted at 60° C. LwaCas13a showed no preference for collateral activity of either reporter over the other, while TccCas13a showed increased collateral activity at “NN” sites compared to “UU” sites (FIG. 22).

### Example 8: Exemplary Characterization of Additional Candidate Thermostable Cas Enzymes

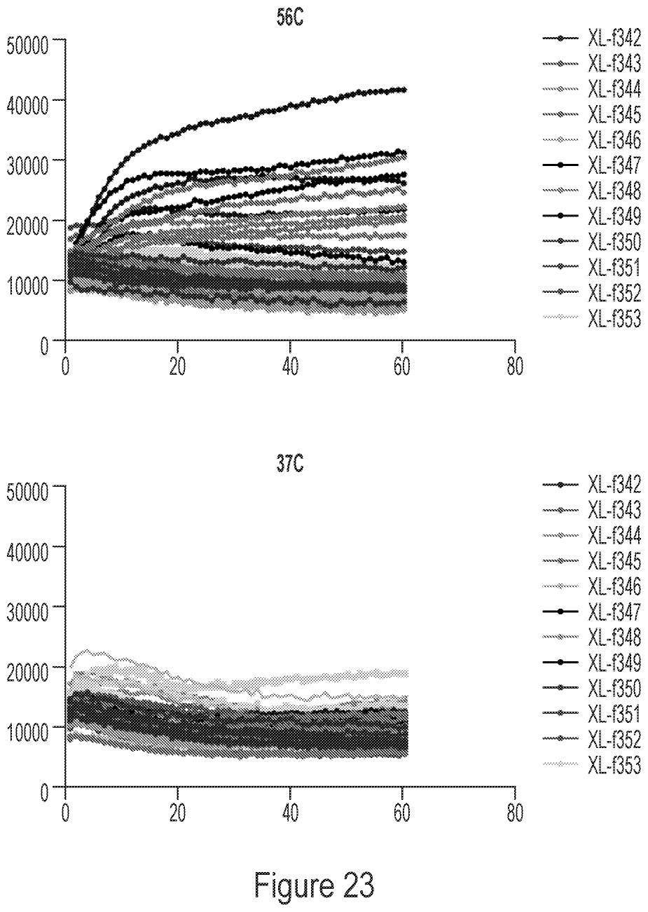

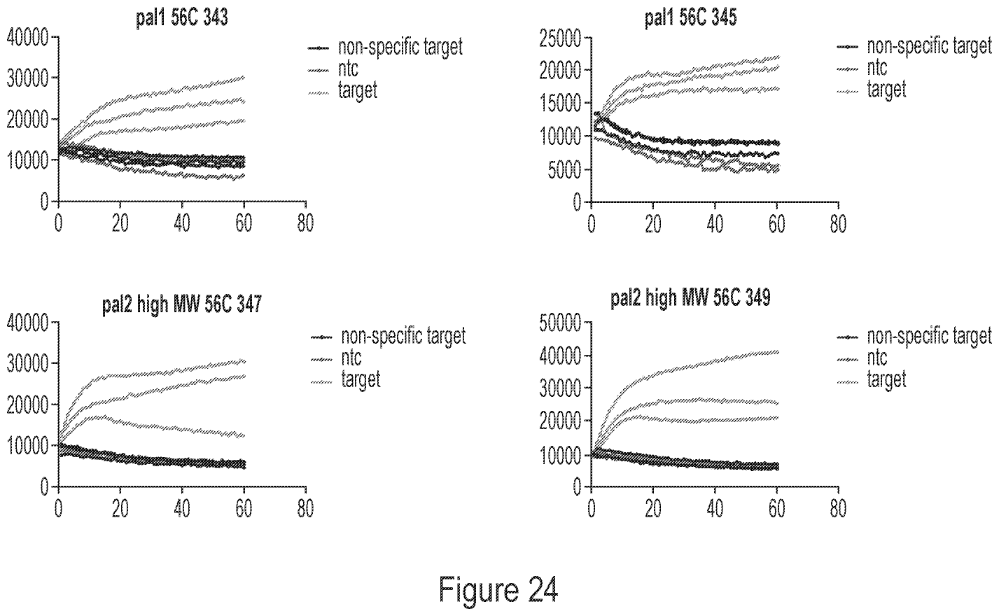

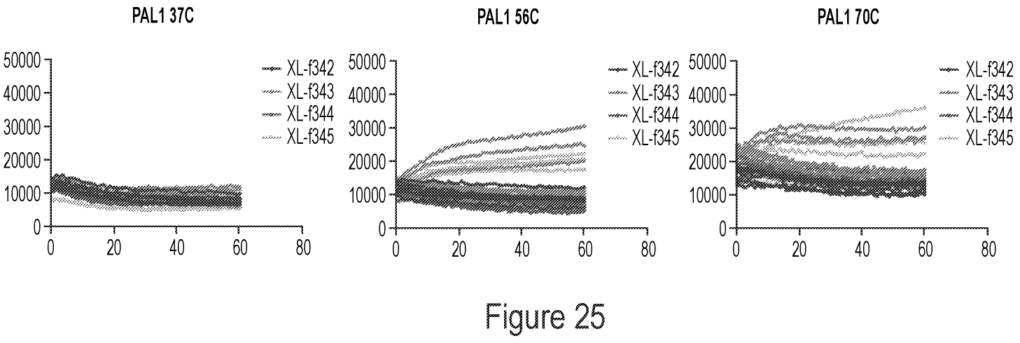

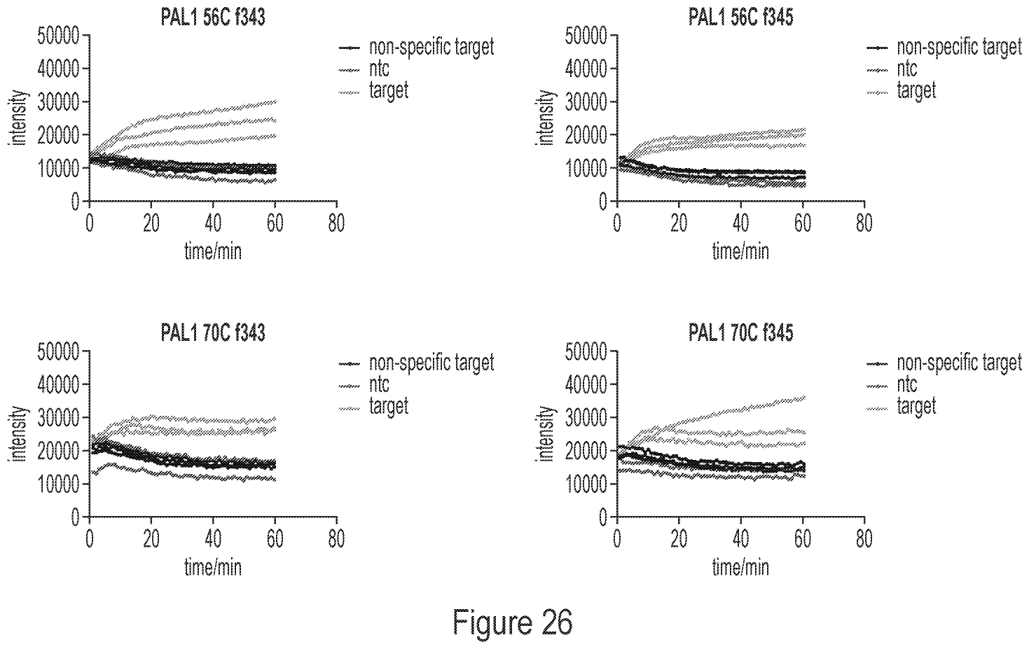

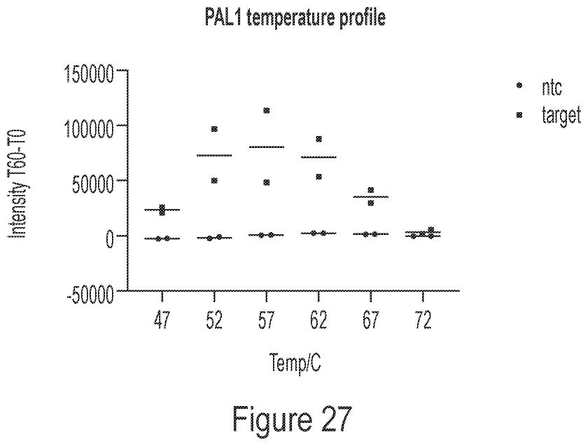

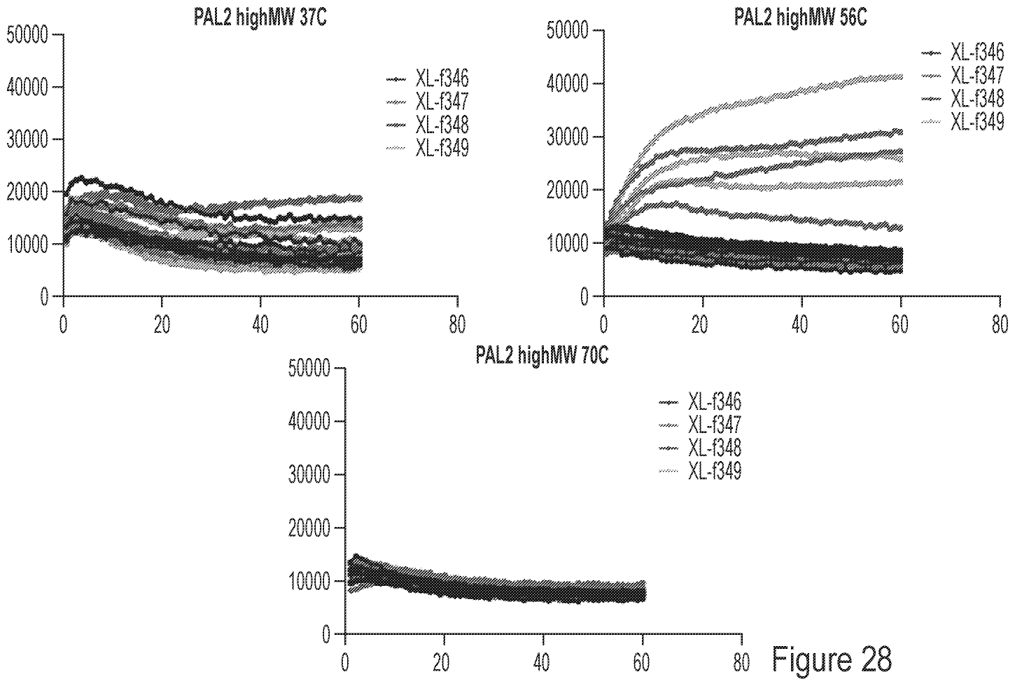

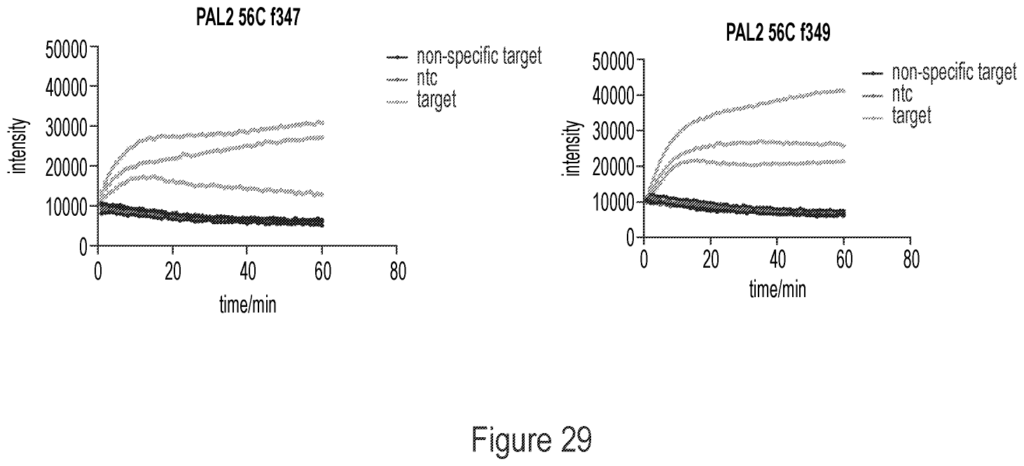

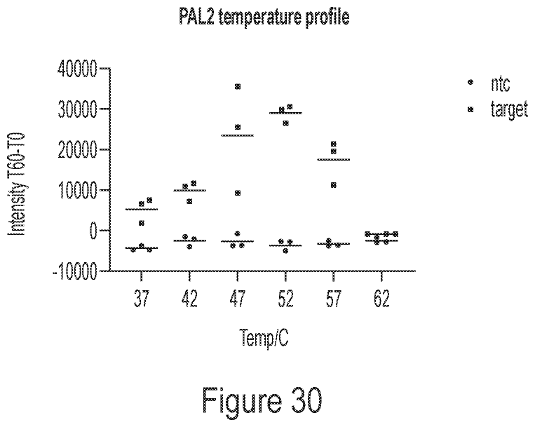

The present example demonstrates characterization of exemplary thermostable Cas enzymes, Pal1 (SEQ ID NO. 274), Pal2 low MW, Pal2 high MW (SEQ ID NO. 275), and Pal3 (SEQ ID NO. 276). Each enzyme was tested with four guides (designated as 342-353) at both 37° C. and 56° C. in a Cas-only reaction with DnaseAlert as a reporter. Fluorescence signal was plotted vs. time for each reaction (FIG. 23). Pal1 showed low activity for two guides at 56° C. No activity was observed for Pal2 low MW or Pal3, while activity was observed for two guides at 56° C. for Pal2 high MW. Pal1 and Pal2 activity at 56° C. is shown in FIG. 24. Additional results of studies with these enzymes are shown in FIGS. 25-30. As can be seen, Pal1 showed activity for 2 guides at 56° C. and 70° C.; Pal1 showed maximum activity at 57° C. and significant activity to at least 67° C. Pal2 high MW showed activity for 2 guides at 56° C.; Pal2 high MW also showed maximum activity at 47-52° C. and significant activity up to at least 57° C. No significant activity was observed for Pal2 low MW, or any of Pal3-6 at 37° C., 56° C., or 70° C. Those skilled in the art thus understand that these enzymes are thermostable at least at about 56° C. and/or within a range of 56° C. and 70° C. These particular exemplified enzymes may also be described as thermoactive as their relevant activity(ies) are dramatically reduced and/or undetected at lower temperatures such as at 37° C. Without wishing to be bound by any particular theory, it is noted that enzymes of thermophilic organisms often may show reduced (or undetectable) activity at such temperatures.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A detection method comprising steps of:
contacting a CRISPR-Cas complex comprising:
a Cas protein with collateral cleavage activity that is thermostable at temperatures above at least 60-65° C.; and
a guide RNA selected or engineered to be complementary to a target sequence; with
a sample potentially comprising a nucleic acid of the target sequence.

2. The method of claim 1, wherein the step of contacting comprises contacting the CRISRP-Cas complex and sample with a reporter susceptible to cleavage by the Cas protein collateral activity.

3. The method of claim 1, wherein the step of contacting comprises incubating for a period of time above the temperature.

4. The method of claim 1, further comprising a step of amplifying nucleic acid present in the sample.

5. The method of claim 4, wherein the step of amplifying utilizes a thermostable nucleic acid polymerase.

6. The method of claim 4, wherein the steps of amplifying and contacting are performed in a single vessel.

7. The method of claim 1, wherein the Cas protein is a Cas12 protein.

8. The method of claim 7, wherein the Cas protein has an amino acid sequence that is at least 80% identical to that of SEQ ID NO: 15.

9. The method of claim 7, wherein the Cas protein has an amino acid sequence having at least 80%, sequence identity to any one of SEQ ID Nos. 3-21, 33-47, 51-56, 68-178, and 274-283.

10. The method of claim 1, wherein the Cas protein has an amino acid sequence having 80%, sequence identity to any one of SEQ ID Nos. 1-283.

11. In a method of performing a detection assay utilizing a Cas protein with collateral cleavage activity, the improvement that comprises utilizing a Cas protein with thermostable collateral cleavage activity.

12. The improvement of claim 11, wherein the Cas protein is a Cas12 protein.

13. The improvement of claim 12, wherein the Cas protein has an amino acid sequence that is at least 80% identical to that of SEQ ID NO: 15.

14. The improvement of claim 12, wherein the Cas protein has an amino acid sequence having at least 80%, sequence identity to any one of SEQ ID Nos. 3-21, 33-47, 51-56, 68-178, and 274-283.

15. The improvement of claim 11, wherein a method of performing a detection assay is conducted in a single reaction vessel.

16. The improvement of claim 11, wherein the thermostable collateral cleavage activity is thermostable above a temperature of about 60° C.

17. The improvement of claim 11, wherein the thermostable collateral cleavage activity is thermostable above a temperature of about 65° C.

18. The improvement of claim 11, wherein the Cas protein has an amino acid sequence having at least 80% sequence identity to any one of SEQ ID Nos. 1-283.
